(19) United States
(12) Patent Application Publication
(10) Pub. No.: US 2024/0209396 A1
(43) Pub. Date: Jun. 27, 2024

(54) SMALL CAS PROTEINS AND USES THEREOF

(71) Applicant: HuiGene Therapeutics Co., Ltd., Shanghai (CN)

(72) Inventors: Hui Yang, Shanghai (CN); Yingsi Zhou, Shanghai (CN); Renxia Zhang, Shanghai (CN); Xing Wang, Shanghai (CN)

(21) Appl. No.: 18/023,761

(22) PCT Filed: Sep. 1, 2020
(86) PCT No.: PCT/CN2020/112868

§ 371 Date: Feb. 28, 2023

(51) Int. Cl. C12N 15/87; C12N 9/22; C12N 9/78; C12N 15/11

(52) U.S. Cl. CPC C12N 15/87; C12N 9/22; C12N 9/78; C12N 15/11; C12Y 305/04004; C12N 2310/20; C12N 2310/531

(57) ABSTRACT

The invention described herein provides a novel class of RNA base editors comprising a fusion protein that comprises a small Class I CRISPR/Cas effector enzyme, such as Cas5, Cas6, and Csf5 and an RNA based editing functional domain. The invention also provides polynucleotides encoding the fusion proteins, and method of using the fusion proteins for RNA base editing.

### RELATED APPLICATIONS

This application is a U.S. national stage application, filed under 35 U.S.C. § 371(c), of International Patent Application No. PCT/CN2020/112868, filed on Sep. 1, 2020. The entire contents of the aforementioned application, including all drawings and sequences, are incorporated herein by reference.

### REFERENCE TO SEQUENCE LISTING

This application includes an electronically submitted sequence listing in .txt format. The .txt file contains a sequence listing entitled “Seq_Listing_132045_00301.txt” created on Aug. 27, 2023 and is 108,622 bytes in size. The sequence listing contained in this .txt file is part of the specification and is hereby incorporated by reference herein in its entirety.

### BACKGROUND OF THE INVENTION

CRISPR (clustered regularly interspaced short palindromic repeats) is a family of DNA sequences found within the genomes of prokaryotic organisms such as bacteria and archaea. These sequences are understood to be derived from DNA fragments of bacteriophages that have previously infected the prokaryote, and are used to detect and destroy DNA from similar bacterialphages during subsequent infections of the prokaryotes.

CRISPR-associated systems is a set of homologous genes, or Cas genes, some of which encode Cas protein having helicase and nuclease activities. The Cas proteins are enzymes that utilize RNA derived from the CRISPR sequences (crRNA) as guide sequences to recognize and cleave specific strands of polynucleotide (e.g., DNA) that are complementary to the crRNA.

Together, the CRISPR-Cas system constitutes a primitive prokaryotic “immune system” that confers resistance or acquired immunity to foreign pathogenic genetic elements, such as those present within extrachromosomal DNA (e.g., plasmids) and bacterialphages, or foreign RNA encoded by foreign DNA.

In nature, the CRISPR/Cas system appears to be a widespread prokaryotic defense mechanism against foreign genetic materials, and is found in approximately 50% of sequenced bacterial genomes and nearly 90% of sequenced archaea. This prokaryotic system has since been developed to form the basis of a technology known as CRISPR-Cas that found extensive use in numerous eukaryotic organisms including human, in a wide variety of applications including basic biological research, development of biotechnology products, and disease treatment.

The prokaryotic CRISPR-Cas systems comprise an extremely diverse group of proteins effectors, non-coding elements, as well as loci architectures, some examples of which have been engineered and adapted to produce important biotechnologies.

The CRISPR locus structure has been studied in many systems. In these systems, the CRISPR array in the genomic DNA typically comprises an AT-rich leader sequence, followed by short DR sequences separated by unique spacer sequences. These CRISPR DR sequences typically range in size from 28 to 37 bps, though the range can be 23-55 bps. Some DR sequences show dyad symmetry, implying the formation of a secondary structure such as a stem-loop (“hairpin”) in the RNA, while others appear unstructured. The size of spacers in different CRISPR arrays is typically 32-38 bps (with a range of 21-72 bps). There are usually fewer than 50 units of the repeat-spacer sequence in a CRISPR array.

Small clusters of cas genes are often found next to such CRISPR repeat-spacer arrays. So far, the 93 identified cas genes have been grouped into 35 families, based on sequence similarity of their encoded proteins. Eleven of the 35 families form the so-called cas core, which includes the protein families Cas1 through Cas9. A complete CRISPR-Cas locus has at least one gene belonging to the cas core.

CRISPR-Cas systems can be broadly divided into two classes—Class 1 systems use a complex of multiple Cas proteins to degrade foreign nucleic acids, while Class 2 systems use a single large Cas protein for the same purpose. The single-subunit effector compositions of the Class 2 systems provide a simpler component set for engineering and application translation, and has thus far been important sources of discovery, engineering, and optimization of novel powerful programmable technologies for genome engineering and beyond.

Class 1 system is further divided into types I, III, and IV; and Class 2 system is divided into types II, V, and VI. These 6 system types are additionally divided into 19 subtypes. Classification is also based on the complement of cas genes that are present. Most CRISPR-Cas systems have a Cas1 protein. Many prokaryotes contain multiple CRISPR-Cas systems, suggesting that they are compatible and may share components.

One of the first and best characterized Cas proteins—Cas9—is a prototypical member of Class 2, type II, and originates from Streptococcus pyogenes (SpCas9). Cas9 is a DNA endonuclease activated by a small crRNA molecule that complements a target DNA sequence, and a separate trans-activating CRISPR RNA (tracrRNA). The crRNA consists of a direct repeat (DR) sequence responsible for protein binding to the crRNA and a spacer sequence, which may be engineered to be complementary to any desired nucleic acid target sequence. In this way, CRISPR systems can be programmed to target DNA or RNA targets by modifying the spacer sequence of the crRNA. The crRNA and tracrRNA have been fused to form a single guide RNA (sgRNA) for better practical utility. When combined with Cas9, sgRNA hybridizes with its target DNA, and guides Cas9 to cut the target DNA. Other Cas9 effector protein from other species have also been identified and used similarly, including Cas9 from the S. thermophilus CRISPR system. These CRISPR/Cas9 systems have been widely used in numerous eukaryotic organisms, including baker's yeast (Saccharomyces cerevisiae), the opportunistic pathogen Candida albicans, zebrafish (Danio rerio), fruit flies (Drosophila melanogaster), ants (Harpegnathos saltator and Ooceraea biroi), mosquitoes (Aedes aegypti), nematodes (Caenorhabditis elegans), plants, mice, monkeys, and human embryos.

ADAR (Adenosine Deaminase Action on RNA) is a class of deaminases broadly expressed in mammalian (e.g., human) tissues. They catalyze the deamination of adenosine (A) in RNA to convert them to inosine (I), which is equivalent to guanosine (G). Compared to DNA editing, RNA editing does not cause permanent genomic change. Thus such reversible, controllable editing is safer in practice.

Current existing RNA editing technologies all utilize a guide RNA to position an ADAR functional domain near a target RNA location to effect sequence- or site-specific editing. These methods, however, all suffer from one or more limitations, and cannot achieve high efficiency, precision, and convenience simultaneously. For example, in 2017, a report in Science showed that a Cas13b-ADAR fusion achieved high efficiency site-directed RNA base editing. However, that system suffered from high frequency of off-site targeting. Although a refined ver. 2 ADAR based on the same system has reduced off-target editing, it also lost significant editing efficiency as a result. Meanwhile, because of the relatively large size of the Cas13b protein, it is difficult to package the coding sequence for the Cas13b-ADAR fusion protein into vectors with limited packaging capacity, such as the widely used AAV vector with a packaging limit of about 4.7 kb, thus severely hampering in vivo delivery of such RNA base editors.

Current existing RNA base editors generally fall within three categories. The first category of base editors rely on naturally existing ADAR in the target cells. However, all target cells do not express endogenous ADAR, and even for cells that do express endogenous ADAR, expression level varies among the different target cells, thus having wide variations in editing efficiency. The second category of base editors utilize a fusion of ADAR with a nuclease-dead version of Cas13, such as the dCas13-ADAR2dd fusion. The advantage of this system is that dCas13 can bind to/process pre-crRNA, thus enabling multiplexing RNA editing to target multiple target RNAs. The disadvantage of this system is that dCas13 has the ability to bind double-stranded RNA non-specifically, thus it is prone to off-target editing. Further, the size of the dCas13-ADARdd fusion is usually above 1100 amino acids, which is difficult for packaging in most AAV vectors. The third category of base editors fuse other (non-Cas) RNA-binding proteins with ADAR functional domain. Such other RNA-binding proteins include λN peptide, SNAP tag, MS2, GluR2, TAR RNA binding protein, etc. The advantage of this system is that these non-Cas RNA-binding proteins are usually smaller in size, and their fusions with ADAR are better suited for AAV packaging. However, they suffer from targeting specificity and efficiency, and are unable to process pre-crRNA, thus are not suitable for multiplex RNA editing.

Thus, there is a need to develop further smaller, high efficiency, high fidelity RNA single-base editing system that is preferably also suitable for multiplexing editing, to provide an important improvement in the field of gene editing for use in, for example, gene therapy.

### SUMMARY OF THE INVENTION

In one aspect, the invention provides a CasPR (CRISPR-associated Protein for Class 1 pre-crRNA processing) fusion protein, comprising a CasPR (or a homolog, an ortholog, a paralog, a variant, a derivative, or a functional fragment thereof) fused to a heterologous functional domain.

In certain embodiments, the CasPR is Cas5d, Cas6, or Csf5.

In certain embodiments, the CasPR is SsoCas6 (I-A) (SEQ ID NO: 1), MmCas6 (I-B) (SEQ ID NO: 2), SpCas5d (I-C1) (SEQ ID NO: 3), BhCas5d (I-C2) (SEQ ID NO: 4), SaCas6 (I-D) (SEQ ID NO: 5), EcCas6e (I-E) (SEQ ID NO: 6), PaCas6f (I-F) (SEQ ID NO: 7), MtCas6 (III-A) (SEQ ID NO: 8), PfCas6 (III-B) (SEQ ID NO: 9), PaCsf5 (IV-A1) (SEQ ID NO: 10), or MtCsf5 (IV-A2) (SEQ ID NO: 11).

In certain embodiments, the heterologous functional domain comprises: a nuclear localization signal (NLS), a reporter protein or a detection label (e.g., GST, HRP, CAT, GFP, HcRed, DsRed, CFP, YFP, BFP), a localization signal, a protein targeting moiety, a DNA binding domain (e.g., MBP, Lex A DBD, Gal4 DBD), an epitope tag (e.g., His, myc, V5, FLAG, HA, VSV-G, Trx, etc), a transcription activation domain (e.g., VP64 or VPR), a transcription inhibition domain (e.g., KRAB moiety or SID moiety), a nuclease (e.g., FokI), a deamination domain (e.g., ADAR1, ADAR2, APOBEC, AID, or TAD), a methylase, a demethylase, a transcription release factor, an HDAC, a polypeptide having ssRNA cleavage activity, a polypeptide having dsRNA cleavage activity, a polypeptide having ssDNA cleavage activity, a polypeptide having dsDNA cleavage activity, a DNA or RNA ligase, or any combination thereof.

In certain embodiments, the heterologous functional domain comprises an RNA base editor.

In certain embodiments, the RNA base editor edits A→G single base change.

In certain embodiments, the RNA base editor edits C→U single base change.

In certain embodiments, the RNA base editor comprises ADAR2DD or a derivative thereof.

In certain embodiments, the ADAR2DD derivative comprises the E488Q/T375A double mutations.

In certain embodiments, the fusion protein has the amino acid sequence of any one of SEQ ID NOs: 45-55.

In certain embodiments, the heterologous functional domain is fused N-terminally, C-terminally, or internally in the fusion protein.

Another aspect of the invention provides a CasPR (CRISPR-associated Protein for Class 1 pre-crRNA processing) conjugate, comprising a CasPR (or a homolog, an ortholog, a paralog, a variant, a derivative, or a functional fragment thereof) conjugated to a heterologous functional domain; optionally, the CasPR is Cas5d, Cas6, or Csf5, such as SsoCas6 (I-A) (SEQ ID NO: 1), MmCas6 (I-B) (SEQ ID NO: 2), SpCas5d (I-C1) (SEQ ID NO: 3), BhCas5d (I-C2) (SEQ ID NO: 4), SaCas6 (I-D) (SEQ ID NO: 5), EcCas6e (I-E) (SEQ ID NO: 6), PaCas6f (I-F) (SEQ ID NO: 7), MtCas6 (III-A) (SEQ ID NO: 8), PfCas6 (III-B) (SEQ ID NO: 9), PaCsf5 (IV-A1) (SEQ ID NO: 10), or MtCsf5 (IV-A2) (SEQ ID NO: 11).

In certain embodiments, the heterologous functional domain comprises: a nuclear localization signal (NLS), a reporter protein or a detection label (e.g., GST, HRP, CAT, GFP, HcRed, DsRed, CFP, YFP, BFP), a localization signal, a protein targeting moiety, a DNA binding domain (e.g., MBP, Lex A DBD, Gal4 DBD), an epitope tag (e.g., His, myc, V5, FLAG, HA, VSV-G, Trx, etc), a transcription activation domain (e.g., VP64 or VPR), a transcription inhibition domain (e.g., KRAB moiety or SID moiety), a nuclease (e.g., FokI), a deamination domain (e.g., ADAR1, ADAR2, APOBEC, AID, or TAD), a methylase, a demethylase, a transcription release factor, an HDAC, a polypeptide having ssRNA cleavage activity, a polypeptide having dsRNA cleavage activity, a polypeptide having ssDNA cleavage activity, a polypeptide having dsDNA cleavage activity, a DNA or RNA ligase, or any combination thereof.

In certain embodiments, the heterologous functional domain is conjugated N-terminally, C-terminally, or internally with respect to the CasPR (or homolog, ortholog, paralog, variant, derivative, or functional fragment thereof).

Another aspect of the invention provides a protein having the amino acid sequence of: (1) any one of SEQ ID NOs: 1-11; (2) any one of SEQ ID NOs: 1-11, except for 1, 2, 3, 4, 5, 6, 7, 8, 9, or 10 amino acid substitutions, additions, or deletions, wherein the protein maintains the ability of one of SEQ ID NOs: 1-11 for binding to a direct repeat sequence of a Class 1, type I, III, or IV CRISPR system (e.g., any one of SEQ ID NOs: 12-33); or, (3) at least 80%, 85%, 90%, 91%, 92%, 93%, 94%, 95%, 96%, 97%, 98%, 99% or more sequence identity with any one of SEQ ID NOs: 1-11, wherein the protein maintains the ability of one of the CasPRs of SEQ ID NOs: 1-11 for binding to a direct repeat sequence of a Class 1, type I, III, or IV CRISPR system (e.g., any one of SEQ ID NOs: 12-33); optionally, with the proviso that the protein is not any one of SEQ ID NOs: 1-11.

Another aspect of the invention provides a CasPR complex, comprising: (1) a guide RNA comprising a guide sequence capable of hybridizing to a target RNA, and a direct repeat (DR) sequence 3′ (or 5′) to the guide sequence; and, (2) a CasPR having an amino acid sequence of any one of SEQ ID NOs: 1-11, or a homolog, an ortholog, a paralog, a variant, a derivative, or a functional fragment thereof, wherein the CasPR (or homolog, ortholog, paralog, variant, derivative, or functional fragment thereof) is fused or conjugated to a heterologous functional domain; wherein the CasPR (or homolog, ortholog, paralog, variant, derivative, or functional fragment thereof) is capable of (i) binding to the guide sequence and (ii) targeting the target RNA.

In certain embodiments, the DR sequence has substantially the same secondary structure as the secondary structure of any one of SEQ ID NOs: 12-33.

In certain embodiments, the DR sequence is encoded by any one of SEQ ID NOs: 12-33, or a functional portion thereof that binds to a cognate wild-type CasPR.

In certain embodiments, the target RNA is encoded by a eukaryotic DNA.

In certain embodiments, the eukaryotic DNA is a non-human mammalian DNA, a non-human primate DNA, a human DNA, a plant DNA, an insect DNA, a bird DNA, a reptile DNA, a rodent DNA, a fish DNA, a worm/nematode DNA, a yeast DNA.

In certain embodiments, the target RNA is an mRNA.

In certain embodiments, the guide sequence is between 15-120 nucleotides, between 20-100 nucleotides, between 25-80 nucleotides, between 15-55 nucleotides, between 25-35 nucleotides, or about 30 nucleotides.

In certain embodiments, the guide sequence is 90-100% complementary to the target RNA.

In certain embodiments, the guide RNA results from processing of a pre-crRNA transcript by the CasPR, and wherein the pre-crRNA comprises two or more guide RNAs having different guide sequences for different target RNAs.

In certain embodiments, the variant or derivative comprises conserved amino acid substitutions of one or more residues of any one of SEQ ID NOs: 1-11; optionally, the variant or derivative comprises only conserved amino acid substitutions.

In certain embodiments, the derivative is capable of binding to the guide sequence hybridized to the target RNA, but has no RNase catalytic activity due to a mutation in the RNase catalytic site of the CasPR.

In certain embodiments, the derivative has an N-terminal deletion of no more than 5, 10, 15, 20, 25, 30, 35, 40, 45, or 50 residues, and/or a C-terminal deletion of no more than 5, 10, 15, 20, 25, 30, 35, 40, 45, or 50 residues.

In certain embodiments, the CasPR is a Cas5d, a Cas6, or a Csf5, such as SsoCas6 (I-A), MmCas6 (I-B), SpCas5d (I-C1), BhCas5d (I-C2), SaCas6 (I-D), EcCas6e (I-E), PaCas6f (I-F), MtCas6 (III-A), PfCas6 (III-B), PaCsf5 (IV-A1), or MtCsf5 (IV-A2).

In certain embodiments, the heterologous functional domain comprises an RNA base-editing domain.

In certain embodiments, the RNA base-editing domain comprises an adenosine deaminase and/or a cytidine deaminase, such as a cytidine deaminase acting on RNA (CDAR), such as a double-stranded RNA-specific adenosine deaminase (ADAR) (e.g., ADAR1 or ADAR2), apolipoprotein B mRNA editing enzyme, catalytic polypeptide-like (APOBEC, such as APOBEC1, APOBEC2, APOBEC3A, APOBEC3B, APOBEC3C, APOBEC3D, APOBEC3E, APOBEC3F, APOBEC3G, APOBEC3H, and APOBEC4), activation-induced cytidine deaminase (AID), a cytidine deaminase 1 (CDA1), or a mutant thereof.

In certain embodiments, the ADAR comprises E488Q/T375G double mutation or comprises ADAR2DD.

In certain embodiments, the base-editing domain is further fused to an RNA-binding domain, such as MS2.

In certain embodiments, the derivative further comprises an RNA methyltransferase, a RNA demethylase, an RNA splicing modifier, a localization factor, or a translation modification factor.

In certain embodiments, the CasPR, the homolog, the ortholog, the paralog, the variant, the derivative, or the functional fragment comprises a nuclear localization signal (NLS) sequence or a nuclear export signal (NES).

In certain embodiments, targeting of the target RNA results in a modification of the target RNA.

In certain embodiments, the modification of the target RNA is a cleavage of the target RNA.

In certain embodiments, the modification of the target RNA is deamination of an adenosine (A) to an inosine (I), and/or deamination of a cytidine (C) to a uracil (U).

In certain embodiments, the CasPR complex further comprises a target RNA comprising a sequence capable of hybridizing to the guide sequence.

Another aspect of the invention provides a codon-optimized polynucleotide encoding a wild-type CasPR (e.g., Cas5d, Cas6, or Csf5), a homolog thereof, an ortholog thereof, a paralog thereof, a variant or derivative thereof, or a functional fragment thereof, wherein the polynucleotide is codon-optimized for mammalian (e.g., human) expression, optionally, the wild-type CasPR has the amino acid sequence of any one of SEQ ID NOs: 1-11.

In certain embodiments, the codon-optimized polynucleotide has the amino acid sequence of any one of SEQ ID NOs: 34-44.

In certain embodiments, the codon-optimized polynucleotide further comprises sequence encoding a heterologous functional domain.

In certain embodiments, the heterologous functional domain comprises an RNA base editor.

Another aspect of the invention provides a non-naturally occurring polynucleotide encoding a CasPR of any one of SEQ ID NOs: 1-11, or a homolog thereof, an ortholog thereof, a paralog thereof, a variant thereof, a derivative thereof, or a functional fragment thereof, or a fusion protein thereof.

In certain embodiments, the polynucleotide is codon-optimized for expression in a cell.

In certain embodiments, the cell is a eukaryotic cell.

Another aspect of the invention provides a non-naturally occurring polynucleotide comprising a derivative of any one of SEQ ID NOs: 12-33, wherein said derivative (i) has one or more (e.g., 1, 2, 3, 4, 5, 6, 7, 8, 9 or 10) nucleotides additions, deletions, or substitutions compared to any one of SEQ ID NOs: 12-33; (ii) has at least 20%, 30%, 40%, 50%, 60%, 70%, 80%, 90%, 95%, or 97% sequence identity to any one of SEQ ID NOs: 12-33; (iii) hybridize under stringent conditions with any one of SEQ ID NOs: 12-33 or any of (i) and (ii); or (iv) is a complement of any of (i)-(iii), provided that the derivative is not any one of SEQ ID NOs: 12-33, and that the derivative encodes an RNA (or is an RNA) that has maintained substantially the same secondary structure (e.g., stems, loops, bulges, single-stranded regions) as any of the RNA encoded by SEQ ID NOs: 12-33.

In certain embodiments, the derivative functions as a DR sequence for any one of the CasPR, the ortholog thereof, the paralog thereof, the variant thereof, the derivative thereof, or the functional fragment thereof, of the invention.

Another aspect of the invention provides a vector comprising the polynucleotide of the invention.

In certain embodiments, the polynucleotide is operably linked to a promoter and optionally an enhancer.

In certain embodiments, the promoter is a constitutive promoter, an inducible promoter, a ubiquitous promoter, or a tissue specific promoter.

In certain embodiments, the vector is a plasmid.

In certain embodiments, the vector is a retroviral vector, a phage vector, an adenoviral vector, a herpes simplex viral (HSV) vector, an AAV vector, or a lentiviral vector.

In certain embodiments, the AAV vector is a recombinant AAV vector of the serotype AAV1, AAV2, AAV4, AAV5, AAV6, AAV7, AAVrh74, AAV8, AAV9, AAV10, AAV 11, AAV 12, or AAV 13.

Another aspect of the invention provides a delivery system comprising (1) a delivery vehicle, and (2) the CasPR complex of the invention, the fusion protein of the invention, the conjugate of the invention, the protein of the invention, the polynucleotide of the invention, or the vector of the invention.

In certain embodiments, the delivery vehicle is a nanoparticle, a liposome, an exosome, a microvesicle, or a gene-gun.

Another aspect of the invention provides a cell or a progeny thereof, comprising the fusion protein of the invention, the conjugate of the invention, the protein of the invention, the CasPR complex of the invention, the polynucleotide of the invention, or the vector of the invention.

In certain embodiments, the cell or progeny thereof is a eukaryotic cell (e.g., a non-human mammalian cell, a human cell, or a plant cell) or a prokaryotic cell (e.g., a bacteria cell).

Another aspect of the invention provides a non-human multicellular eukaryote comprising the cell of the invention.

In certain embodiments, the non-human multicellular eukaryote of the invention is an animal (e.g., rodent or primate) model for a human genetic disorder.

Another aspect of the invention provides a method of modifying a target RNA, the method comprising contacting the target RNA with the CasPR complex of the invention, wherein the guide sequence is complementary to at least 15 nucleotides of the target RNA; wherein the CasPR, the ortholog, the paralog, the variant, the derivative, or the functional fragment associates with the guide sequence to form the complex; wherein the complex binds to the target RNA; and wherein upon binding of the complex to the target RNA, the CasPR, the ortholog, the paralog, the variant, the derivative, or the functional fragment modifies the target RNA.

In certain embodiments, the target RNA is modified by cleavage by the CasPR complex.

In certain embodiments, the target RNA is modified by deamination by a derivative comprising a double-stranded RNA-specific adenosine and/or cytidine deaminase.

In certain embodiments, the target RNA is an mRNA, a tRNA, an rRNA, a non-coding RNA, an lncRNA, or a nuclear RNA.

In certain embodiments, the target RNA is within a cell.

In certain embodiments, the cell is a cancer cell.

In certain embodiments, the cell is infected with an infectious agent.

In certain embodiments, the infectious agent is a virus, a prion, a protozoan, a fungus, or a parasite.

In certain embodiments, the CasPR complex is encoded by a first polynucleotide encoding any one of SEQ ID NOs: 1-11, or an ortholog, a paralog, a variant, a derivative, or a functional fragment thereof, and a second polynucleotide comprising any one of SEQ ID NOs: 12-33 and a sequence encoding a guide sequence capable of binding to the target RNA, wherein the first and the second polynucleotides are introduced into the cell; optionally, said second polynucleotide comprises a sequence encoding more than one guide sequences, each capable of targeting a different target RNA or different regions of the same target RNA.

In certain embodiments, the first and the second polynucleotides are introduced into the cell by the same vector, or by different vectors.

In certain embodiments, the method of the invention causes one or more of: (i) in vitro or in vivo induction of cellular senescence; (ii) in vitro or in vivo cell cycle arrest; (iii) in vitro or in vivo cell growth inhibition and/or cell growth inhibition; (iv) in vitro or in vitro induction of anergy; (v) in vitro or in vitro induction of apoptosis; and (vi) in vitro or in vitro induction of necrosis.

Another aspect of the invention provides a method of treating a condition or disease in a subject in need thereof, the method comprising administering to the subject a composition comprising the CasPR complex of the invention or a polynucleotide encoding the same; wherein the guide sequence is complementary to at least 15 nucleotides of a target RNA associated with the condition or disease; wherein the CasPR, the ortholog, the paralog, the variant, the derivative, or the functional fragment associates with the guide sequence to form the complex; wherein the complex binds to the target RNA; and wherein upon binding of the complex to the target RNA, the CasPR, the ortholog, the paralog, the variant, the derivative or the functional fragment cleaves the target RNA, thereby treating the condition or disease in the subject.

In certain embodiments, the condition or disease is a cancer or an infectious disease.

In certain embodiments, the cancer is Wilms' tumor, Ewing sarcoma, a neuroendocrine tumor, a glioblastoma, a neuroblastoma, a melanoma, skin cancer, breast cancer, colon cancer, rectal cancer, prostate cancer, liver cancer, renal cancer, pancreatic cancer, lung cancer, biliary cancer, cervical cancer, endometrial cancer, esophageal cancer, gastric cancer, head and neck cancer, medullary thyroid carcinoma, ovarian cancer, glioma, lymphoma, leukemia, myeloma, acute lymphoblastic leukemia, acute myelogenous leukemia, chronic lymphocytic leukemia, chronic myelogenous leukemia, Hodgkin's lymphoma, non-Hodgkin's lymphoma, or urinary bladder cancer.

In certain embodiments, the method is an in vitro method, an in vivo method, or an ex vivo method.

Another aspect of the invention provides a cell or a progeny thereof, obtained by the method of the invention, wherein the cell and the progeny comprises a non-naturally existing modification (e.g., a non-naturally existing modification in a transcribed RNA of the cell/progeny).

Another aspect of the invention provides a method to detect the presence of a target RNA, the method comprising contacting the target RNA with a composition comprising a fusion protein of the invention, or a conjugate of the invention, or a polynucleotide encoding the fusion protein, wherein the fusion protein or the conjugate comprises a detectable label (e.g., one that can be detected by fluorescence, Northern blot, or FISH) and a complexed spacer sequence capable of binding to the target RNA.

Another aspect of the invention provides q eukaryotic cell comprising a CasPR complex, said CasPR complex comprising: (1) a guide RNA comprising a guide sequence capable of hybridizing to a target RNA, and a direct repeat (DR) sequence 3′ (or 5′) to the guide sequence; and, (2) a CasPR having an amino acid sequence of any one of SEQ ID NOs: 1-11, or a homolog, an ortholog, a paralog, a variant, a derivative, or a functional fragment of said CasPR; wherein the CasPR, the homolog, the ortholog, the paralog, the variant, the derivative, and the functional fragment of said CasPR, are capable of (i) binding to the DR sequence of the guide RNA and (ii) targeting the target RNA.

It should be understood that any one embodiment of the invention described herein, including those described only in the examples or claims, can be combined with one or more other embodiments of the invention unless explicitly disclaimed or otherwise improper.

### BRIEF DESCRIPTION OF THE DRAWINGS

FIG. 1 shows the relationship among the different effector enzymes in the Class 1 CRISPR systems, including types I, III, and IV effector enzymes.

FIG. 2 shows known mechanism of actions for Class 1 types I and III proteins.

FIG. 3 shows a phylogenetic tree that illustrates the evolutional relationship among the various CasPR proteins for the types and subtypes within the Class 1 CRISPR/Cas systems.

FIG. 4 is a schematic drawing showing the structure of the reporter and base-editing plasmids for testing the base editing activity of the CasPR-ADAR2DD* type base editors.

FIG. 5 shows the summary of based editing activities of the various CasPR-ADAR2DD* based editors at three testing sites.

FIG. 6 shows the results of optimizing the mismatch base location within the crRNA for the RNA-based base editors.

FIG. 7 is a schematic drawing showing the reporter and exon skipping plasmid constructs for measuring the activity of crRNA-guided PaCsf5 to suppress pre-mRNA splicing at exon 51 of a DMD minigene.

FIG. 8 shows the results of crRNA-guided PaCsf5-mediated suppression of pre-mRNA splicing at exon 51 of a DMD minigene.

### DETAILED DESCRIPTION OF THE INVENTION

### 1. Overview

The present invention is partly based on the discovery that certain Class 1 Cas proteins from various prokaryotes, such as Cas5d, Cas6, and Csf5, can bind to their respective direct repeat (DR) sequences, such as elements of the DR hairpin RNA structures. When the DR hairpin RNA structure is linked to a spacer or guide RNA specifically designed to bind to a target polynucleotide (e.g., target RNA, such as target mRNA), the Class 1 Cas proteins bound to the DR hairpin RNA structure is brought to the proximity of the target RNA recognized by the guide RNA. If the Class 1 Cas protein is fused to a functional domain, such as an RNA base editor (e.g., amino deaminase), a mismatch designed into the guide RNA sequence can be used to modify the target RNA to achieve site-specific base editing. Since these Class 1 Cas proteins can process pre-crRNA into crRNA (or guide RNA), the system can be used for multiplexing targeting/base editing of multiple target sequences, each complementary to one guide sequence in the mature crRNA processed by the Class 1 Cas protein of the invention.

Likewise, if the Class 1 Cas protein of the invention is fused to other functional domains, such as methylase or demethylase, such functional domains can be brought to the proximity of the (multiple) target RNA(s) recognized by the guide RNA(s)/crRNA(s) and act on any substrate(s) in a sequence specific manner.

Thus the invention disclosed herein include systems and uses for such Class 1 Cas proteins (“CasPR” herein), including but not limited to base editing therapeutics and diagnostics. Fusion proteins comprising nucleotide deaminase and such CasPR, homologs, variants, derivatives, functional fragment thereof, and endonuclease-dead version thereof, including those disclosed herein (collectively referred to as “CasPR” for simplicity unless indicated otherwise), may be useful for base editing. Delivery of the proteins and systems disclosed herein is also provided, including to a variety of cells and via a variety of particles, vesicles and vectors.

For example, in one aspect, the invention provides methods for using one or more elements of a CasPR-based RNA-targeting system. The CasPR RNA-targeting complex of the invention provides an effective means for modifying a target RNA single or double stranded, linear or super-coiled. The CasPR RNA-targeting complex of the invention has a wide variety of utility including modifying (e.g., deleting, inserting, translocating, inactivating, activating) a target RNA in a multiplicity of cell types. As such the CasPR RNA-targeting complex of the invention has a broad spectrum of applications in, e.g., gene therapy, drug screening, disease diagnosis, and prognosis. An exemplary CasPR RNA-targeting complex comprises a CasPR RNA-targeting effector protein complexed with a guide RNA or crRNA hybridized to a target sequence within the target locus of interest.

It should be understood that any one embodiment of the invention described herein, including those described only in one aspect of the invention or only in the examples or claims can be combined with any other embodiments, unless expressly disclaimed or improper.

### 2. CasPR

CRISPR clusters contain space sequences (or “spacers”) located between direct repeat (DR) sequences. The natural spacers in the CRISPR loci of bacteria are sequences complementary to antecedent mobile elements and target invading nucleic acids. CRISPR clusters are initially transcribed into long primary transcripts called pre-CRISPR RNAs (pre-crRNAs), which are subsequently processed into CRISPR RNAs (crRNAs) by sequence-specific CRISPR-associated (Cas) endonucleases that cleave the initial long primary transcripts (pre-crRNAs), usually at the base of the direct repeat hairpin RNA structures, into smaller, mature crRNAs. Such sequence-specific endonucleases are collectively referred to herein as CasPRs (Cas pre-crRNA processing/maturation endonucleases).

Most multi-subunit Class 1 systems process crRNAs with a CRISPR associated endonucleases called Cas6, which share conserved structural motifs that bind crRNAs. In general, Cas6 use a metal-ion-independent mechanism to cleave crRNAs on the 3′-side of stem-loops formed within the palindromic CRISPR repeat sequence. Cleavage is generally catalyzed by stabilizing nucleophilic attack from the 2′-OH group located upstream from the scissile phosphate. Although different Cas6 enzymes from different species tend to be diverse in sequence, this cleavage mechanism appears to be conserved, despite some structural and mechanistic differences. Often, a His residue is used to catalyze cleavage, though other residues, such as Lys, have been shown to catalyze the reaction when histidine is not present (e.g., in subtype I-A). In subtypes I-B, I-E, I-D and I-F, Cas6 makes structural and base specific interactions with the stable stem-loop formed by the palindromic CRISPR repeat and typically stays bound even after cleavage to form a component of the multi-subunit interference complex. In contrast, the repeats of subtypes I-A, III-A, and III-B are less stable, allowing Cas6 to dissociate from the processed crRNA and to perform multi-turnover crRNA cleavage.

Type IV CRISPR systems are also categorized as Class 1 as they are predicted to form multi-subunit crRNA-guided complexes. Distinct Type IV-A systems contain diverse cas6 gene sequences, including genes designated as cas6e and cas6f (cas6 sequences observed in subtypes I-E and I-F, also generally referred hereto as Cas6), and a Type IV-specific Cas6-like Csf5. The presence of Cas6 homologs suggests that Type IV-A systems process crRNAs through a Cas6-mediated mechanism. Indeed, although various mechanisms exist, Cas6-mediated metal-independent processing of crRNA is a conserved process across diverse Class 1 systems, including in Type IV systems. Type IV crRNA is cleaved on the 3′ side of the predicted stem-loop structure, with nucleophilic attack on the scissile phosphate coming from the 2′ hydroxyl of base G22 of the repeat.

The following sections describe three prototypical CasPR that can be used in the methods and systems of the invention, though other related CasPRs, particularly those related to SEQ ID NOs: 1-11, are within the scope of the invention.

Cas5d The Cas5d Cas processing enzyme (CasPR) is a Class 1 type I-C CasPR that processes pre-crRNA in crRNA. It has about 250 residues, including a conserved 43-residue N-terminal region. When processing pre-crRNA, Cas5d initiates an intramolecular attack of the 2′-hydroxyl group of G26 (the 3-′end base of the predicted hairpin stem) on the scissile phosphodiester, cutting the precursor 3′ to G26 residue, yielding 5′-hydroxyl and 2′ and/or 3′ ends lacking a hydroxyl group (perhaps a 2′/3′ cyclic phosphodiester). It is believed to require between 4 and 8 nt downstream of the cleavage site for both binding and cleavage of the pre-crRNA. Substitution with dG at this G26 position abolishes cleavage but not RNA binding.

The high-resolution X-ray structure of Cas5d from Mannheimia succiniciproducens has been published (see Garside et al., RNA 18(11):2020-2028, 2012). The M. succiniciproducens Cas5d shares strong sequence similarity with the Cas5d family of Dvulg-type Cas proteins, and a Cas5d ortholog from Thermus thermophilus is also an RNA endonuclease that specifically binds and cleaves pre-crRNA. Comparison of Cas5d by structural alignment with the Class 1 type I crRNA CasPR Cse3 suggested that there is a conserved mechanism of RNA recognition among diverse CRISPR RNA processing enzymes. In addition, primary sequence alignments revealed that the T. thermophilus Cas5d is ˜40% identical and ˜65% similar to that of M. succiniciproducens Cas5d, indicating the known structure of the M. succiniciproducens Cas5d forms an excellent basis for homology modeling of the structure of the other Cas5d with at least about 25%, or about 35-40% sequence identity, and/or at least about 60% sequence similarity.

BLASTp search in the NCBI nr database using the SpCas5d (I-C1) protein sequence (SEQ ID NO: 3) retrieved, in addition to the Streptococcus pyogenes query sequence, at least 100 homologous sequences sharing at least 80% sequence identity over the entire length of the query sequence, all within the Streptococcus genus, and most with more than 90% sequence identity.

Similarly, BLASTp search in the NCBI nr database using the BhCas5d (I-C2) protein sequence (SEQ ID NO: 4) retrieved, in addition to the Bacillus halodurans C-125 query sequence, at least 100 homologous sequences sharing at least 69% sequence identity over the entire length of the query sequence.

Thus one aspect of the invention provide a wild-type Class 1 type I-C or Cas5d type CasPR protein (e.g., homologs, orthologs, paralogs) that shares at least about 65%, 69%, 70%, 75%, 80%, 85%, 90%, 91%, 92%, 93%, 94%, 95%, 96%, 97%, 98%, 99% or more sequence identity to SEQ ID NOs: 3 or 4, such as those that are currently available in the NCBI nr database and can be readily retrieved using SEQ ID NO: 3 or 4 as protein query sequence.

The terms “homologue” and “homolog” are used interchangeably herein and are well known in the art. A “homologue” as used herein also includes a protein of the same species which performs the same or a similar function as the protein it is a homologue of. Homologous proteins may but need not be structurally related, or are only partially structurally related. Homolog also encompasses “orthologue”/“ortholog” and “paralogue”/“paralog,” which arise from speciation event and multiplication event, respectively. That is, an “orthologue” of a protein is a protein of a different species which performs the same or a similar function as the protein it is an orthologue of, a “paralogue” of a protein is a protein of the same species that originates from gene multiplication and which performs the same or a similar function as the protein it is a paralog of. Orthologous/paralogous proteins may but need not be structurally related, or are only partially structurally related. In particular embodiments, the homologue or orthologue or paralogue of a CasPR protein as referred to herein (e.g., Cas5d, Cas6, or Csf5) has a sequence homology or identity of at least 40%, at least 50%, at least 60%, at least 70%, at least 80%, at least 85%, at least 90%, such as for instance at least 95% with a CasPR effector protein herein.

In a related aspect, the invention provides a Class 1 type I-C or Cas5d type variant/derivative CasPR protein, including a functional fragment thereof (e.g., at least the N-terminal 120, 130, 140, 150, 160, 170, 180, 190, 200, 210 or 220 residues), that shares at least about 65%, 69%, 70%, 75%, 80%, 85%, 90%, 91%, 92%, 93%, 94%, 95%, 96%, 97%, 98%, 99% or more (e.g., 100%) sequence identity to any one of the wild-type Cas5d CasPR described above. In certain embodiments, the functional fragment thereof retains the ability to bind to the DR sequence bound by the respective wild-type Cas5d sequences. In certain embodiments, the functional fragments comprise up to 99%, 95%, 90%, 85%, 80%, 75%, 70%, 65%, 60%, 55% or 50% of the respective wild-type Cas5d sequences.

As used herein, a “variant” of a protein has qualities or characteristics that have a pattern that deviates from what occurs in nature. A “derivative” derives from a protein and may have similar function, different function, a partial function of the protein from which it derives from.

In a related aspect, the invention provides a Class 1 type I-C or Cas5d type variant/derivative CasPR protein that contains up to 1, 2, 3, 4, 5, 6, 7, 8, 9, or 10 amino acid substitutions (e.g., conserved substitutions), additions, or deletions compared to any one of the wild-type Cas5d CasPR described above. When there is more than 1 substitutions (e.g., conserved substitutions), additions, or deletions, the substitutions (e.g., conserved substitutions), additions, or deletions can be on consecutive or non-consecutive residues.

In certain embodiments, the variant/derivative thereof at least preserves the RNA-binding ability of the wild-type Class 1 type I-C or Cas5d protein from which the variant/derivative is derived, such as the ability to bind to a cognate DR sequence in crRNA. The Class 1 type I-C or Cas5d type variant/derivative thereof does not include any naturally existing or wild-type Cas5d from which the variant/derivative is derived.

In certain embodiments, the variant/derivative thereof further preserves the ability of the wild-type Class 1 type I-C or Cas5d from which the variant/derivative is derived, to process pre-crRNA to mature crRNA, e.g., the endonuclease activity.

In certain embodiments, the variant/derivative thereof retains the ability to bind, but not the ability to cleave (e.g., the endonuclease activity) pre-crRNA to mature crRNA, compared to the wild-type Class 1 type I-C or Cas5d from which the variant/derivative is derived. Cas5d structure reveals a ferredoxin domain-based architecture and a catalytic triad formed by Y46, K116, and H117 residues. See Nam et al., Structure 20:1574-84, 2012. Thus Cas5d (from Bacillus halodurans) mutant lacking endonuclease activity (or “dCas5d”) can be produced by mutating any one or more of the three residues in the catalytic triad. Other dCas5d from different species can be produced based on catalytic triad mutations corresponding to that in Bacillus halodurans.

For example, the catalytic residues of BhCAs5d and SpCas5d are Y46/K116/H117 and Y48/K118/H119, respectively. Thus a dCas5d protein based on these CasPR can be: dead BhCas5d (Y46A, K116A and/or H117A), and dead SpCas5d (Y48A, K118A and/or H119A). In certain embodiments, one, two, or three residues of the catalytic triad residues is/are mutated to create the “dead” nucleases, and the mutations can be, but are not limited to Ala, so long as the side chain of the mutated residue is substantially different from the original Y, K or H residue(s).

The endonuclease activity or lack thereof can be tested using any art recognized method, such as the gel mobility shift assay as described in Garside et al., RNA 18(11):2020-2028, 2012 (incorporated herein by reference).

The DR sequences for the Cas5d of SEQ ID NOs: 3 and 4 are SEQ ID NOs: 14 and 15. The DR sequences of the other Class 1 type I-C or Cas5d endonucleases can be obtained from the respective CRISPR locus from which the Cas5d sequences originate.

In certain embodiments, the Cas5d CasPR, the variant or derivative thereof (including dCas5d mutant), or the functional fragment thereof binds to not just the full length or the natural DR hairpin RNA structure of the CRISPR locus to which they belong, but also binds to a truncated version of the DR hairpin RNA structure. In certain embodiments, the truncated version comprises the stem of the natural DR hairpin RNA structure, and optionally at least 4-8 nts (e.g., 4, 5, 6, 7, or 8 nts) of single-stranded sequence 3′ to the stem.

The truncated DR with the single-stranded sequence can be processed by Cas5d, and is thus useful for multiplexing targeting when the pre-crRNA processing activity of Cas5d is used to process and release individual crRNAs in the pre-crRNA transcript. When the processing function of Cas5d is not needed, however, the truncated DR can comprise only the hairpin region sequence but not the single-stranded sequence yet still preserving the ability for Cas5d binding. In a related aspect, the invention provides a polynucleotide encoding any one of the Class 1 type I-C or Cas5d CasPR proteins herein, including wild-type, derivative/variant (including dCas5d mutant), or functional fragment thereof.

In another related aspect, the invention provides reverse complement sequence of the above polynucleotides encoding any one of the Class 1 type I-C or Cas5d CasPR proteins herein, including wild-type, derivative/variant thereof (including dCas5d mutant), and functional fragment thereof.

In certain embodiments, the polynucleotide is not a naturally occurring polynucleotide that encodes a wild-type Class 1 type I-C or Cas5d CasPR protein herein.

In certain embodiments, the polynucleotide is codon-optimized, such as codon-optimized for eukaryotic or mammalian expression, e.g., human expression. It will be appreciated that, while codon-optimization for human is routinely available, codon optimization for a host of other species other than human, or for codon optimization for specific organs is known. In some embodiments, an enzyme coding sequence encoding a CasPR is codon optimized for expression in particular cells, such as eukaryotic cells. The eukaryotic cells may be those of or derived from a particular organism, such as a mammal, including but not limited to human, or non-human eukaryote or animal or mammal as herein discussed, e.g., mouse, rat, rabbit, dog, livestock, or non-human mammal or primate.

In general, codon optimization refers to a process of modifying a nucleic acid sequence for enhanced expression in the host cells of interest by replacing at least one codon (e.g. about or more than about 1, 2, 3, 4, 5, 10, 15, 20, 25, 50, or more codons) of the native sequence with codons that are more frequently or most frequently used in the genes of that host cell while maintaining the native amino acid sequence. Various species exhibit particular bias for certain codons of a particular amino acid. Codon bias (differences in codon usage between organisms) often correlates with the efficiency of translation of messenger RNA (mRNA), which is in turn believed to be dependent on, among other things, the properties of the codons being translated and the availability of particular transfer RNA (tRNA) molecules. The predominance of selected tRNAs in a cell is generally a reflection of the codons used most frequently in peptide synthesis. Accordingly, genes can be tailored for optimal gene expression in a given organism based on codon optimization. Codon usage tables are readily available, for example, at the “Codon Usage Database” available at www.kazusa.orjp/codon/ and these tables can be adapted in a number of ways. See Nakamura et al., “Codon usage tabulated from the international DNA sequence databases: status for the year 2000” Nucl. Acids Res. 28:292 (2000). Computer algorithms for codon optimizing a particular sequence for expression in a particular host cell are also available, such as Gene Forge (Aptagen; Jacobus, PA), are also available. In some embodiments, one or more codons (e.g. 1, 2, 3, 4, 5, 10, 15, 20, 25, 50, or more, or all codons) in a sequence encoding a CasPR correspond to the most frequently used codon for a particular amino acid.

In certain embodiments, the polynucleotide has the polynucleotide sequence of SEQ ID NO: 36 or 37.

### Cas6

Cas6 is one of the six highly conserved or core Cas proteins, and is among the most widely distributed Cas proteins found in numerous archaea and bacteria. It is an endoribonuclease that cleaves the primary transcripts of the CRISPR pre-crRNAs, within each of the direct repeat sequences, in a sequence-specific manner to release individual crRNAs encoded by the CRISPR locus. Cas6 interacts with a specific sequence motif in the 5′ region of the CRISPR repeat element (e.g., 20-30 nucleotides from the 5′ end of the DR sequence) and cleaves at a defined site within the 3′ region of the repeat (which is about 20-25 nucleotides form the 5′ end of the DR sequence). The Cas6 cleavage products then undergo further processing to generate smaller mature psiRNA species.

The 1.8 angstrom crystal structure of the Pyrococcus furiosus Cas6 reveals two ferredoxin-like folds that are found in other RNA-binding proteins. The predicted active site of the enzyme is similar to that of tRNA splicing endonucleases. Like the functionally similar Cse3 (CRISPR-Cas system) protein of E. coli, Cas6 is a member of the RAMP (repeat-associated mysterious protein) superfamily proteins which contain G-rich loops and are predicted to be RNA-binding proteins. Cas6 is distinguished from the many other RAMP family members by a conserved sequence motif within the predicted C-terminal G-rich loop (consensus GhGxxxxxGhG, where h is hydrophobic and xxxxx has at least one lysine or arginine).

Mutation of 2 nt spanning the cleavage site drastically reduced the cleavage activity of PfCas6 without preventing binding of Cas6 to the DR RNA based on RNA gel mobility shift assay. The Cas6 cleavage site is at a junction within a potential stem-loop structure that may form by base-pairing between weakly palindromic sequences commonly found at the 5′ and 3′ termini of CRISPR DR sequences.

The RNA sequence requirements of Cas6 binding and endonucleolytic cleavage have been elucidated. RNA gel mobility shift assay showed that sequences in the 5′ region of the CRISPR DR sequence, especially the 5′ most 12 nt, most importantly the first 8 nt, are important for PfCas6 binding. Meanwhile, cleavage by Cas6 appears to involve additional elements, because there are mutations that dramatically reduce cleavage efficiency without disrupting PfCas6 binding. Specifically, substitution of 2 nt at the cleavage site disrupts cleavage but not binding. Substitution of the last 8 nt of the DR, small (4-nt) insertions or deletions, or substitution of 6 nt between the PfCas6-binding site and cleavage site, specifically disrupted cleavage. No cleavage activity was observed with a DNA repeat sequence. These results suggest that cleavage depends upon sequence elements along the length of the repeat and perhaps upon the distance between the binding and cleavage sites, and are consistent with a requirement for a specific RNA fold such as the predicted hairpin structure.

BLASTp search in the NCBI nr database using the SsoCas6 (I-A) protein sequence (SEQ ID NO: 1) retrieved about 16 of the top 100 homologous sequences each sharing at least 80% sequence identity over the entire length of the query sequence, most over 95% identity.

BLASTp search in the NCBI nr database using the MmCas6 protein sequence (SEQ ID NO: 2) retrieved, in addition to the Methanococcus maripaludis query sequence, 3 other homologous sequences sharing at least 63-70% sequence identity over the entire length of the query sequence.

BLASTp search in the NCBI nr database using the SaCas6 protein sequence (SEQ ID NO: 5) retrieved, in addition to the Synechococcus a. query sequence, another homologous sequences sharing at least 70% sequence identity over the entire length of the query sequence.

BLASTp search in the NCBI nr database using the EcCas6e protein sequence (SEQ ID NO: 6) retrieved, in addition to the E. coli query sequence, 99 other homologous sequences sharing at least 97% sequence identity over the entire length of the query sequence.

BLASTp search in the NCBI nr database using the PaCas6f protein sequence (SEQ ID NO: 7) retrieved, in addition to the Pseudomonas aeruginosa query sequence, about 60 other homologous sequences sharing at least 97% sequence identity over the entire length of the query sequence.

BLASTp search in the NCBI nr database using the MtCas6 protein sequence (SEQ ID NO: 8) retrieved, in addition to the Mycobacterium tuberculosis query sequence, about 35 homologous sequences sharing at least 99% sequence identity over the entire length of the query sequence, and another 50 or so sharing at least 99% sequence identity over at least 50-85% of the query sequence.

BLASTp search in the NCBI nr database using the PfCas6 protein sequence (SEQ ID NO: 9) retrieved, in addition to the Pyrococcus furiosus query sequence, about 4 sequences sharing at least 99% sequence identity over the entire length of the query sequence, and another 50 or so sharing at least 60-70% sequence identity over at least 90% of the query sequence.

Thus one aspect of the invention provide a wild-type Class 1 type I or Cas6 type CasPR protein (e.g., homologs, orthologs, paralogs) that shares at least about 65%, 69%, 70%, 75%, 80%, 85%, 90%, 91%, 92%, 93%, 94%, 95%, 96%, 97%, 98%, 99% or more sequence identity to SEQ ID NOs: 1, 2, 5, 6, 7, 8, or 9, such as those that are currently available in the NCBI nr database and can be readily retrieved using SEQ ID NO: 1, 2, 5, 6, 7, 8, or 9 as protein query sequence.

In a related aspect, the invention provides a Class 1 type I or Cas6 type variant/derivative CasPR protein, including a functional fragment thereof (e.g., at least the N-terminal 100, 110, 120, 130, 140, 150, 160, 170, 180, 190, or 200 residues), that shares at least about 65%, 69%, 70%, 75%, 80%, 85%, 90%, 91%, 92%, 93%, 94%, 95%, 96%, 97%, 98%, 99% or more sequence identity to any one of the wild-type Cas6 CasPR described above. In certain embodiments, the functional fragment thereof retains the ability to bind to the DR sequence bound by the respective wild-type Cas6 sequences. In certain embodiments, the functional fragments comprise up to 99%, 95%, 90%, 85%, 80%, 75%, 70%, 65%, 60%, 55% or 50% of the respective wild-type Cas6 sequences.

In a related aspect, the invention provides a Class 1 type I or Cas6 type variant/derivative CasPR protein that contains up to 1, 2, 3, 4, 5, 6, 7, 8, 9, or 10 amino acid substitutions (e.g., conserved substitutions), additions, or deletions compared to any one of the wild-type Cas6 CasPR described above. When there is more than 1 substitutions (e.g., conserved substitutions), additions, or deletions, the substitutions (e.g., conserved substitutions), additions, or deletions can be on consecutive or non-consecutive residues.

In certain embodiments, the variant/derivative thereof at least preserves the RNA-binding ability of the wild-type Class 1 type I or Cas6 protein from which the variant/derivative is derived, such as the ability to bind to a cognate DR sequence in crRNA. The Class 1 type I or Cas6 type variant/derivative thereof does not include any naturally existing or wild-type Cas6 from which the variant/derivative is derived.

In certain embodiments, the variant/derivative thereof further preserves the ability of the wild-type Class 1 type I or Cas6 from which the variant/derivative is derived, to process pre-crRNA to mature crRNA, e.g., the endonuclease activity.

In certain embodiments, the variant/derivative thereof retains the ability to bind, but not the ability to cleave (e.g., the endonuclease activity) pre-crRNA to mature crRNA, compared to the wild-type Class 1 type I or Cas6 from which the variant/derivative is derived.

Three conserved residues (Y31, H46, and K52) that are separated from one another in the primary sequences of Cas6 proteins from diverse organisms were found to cluster in the crystal structure of Cas6 from P. furiosus (Carte et al., Genes Dev 22:3489-3496, 2008), which structure was also found to be similar to the configuration of archaeal tRNA splicing endonuclease. Substitution of any of the three triad amino acids with Alanine led to a significant decrease in cleavage activity relative to wild-type Cas6. No cleavage activity was observed with the Y31A and H46A Cas6 mutants. The cleavage activity was reduced ˜40-fold at the highest tested concentration (500 nM) of K52A Cas6 mutant relative to wild-type Cas6. Meanwhile, based on gel mobility shift assay, Tyr31, His46, and Lys52 were found to be not required for binding to CRISPR repeat RNA (Carte et al., RNA 16(11):2181-2188, 2010). Thus these three conserved amino acids comprise a catalytic triad required for Cas6 cleavage of the CRISPR crRNA. Cas6 mutants lacking cleavage activity from P. furiosus and other species can be readily produced based on mutating the corresponding residues of Y31, H46, and K52 in P. furiosus.

The catalytic residues of four Cas6 include at least: MtCas6: Y29, K51; MmCas6: Y34, K56; EcCas6e: H18; and PaCas6f: Y31, H36, K52. Thus a dCas6 protein based on these CasPR can be: dead MtCas6 (Y29A and/or K51A); dead MmCas6 (Y34A and/or K56A); dead EcCas6e: H18A; and dead PaCas6f: Y31A, H36A, and/or K52A. In certain embodiments, one, two, or three residues of the catalytic residues is/are mutated to create the “dead” nucleases, and the mutations can be, but are not limited to Ala, so long as the side chain of the mutated residue is substantially different from the original (e.g., Y, K or H) residue(s).

The endonuclease activity or lack thereof can be tested using any art recognized method, such as the gel mobility shift assay as described in Carte et al., RNA 16(11):2181-2188, 2010 (incorporated herein by reference).

The DR sequences for the Cas6 of SEQ ID NOs: 1, 2, 5, 6, 7, 8, and 9 are SEQ ID NOs: 12, 13, 16, 17, 18, 19, or 20, respectively. The DR sequences of the other Class 1 type I or Cas6 endonucleases can be obtained from the respective CRISPR locus from which the Cas6 sequences originate.

In certain embodiments, the Cas6 CasPR, the variant or derivative thereof (including dCas5d mutant), or the functional fragment thereof binds to not just the full length or the natural DR hairpin RNA structure of the CRISPR locus to which they belong, but also binds to a truncated version of the DR hairpin RNA structure. In certain embodiments, the truncated version comprises the most 5′ 8-12 nt (e.g., 8, 9, 10, 11, or 12 nts) of the cognate DR sequence for the respective Cas6, such as the most 5′ 22-25 nts of the cognate DR sequence for the respective Cas6.

In a related aspect, the invention provides a polynucleotide encoding any one of the Class 1 type I or Cas6 CasPR proteins herein, including wild-type, derivative/variant (including dCas5d mutant), or functional fragment thereof.

In another related aspect, the invention provides reverse complement sequence of the above polynucleotides encoding any one of the Class 1 type I or Cas6 CasPR proteins herein, including wild-type, derivative/variant thereof (including dCas5d mutant), and functional fragment thereof.

In certain embodiments, the polynucleotide is not a naturally occurring polynucleotide that encodes a wild-type Class 1 type I or Cas6 CasPR protein herein.

In certain embodiments, the polynucleotide is codon-optimized for mammalian expression.

In certain embodiments, the polynucleotide has the polynucleotide sequence of SEQ ID NO: 34, 35, 38, 39, 40, 41, or 42.

### Csf5

Csf5 is also known as the CRISPR-Cas type IV Cas6 crRNA endonuclease (see Ozcan et al., Nat Microbiol. 4(1):89-96, 2019). It processes CRISPR pre-crRNA into mature crRNAs that are specifically incorporated into type IV CRISPR-ribonucleoprotein (crRNP) complexes. Structures of RNA-bound Csf5 have been obtained and studied.

At least in M. australiensis Type IV CRISPR system (Ma Cas6-IV), the stem of the DR hairpin RNA structure may be recognized primarily through shape rather than base-specific interactions, because base switches at the base of the DR hairpin RNA stem would not disrupt base pairing and are acceptable for Ma Cas6-IV binding if both Watson Crick and G-U wobble base pairs are preserved. Other base switches in the arms and loop of the hairpin likewise suggest that those positions are recognized through shape, or are not necessary at all for binding.

Structural comparisons between the Ma Cas6-IV and Csf5 from Aromatoleum aromaticum (PDB 6H9I) reveal that they both contain the dual RRM domain scaffold generally observed in Cas6 proteins. The C-terminal RRM domains of both enzymes contain the motifs that bind crRNA (groove-binding element or GBE, β-hairpin, and G-loop), but the C-terminal domain of Csf5 differs from Ma Cas6-IV in that the second alpha helix (α2) of the canonical RRM fold is absent. In both Csf5 and Ma Cas6-IV, the α1 helices of the N-terminal RRM domains have been replaced with helix-turn-helix motifs that house putative active-site residues. However, instead of the small loop sequence observed in Ma Cas6-IV that connects the helix-loop-helix to β2, Csf5 has an insertion of ˜40 amino acids called the α-helical finger domain (α-HFD) that contains two additional helices. One of these helices interacts with the minor groove of the crRNA stem-loop, providing additional contacts for binding the crRNA that may provide additional specificity toward Type IV crRNA repeats.

BLASTp search in the NCBI nr database using the PaCsf5 protein sequence (SEQ ID NO: 10) retrieved, in addition to the Pseudomonas aeruginosa query sequence, about 6 homologous sequences sharing at least 80% sequence identity over the entire length of the query sequence, and 4 of which over 98% identical.

Thus one aspect of the invention provide a wild-type Class 1 type IV or Csf5 type CasPR protein (e.g., homologs, orthologs, paralogs) that shares at least about 65%, 69%, 70%, 75%, 80%, 85%, 90%, 91%, 92%, 93%, 94%, 95%, 96%, 97%, 98%, 99% or more sequence identity to SEQ ID NO: 10 or 11, such as those that are currently available in the NCBI nr database and can be readily retrieved using SEQ ID NO: 10 or 11 as protein query sequence.

In a related aspect, the invention provides a Class 1 type IV or Csf5 type variant/derivative CasPR protein, including a functional fragment thereof (e.g., at least the N-terminal 120, 130, 140, 150, 160, 170, 180, 190, 200, 210 or 220 residues), that shares at least about 65%, 69%, 70%, 75%, 80%, 85%, 90%, 91%, 92%, 93%, 94%, 95%, 96%, 97%, 98%, 99% or more sequence identity to any one of the wild-type Class 1 type IV or Csf5 CasPR described above. In certain embodiments, the functional fragment thereof retains the ability to bind to the DR sequence bound by the respective wild-type Csf5 sequences. In certain embodiments, the functional fragments comprise up to 99%, 95%, 90%, 85%, 80%, 75%, 70%, 65%, 60%, 55% or 50% of the respective wild-type Csf5 sequences.

In a related aspect, the invention provides a Class 1 type IV or Csf5 type variant/derivative CasPR protein that contains up to 1, 2, 3, 4, 5, 6, 7, 8, 9, or 10 amino acid substitutions (e.g., conserved substitutions), additions, or deletions compared to any one of the wild-type Class 1 type IV or Csf5 CasPR described above. When there is more than 1 substitutions (e.g., conserved substitutions), additions, or deletions, the substitutions (e.g., conserved substitutions), additions, or deletions can be on consecutive or non-consecutive residues.

In certain embodiments, the variant/derivative thereof at least preserves the RNA-binding ability of the wild-type Class 1 type IV or Csf5 protein from which the variant/derivative is derived, such as the ability to bind to a cognate DR sequence in crRNA. The Class 1 type IV or Csf5 type variant/derivative thereof does not include any naturally existing or wild-type Class 1 type IV or Csf5 from which the variant/derivative is derived.

In certain embodiments, the variant/derivative thereof further preserves the ability of the wild-type Class 1 type IV or Csf5 from which the variant/derivative is derived, to process pre-crRNA to mature crRNA, e.g., the endonuclease activity.

In certain embodiments, the variant/derivative thereof retains the ability to bind, but not the ability to cleave (e.g., the endonuclease activity) pre-crRNA to mature crRNA, compared to the wild-type Class 1 type IV or Csf5 from which the variant/derivative is derived.

Both Csf5 and Ma Cas6-IV contain a histidine in the N-terminal RRM at the same sequence position (H44), but the Csf5 H44 is within the 40 amino acid insert α-HFD and is several Ångstroms away from the scissile phosphate, and does not participate in nuclease activity. Rather, mutation of arginine residues located on the Csf5 helix-turn-helix and the G-loop (R23A, R38A, R242A) impaired cleavage. Notably several of these arginines are located in similar positions to the active site residues of Ma Cas6-IV (His44 and Tyr31), supporting the notion that these Type IV Cas proteins rely on similar structural themes to bind and cleave crRNA substrates despite their diverse sequences. See Taylor et al., RNA Biol. 16(10):1438-1447, 2019. Thus Csf5 mutant lacking endonuclease activity (or “dCsf5”) can be produced by mutating any one or more of the three residues corresponding to the catalytic triad (R23, R38, and R242) of Csf5 from Aromatoleum aromaticum (PDB 6H9I), including other dCsf5 from different species.

The endonuclease activity or lack thereof can be tested using any art recognized method, such as the gel mobility shift assay as described in Garside et al., RNA 18(11):2020-2028, 2012 (incorporated herein by reference).

The DR sequences for the Csf5 of SEQ ID NOs: 10 and 11 are SEQ ID NOs: 21 and 22, respectively. The DR sequences of the other Class 1 type IV or Csf5 endonucleases can be obtained from the respective CRISPR locus from which the Csf5 sequences originate.

In certain embodiments, the Csf5 CasPR, the variant or derivative thereof (including dCsf5 mutant), or the functional fragment thereof binds to not just the full length or the natural DR hairpin RNA structure of the CRISPR locus to which they belong, but also binds to a truncated version of the DR hairpin RNA structure. In certain embodiments, the truncated version comprises at least the stem of the natural DR hairpin RNA structure. In certain embodiments, the Csf5 CasPR, the variant or derivative thereof (including dCsf5 mutant), or the functional fragment thereof binds to a variant DR hairpin RNA structure that preserves substantially all the structural features (e.g., stems, loops, bulges in the stem, etc.) but having different nucleotide sequences (e.g., at least 1, 2, 3, 4, 5, 6, 7, 8, 9, 10, 11, 12, 13, 14, 15, 16, 17, 18, 19, 20 or more nucleotide sequence differences compared to the wild-type DR sequence).

In a related aspect, the invention provides a polynucleotide encoding any one of the Class 1 type IV or Csf5 CasPR proteins herein, including wild-type, derivative/variant (including dCsf5 mutant), or functional fragment thereof.

In another related aspect, the invention provides reverse complement sequence of the above polynucleotides encoding any one of the Class 1 type IV or Csf5 CasPR proteins herein, including wild-type, derivative/variant (including dCsf5 mutant), or functional fragment thereof.

In certain embodiments, the polynucleotide is not a naturally occurring polynucleotide that encodes a wild-type Class 1 type IV or Csf5 CasPR protein herein.

In certain embodiments, the polynucleotide is codon-optimized for mammalian expression.

In certain embodiments, the polynucleotide has the polynucleotide sequence of SEQ ID NO: 43 or 44.

SEQ ID NOs: 1-33 are provided below.

| SEQ ID NO: | Description |
|---|---|
| 1 | SsoCas6 Amino Acid Sequence |
| 2 | MmCas6 Amino Acid Sequence |
| 3 | SpCas5d Amino Acid Sequence |
| 4 | BhCas5d Amino Acid Sequence |
| 5 | SaCas6 Amino Acid Sequence |
| 6 | EcCas6e Amino Acid Sequence |
| 7 | PaCas6f Amino Acid Sequence |
| 8 | MtCas6 Amino Acid Sequence |
| 9 | PfCas6 Amino Acid Sequence |
| 10 | PaCsf5 Amino Acid Sequence |
| 11 | MtCsf5 Amino Acid Sequence |
| 12 | SsoCas6 Direct Repeat (DR) Sequence |
| 13 | MmCas6 Direct Repeat (DR) Sequence |
| 14 | SpCas5d Direct Repeat (DR) Sequence |
| 15 | BhCas5d Direct Repeat (DR) Sequence |
| 16 | SaCas6 Direct Repeat (DR) Sequence |
| 17 | EcCas6e Direct Repeat (DR) Sequence |
| 18 | PaCas6f Direct Repeat (DR) Sequence |
| 19 | MtCas6 Direct Repeat (DR) Sequence |
| 20 | PfCas6 Direct Repeat (DR) Sequence |
| 21 | PaCsf5 Direct Repeat (DR) Sequence |
| 22 | MtCsf5 Direct Repeat (DR) Sequence |
| 23 | SsoCas6 Direct Repeat (DR) Transcript Sequence |
| 24 | MmCas6 Direct Repeat (DR) Transcript Sequence |
| 25 | SpCas5d Direct Repeat (DR) Transcript Sequence |
| 26 | BhCas5d Direct Repeat (DR) Transcript Sequence |
| 27 | SaCas6 Direct Repeat (DR) Transcript Sequence |
| 28 | EcCas6e Direct Repeat (DR) Transcript Sequence |
| 29 | PaCas6f Direct Repeat (DR) Transcript Sequence |
| 30 | MtCas6 Direct Repeat (DR) Transcript Sequence |
| 31 | PfCas6 Direct Repeat (DR) Transcript Sequence |
| 32 | PaCsf5 Direct Repeat (DR) Transcript Sequence |
| 33 | MtCsf5 Direct Repeat (DR) Transcript Sequence |

(SEQ ID NO: 1)
MPLIFKIGYNVIPLQDVILPTPSSKVLKYLIQSGKLIPSLKDLITSRDKYKPIFISHLGENQRRIFQING
NLKTITKGSRLSSIIAFSTQANVLSEVADEGIFETVYGKFHIMIESIEIVEVEKLKEEVEKHMNDNIRVR
FVSPILLSSKVLLPPSLSERYKKIHAGYSTLPSVGLIVAYAYNVYCNLIGKKEVEVRAFKFGILSNALSR
IIGYDLHPVTVAIGEDSKGNLRKARGVMGWIEFDIPDERLKRRALNYLLTSSYLGIGRSRGIGFGEIRLE
FRKIEEKEG
(SEQ ID NO: 2)
MDLEYMHISYPNILLNMRDGSKLRGYFAKKYIDEEIVHNHRDNAFVYKYPQIQFKIIDRSPLIIGIGSLG
INFLESKRIFFEKELIISNDINDITEVNVHKDMDHFGTTDKILKYQFKTPWMALNAKNSEIYKNSDEIDR
EEFLKRVLIGNILSMSKSLGYTIEEKLKVKINLKEVPVKFKNQNMVGFRGEFYINFDIPQYLGIGRNVSR
GFGTVVKV
(SEQ ID NO: 3)
MYRSRDFYVRVSGQRALFTNPATKGGSERSSYSVPTRQALNGIVDAIYYKPTFTNIVTEVKVINQIQTEL
QGVRALLHDYSADLSYVSYLSDVVYLIKFHFVWNEDRKDLNSDRLPAKHEAIMERSIRKGGRRDVFLGTR
ECLGLLDDISQEEYETTVSYYNGVNIDLGIMFHSFAYPKDKKTPLKSYFTKTVMKNGVITFKAQSECDIV
NTLSSYAFKAPEEIKSVNDECMEYDAMEKGEN
(SEQ ID NO: 4)
MRNEVQFELFGDYALFTDPLTKIGGEKLSYSVPTYQALKGIAESIYWKPTIVFVIDELRVMKPIQMESKG
VRPIEYGGGNTLAHYTYLKDVHYQVKAHFEFNLHRPDLAFDRNEGKHYSILQRSLKAGGRRDIFLGAREC
QGYVAPCEFGSGDGFYDGQGKYHLGTMVHGFNYPDETGQHQLDVRLWSAVMENGYIQFPRPEDCPIVRPV
KEMEPKIFNPDNVQSAEQLLHDLGGE
(SEQ ID NO: 5)
MPNDPYSLYSIVIELGAAEKGFPTGILGRSLHSQVLQWFKQDNPFLATELHQSQISPFSISPLMGKRHAK
LTKAGDRLFFRICLLRGDLLQPLLNGIEQTVNQSVCLDKFRFRLCQTHILPGSHPLAGASHYSLISQTPV
SSKITLDFKSSTSFKVDRKIIQVFPLGEHVENSLLRRWNNFAPEDLHFSQVDWSIPIAAFDVKTIPIHLK
KVEIGAQGWVTYIFPNTEQAKIASVLSEFAFFSGVGRKTTMGMGQVQVRS
(SEQ ID NO: 6)
MYLSKVIIARAWSRDLYQLHQGLWHLFPNRPDAARDFLFHVEKRNTPEGCHVLLQSAQMPVSTAVATVIK
TKQVEFQLQVGVPLYFRLRANPIKTILDNQKRLDSKGNIKRCRVPLIKEAEQIAWLQRKLGNAARVEDVH
PISERPQYFSGDGKSGKIQTVCFEGVLTINDAPALIDLVQQGIGPAKSMGCGLLSLAPL
(SEQ ID NO: 7)
MDHYLDIRLRPDPEFPPAQLMSVLFGKLHQALVAQGGDRIGVSFPDLDESRSRLGERLRIHASADDLRAL
LARPWLEGLRDHLQFGEPAVVPHPTPYRQVSRVQAKSNPERLRRRLMRRHDLSEEEARKRIPDTVARALD
LPFVTLRSQSTGQHFRLFIRHGPLQVTAEEGGFTCYGLSKGGFVPWF
(SEQ ID NO: 8)
MAARRGGIRRTDLLRRSGQPRGRHRASAAESGLTWISPTLILVGFSHRGDRRMTEHLSRLTLTLEVDAPL
ERARVATLGPHLHGVLMESIPADYVQTLHTVPVNPYSQYALARSTTSLEWKISTLINEARQQIVGPINDA
AFAGFRLRASGIATQVTSRSLEQNPLSQFARIFYARPETRKFRVEFLTPTAFKQSGEYVFWPDPRLVFQS
LAQKYGAIVDGEEPDPGLIAEFGQSVRLSAFRVASAPFAVGAARVPGFTGSATFTVRGVDTFASYIAALL
WEGEFSGCGIKASMGMGAIRVQPLAPREKCVPKP
(SEQ ID NO: 9)
MRFLIRLVPEDKDRAFKVPYNHQYYLQGLIYNAIKSSNPKLATYLHEVKGPKLFTYSLFMAEKREHPKGL
PYFLGYKKGFFYFSTCVPEIAEALVNGLLMNPEVRLWDERFYLHEIKVLREPKKFNGSTFVILSPIAVTV
VRKGKSYDVPPMEKEFYSIIKDDLQDKYVMAYGDKPPSEFEMEVLIAKPKRFRIKPGIYQTAWHLVFRAY
GNDDLLKVGYEVGFGEKNSLGFGMVKVEGNKTTKEAEEQEKITENSREELKTGV
(SEQ ID NO: 10)
MFVTQVIFNIGERTYPDRARAMVAELMDGVQPGLVATLMNYIPGTSTSRTEFPTVQFGGASDGFCLLGFG
DGGGAIVRDAVPLIHAALARRMPDRIIQVEHKEHSLSAEARPYVLSYTVPRMVVQKKQRHAERLLHEAEG
KAHLEGLFLRSLQRQAAAVGLPLPENLEVEFKGAVGDFAAKHNPNSKVAYRGLRGAVEDVNARLGGIWTA
GFMLSKGYGQFNATHQLSGAVNALSE
(SEQ ID NO: 11)
MHQTLIRINWPKGFKCPPAEFREKLAKSEMFPPEFFHYGTELAVWDKQTAEVEGKIKTVSKEKIIKTEDK
PIPLNGRAPVRVIGGQAWAGVIADPEMEGMLIPHLGSILKVASSAAGCAVKIELEQRKFGISYTEYPVKY
NLRELVLKRRCEDARSTDIESLIADRIWGGVSGESYYGIDGTCAKFGFEPPSREQLELRIFPMKNIGLHM
KSSDGLSKEYMSLIDAEVWMNAKLEGVWQVGNLISRGYGRFIKSIGAQS
SEQ ID NO: 12:
GATAATCTCTTATAGAATTGAAAG
SEQ ID NO: 13:
CTAAAAGAATAACTTGCAAAATAACAAGCATTGAAAC
SEQ ID NO: 14:
GTCTCACCCTTCATGGGTGAGTGGATTGAAAT
SEQ ID NO: 15:
GTCGCACTCTTCATGGGTGCGTGGATTGAAAT
SEQ ID NO: 16:
GTTTCAGTCCCGTAGTCGGGATTTAGTGGTTGGAAAG
SEQ ID NO: 17:
GAGTTCCCCGCGCCAGCGGGGATAAACCG
SEQ ID NO: 18:
GTTCACTGCCGTATAGGCAGCTAAGAAA
SEQ ID NO: 19:
GTCGTCAGACCCAAAACCCCGAGAGGGGACGGAAAC
SEQ ID NO: 20:
GTTACAATAAGACTAAAATAGAATTGAAAG
SEQ ID NO: 21:
GTATTTCCCGCGTGCGCGGGGGTGAGCGG
SEQ ID NO: 22:
TATTGGATACACCCACTCATTGGTGGGTGGTTAGAAC
SEQ ID NO: 23:
GAUAAUCUCUUAUAGAAUUGAAAG
SEQ ID NO: 24:
CUAAAAGAAUAACUUGCAAAAUAACAAGCAUUGAAAC
SEQ ID NO: 25:
GUCUCACCCUUCAUGGGUGAGUGGAUUGAAAU
SEQ ID NO: 26:
GUCGCACUCUUCAUGGGUGCGUGGAUUGAAAU
SEQ ID NO: 27:
GUUUCAGUCCCGUAGUCGGGAUUUAGUGGUUGGAAAG
SEQ ID NO: 28:
GAGUUCCCCGCGCCAGCGGGGAUAAACCG
SEQ ID NO: 29:
GUUCACUGCCGUAUAGGCAGCUAAGAAA
SEQ ID NO: 30:
GUCGUCAGACCCAAAACCCCGAGAGGGGACGGAAAC
SEQ ID NO: 31:
GUUACAAUAAGACUAAAAUAGAAUUGAAAG
SEQ ID NO: 32:
GUAUUUCCCGCGUGCGCGGGGGUGAGCGG
SEQ ID NO: 33:
UAUUGGAUACACCCACUCAUUGGUGGGUGGUUAGAAC

### Functional Fragments

Functional fragments of the subject CasPRs (e.g., Cas5d, Cas6, and Csf5), including wild-type, variant, and derivative thereof, are also provided. The functional fragments of the invention preserve or maintain at least one function of the full-length protein from which they originate. For example, in some embodiments, the preserved function is binding to cognate crRNA particularly the DR sequence or structural elements therein responsible for CasPR binding. In other embodiments, the preserved function is catalytic activity towards pre-crRNA. In some embodiments, both binding to DR sequence and catalytic activity are preserved.

For example, in certain embodiments, to reduce the size of a fusion protein of the subject CasPR and the one or more functional domains (see below), the C-terminus of the CasPR (e.g., Cas5d, Cas6, and Csf5) can be truncated while still maintaining its RNA binding function. For example, at least or no more than 5 amino acids, 10 amino acids, 15 amino acids, 20 amino acids, 25 amino acids, 30 amino acids, 35 amino acids, 40 amino acids, 45 amino acids, 50 amino acids, 55 amino acids, 60 amino acids, 65 amino acids, 70 amino acids, 75 amino acids, 80 amino acids, 85 amino acids, 90 amino acids, or 100 amino acid, may be truncated at the C-terminus of the CasPR.

In some embodiments, the N-terminus of the CasPR (e.g., Cas5d, Cas6, and Csf5) may be truncated. For example, at least or no more than 5 amino acids, 10 amino acids, 15 amino acids, 20 amino acids, 25 amino acids, 30 amino acids, 35 amino acids, 40 amino acids, 45 amino acids, 50 amino acids, 55 amino acids, 60 amino acids, 65 amino acids, 70 amino acids, 75 amino acids, 80 amino acids, 85 amino acids, 90 amino acids, or 100 amino acid may be truncated at the N-terminus of the subject CasPR.

In some embodiments, both the N- and the C-termini of the subject CasPR may be truncated. Not specifically recited herein but are explicitly incorporated is a permutation and combination of each N- and each C-terminal deletions mentioned above, such as C-terminal deletion of at least/no more than 5 residues AND N-terminal deletions of at least/no more than 5, 10, 15, 20, 20, 25, 30, 35, 40, 45, 50, 55, 60, 65, 70, 75, 80, 85, 90, 95, or 100 residues; . . . and, C-terminal deletion of at least/no more than 100 residues AND N-terminal deletions of at least/no more than 5, 10, 15, 20, 20, 25, 30, 35, 40, 45, 50, 55, 60, 65, 70, 75, 80, 85, 90, 95, or 100 residues.

### Split Proteins

In certain embodiments, the functional fragment is a so-called “split protein,” in that it contains one of two parts of the full length CasPR enzyme—the RNA binding domain or the endonuclease domain, which together substantially comprise a functional CasPR. The split should always be so that the catalytic domain(s) are unaffected. The use of a split version of the CasPR may not only allow increased specificity but may also be advantageous for delivery (e.g., smaller size). Thus in certain embodiments, the split CasPR may function as a nuclease. In another embodiment, the split CasPR may be a nuclease dead-CasPR which is essentially an RNA-binding protein with very little or no catalytic activity, due to typically mutation(s) in its catalytic domains or the lack of the catalytic domain altogether. The nuclease dead-split CasPR can be fused to other heterologous functional domains described herein to target such heterologous functional domains to a specific site on a target RNA.

In certain embodiments, each half of the split CasPR may be fused to a dimerization partner, such as the rapamycin-sensitive dimerization domains, which allow the generation of a chemically inducible split CasPR temporal control of CasPR activity. For example, the split CasPR RNA binding domain may bind to the guide RNA at the target site, and the split CasPR nuclease domain (or nuclease-dead version of the nuclease domain) may be fused to a heterologous functional domain, such as a deaminase. Thus CasPR can be rendered chemically inducible by being split into two fragments and that rapamycin-sensitive dimerization domains may be used for controlled reassembly of the CasPR or fusion thereof.

### Conservative Substitutions

In certain embodiments, derivatives or variants of the CasPRs (e.g., Cas5d, Cas6, and Csf5) include proteins that differ from the wild-type sequence by one or more conservative substitutions, include substitutions inside or outside the RNA binding or catalytic domain. In certain embodiments, the substitution does not include substitution of the catalytic triad residues. In certain embodiments, the substitution includes substitution of the catalytic triad residues.

Such amino acid substitutions may be made based on the differences or similarities in amino acid properties, such as polarity, charge, solubility, hydrophobicity, hydrophilicity, and/or the amphipathic nature of the residues. For this purpose, amino acids have been grouped together based on the functional groups they carry, i.e., based on the properties of their side chains alone. Typically, a grouping as shown below can be used for conservative substitution.

| Set | | Sub-set | |
|---|---|---|---|
| Hydrophobic | F W Y H K M I L V A G C | Aromatic | F W Y H |
| | (SEQ ID NO: 240) | Aliphatic | I L V |
| Polar | W Y H K R E D C S T N Q | Charged | H K R E D |
| | (SEQ ID NO: 241) | Positively | H K R |
| | | charged | |
| | | Negatively | E D |
| | | charged | |
| Small | V C A G S P T N D | Tiny | A G S |
| | (SEQ ID NO: 242) | | |

Homology Modeling Numerous subject CasPR protein sequences have been described herein, including publically available database sequences incorporated herein that satisfy certain threshold sequence identity requirements to the subject CasPRs (e.g., SEQ ID NOs: 1-11). Homology modeling can be used to predict the structure of the related CasPRs, such as homologs, orthologs, paralogs, variants, derivatives, and functional fragments thereof, partly based on the known structures of certain CasPRs within a subfamily, and the sequence homology/identity between the related CasPRs.

For example, corresponding residues in other CasPR orthologs can be identified by the methods of Zhang et al. (Nature 490(7421):556-60, 2012, incorporated herein by reference) and Chen et al. (PLoS Comput Biol. 11(5):e1004248, 2015, incorporated herein by reference). The method involves taking a pair a query proteins and using structural alignment to identify structural representatives that correspond to either their experimentally determined structures or homology models. Structural alignment is further used to identify both close and remote structural neighbors by considering global and local geometric relationships. Whenever two neighbors of the structural representatives form a complex reported in the Protein Data Bank, this defines a template for modelling the interaction between the two query proteins. Models of a complex are created by superimposing the representative structures on their corresponding structural neighbor in the template. Also see Dey et al., Prot Sci. 22:359-66, 2013.

### 3. Guide RNA/crRNA

As used herein, “guide sequence,” “space(r) sequence,” or “spacer” are used interchangeably to refer to RNA capable of guiding the subject CasPR proteins (e.g., Cas5d, Cas6, or Csf5) to a target locus, wherein the guide sequence is substantially complementary in sequence to the polynucleotide (e.g., mRNA) at the target locus, and wherein the guide sequence is linked to the DR sequence or structural elements thereof that can bind to the CasPR to form a crRNA or “guide RNA.”

In certain embodiments, the guide RNA (or crRNA) capable of guiding a subject CasPR to a target locus comprises: (1) a guide sequence capable of hybridizing to a target locus (a polynucleotide target locus, such as an mRNA target locus) in a eukaryotic cell; (2) a direct repeat (DR) sequence or a functional portion thereof capable of binding to the subject CasPR, wherein (1) and (2) are linked in a single RNA, which may sometimes also be referred to as a sgRNA (single guide RNA) or crRNA. In certain embodiments, (1) is 5′ to (2). In certain embodiments, (1) is 3′ to (2). In certain embodiments, there is no linker between (1) and (2). In certain embodiments, there is a linker between (1) and (2). The linker may be 1, 2, 3, 4, 5, 6, 7, 8, 9, or 10 nt in length, which can be random sequences.

In certain embodiments, a guide RNA or crRNA may comprise, consist essentially of, or consist of a direct repeat (DR) sequence and a guide sequence or spacer sequence. In certain embodiments, the guide RNA or crRNA may comprise, consist essentially of, or consist of a direct repeat sequence fused or linked to a guide sequence or spacer sequence. In certain embodiments, the direct repeat sequence may be located upstream (i.e., 5′) from the guide sequence or spacer sequence. In other embodiments, the direct repeat sequence may be located downstream (i.e., 3′) from the guide sequence or spacer sequence. In other embodiments, multiple DRs (such as dual DRs) may be present.

In certain embodiments, the crRNA comprises a stem loop, e.g., a single stem loop. In certain embodiments, the direct repeat sequence forms a stem loop, e.g., a single stem loop.

### Guide Sequence

In general, a guide sequence is any polynucleotide sequence having sufficient complementarity with a target polynucleotide sequence (e.g., mRNA) to hybridize with the target sequence and direct sequence-specific binding of a subject CasPR or fusion thereof to the target sequence. In some embodiments, the degree of complementarity between a guide sequence and its corresponding target sequence, when optimally aligned using a suitable alignment algorithm, is about or more than about 50%, 60%, 75%, 80%, 85%, 90%, 95%, 97.5%, 99%, or more. Optimal alignment may be determined with the use of any suitable algorithm for aligning sequences, non-limiting example of which include the Smith-Waterman algorithm, the Needleman-Wunsch algorithm, algorithms based on the Burrows-Wheeler Transform (e.g., the Burrows Wheeler Aligner), ClustalW, Clustal X, BLAT, Novoalign (Novocraft Technologies; available at novocraft.com), ELAND (Illumina, San Diego, CA), SOAP (available at soap.genomics.org.cn), and Maq (available at maq.sourceforge.net).

In some embodiments, a guide sequence is about or more than about 5, 10, 11, 12, 13, 14, 15, 16, 17, 18, 19, 20, 21, 22, 23, 24, 25, 26, 27, 28, 29, 30, 35, 40, 45, 50, 75, or more nucleotides in length. In some embodiments, a guide sequence is less than about 75, 50, 45, 40, 35, 30, 25, 20, 15, 12, or fewer nucleotides in length. In certain embodiments, the guide sequence is 10-40 nucleotides long, such as 10-30, 20-30, or 20-40 nucleotides long. In certain embodiments, the guide sequence is about 30 nucleotides long.

In certain embodiments, the spacer length of the guide RNA is from 15 to 35 nt. In certain embodiments, the spacer length of the guide RNA is at least 15 nucleotides. In certain embodiments, the spacer length is from 15 to 17 nt, e.g., 15, 16, or 17 nt, from 17 to 20 nt, e.g., 17, 18, 19, or 20 nt, from 20 to 24 nt, e.g., 20, 21, 22, 23, or 24 nt, from 23 to 25 nt, e.g., 23, 24, or 25 nt, from 24 to 27 nt, e.g., 24, 25, 26, or 27 nt, from 27-30 nt, e.g., 27, 28, 29, or 30 nt, from 30-35 nt, e.g., 30, 31, 32, 33, 34, or 35 nt, or 35 nt or longer.

In certain embodiments, the guide sequence is able to distinguish between target and off-target sequences that have greater than 80% to about 95% complementarity, e.g., 83%-84% or 88-89% or 94-95% complementarity (for instance, distinguishing between a target having 18 nucleotides from an off-target of 18 nucleotides having 1, 2 or 3 mismatches).

### Dead Guide Sequence

In certain embodiments, the guide sequence is modified in a manner which allows for formation of the CasPR-guide RNA complex and successful binding to the target sequence, while at the same time, not allowing for successful nuclease activity of the CasPR, even for wild-type CasPR. Such guide sequence is also referred to as “dead guide sequence.” These dead guide sequences can be thought of as catalytically inactive or conformationally inactive with regard to nuclease activity. For example, dead guide sequences may not sufficiently engage in productive base pairing with respect to the ability to promote catalytic activity of the CasPR, or to distinguish on-target and off-target binding activity. Such dead guide sequence can be identified using an assay that involves synthesizing a target RNA and a guide RNA comprising mismatches with the target RNA, and combining these with the CasPR of interest and analyzing cleavage based on gel mobility shift assays or equivalent assays to detect the presence/absence of bands generated by cleavage products, and quantifying cleavage based upon relative band intensities. Such dead guide sequence, when used in guide RNA comprising the same, can be used with wild-type or mutant CasPR of the invention that binds the dead guide sequence but fails to cleave the target, thus allowing any fused functional domain to act on the target.

Thus, in a related aspect, the invention provides a non-naturally occurring or engineered composition RNA targeting a subject CasPR comprising a functional RNA targeting enzyme as described herein, and a guide RNA or crRNA comprising a dead guide sequence, whereby the guide RNA/crRNA is capable of hybridizing to a target sequence such that the RNA targeting CasPR or fusion thereof is directed to the target sequence (e.g., mRNA) in a cell without detectable RNA cleavage activity by a non-mutant CasPR RNA targeting enzyme of the invention. It is to be understood that any of the guide RNAs or crRNAs according to the invention as described herein elsewhere may be a dead guide RNAs/crRNAs comprising a dead guide sequence.

The ability of a dead guide sequence to direct sequence-specific binding of a subject CasPR or fusion thereof to an RNA target sequence may be assessed by any suitable assay. For example, the components of a subject CasPR system sufficient to form a CasPR-guide RNA complex, including the dead guide sequence to be tested, may be provided to a host cell having the corresponding target sequence, such as by transfection with vectors encoding the components of the system, followed by an assessment of preferential cleavage within the target sequence.

Several structural parameters allow for a proper framework to arrive at such dead guides. Dead guide sequences can be typically shorter than respective guide sequences which result in active RNA cleavage. In particular embodiments, dead guides are 5%, 10%, 20%, 30%, 40%, 50%, shorter than respective guides directed to the same.

Structural data available for validated dead guide sequences may be used for designing specific equivalents. Structural similarity between, e.g., the orthologous nuclease domains of two or more CasPR proteins may be used to transfer design equivalent dead guides. Thus, the dead guide herein may be appropriately modified in length and sequence to reflect such CasPR specific equivalents, allowing for formation of the CasPR complex and successful binding to the target RNA, while at the same time, not allowing for successful nuclease activity.

Dead guides allow one to use guide RNA or crRNA as a means for gene targeting, without the consequence of nuclease activity, while at the same time providing directed means for activation or repression. Guide RNA or crRNA comprising a dead guide may be modified to further include elements in a manner which allow for activation or repression of gene activity, in particular protein adaptors (e.g. aptamers) allowing for functional placement of gene effectors (e.g. activators or repressors of gene activity). One example is the incorporation of aptamers, as in the state of the art. By engineering the guide RNA or crRNA comprising a dead guide to incorporate protein-interacting aptamers (Konermann et al., “Genome-scale transcription activation by an engineered CRISPR-Cas9 complex,” doi: 10. 1038/nature14136, incorporated herein by reference), one may assemble multiple distinct effector domains. Such may be modeled after natural processes.

### Chemically Synthesized Guide Sequence and Guide RNA

In some embodiments, one or more portions of a guide RNA can be chemically synthesized. In some embodiments, the chemical synthesis uses automated, solid-phase oligonucleotide synthesis machines with 2′-acetoxyethyl orthoester (2′-ACE) (Scaringe et al., J. Am. Chem. Soc. 120:11820-11821, 1998; Scaringe, Methods Enzymol. 317:3-18, 2000) or 2′-thionocarbamate (2′-TC) chemistry (Dellinger et al., J. Am. Chem. Soc. 133:11540-11546, 2011; Hendel et al., Nat. Biotechnol. 33:985-989, 2015).

In some embodiments, the guide portions can be covalently linked using various bioconjugation reactions, loops, bridges, and non-nucleotide links via modifications of sugar, internucleotide phosphodiester bonds, purine and pyrimidine residues. Sletten et al., Angew. Chem. Int. Ed. (2009) 48:6974-6998; Manoharan, M. Curr. Opin. Chem. Biol. 8:570-9, 2004; Behlke et al., Oligonucleotides 18:305-19, 2008; Watts et al., Drug. Discov. Today 13:842-55, 2008; Shukla et al., ChemMedChem 5:328-49, 2010.

In some embodiments, the guide portions can be covalently linked using click chemistry. In some embodiments, guide portions can be covalently linked using a triazole linker. In some embodiments, guide portions can be covalently linked using Huisgen 1,3-dipolar cycloaddition reaction involving an alkyne and azide to yield a highly stable triazole linker (He et al., ChemBioChem (2015) 17: 1809-1812; WO 2016/186745). In some embodiments, guide portions are covalently linked by ligating a 5′-hexyne portion and a 3′-azide portion. In some embodiments, either or both of the 5′-hexyne guide portion and a 3′-azide guide portion can be protected with 2′-acetoxyethl orthoester (T-ACE) group, which can be subsequently removed using Dharmacon protocol (Scaringe et al., J. Am. Chem. Soc. (1998) 120: 11820-11821; Scaringe, Methods Enzymol. (2000) 317: 3-18).

In some embodiments, guide portions can be covalently linked via a linker (e.g., a non-nucleotide loop) that comprises a moiety such as spacers, attachments, bioconjugates, chromophores, reporter groups, dye labeled RNAs, and non-naturally occurring nucleotide analogues. More specifically, suitable spacers for purposes of this invention include, but are not limited to, polyethers (e.g., polyethylene glycols, polyalcohols, polypropylene glycol or mixtures of ethylene and propylene glycols), polyamines group (e.g., spennine, spermidine and polymeric derivatives thereof), polyesters (e.g., poly(ethyl acrylate)), polyphosphodiesters, alkylenes, and combinations thereof. Suitable attachments include any moiety that can be added to the linker to add additional properties to the linker, such as but not limited to, fluorescent labels. Suitable bioconjugates include, but are not limited to, peptides, glycosides, lipids, cholesterol, phospholipids, diacyl glycerols and dialkyl glycerols, fatty acids, hydrocarbons, enzyme substrates, steroids, biotin, digoxigenin, carbohydrates, polysaccharides. Suitable chromophores, reporter groups, and dye-labeled RNAs include, but are not limited to, fluorescent dyes such as fluorescein and rhodamine, chemiluminescent, electrochemiluminescent, and bioluminescent marker compounds. The design of example linkers conjugating two RNA components are also described in WO 2004/015075, incorporated herein by reference.

The linker (e.g., a non-nucleotide loop) can be of any length. In some embodiments, the linker has a length equivalent to about 0-16 nucleotides. In some embodiments, the linker has a length equivalent to about 0-8 nucleotides. In some embodiments, the linker has a length equivalent to about 0-4 nucleotides. In some embodiments, the linker has a length equivalent to about 2 nucleotides. Example linker design is also described in WO2011/008730, incorporated herein by reference.

In certain embodiments, the CasPR proteins can bind to guide RNA or crRNA or analogous polynucleotide comprising a guide sequence, wherein the polynucleotide can be completely or partly an RNA, a DNA or a mixture of RNA and DNA, and/or wherein the polynucleotide comprises one or more nucleotide analogs. The sequence can comprise any structure, including but not limited to a structure of a native crRNA, such as a bulge, a hairpin or a stem loop structure.

### Direct Repeat (DR) Sequence

The guide RNA or crRNA comprises a direct repeat (DR) sequence, which is linked to the guide sequence (for binding to the target sequence) and which is capable of binding the subject CasPR or fusion thereof. This implies that the direct repeat sequences are designed dependent on the origin of the CasPR RNA targeting enzyme.

Exemplary DR sequences for selected CasPR endonenleases have been provided herein (see SEQ ID NOs: 12-22), as well as their transcript sequences (see SEQ ID NOs: 23-33). Additional DR sequences for specific CasPR homologs, orthologs or paralogs can be obtained from the CRISPR loci for the CasPR homologs, orthologs or paralogs. The minimum effective DR sequence elements required for binding to the respective CasPR proteins are taught above with respect to the prototypical CasPRs, such as Cas5d, Cas6, and Csf5.

### 4. Target Sequence

A guide sequence (including a dead guide), and hence a RNA-targeting guide RNA or crRNA encompassing the guide sequence, may be selected to target any target nucleic acid sequence or target sequence. That is, the target RNA can be any RNA molecule of interest, including naturally-occurring and engineered RNA molecules.

In certain embodiments, the target RNA can be an mRNA, a tRNA, a ribosomal RNA (rRNA), a microRNA (miRNA), an interfering RNA (siRNA), a ribozyme, a riboswitch, a satellite RNA, a microswitch, a microzyme, or a viral RNA.

In some embodiments, the target sequence may be a sequence within a RNA molecule selected from the group consisting of messenger RNA (mRNA), pre-mRNA, ribosomal RNA (rRNA), transfer RNA (tRNA), micro-RNA (miRNA), small interfering RNA (siRNA), small nuclear RNA (snRNA), small nucleolar RNA (snoRNA), double stranded RNA (dsRNA), non-coding RNA (ncRNA), long non-coding RNA (lncRNA), and small cytoplasmatic RNA (scRNA). In some embodiments, the target sequence may be a sequence within a RNA molecule selected from the group consisting of mRNA, pre-mRNA, and rRNA. In some embodiments, the target sequence may be a sequence within a RNA molecule selected from the group consisting of ncRNA, and lncRNA. In some embodiments, the target sequence may be a sequence within an mRNA molecule or a pre-mRNA molecule.

In some embodiments, the target nucleic acid is associated with a condition or disease (e.g., an infectious disease or a cancer).

Thus, in some embodiments, the systems described herein can be used to treat a condition or disease by targeting these nucleic acids. For instance, the target nucleic acid associated with a condition or disease may be an RNA molecule that is overexpressed in a diseased cell (e.g., a cancer or tumor cell). The target nucleic acid may also be a toxic RNA and/or a mutated RNA (e.g., an mRNA molecule having a splicing defect or a mutation). The target nucleic acid may also be an RNA that is specific for a particular microorganism (e.g., a pathogenic bacteria).

### 5. Heterologous Functional Domains and Fusion Partners

In some embodiments, the CasPR of the invention (e.g., Cas5d, Cas6, Csf5), including variants, derivatives, endonuclease deficient/dead version thereof, or functional fragment thereof is fused to a heterologous functional domain or fusion partner.

In certain embodiments, the heterologous functional domain comprises: a nuclear localization signal (NLS), a reporter protein or a detection label (e.g., GST, HRP, CAT, GFP, HcRed, DsRed, CFP, YFP, BFP), a localization signal, a protein targeting moiety, a DNA binding domain (e.g., MBP, Lex A DBD, Gal4 DBD), an epitope tag (e.g., His, myc, V5, FLAG, HA, VSV-G, Trx, etc), a transcription activation domain (e.g., VP64 or VPR), a transcription inhibition domain (e.g., KRAB moiety or SID moiety), a translation modification factor, a nuclease (e.g., FokI), a deamination domain (e.g., ADAR1, ADAR2, APOBEC, AID, or TAD), a methylase, a demethylase (e.g., an RNA demethylase), an RNA methyltransferase, an RNA splicing modifier, a transcription release factor, an HDAC, a polypeptide having ssRNA cleavage activity, a polypeptide having dsRNA cleavage activity, a polypeptide having ssDNA cleavage activity, a polypeptide having dsDNA cleavage activity, a DNA or RNA ligase, or any combination thereof.

In a related aspect, the invention provides conjugates of the subject CasPR proteins based on any one of SEQ ID NOs: 1-11, or orthologs, homologs, variants/derivatives (including nuclease dead) and functional fragments thereof, which are conjugated with moieties such as other proteins or polypeptides, detectable labels, or combinations thereof. Such conjugated moieties may include, without limitation, localization signals, reporter genes (e.g., GST, HRP, CAT, GFP, HcRed, DsRed, CFP, YFP, BFP), labels (e.g., fluorescent dye such as FITC, or DAPI), NLS, targeting moieties, DNA binding domains (e.g., MBP, Lex A DBD, Gal4 DBD), epitope tags (e.g., His, myc, V5, FLAG, HA, VSV-G, Trx, etc), transcription activation domains (e.g., VP64 or VPR), transcription inhibition domains (e.g., KRAB moiety or SID moiety), nucleases (e.g., FokI), deamination domain (e.g., ADAR1, ADAR2, APOBEC, AID, or TAD), methylase, demethylase, transcription release factor, HDAC, ssRNA cleavage activity, dsRNA cleavage activity, ssDNA cleavage activity, dsDNA cleavage activity, DNA or RNA ligase, any combination thereof, etc.

The inactivated or nuclease dead CasPR or derivative or functional fragment thereof can be fused or associated with one or more heterologous/functional domains (e.g., via fusion protein, linker peptides, “GS” linkers, etc.). These functional domains can have various activities, e.g., methylase activity, demethylase activity, transcription activation activity, transcription repression activity, transcription release factor activity, histone modification activity, RNA cleavage activity, DNA cleavage activity, nucleic acid binding activity, base-editing activity, and switch activity (e.g., light inducible). In some embodiments, the functional domains are Krüppel associated box (KRAB), SID (e.g. SID4X), VP64, VPR, VP16, Fok1, P65, HSF1, MyoD1, Adenosine Deaminase Acting on RNA such as ADAR1, ADAR2, APOBEC, cytidine deaminase (AID), TAD, mini-SOG, APEX, and biotin-APEX.

In some embodiments, the CasPR described herein is fused to one or more peptide tags, including a His-tag, GST-tag, a V5-tag, FLAG-tag, HA-tag, VSV-G-tag, Trx-tag, or myc-tag.

In some embodiments, the CasPR described herein is fused to a detectable moiety such as GST, a fluorescent protein (e.g., GFP, HcRed, DsRed, CFP, YFP, or BFP), or an enzyme (such as HRP or CAT).

In some embodiments, the CasPR described herein is fused to MBP, LexA DNA binding domain, or Gal4 DNA-binding domain.

In some embodiments, the CasPR described herein is linked to or conjugated with a detectable label such as a fluorescent dye, including FITC and DAPI.

In some embodiments, multiple (e.g., two, three, four, five, six, seven, eight, or more) identical or different functional domains are present.

The positioning of the one or more functional domains on the inactivated CasPR proteins is one that allows for correct spatial orientation for the functional domain to affect the target with the attributed functional effect. For example, if the functional domain is a transcription activator (e.g., VP16, VP64, or p65), the transcription activator is placed in a spatial orientation that allows it to affect the transcription of the target. Likewise, a transcription repressor is positioned to affect the transcription of the target, and a nuclease (e.g., Fok1) is positioned to cleave or partially cleave the target. In some embodiments, the functional domain is positioned at the N-terminus of the CasPR/dCasPR. In some embodiments, the functional domain is positioned at the C-terminus of the CasPR/dCasPR. In some embodiments, the inactivated CasPR (dCasPR) is modified to comprise a first functional domain at the N-terminus and a second functional domain at the C-terminus.

Various examples of inactivated Cas proteins fused with one or more functional domains and methods of using the same are described, e.g., in International Publication No. WO 2017/219027, which is incorporated herein by reference in its entirety, and in particular with respect to the features described herein.

In some embodiments, the functional domain (e.g., a base editing domain) is further fused to an RNA-binding domain (e.g., MS2).

In certain embodiments, the heterologous functional domain is fused N-terminally, C-terminally, or internally in the fusion protein.

In some embodiments, the heterologous functional domain comprises an NLS (nuclear localization signal) or a nuclear export signal (NES).

In certain embodiments, the heterologous functional domain comprises one or more nuclear localization sequences (NLSs), such as about or more than about 1, 2, 3, 4, 5, 6, 7, 8, 9, 10, or more NLSs. In some embodiments, the CasPR comprises about or more than about 1, 2, 3, 4, 5, 6, 7, 8, 9, 10, or more NLSs at or near the amino-terminus, about or more than about 1, 2, 3, 4, 5, 6, 7, 8, 9, 10, or more NLSs at or near the carboxy-terminus, or a combination of these (e.g. zero or at least one or more NLS at the amino-terminus and zero or at one or more NLS at the carboxy terminus). When more than one NLS is present, each may be selected independently of the others, such that a single NLS may be present in more than one copy and/or in combination with one or more other NLSs present in one or more copies. In one embodiment, the CasPR comprises at most 6 NLSs. In some embodiments, an NLS is considered near the N- or C-terminus when the nearest amino acid of the NLS is within about 1, 2, 3, 4, 5, 10, 15, 20, 25, 30, 40, 50, or more amino acids along the polypeptide chain from the N- or C-terminus.

Non-limiting examples of NLSs include an NLS sequence derived from: the NLS of the SV40 virus large T-antigen, having the amino acid sequence PKKKRKV (SEQ ID NO: 134); the NLS from nucleoplasmin (e.g. the nucleoplasmin bipartite NLS with the sequence KRPAATKKAGQAKKKK (SEQ ID NO: 135)); the c-myc NLS having the amino acid sequence PAAKRVKLD (SEQ ID NO: 136) or RQRRNELKRSP (SEQ ID NO: 137); the hRNPA1 M9 NLS having the sequence NQSSNFGPMKGGNFGGRSSGPYGG GGQYFAKPRNQGGY (SEQ ID NO: 138); the sequence RMRIZFKNKGKDTAELRRRRVEVSVELRKAKKDEQILKRR (SEQ ID NO: 139) NV of the IBB domain from importin-alpha; the sequences VSRKRPRP (SEQ ID NO: 140) and PPKKARED (SEQ ID NO: 141) of the myoma T protein; the sequence PQPKKKPL (SEQ ID NO: 142) ofhumanp53; the sequence SALIKKKKKMAP (SEQ ID NO: 143) of mouse c-abl IV; the sequences DRLRR (SEQ ID NO: 144) and PKQKKRK (SEQ ID NO: 145) of the influenza virus NS1; the sequence RKLKKKIKKL (SEQ ID NO: 146) of the Hepatitis virus delta antigen; the sequence REKKKFLKRR (SEQ ID NO: 147) of the mouse Mxl protein; the sequence KRKGDEVDGVDEVAKKKSKK (SEQ ID NO: 148) of the human poly(ADP-ribose) polymerase; and the sequence RKCLQAGMNLEARKTKK (SEQ ID NO: 149) of the steroid hormone receptors (human) glucocorticoid. In general, the one or more NLSs are of sufficient strength to drive accumulation of the CasPR in a detectable amount in the nucleus of a eukaryotic cell. In general, strength of nuclear localization activity may derive from the number of NLSs in the CasPR, the particular NLS(s) used, or a combination of these factors.

Detection of accumulation in the nucleus may be performed by any suitable technique. For example, a detectable marker may be fused to the CasPR, such that location within a cell may be visualized, such as in combination with a means for detecting the location of the nucleus (e.g. a stain specific for the nucleus such as DAPI). Cell nuclei may also be isolated from cells, the contents of which may then be analyzed by any suitable process for detecting protein, such as immunohistochemistry, Western blot, or enzyme activity assay.

In certain embodiments, the subject CasPR protein is conjugated with or fused to a destabilization domain (DD). In some embodiments, the DD is ER50. A corresponding stabilizing ligand for this DD is, in some embodiments, 4HT. As such, in some embodiments, one of the at least one DDs is ER50 and a stabilizing ligand therefor is 4HT or CMP8. In some embodiments, the DD is DHFR50. A corresponding stabilizing ligand for this DD is, in some embodiments, TMP. As such, in some embodiments, one of the at least one DDs is DHFR50 and a stabilizing ligand therefor is TMP. CMP8 may therefore be an alternative stabilizing ligand to 4HT in the ER50 system. While it may be possible that CMP8 and 4HT can/should be used in a competitive matter, some cell types may be more susceptible to one or the other of these two ligands, and from this disclosure and the knowledge in the art the skilled person can use CMP8 and/or 4HT.

In certain embodiments, the CasPR RNA-targeting protein-guide RNA complex as a whole may be associated with two or more functional domains. For example, there may be two or more functional domains associated with the CasPR RNA-targeting protein, or there may be two or more functional domains associated with the guide RNA or crRNA (via one or more adaptor proteins), or there may be one or more functional domains associated with the CasPR RNA-targeting protein and one or more functional domains associated with the guide RNA or crRNA (via one or more adaptor proteins).

In any of the fusions herein, a linker may be present between the heterologous domains. For example, in some embodiments, the GlySer linker GGGS can be used. They can be used in repeats of 3, 6, 9 or even 12 or more, to provide suitable lengths, as required to engineer appropriate amounts of “mechanical flexibility” into the fusion.

### 6. Nucleotide (Adenosine/Cytidine) Deaminases

In certain embodiments, the CasPR is linked, conjugated to or fused with a nucleotide deaminase, which can be used for RNA base editing. The nucleotide deaminase can be wild-type protein or engineered/mutant with altered or enhanced characteristics.

In certain embodiments, the nucleotide deaminase is an adenosine deaminase, or a catalytic domain thereof.

In certain embodiments, the nucleotide deaminase is a cytidine deaminase, or a catalytic domain thereof.

In certain embodiments, the nucleotide deaminase is an adenosine deaminase engineered to comprise cytidine deaminase activity, or a catalytic domain thereof.

In some embodiments, the nucleotide deaminase comprises one or more mutations selected from: E488Q, V351G, S486A, T375S, S370C, P462A, N597I, L332I, I398V, K350I, M383L, D619G, S582T, V440I, S495N, K418E, and S661T, based on amino acid sequence positions of hADAR2-D, and corresponding mutations in a homologous ADAR protein.

In a related aspect, the invention provides an engineered cell expressing the engineered adenosine deaminase or a catalytic domain thereof. In some embodiments, the cell transiently expresses the engineered adenosine deaminase or the catalytic domain thereof. In some embodiments, the cell non-transiently expresses the engineered adenosine deaminase or the catalytic domain thereof.

In another related aspect, the invention provides a method for modifying nucleotide in a target nucleic acid, comprising: delivering to said target nucleic acid the engineered adenosine deaminase, or the system, wherein the deaminase deaminates a nucleotide at one or more target loci on the target nucleic acid.

In some embodiments, said nucleotide deaminase protein or catalytic domain thereof has been modified to increase activity against a DNA-RNA heteroduplex. In some embodiments, said nucleotide deaminase protein or catalytic domain thereof has been modified to reduce off-target effects.

In some embodiments, the target nucleic acid is within a cell. In some embodiments, said cell is a eukaryotic cell. In some embodiments, said cell is a non-human animal cell. In some embodiments, said cell is a human cell. In some embodiments, said cell is a plant cell. In some embodiments, said target nucleic acid is within an animal. In some embodiments, said target nucleic acid is within a plant. In some embodiments, said target nucleic acid is comprised in a DNA molecule in vitro.

In some embodiments, the engineered adenosine deaminase, or one or more components of the system are delivered to the cell as a ribonucleoprotein complex. In some embodiments, the engineered adenosine deaminase, or one or more components of the system are delivered via one or more particles, one or more vesicles, or one or more viral vectors. In some embodiments, said one or more particles comprise a lipid, a sugar, a metal or a protein. In some embodiments, said one or more particles comprise lipid nanoparticles. In some embodiments, said one or more vesicles comprise exosomes or liposomes. In some embodiments, said one or more viral vectors comprise one or more adenoviral vectors, one or more lentiviral vectors, or one or more adeno-associated viral vectors.

In some embodiments, said method modifies a cell, a cell line or an organism by manipulation of one or more target mRNA sequences of interest.

In some embodiments, said deamination of said nucleotide at said target locus of interest remedies a disease caused by a GA or CT point mutation or a pathogenic SNP.

In some embodiments, said disease is selected from cancer, haemophilia, beta-thalassemia, Marfan syndrome and Wiskott-Aldrich syndrome. In some embodiments, said deamination of said nucleotide at said target locus of interest remedies a disease caused by a TC or AG point mutation or a pathogenic SNP. In some embodiments, said deamination of said nucleotide at said target locus of interest inactivates a target gene at said target locus.

In some embodiments, the engineered adenosine deaminase, or one or more components of the system are delivered by liposomes, nanoparticles, exosomes, microvesicles, nucleic acid nanoassemblies, a gene gun, an implantable device, or the vector system. In some embodiments, modification of the nucleotide modifies gene product encoded at the target locus or expression of the gene product.

### Base Editing

One aspect of the invention provides an RNA base editing system. In general, such a system may comprise a deaminase (e.g., an adenosine deaminase or cytidine deaminase) fused with a subject CasPR protein. The CasPR protein may be a dead Cas protein. In certain examples, the system comprises a mutated form of an adenosine deaminase fused with a dead CasPR. The mutated form of the adenosine deaminase may have both adenosine deaminase and cytidine deaminase activities.

### Adenosine Deaminase

The term “adenosine deaminase” or “adenosine deaminase protein” as used herein refers to a protein, a polypeptide, or one or more functional domain(s) of a protein or a polypeptide that is capable of catalyzing a hydrolytic deamination reaction that converts an adenine (or an adenine moiety of a molecule) to a hypoxanthine (or a hypoxanthine moiety of a molecule). In some embodiments, the adenine-containing molecule is an adenosine (A), and the hypoxanthine-containing molecule is an inosine (I). The adenine-containing molecule can be deoxyribonucleic acid (DNA) or ribonucleic acid (RNA).

According to the present disclosure, adenosine deaminases that can be used in connection with the present disclosure include, but are not limited to, members of the enzyme family known as adenosine deaminases that act on RNA (ADARs), members of the enzyme family known as adenosine deaminases that act on tRNA (ADATs), and other adenosine deaminase domain-containing (AD AD) family members. According to the present disclosure, the adenosine deaminase is capable of targeting adenine in a RNA/DNA and RNA duplexes. Indeed, Zheng et al. (Nucleic Acids Res. 45(6):3369-3377, 2017) demonstrate that ADARs can carry out adenosine to inosine editing reactions on RNA/DNA and RNA/RNA duplexes. In particular embodiments, the adenosine deaminase has been modified to increase its ability to edit DNA in a RNA/DNA heteroduplex of in an RNA duplex as detailed herein below.

In some embodiments, the adenosine deaminase is derived from one or more metazoa species, including but not limited to, mammals, birds, frogs, squids, fish, flies and worms. In some embodiments, the adenosine deaminase is a human, squid or Drosophila adenosine deaminase.

In some embodiments, the adenosine deaminase is a human ADAR, including hADAR1, hADAR2, hADAR3. In some embodiments, the adenosine deaminase is a Caenorhabditis elegans ADAR protein, including ADR-1 and ADR-2. In some embodiments, the adenosine deaminase is a Drosophila ADAR protein, including dAdar. In some embodiments, the adenosine deaminase is a squid Loligo pealeii ADAR protein, including sqADAR2a and sqADAR2b. In some embodiments, the adenosine deaminase is a human AD AT protein. In some embodiments, the adenosine deaminase is a Drosophila AD AT protein. In some embodiments, the adenosine deaminase is a human AD protein, including TENR (hADAD1) and TENRL (hADAD2).

In some embodiments, the adenosine deaminase is a TadA protein such as E. coli TadA. See Kim et al., Biochemistry 45:6407-6416 (2006); Wolf et al., EMBO J. 21:3841-3851, 2002. In some embodiments, the adenosine deaminase is mouse ADA. See Grunebaum et al., Curr. Opin. Allergy Clin. Immunol. 13:630-638, 2013. In some embodiments, the adenosine deaminase is human ADAT2. See Fukui et al., J. Nucleic Acids 2010:260512, 2010. In some embodiments, the deaminase (e.g., adenosine or cytidine deaminase) is one or more of those described in Cox et al., Science 358(6366):1019-1027, 2017; Komore et al., Nature 533(7603):420-4, 2016; and Gaudelli et al., Nature 551(7681):464-471, 2017.

In some embodiments, the adenosine deaminase protein recognizes and converts one or more target adenosine residue(s) in a double-stranded nucleic acid substrate (such as RNA:RNA duplex) into inosine residues. In some embodiments, the adenosine deaminase protein recognizes a binding window on the double-stranded substrate. In some embodiments, the binding window contains at least one target adenosine residue(s). In some embodiments, the binding window is in the range of about 3 bp to about 100 bp. In some embodiments, the binding window is in the range of about 5 bp to about 50 bp. In some embodiments, the binding window is in the range of about 10 bp to about 30 bp. In some embodiments, the binding window is about 1 bp, 2 bp, 3 bp, 5 bp, 7 bp, 10 bp, 15 bp, 20 bp, 25 bp, 30 bp, 40 bp, 45 bp, 50 bp, 55 bp, 60 bp, 65 bp, 70 bp, 75 bp, 80 bp, 85 bp, 90 bp, 95 bp, or 100 bp.

In some embodiments, the adenosine deaminase protein comprises one or more deaminase domains. The deaminase domain is believed to function to recognize and convert one or more target adenosine (A) residue(s) contained in a double-stranded nucleic acid substrate into inosine (I) residue(s). In some embodiments, the deaminase domain comprises an active center. In some embodiments, the active center comprises a zinc ion. In some embodiments, during the A-to-I editing process, base pairing at the target adenosine residue is disrupted, and the target adenosine residue is “flipped” out of the double helix to become accessible by the adenosine deaminase. In some embodiments, amino acid residues in or near the active center interact with one or more nucleotide(s) 5′ to a target adenosine residue. In some embodiments, amino acid residues in or near the active center interact with one or more nucleotide(s) 3′ to a target adenosine residue. In some embodiments, amino acid residues in or near the active center further interact with the nucleotide complementary to the target adenosine residue on the opposite strand. In some embodiments, the amino acid residues form hydrogen bonds with the T hydroxyl group of the nucleotides.

In some embodiments, the adenosine deaminase comprises human ADAR2 full protein (hADAR2) or the deaminase domain thereof (hADAR2-D). In some embodiments, the adenosine deaminase is an ADAR family member that is homologous to hADAR2 or hADAR2-D.

Particularly, in some embodiments, the homologous ADAR protein is human ADAR1 (hADAR1) or the deaminase domain thereof (hADAR1-D). In some embodiments, glycine 1007 of hADAR1-D corresponds to glycine 487 hADAR2-D, and glutamic Acid 1008 of hADAR1-D corresponds to glutamic acid 488 of hADAR2-D.

In some embodiments, the adenosine deaminase comprises the wild-type amino acid sequence of hADAR2-D. In some embodiments, the adenosine deaminase comprises one or more mutations in the hADAR2-D sequence, such that the editing efficiency, and/or substrate editing preference of hADAR2-D is changed according to specific needs. The engineered adenosine deaminase may be fused with a CasPR protein, e.g., Cas5d, Cas6 (e.g., Cas6e, Cas6f, etc.), Csf5, or an engineered form of the CasPR protein (e.g., an inactive, dead form, etc). In some examples, provided herein include an adenosine deaminase fused with a dead CasPR.

Certain mutations of hADAR1 and hADAR2 proteins have been described in Kuttan et al., Proc Natl Acad Sci USA 109(48):E3295-304, 2012; Want et al., ACS Chem Biol. 10(11):2512-9, 2015; and Zheng et al., Nucleic Acids Res. 45(6):3369-337, 2017, each of which is incorporated herein by reference in its entirety.

In some embodiments, the adenosine deaminase comprises a mutation at G336 of the hADAR2-D amino acid sequence, or a corresponding position in a homologous ADAR protein. In some embodiments, G336 is replaced by an aspartic acid residue (G336D).

In some embodiments, the adenosine deaminase comprises a mutation at G487 of the hADAR2-D amino acid sequence, or a corresponding position in a homologous ADAR protein.

In some embodiments, the glycine residue at position 487 is replaced by a non-polar amino acid residue with relatively small side chains. For example, in some embodiments, the glycine residue at position 487 is replaced by an alanine residue (G487A). In some embodiments, G487 is replaced by a valine residue (G487V). In some embodiments, G487 is replaced by an amino acid residue with relatively large side chains. In some embodiments, G487 is replaced by a arginine residue (G487R). In some embodiments, G487 is replaced by a lysine residue (G487K). In some embodiments, G487 is replaced by a tryptophan residue (G487W). In some embodiments, G487 is replaced by a tyrosine residue (G487Y).

In some embodiments, the adenosine deaminase comprises a mutation at E488 of the hADAR2-D amino acid sequence, or a corresponding position in a homologous ADAR protein. In some embodiments, E488 is replaced by a glutamine residue (E488Q). In some embodiments, E488 is replaced by a histidine residue (E488H). In some embodiments, E488 is replace by an arginine residue (E488R). In some embodiments, E488 is replace by a lysine residue (E488K). In some embodiments, E488 is replace by an asparagine residue (E488N). In some embodiments, E488 is replace by an alanine residue (E488A). In some embodiments, E488 is replace by a Methionine residue (E488M). In some embodiments, E488 is replace by a serine residue (E488S). In some embodiments, E488 is replace by a phenylalanine residue (E488F). In some embodiments, E488 is replace by a lysine residue (E488L). In some embodiments, E488 is replace by a tryptophan residue (E488W).

In some embodiments, the adenosine deaminase comprises a mutation at T490 of the hADAR2-D amino acid sequence, or a corresponding position in a homologous ADAR protein. In some embodiments, T490 is replaced by a cysteine residue (T490C), by a serine residue (T490S), by an alanine residue (T490A), by a phenylalanine residue (T490F), by a tyrosine residue (T490Y), by a serine residue (T490R), by an alanine residue (T490K), by a phenylalanine residue (T490P), or by a tyrosine residue (T490E).

In some embodiments, the adenosine deaminase comprises a mutation at V493 of the hADAR2-D amino acid sequence, or a corresponding position in a homologous ADAR protein. In some embodiments, V493 is replaced by an alanine residue (V493A), by a serine residue (V493S), by a threonine residue (V493T), by an arginine residue (V493R), by an aspartic acid residue (V493D), by a proline residue (V493P), or by a glycine residue (V493G).

In some embodiments, the adenosine deaminase comprises a mutation at A589 of the hADAR2-D amino acid sequence, or a corresponding position in a homologous ADAR protein. In some embodiments, A589 is replaced by a valine residue (A589V).

In some embodiments, the adenosine deaminase comprises a mutation at N597 of the hADAR2-D amino acid sequence, or a corresponding position in a homologous ADAR protein. In some embodiments, N597 is replaced by a lysine residue (N597K). In some embodiments, the adenosine deaminase comprises a mutation at position 597 of the amino acid sequence, which has an asparagine residue in the wild type sequence. In some embodiments, N597 is replaced by an arginine residue (N597R), by an alanine residue (N597A), by a glutamic acid residue (N597E), by a histidine residue (N597H), by a glycine residue (N597G), by a tyrosine residue (N597Y), or by a phenylalanine residue (N597F). In some embodiments, the adenosine deaminase comprises mutation N597I, N597L, N597V, N597M, N597C, N597P, N597T, N597S, N597Q, or N597D. In certain embodiments, the mutations at N597 are further made in the context of an E488Q background.

In some embodiments, the adenosine deaminase comprises a mutation at serine 599 of the hADAR2-D amino acid sequence, or a corresponding position in a homologous ADAR protein. In some embodiments, S599 is replaced by a threonine residue (S599T).

In some embodiments, the adenosine deaminase comprises a mutation at N613 of the hDAR2-D amino acid sequence, or a corresponding position in a homologous ADAR protein. In some embodiments, N613 is replaced by a lysine residue (N613K), by an arginine residue (N613R), by an alanine residue (N613A), by a glutamic acid residue (N613E). In some embodiments, the adenosine deaminase comprises mutation N613I, N613L, N613V, N613F, N613M, N613G, N613T, N613S, N613Y, N613W, N613Q, N613H, or N613D. In some embodiments, the mutations at N613 described above are further made in combination with a E488Q mutation.

In some embodiments, to improve editing efficiency, the adenosine deaminase may comprise one or more of the mutations: G336D, G487A, G487V, E488Q, E488H, E488R, E488N, E488A, E488S, E488M, T490C, T490S, V493T, V493S, V493A, V493R, V493D, V493P, V493G, N597K, N597R, N597A, N597E, N597H, N597G, N597Y, A589V, S599T, N613K, N613R, N613A, N613E, based on amino acid sequence positions of hADAR2-D, and mutations in a homologous ADAR protein corresponding to the above.

In some embodiments, to reduce editing efficiency, the adenosine deaminase may comprise one or more of the mutations: E488F, E488L, E488W, T490A, T490F, T490Y, T490R, T490K, T490P, T490E, N597F, based on amino acid sequence positions of hADAR2-D, and mutations in a homologous ADAR protein corresponding to the above. In particular embodiments, it can be of interest to use an adenosine deaminase enzyme with reduced efficacy to reduce off-target effects.

In some embodiments, to reduce off-target effects, the adenosine deaminase comprises one or more of mutations at R348, V351, T375, K376, E396, C451, R455, N473, R474, K475, R477, R481, S486, E488, T490, S495, R510, based on amino acid sequence positions of hADAR2-D, and mutations in a homologous ADAR protein corresponding to the above. In some embodiments, the adenosine deaminase comprises mutation at E488 and one or more additional positions selected from R348, V351, T375, K376, E396, C451, R455, N473, R474, K475, R477, R481, S486, T490, S495, R510. In some embodiments, the adenosine deaminase comprises mutation at T375, and optionally at one or more additional positions. In some embodiments, the adenosine deaminase comprises mutation at N473, and optionally at one or more additional positions. In some embodiments, the adenosine deaminase comprises mutation at V351, and optionally at one or more additional positions. In some embodiments, the adenosine deaminase comprises mutation at E488 and T375, and optionally at one or more additional positions. In some embodiments, the adenosine deaminase comprises mutation at E488 and N473, and optionally at one or more additional positions. In some embodiments, the adenosine deaminase comprises mutation E488 and V351, and optionally at one or more additional positions. In some embodiments, the adenosine deaminase comprises mutation at E488 and one or more of T375, N473, and V351.

In some embodiments, to reduce off-target effects, the adenosine deaminase comprises one or more of mutations selected from R348E, V351L, T375G, T375S, R455G, R455S, R455E, N473D, R474E, K475Q, R477E, R481E, S486T, E488Q, T490A, T490S, S495T, and R510E, based on amino acid sequence positions of hADAR2-D, and mutations in a homologous ADAR protein corresponding to the above. In some embodiments, the adenosine deaminase comprises mutation E488Q and one or more additional mutations selected from R348E, V351L, T375G, T375S, R455G, R455S, R455E, N473D, R474E, K475Q, R477E, R481E, S486T, T490A, T490S, S495T, and R510E. In some embodiments, the adenosine deaminase comprises mutation T375G or T375S, and optionally one or more additional mutations. In some embodiments, the adenosine deaminase comprises mutation N473D, and optionally one or more additional mutations. In some embodiments, the adenosine deaminase comprises mutation V351L, and optionally one or more additional mutations. In some embodiments, the adenosine deaminase comprises mutation E488Q, and T375G or T375G, and optionally one or more additional mutations. In some embodiments, the adenosine deaminase comprises mutation E488Q and N473D, and optionally one or more additional mutations. In some embodiments, the adenosine deaminase comprises mutation E488Q and V351L, and optionally one or more additional mutations. In some embodiments, the adenosine deaminase comprises mutation E488Q and one or more of T375G/S, N473D and V351L.

In certain examples, the adenosine deaminase protein or catalytic domain thereof has been modified to comprise a mutation at E488, preferably E488Q, of the hADAR2-D amino acid sequence, or a corresponding position in a homologous ADAR protein and/or wherein the adenosine deaminase protein or catalytic domain thereof has been modified to comprise a mutation at T375, preferably T375G of the hADAR2-D amino acid sequence, or a corresponding position in a homologous ADAR protein. In certain examples, the adenosine deaminase protein or catalytic domain thereof has been modified to comprise a mutation at E1008, preferably E1008Q, of the hADAR1d amino acid sequence, or a corresponding position in a homologous ADAR protein.

Crystal structures of the human ADAR2 deaminase domain bound to duplex RNA reveal a protein loop that binds the RNA on the 5′ side of the modification site. This 5′ binding loop is one contributor to substrate specificity differences between ADAR family members. See Wang et al., Nucleic Acids Res., 44(20):9872-9880 (2016), the content of which is incorporated herein by reference in its entirety. In addition, an ADAR2-specific RNA-binding loop was identified near the enzyme active site. See Mathews et al., Nat. Struct. Mol. Biol., 23(5):426-33 (2016), the content of which is incorporated herein by reference in its entirety. In some embodiments, the adenosine deaminase comprises one or more mutations in the RNA binding loop to improve editing specificity and/or efficiency.

In some embodiments, the adenosine deaminase comprises a mutation at A454 of the hADAR2-D amino acid sequence, or a corresponding position in a homologous ADAR protein. In some embodiments, A454 is replaced by a serine residue (A454S), by a cysteine residue (A454C), or by an aspartic acid residue (A454D).

In some embodiments, the adenosine deaminase comprises a mutation at R455 of the hADAR2-D amino acid sequence, or a corresponding position in a homologous ADAR protein. In some embodiments, R455 is replaced by an alanine residue (R455A), by a valine residue (R455V), by a histidine residue (R455H), by a glycine residue (R455G), by a serine residue (R455S), by a glutamic acid residue (R455E). In some embodiments, the adenosine deaminase comprises mutation R455C, R455I, R455K, R455L, R455M, R455N, R455Q, R455F, R455W, R455P, R455Y, R455E, or R455D. In some embodiments, the mutations at R455 described above are further made in combination with a E488Q mutation.

In some embodiments, the adenosine deaminase comprises a mutation at 1456 of the hADAR2-D amino acid sequence, or a corresponding position in a homologous ADAR protein. In some embodiments, 1456 is replaced by a valine residue (I456V), by a leucine residue (I456L), or by an aspartic acid residue (I456D).

In some embodiments, the adenosine deaminase comprises a mutation at F457 of the hADAR2-D amino acid sequence, or a corresponding position in a homologous ADAR protein. In some embodiments, F457 is replaced by a tyrosine residue (F457Y), by an arginine residue (F457R), or by a glutamic acid residue (F457E).

In some embodiments, the adenosine deaminase comprises a mutation at S458 of the hADAR2-D amino acid sequence, or a corresponding position in a homologous ADAR protein. In some embodiments, S458 is replaced by a valine residue (S458V), by a phenylalanine residue (S458F), by a proline residue (S458P). In some embodiments, the adenosine deaminase comprises mutation S458I, S458L, S458M, S458C, S458A, S458G, S458T, S458Y, S458W, S458Q, S458N, S458H, S458E, S458D, S458K, or S458R. In some embodiments, the mutations at S458 described above are further made in combination with a E488Q mutation.

In some embodiments, the adenosine deaminase comprises a mutation at P459 of the hADAR2-D amino acid sequence, or a corresponding position in a homologous ADAR protein. In some embodiments, P459 is replaced by a cysteine residue (P459C), by a histidine residue (P459H), or by a tryptophan residue (P459W).

In some embodiments, the adenosine deaminase comprises a mutation at H460 of the hADAR2-D amino acid sequence, or a corresponding position in a homologous ADAR protein. In some embodiments, H460 is replaced by an arginine residue (H460R), by an isoleucine residue (H460I), by a proline residue (H460P). In some embodiments, the adenosine deaminase comprises mutation H460L, H460V, H460F, H460M, H460C, H460A, H460G, H460T, H460S, H460Y, H460W, H460Q, H460N, H460E, H460D, or H460K. In some embodiments, the mutations at H460 described above are further made in combination with a E488Q mutation.

In some embodiments, the adenosine deaminase comprises a mutation at P462 of the hADAR2-D amino acid sequence, or a corresponding position in a homologous ADAR protein. In some embodiments, P462 is replaced by a serine residue (P462S), by a tryptophan residue (P462W), or by a glutamic acid residue (P462E).

In some embodiments, the adenosine deaminase comprises a mutation at aspartic acid 469 of the hADAR2-D amino acid sequence, or a corresponding position in a homologous ADAR protein. In some embodiments, the aspartic acid residue at position 469 is replaced by a glutamine residue (D469Q). In some embodiments, the aspartic acid residue at position 469 is replaced by a serine residue (D469S). In some embodiments, the aspartic acid residue at position 469 is replaced by a tyrosine residue (D469Y).

In some embodiments, the adenosine deaminase comprises a mutation at R470 of the hADAR2-D amino acid sequence, or a corresponding position in a homologous ADAR protein. In some embodiments, R470 is replaced by an alanine residue (R470A), by an isoleucine residue (R470I), or by an aspartic acid residue (R470D).

In some embodiments, the adenosine deaminase comprises a mutation at H471 of the hADAR2-D amino acid sequence, or a corresponding position in a homologous ADAR protein. In some embodiments, H471 is replaced by a lysine residue (H471K), by a threonine residue (H471T), or by a valine residue (H471V).

In some embodiments, the adenosine deaminase comprises a mutation at P472 of the hADAR2-D amino acid sequence, or a corresponding position in a homologous ADAR protein. In some embodiments, P472 is replaced by a lysine residue (P472K), by a threonine residue (P472T), or by an aspartic acid residue (P472D).

In some embodiments, the adenosine deaminase comprises a mutation at N473 of the hADAR2-D amino acid sequence, or a corresponding position in a homologous ADAR protein. In some embodiments, N473 is replaced by an arginine residue (N473R), by a tryptophan residue (N473W), by a proline residue (N473P), or by an aspartic acid residue (N473D).

In some embodiments, the adenosine deaminase comprises a mutation at R474 of the hADAR2-D amino acid sequence, or a corresponding position in a homologous ADAR protein. In some embodiments, R474 is replaced by a lysine residue (R474K), by a glycine residue (R474G), by an aspartic acid residue (R474D), or by a glutamic acid residue (R474E).

In some embodiments, the adenosine deaminase comprises a mutation at K475 of the hADAR2-D amino acid sequence, or a corresponding position in a homologous ADAR protein. In some embodiments, K475 is replaced by a glutamine residue (K475Q), by an asparagine residue (K475N), or by an aspartic acid residue (K475D).

In some embodiments, the adenosine deaminase comprises a mutation at A476 of the hADAR2-D amino acid sequence, or a corresponding position in a homologous ADAR protein. In some embodiments, A476 is replaced by a serine residue (A476S), by an arginine residue (A476R), or by a glutamic acid residue (A476E).

In some embodiments, the adenosine deaminase comprises a mutation at R477 of the hADAR2-D amino acid sequence, or a corresponding position in a homologous ADAR protein. In some embodiments, R477 is replaced by a lysine residue (R477K), by a threonine residue (R477T), by a phenylalanine residue (R477F), or by a glutamic acid residue (R477E).

In some embodiments, the adenosine deaminase comprises a mutation at G478 of the hADAR2-D amino acid sequence, or a corresponding position in a homologous ADAR protein. In some embodiments, G478 is replaced by an alanine residue (G478A), by an arginine residue (G478R), or by a tyrosine residue (G478Y). In some embodiments, the adenosine deaminase comprises mutation G478I, G478L, G478V, G478F, G478M, G478C, G478P, G478T, G478S, G478W, G478Q, G478N, G478H, G478E, G478D, or G478K. In some embodiments, the mutations at G478 described above are further made in combination with a E488Q mutation.

In some embodiments, the adenosine deaminase comprises a mutation at E479 of the hADAR2-D amino acid sequence, or a corresponding position in a homologous ADAR protein. In some embodiments, Q479 is replaced by an asparagine residue (Q479N), by a serine residue (Q479S), or by a proline residue (Q479P).

In some embodiments, the adenosine deaminase comprises a mutation at R348 of the hADAR2-D amino acid sequence, or a corresponding position in a homologous ADAR protein. In some embodiments, R348 is replaced by an alanine residue (R348A), or by a glutamic acid residue (R348E).

In some embodiments, the adenosine deaminase comprises a mutation at V351 of the hADAR2-D amino acid sequence, or a corresponding position in a homologous ADAR protein. In some embodiments, V351 is replaced by a leucine residue (V351L). In some embodiments, the adenosine deaminase comprises mutation V351Y, V351M, V351T, V351G, V351A, V351F, V351E, V351I, V351C, V351H, V351P, V351 S, V351K, V351N, V351W, V351Q, V351D, or V351R. In some embodiments, the mutations at V351 described above are further made in combination with a E488Q mutation.

In some embodiments, the adenosine deaminase comprises a mutation at T375 of the hADAR2-D amino acid sequence, or a corresponding position in a homologous ADAR protein. In some embodiments, T375 is replaced by a glycine residue (T375G), or by a serine residue (T375S). In some embodiments, the adenosine deaminase comprises mutation T375H, T375Q, T375C, T375N, T375M, T375A, T375W, T375V, T375R, T375E, T375K, T375F, T375I, T375D, T375P, T375L, or T375Y. In some embodiments, the mutations at T375Y described above are further made in combination with an E488Q mutation.

In some embodiments, the adenosine deaminase comprises a mutation at R481 of the hADAR2-D amino acid sequence, or a corresponding position in a homologous ADAR protein. In some embodiments, R481 is replaced by a glutamic acid residue (R481E).

In some embodiments, the adenosine deaminase comprises a mutation at S486 of the hADAR2-D amino acid sequence, or a corresponding position in a homologous ADAR protein. In some embodiments, S486 is replaced by a threonine residue (S486T).

In some embodiments, the adenosine deaminase comprises a mutation at T490 of the hADAR2-D amino acid sequence, or a corresponding position in a homologous ADAR protein. In some embodiments, T490 is replaced by an alanine residue (T490A), or by a serine residue (T490S).

In some embodiments, the adenosine deaminase comprises a mutation at S495 of the hADAR2-D amino acid sequence, or a corresponding position in a homologous ADAR protein. In some embodiments, S495 is replaced by a threonine residue (S495T).

In some embodiments, the adenosine deaminase comprises a mutation at R510 of the hADAR2-D amino acid sequence, or a corresponding position in a homologous ADAR protein. In some embodiments, R510 is replaced by a glutamine residue (R510Q), by an alanine residue (R510A), or by a glutamic acid residue (R510E).

In some embodiments, the adenosine deaminase comprises a mutation at G593 of the hADAR2-D amino acid sequence, or a corresponding position in a homologous ADAR protein. In some embodiments, G593 is replaced by an alanine residue (G593A), or by a glutamic acid residue (G593E).

In some embodiments, the adenosine deaminase comprises a mutation at K594 of the hADAR2-D amino acid sequence, or a corresponding position in a homologous ADAR protein. In some embodiments, K594 is replaced by an alanine residue (K594A).

In some embodiments, the adenosine deaminase comprises a mutation at any one or more of positions A454, R455, 1456, F457, S458, P459, H460, P462, D469, R470, H471, P472, N473, R474, K475, A476, R477, G478, Q479, R348, R510, G593, K594 of the hADAR2-D amino acid sequence, or a corresponding position in a homologous ADAR protein.

In some embodiments, the adenosine deaminase comprises any one or more of mutations A454S, A454C, A454D, R455A, R455V, R455H, I456V, I456L, I456D, F457Y, F457R, F457E, S458V, S458F, S458P, P459C, P459H, P459W, H460R, H460I, H460P, P462S, P462W, P462E, D469Q, D469S, D469Y, R470A, R470I, R470D, H471K, H471T, H471V, P472K, P472T, P472D, N473R, N473W, N473P, R474K, R474G, R474D, K475Q, K475N, K475D, A476S, A476R, A476E, R477K, R477T, R477F, G478A, G478R, G478Y, Q479N, Q479S, Q479P, R348A, R510Q, R510A, G593A, G593E, K594A of the hADAR2-D amino acid sequence, or a corresponding position in a homologous ADAR protein.

In some embodiments, the adenosine deaminase comprises a mutation at any one or more of positions T375, V351, G478, S458, H460 of the hADAR2-D amino acid sequence, or a corresponding position in a homologous ADAR protein, optionally in combination a mutation at E488. In some embodiments, the adenosine deaminase comprises one or more of mutations selected from T375G, T375C, T375H, T375Q, V351M, V351T, V351Y, G478R, S458F, H460I, optionally in combination with E488Q.

In some embodiments, the adenosine deaminase comprises one or more of mutations selected from T375H, T375Q, V351M, V351Y, H460P, optionally in combination with E488Q.

In some embodiments, the adenosine deaminase comprises mutations T375S and S458F, optionally in combination with E488Q.

In some embodiments, the adenosine deaminase comprises a mutation at two or more of positions T375, N473, R474, G478, S458, P459, V351, R455, R455, T490, R348, Q479 of the hADAR2-D amino acid sequence, or a corresponding position in a homologous ADAR protein, optionally in combination a mutation at E488. In some embodiments, the adenosine deaminase comprises two or more of mutations selected from T375G, T375S, N473D, R474E, G478R, S458F, P459W, V351L, R455G, R455S, T490A, R348E, Q479P, optionally in combination with E488Q.

In some embodiments, the adenosine deaminase comprises mutations T375G and V351L. In some embodiments, the adenosine deaminase comprises mutations T375G and R455G. In some embodiments, the adenosine deaminase comprises mutations T375G and R455S. In some embodiments, the adenosine deaminase comprises mutations T375G and T490A. In some embodiments, the adenosine deaminase comprises mutations T375G and R348E. In some embodiments, the adenosine deaminase comprises mutations T375S and V351L. In some embodiments, the adenosine deaminase comprises mutations T375S and R455G. In some embodiments, the adenosine deaminase comprises mutations T375S and R455S. In some embodiments, the adenosine deaminase comprises mutations T375S and T490A. In some embodiments, the adenosine deaminase comprises mutations T375S and R348E. In some embodiments, the adenosine deaminase comprises mutations N473D and V351L. In some embodiments, the adenosine deaminase comprises mutations N473D and R455G. In some embodiments, the adenosine deaminase comprises mutations N473D and R455S. In some embodiments, the adenosine deaminase comprises mutations N473D and T490A. In some embodiments, the adenosine deaminase comprises mutations N473D and R348E. In some embodiments, the adenosine deaminase comprises mutations R474E and V351L. In some embodiments, the adenosine deaminase comprises mutations R474E and R455G. In some embodiments, the adenosine deaminase comprises mutations R474E and R455S. In some embodiments, the adenosine deaminase comprises mutations R474E and T490A. In some embodiments, the adenosine deaminase comprises mutations R474E and R348E. In some embodiments, the adenosine deaminase comprises mutations S458F and T375G. In some embodiments, the adenosine deaminase comprises mutations S458F and T375S. In some embodiments, the adenosine deaminase comprises mutations S458F and N473D. In some embodiments, the adenosine deaminase comprises mutations S458F and R474E. In some embodiments, the adenosine deaminase comprises mutations S458F and G478R. In some embodiments, the adenosine deaminase comprises mutations G478R and T375G. In some embodiments, the adenosine deaminase comprises mutations G478R and T375S. In some embodiments, the adenosine deaminase comprises mutations G478R and N473D. In some embodiments, the adenosine deaminase comprises mutations G478R and R474E. In some embodiments, the adenosine deaminase comprises mutations P459W and T375G. In some embodiments, the adenosine deaminase comprises mutations P459W and T375S. In some embodiments, the adenosine deaminase comprises mutations P459W and N473D. In some embodiments, the adenosine deaminase comprises mutations P459W and R474E. In some embodiments, the adenosine deaminase comprises mutations P459W and G478R. In some embodiments, the adenosine deaminase comprises mutations P459W and S458F. In some embodiments, the adenosine deaminase comprises mutations Q479P and T375G. In some embodiments, the adenosine deaminase comprises mutations Q479P and T375S. In some embodiments, the adenosine deaminase comprises mutations Q479P and N473D. In some embodiments, the adenosine deaminase comprises mutations Q479P and R474E. In some embodiments, the adenosine deaminase comprises mutations Q479P and G478R. In some embodiments, the adenosine deaminase comprises mutations Q479P and S458F. In some embodiments, the adenosine deaminase comprises mutations Q479P and P459W. All mutations described in this paragraph may also further be made in combination with a E488Q mutations.

In some embodiments, the adenosine deaminase comprises a mutation at any one or more of positions K475, Q479, P459, G478, S458 of the hADAR2-D amino acid sequence, or a corresponding position in a homologous ADAR protein, optionally in combination a mutation at E488. In some embodiments, the adenosine deaminase comprises one or more of mutations selected from K475N, Q479N, P459W, G478R, S458P, S458F, optionally in combination with E488Q.

In some embodiments, the adenosine deaminase comprises a mutation at any one or more of positions T375, V351, R455, H460, A476 of the hADAR2-D amino acid sequence, or a corresponding position in a homologous ADAR protein, optionally in combination a mutation at E488. In some embodiments, the adenosine deaminase comprises one or more of mutations selected from T375G, T375C, T375H, T375Q, V351M, V351T, V351Y, R455H, H460P, H460I, A476E, optionally in combination with E488Q.

In certain embodiments, improvement of editing and reduction of off-target modification is achieved by chemical modification of gRNAs. gRNAs which are chemically modified as exemplified in Vogel et al. (2014), Angew Chem Int Ed, 53:6267-6271, doi: 10. 1002/anie.201402634 (incorporated herein by reference in its entirety) reduce off-target activity and improve on-target efficiency. 2′-O-methyl and phosphothioate modified guide RNAs in general improve editing efficiency in cells.

ADAR has been known to demonstrate a preference for neighboring nucleotides on either side of the edited A (Matthews et al., Nature Structural Mol Biol 23(5):426-433, 2017, incorporated herein by reference in its entirety). Accordingly, in certain embodiments, the gRNA, target, and/or ADAR is selected optimized for motif preference.

Intentional mismatches have been demonstrated in vitro to allow for editing of non-preferred motifs (academic.oup.com/nar/article-lookup/doi/10. 1093/nar/gku272; Schneider et al (2014), Nucleic Acid Res, 42(10):e87); Fukuda et al. (2017), Scientific Reports, 7, doi: 10. 1038/srep41478, incorporated herein by reference in its entirety). Accordingly, in certain embodiments, to enhance RNA editing efficiency on non-preferred 5′ or 3′ neighboring bases, intentional mismatches in neighboring bases are introduced.

In some embodiments, the adenosine deaminase may be a tRNA-specific adenosine deaminase or a variant thereof. In some embodiments, the adenosine deaminase may comprise one or more of the mutations: W23L, W23R, R26G, H36L, N37S, P48S, P48T, P48A, I49V, R51L, N72D, L84F, S97C, A106V, D108N, H123Y, G125A, A142N, S146C, D147Y, R152H, R152P, E155V, I156F, K157N, K161T, based on amino acid sequence positions of E. coli TadA, and mutations in a homologous deaminase protein corresponding to the above. In some embodiments, the adenosine deaminase may comprise one or more of the mutations: D108N based on amino acid sequence positions of E. coli TadA, and mutations in a homologous deaminase protein corresponding to the above. In some embodiments, the adenosine deaminase may comprise one or more of the mutations: A106V, D108N, based on amino acid sequence positions of E. coli TadA, and mutations in a homologous deaminase protein corresponding to the above. In some embodiments, the adenosine deaminase may comprise one or more of the mutations: A106V, D108N, D147Y, E155V, based on amino acid sequence positions of E. coli TadA, and mutations in a homologous deaminase protein corresponding to the above. In some embodiments, the adenosine deaminase may comprise one or more of the mutations: A106V, D108N, based on amino acid sequence positions of E. coli TadA, and mutations in a homologous deaminase protein corresponding to the above. In some embodiments, the adenosine deaminase may comprise one or more of the mutations: A106V, D108N, D147Y, E155V, L84F, H123Y, I156F, based on amino acid sequence positions of E. coli TadA, and mutations in a homologous deaminase protein corresponding to the above. In some embodiments, the adenosine deaminase may comprise one or more of the mutations: A106V, D108N, D147Y, E155V, L84F, H123Y, I156F, A142N, based on amino acid sequence positions of E. coli TadA, and mutations in a homologous deaminase protein corresponding to the above. In some embodiments, the adenosine deaminase may comprise one or more of the mutations: A106V, D108N, D147Y, E155V, L84F, H123Y, I156F, H36L, R51L, S146C, K157N, based on amino acid sequence positions of E. coli TadA, and mutations in a homologous deaminase protein corresponding to the above. In some embodiments, the adenosine deaminase may comprise one or more of the mutations: A106V, D108N, D147Y, E155V, L84F, H123Y, I156F, H36L, R51L, S146C, K157N, P48S, based on amino acid sequence positions of E. coli TadA, and mutations in a homologous deaminase protein corresponding to the above. In some embodiments, the adenosine deaminase may comprise one or more of the mutations: A106V, D108N, D147Y, E155V, L84F, H123Y, I156F, H36L, R51L, S146C, K157N, P48S, A142N, based on amino acid sequence positions of E. coli TadA, and mutations in a homologous deaminase protein corresponding to the above. In some embodiments, the adenosine deaminase may comprise one or more of the mutations: A106V, D108N, D147Y, E155V, L84F, H123Y, I156F, H36L, R51L, S146C, K157N, P48S, W23R, P48A, based on amino acid sequence positions of E. coli TadA, and mutations in a homologous deaminase protein corresponding to the above. In some embodiments, the adenosine deaminase may comprise one or more of the mutations: A106V, D108N, D147Y, E155V, L84F, H123Y, I156F, H36L, R51L, S146C, K157N, P48S, W23R, P48A, A142N, based on amino acid sequence positions of E. coli TadA, and mutations in a homologous deaminase protein corresponding to the above. In some embodiments, the adenosine deaminase may comprise one or more of the mutations: A106V, D108N, D147Y, E155V, L84F, H123Y, I156F, H36L, R51L, S146C, K157N, P48S, W23R, P48A, R152P, based on amino acid sequence positions of E. coli TadA, and mutations in a homologous deaminase protein corresponding to the above. In some embodiments, the adenosine deaminase may comprise one or more of the mutations: A106V, D108N, D147Y, E155V, L84F, H123Y, I156F, H36L, R51L, S146C, K157N, P48S, W23R, P48A, R152P, A142N, based on amino acid sequence positions of E. coli TadA, and mutations in a homologous deaminase protein corresponding to the above.

Results suggest that A's opposite C's in the targeting window of the ADAR deaminase domain are preferentially edited over other bases. Additionally, A's base-paired with U's within a few bases of the targeted base show low levels of editing by CRISPR-Cas-ADAR fusions, suggesting that there is flexibility for the enzyme to edit multiple A's. These two observations suggest that multiple A's in the activity window of CasPR-ADAR fusions could be specified for editing by mismatching all A's to be edited with C's. Accordingly, in certain embodiments, multiple A:C mismatches in the activity window are designed to create multiple A:I edits. In certain embodiments, to suppress potential off-target editing in the activity window, non-target A's are paired with A's or G's.

The terms “editing specificity” and “editing preference” are used interchangeably herein to refer to the extent of A-to-I editing at a particular adenosine site in a double-stranded substrate. In some embodiment, the substrate editing preference is determined by the 5′ nearest neighbor and/or the 3′ nearest neighbor of the target adenosine residue. In some embodiments, the adenosine deaminase has preference for the 5′ nearest neighbor of the substrate ranked as U>A>C>G (“>” indicates greater preference). In some embodiments, the adenosine deaminase has preference for the 3′ nearest neighbor of the substrate ranked as G>C˜A>U (“>” indicates greater preference; indicates similar preference). In some embodiments, the adenosine deaminase has preference for the 3′ nearest neighbor of the substrate ranked as G>C>U˜A (“>” indicates greater preference; indicates similar preference). In some embodiments, the adenosine deaminase has preference for the 3′ nearest neighbor of the substrate ranked as G>C>A>U (“>” indicates greater preference). In some embodiments, the adenosine deaminase has preference for the 3′ nearest neighbor of the substrate ranked as C˜G˜A>U (“>” indicates greater preference; indicates similar preference). In some embodiments, the adenosine deaminase has preference for a triplet sequence containing the target adenosine residue ranked as TAG>AAG>CAC>AAT>GAA>GAC (“>” indicates greater preference), the center A being the target adenosine residue.

In some embodiments, the substrate editing preference of an adenosine deaminase is affected by the presence or absence of a nucleic acid binding domain in the adenosine deaminase protein. In some embodiments, to modify substrate editing preference, the deaminase domain is connected with a double-strand RNA binding domain (dsRBD) or a double-strand RNA binding motif (dsRBM). In some embodiments, the dsRBD or dsRBM may be derived from an ADAR protein, such as hADAR1 or hADAR2. In some embodiments, a full length ADAR protein that comprises at least one dsRBD and a deaminase domain is used. In some embodiments, the one or more dsRBM or dsRBD is at the N-terminus of the deaminase domain. In other embodiments, the one or more dsRBM or dsRBD is at the C-terminus of the deaminase domain.

In some embodiments, the substrate editing preference of an adenosine deaminase is affected by amino acid residues near or in the active center of the enzyme. In some embodiments, to modify substrate editing preference, the adenosine deaminase may comprise one or more of the mutations: G336D, G487R, G487K, G487W, G487Y, E488Q, E488N, T490A, V493A, V493T, V493S, N597K, N597R, A589V, S599T, N613K, N613R, based on amino acid sequence positions of hADAR2-D, and mutations in a homologous ADAR protein corresponding to the above.

Particularly, in some embodiments, to reduce editing specificity, the adenosine deaminase can comprise one or more of mutations E488Q, V493A, N597K, N613K, based on amino acid sequence positions of hADAR2-D, and mutations in a homologous ADAR protein corresponding to the above. In some embodiments, to increase editing specificity, the adenosine deaminase can comprise mutation T490A.

In some embodiments, to increase editing preference for target adenosine (A) with an immediate 5′ G, such as substrates comprising the triplet sequence GAC, the center A being the target adenosine residue, the adenosine deaminase can comprise one or more of mutations G336D, E488Q, E488N, V493T, V493S, V493A, A589V, N597K, N597R, S599T, N613K, N613R, based on amino acid sequence positions of hADAR2-D, and mutations in a homologous ADAR protein corresponding to the above.

Particularly, in some embodiments, the adenosine deaminase comprises mutation E488Q or a corresponding mutation in a homologous ADAR protein for editing substrates comprising the following triplet sequences: GAC, GAA, GAET, GAG, CAET, AAEG, ETAC, the center A being the target adenosine residue.

In some embodiments, the adenosine deaminase comprises the wild-type amino acid sequence of hADAR1-D. In some embodiments, the adenosine deaminase comprises one or more mutations in the hADAR1-D sequence, such that the editing efficiency, and/or substrate editing preference of hADAR1-D is changed according to specific needs.

In some embodiments, the adenosine deaminase comprises a mutation at G1007 of the hADAR1-D amino acid sequence, or a corresponding position in a homologous ADAR protein. In some embodiments, G1007 is replaced by a non-polar amino acid residue with relatively small side chains. For example, in some embodiments, G1007 is replaced by an alanine residue (G1007A), or by a valine residue (G1007V). In some embodiments, G1007 is replaced by an amino acid residue with relatively large side chains. In some embodiments, G1007 is replaced by an arginine residue (G1007R), by a lysine residue (G1007K), by a tryptophan residue (G1007W), or by a tyrosine residue (G1007Y). Additionally, in other embodiments, G1007 is replaced by a leucine residue (G1007L), by a threonine residue (G1007T), or by a serine residue (G1007S).

In some embodiments, the adenosine deaminase comprises a mutation at E1008 of the hADAR1-D amino acid sequence, or a corresponding position in a homologous ADAR protein. In some embodiments, E1008 is replaced by a polar amino acid residue having a relatively large side chain. In some embodiments, E1008 is replaced by a glutamine residue (E1008Q), by a histidine residue (E1008H), by an arginine residue (E1008R), or by a lysine residue (E1008K). In some embodiments, E1008 is replaced by a nonpolar or small polar amino acid residue. In some embodiments, E1008 is replaced by a phenylalanine residue (E1008F), by a tryptophan residue (E1008W), by a glycine residue (E1008G), by an isoleucine residue (E1008I), by a valine residue (E1008V), by a proline residue (E1008P), by a serine residue (E1008S), by an asparagine residue (E1008N), by an alanine residue (E1008A), by a Methionine residue (E1008M), or by a leucine residue (E1008L).

In some embodiments, to improve editing efficiency, the adenosine deaminase may comprise one or more of the mutations: E1007S, E1007A, E1007V, E1008Q, E1008R, E1008H, E1008M, E1008N, E1008K, based on amino acid sequence positions of hADAR1-D, and mutations in a homologous ADAR protein corresponding to the above.

In some embodiments, to reduce editing efficiency, the adenosine deaminase may comprise one or more of the mutations: E1007R, E1007K, E1007Y, E1007L, E1007T, E1008G, E1008I, E1008P, E1008V, E1008F, E1008W, E1008S, E1008N, E1008K, based on amino acid sequence positions of hADAR1-D, and mutations in a homologous ADAR protein corresponding to the above.

In some embodiments, the substrate editing preference, efficiency and/or selectivity of an adenosine deaminase is affected by amino acid residues near or in the active center of the enzyme. In some embodiments, the adenosine deaminase comprises a mutation at E1008 position in hADAR1-D sequence, or a corresponding position in a homologous ADAR protein. In some embodiments, the mutation is E1008R, or a corresponding mutation in a homologous ADAR protein. In some embodiments, the E1008R mutant has an increased editing efficiency for target adenosine residue that has a mismatched G residue on the opposite strand.

In some embodiments, the adenosine deaminase protein further comprises or is connected to one or more double-stranded RNA (dsRNA) binding motifs (dsRBMs) or domains (dsRBDs) for recognizing and binding to double-stranded nucleic acid substrates. In some embodiments, the interaction between the adenosine deaminase and the double-stranded substrate is mediated by one or more additional protein factor(s), including a CRISPR/CAS protein factor. In some embodiments, the interaction between the adenosine deaminase and the double-stranded substrate is further mediated by one or more nucleic acid component(s), including a guide RNA.

In certain example embodiments, directed evolution may be used to design modified ADAR proteins capable of catalyzing additional reactions besides deamination of a adenine to a hypoxanthine.

In certain embodiments, the adenosine deaminase protein is a modified adenosine deaminase with C to U deamination activity.

In certain example embodiments, directed evolution may be used to design modified ADAR proteins capable of catalyzing additional reactions besides deamination of an adenine to a hypoxanthine. For example, the modified ADAR protein may be capable of catalyzing deamination of a cytidine to a uracil. While not bound by a particular theory, mutations that improve C to U activity may alter the shape of the binding pocket to be more amenable to the smaller cytidine base. In some cases, the modified ADAR comprise mutations on residues the catalytic core and/or residues that contact the RNA target. Examples of mutations on residues in the catalytic core include V351G and K350L, based on amino acid sequence positions of hADAR2-D, and mutations in a homologous ADAR protein corresponding to the above. Examples of mutations on residues on the residues that contact with the RNA target include S486A and S495N, based on amino acid sequence positions of hADAR2-D, and mutations in a homologous ADAR protein corresponding to the above.

In certain embodiments the adenosine deaminase is engineered to convert the activity to cytidine deaminase. Such engineered adenosine deaminase may also retain its adenosine deaminase activity, i.e., such mutated adenosine deaminase may have both adenosine deaminase and cytidine deaminase activities. Accordingly in some embodiments, the adenosine deaminase comprises one or more mutations in positions selected from E396, C451, V351, R455, T375, K376, S486, Q488, R510, K594, R348, G593, S397, H443, L444, Y445, F442, E438, T448, A353, V355, T339, P539, T339, P539, V525 1520, P462 and N579. In particular embodiments, the adenosine deaminase comprises one or more mutations in a position selected from V351, L444, V355, V525 and 1520. In some embodiments, the adenosine deaminase may comprise one or more of mutations at E488, V351, S486, T375, S370, P462, N597, based on amino acid sequence positions of hADAR2-D, and mutations in a homologous ADAR protein corresponding to the above.

In some embodiments, the adenosine deaminase may comprise one or more of the mutations: E488Q based on amino acid sequence positions of hADAR2-D, and mutations in a homologous ADAR protein corresponding to the above. In some embodiments, the adenosine deaminase may comprise one or more of the mutations: E488Q, V351G, based on amino acid sequence positions of hADAR2-D, and mutations in a homologous ADAR protein corresponding to the above. In some embodiments, the adenosine deaminase may comprise one or more of the mutations: E488Q, V351G, S486A, based on amino acid sequence positions of hADAR2-D, and mutations in a homologous ADAR protein corresponding to the above. In some embodiments, the adenosine deaminase may comprise one or more of the mutations: E488Q, V351G, S486A, T375S, based on amino acid sequence positions of hADAR2-D, and mutations in a homologous ADAR protein corresponding to the above. In some embodiments, the adenosine deaminase may comprise one or more of the mutations: E488Q, V351G, S486A, T375S, S370C, based on amino acid sequence positions of hADAR2-D, and mutations in a homologous ADAR protein corresponding to the above. In some embodiments, the adenosine deaminase may comprise one or more of the mutations: E488Q, V351G, S486A, T375S, S370C, P462A, based on amino acid sequence positions of hADAR2-D, and mutations in a homologous ADAR protein corresponding to the above. In some embodiments, the adenosine deaminase may comprise one or more of the mutations: E488Q, V351G, S486A, T375S, S370C, P462A, N597I, based on amino acid sequence positions of hADAR2-D, and mutations in a homologous ADAR protein corresponding to the above. In some embodiments, the adenosine deaminase may comprise one or more of the mutations: E488Q, V351G, S486A, T375S, S370C, P462A, N597I, L332I, based on amino acid sequence positions of hADAR2-D, and mutations in a homologous ADAR protein corresponding to the above. In some embodiments, the adenosine deaminase may comprise one or more of the mutations: E488Q, V351G, S486A, T375S, S370C, P462A, N597I, L332I, I398V, based on amino acid sequence positions of hADAR2-D, and mutations in a homologous ADAR protein corresponding to the above. In some embodiments, the adenosine deaminase may comprise one or more of the mutations: E488Q, V351G, S486A, T375S, S370C, P462A, N597I, L332I, I398V, K350I, based on amino acid sequence positions of hADAR2-D, and mutations in a homologous ADAR protein corresponding to the above. In some embodiments, the adenosine deaminase may comprise one or more of the mutations: E488Q, V351G, S486A, T375S, S370C, P462A, N597I, L332I, I398V, K350I, M383L, based on amino acid sequence positions of hADAR2-D, and mutations in a homologous ADAR protein corresponding to the above. In some embodiments, the adenosine deaminase may comprise one or more of the mutations: E488Q, V351G, S486A, T375S, S370C, P462A, N597I, L332I, I398V, K350I, M383L, D619G, based on amino acid sequence positions of hADAR2-D, and mutations in a homologous ADAR protein corresponding to the above. In some embodiments, the adenosine deaminase may comprise one or more of the mutations: E488Q, V351G, S486A, T375S, S370C, P462A, N597I, L332I, I398V, K350I, M383L, D619G, S582T, based on amino acid sequence positions of hADAR2-D, and mutations in a homologous ADAR protein corresponding to the above. In some embodiments, the adenosine deaminase may comprise one or more of the mutations: E488Q, V351G, S486A, T375S, S370C, P462A, N597I, L332I, I398V, K350I, M383L, D619G, S582T, V440I based on amino acid sequence positions of hADAR2-D, and mutations in a homologous ADAR protein corresponding to the above. In some embodiments, the adenosine deaminase may comprise one or more of the mutations: E488Q, V351G, S486A, T375S, S370C, P462A, N597I, L332I, I398V, K350I, M383L, D619G, S582T, V440I, S495N based on amino acid sequence positions of hADAR2-D, and mutations in a homologous ADAR protein corresponding to the above. In some embodiments, the adenosine deaminase may comprise one or more of the mutations: E488Q, V351G, S486A, T375S, S370C, P462A, N597I, L332I, I398V, K350I, M383L, D619G, S582T, V440I, S495N, K418E based on amino acid sequence positions of hADAR2-D, and mutations in a homologous ADAR protein corresponding to the above. In some embodiments, the adenosine deaminase may comprise one or more of the mutations: E488Q, V351G, S486A, T375S, S370C, P462A, N597I, L332I, 1398V, K350I, M383L, D619G, S582T, V440I, S495N, K418E, S661T based on amino acid sequence positions of hADAR2-D, and mutations in a homologous ADAR protein corresponding to the above. In some examples, provided herein includes a mutated adenosine deaminase e.g., an adenosine deaminase comprising one or more mutations of E488Q, V351G, S486A, T375S, S370C, P462A, N597I, L332I, I398V, K350I, M383L, D619G, S582T, V440I, S495N, K418E, S661T (based on amino acid sequence positions of hADAR2-D, and mutations in a homologous ADAR protein corresponding to the above), fused with a dead CasPR protein. In a particular example, provided herein includes a mutated adenosine deaminase e.g., an adenosine deaminase comprising E488Q, V351G, S486A, T375S, S370C, P462A, N597I, L332I, I398V, K350I, M383L, D619G, S582T, V440I, S495N, K418E, and S661T (based on amino acid sequence positions of hADAR2-D, and mutations in a homologous ADAR protein corresponding to the above), fused with a dead CasPR protein.

In some embodiments, the modified adenosine deaminase having C-to-U deamination activity comprises a mutation at any one or more of positions V351, T375, R455, and E488 of the hADAR2-D amino acid sequence, or a corresponding position in a homologous ADAR protein. In some embodiments, the adenosine deaminase comprises mutation E488Q. In some embodiments, the adenosine deaminase comprises one or more of mutations selected from V351I, V351L, V351F, V351M, V351C, V351A, V351G, V351P, V351T, V351 S, V351Y, V351W, V351Q, V351N, V351H, V351E, V351D, V351K, V351R, T375I, T375L, T375V, T375F, T375M, T375C, T375A, T375G, T375P, T375S, T375Y, T375W, T375Q, T375N, T375H, T375E, T375D, T375K, T375R, R455I, R455L, R455V, R455F, R455M, R455C, R455A, R455G, R455P, R455T, R455S, R455Y, R455W, R455Q, R455N, R455H, R455E, R455D, R455K. In some embodiments, the adenosine deaminase comprises mutation E488Q, and further comprises one or more of mutations selected from V351I, V351L, V351F, V351M, V351C, V351A, V351G, V351P, V351T, V351 S, V351Y, V351W, V351Q, V351N, V351H, V351E, V351D, V351K, V351R, T375I, T375L, T375V, T375F, T375M, T375C, T375A, T375G, T375P, T375S, T375Y, T375W, T375Q, T375N, T375H, T375E, T375D, T375K, T375R, R455I, R455L, R455V, R455F, R455M, R455C, R455A, R455G, R455P, R455T, R455S, R455Y, R455W, R455Q, R455N, R455H, R455E, R455D, R455K.

In some cases, the modified ADAR may further comprise one or more mutations that reduce off-target activities. In cases where modified ADAR has C-to-U deamination activity, such mutations may reduce A to I off-target activity and increase C-to-U on-target deamination activity. In general, such mutations may be on residues that interact with the RNA target. Examples of such mutations include S375N, S375C, S375A, and N473I, based on amino acid sequence positions of hADAR2-D, and mutations in a homologous ADAR protein corresponding to the above. In one example, the ADAR has S375N mutation. In one example, provided herein includes a mutated adenosine deaminase e.g., an adenosine deaminase comprising E488Q, V351G, S486A, T375S, S370C, P462A, N597I, L332I, I398V, K350I, M383L, D619G, S582T, V440I, S495N, K418E, S661T, and S375N (based on amino acid sequence positions of hADAR2-D, and mutations in a homologous ADAR protein corresponding to the above), fused with a dead CasPR protein.

In connection with the aforementioned modified ADAR protein having C-to-U deamination activity, the invention described herein also relates to a method for deaminating a C in a target RNA sequence of interest, comprising delivering to a target RNA or DNA an AD-functionalized composition disclosed herein.

In certain embodiments, the method for deaminating a C in a target RNA sequence comprises delivering to said target RNA: (a) a catalytically inactive (dead) CasPR; (b) a guide molecule which comprises a guide sequence linked to a direct repeat sequence; and (c) a modified ADAR protein having C-to-U deamination activity or catalytic domain thereof; wherein said modified ADAR protein or catalytic domain thereof is covalently or non-covalently linked to said dead CasPR protein or said guide molecule or is adapted to link thereto after delivery; wherein guide molecule forms a complex with said dead CasPR protein and directs said complex to bind said target RNA sequence of interest; wherein said guide sequence is capable of hybridizing with a target sequence comprising said C to form an RNA duplex; wherein, optionally, said guide sequence comprises a non-pairing A or U at a position corresponding to said C resulting in a mismatch in the RNA duplex formed; and wherein said modified ADAR protein or catalytic domain thereof deaminates said C in said RNA duplex.

In connection with the aforementioned modified ADAR protein having C-to-U deamination activity, the invention described herein further relates to an engineered, non-naturally occurring system suitable for deaminating a C in a target locus of interest, comprising: (a) a guide molecule which comprises a guide sequence linked to a direct repeat sequence, or a nucleotide sequence encoding said guide molecule; (b) a catalytically inactive CasPR protein, or a nucleotide sequence encoding said catalytically inactive CasPR protein; (c) a modified ADAR protein having C-to-U deamination activity or catalytic domain thereof, or a nucleotide sequence encoding said modified ADAR protein or catalytic domain thereof; wherein said modified ADAR protein or catalytic domain thereof is covalently or non-covalently linked to said CasPR protein or said guide molecule or is adapted to link thereto after delivery; wherein said guide sequence is capable of hybridizing with a target RNA sequence comprising a C to form an RNA duplex; wherein, optionally, said guide sequence comprises a non-pairing A or U at a position corresponding to said C resulting in a mismatch in the RNA duplex formed; wherein, optionally, the system is a vector system comprising one or more vectors comprising: (a) a first regulatory element operably linked to a nucleotide sequence encoding said guide molecule which comprises said guide sequence, (b) a second regulatory element operably linked to a nucleotide sequence encoding said catalytically inactive CasPR protein; and (c) a nucleotide sequence encoding a modified ADAR protein having C-to-U deamination activity or catalytic domain thereof which is under control of said first or second regulatory element or operably linked to a third regulatory element; wherein, if said nucleotide sequence encoding a modified ADAR protein or catalytic domain thereof is operably linked to a third regulatory element, said modified ADAR protein or catalytic domain thereof is adapted to link to said guide molecule or said CasPR protein after expression; wherein components (a), (b) and (c) are located on the same or different vectors of the system, optionally wherein said first, second, and/or third regulatory element is an inducible promoter.

In an embodiment of the invention, the substrate of the adenosine deaminase is an RNA/DNA heteroduplex formed upon binding of the guide molecule to its DNA target which then forms the CasPR complex with the CasPR enzyme. The RNA/DNA or DNA/RNA heteroduplex is also referred to herein as the “RNA/DNA hybrid”, “DNA/RNA hybrid” or “double-stranded substrate.”

In an embodiment of the invention, the substrate of the adenosine deaminase is an RNA/RNA heteroduplex formed upon binding of the guide molecule to its RNA target which then forms the CasPR complex with the CasPR enzyme. The RNA/RNA duplex is also referred to as a “double-stranded substrate.”

he term “editing selectivity” as used herein refers to the fraction of all sites on a double-stranded substrate that is edited by an adenosine deaminase. Without being bound by theory, it is contemplated that editing selectivity of an adenosine deaminase is affected by the double-stranded substrate's length and secondary structures, such as the presence of mismatched bases, bulges and/or internal loops.

In some embodiments, when the substrate is a perfectly base-paired duplex longer than 50 bp, the adenosine deaminase may be able to deaminate multiple adenosine residues within the duplex (e.g., 50% of all adenosine residues). In some embodiments, when the substrate is shorter than 50 bp, the editing selectivity of an adenosine deaminase is affected by the presence of a mismatch at the target adenosine site. Particularly, in some embodiments, adenosine (A) residue having a mismatched cytidine (C) residue on the opposite strand is deaminated with high efficiency. In some embodiments, adenosine (A) residue having a mismatched guanosine (G) residue on the opposite strand is skipped without editing.

In particular embodiments, the adenosine deaminase protein or catalytic domain thereof is delivered to the cell or expressed within the cell as a separate protein, but is modified so as to be able to link to either the CasPR protein or the guide molecule. In particular embodiments, this is ensured by the use of orthogonal RNA-binding protein or adaptor protein/aptamer combinations that exist within the diversity of bacteriophage coat proteins. Examples of such coat proteins include but are not limited to: MS2, QP, F2, GA, fr, JP501, M12, R17, BZ13, JP34, JP500, KU1, M11, MX1, TW18, VK, SP, FI, ID2, NL95, TW19, AP205, ϕCb5, ϕCb8r, ϕCb12r, ϕCb23r, 7s and PRR1. Aptamers can be naturally occurring or synthetic oligonucleotides that have been engineered through repeated rounds of in vitro selection or SELEX (systematic evolution of ligands by exponential enrichment) to bind to a specific target.

In particular embodiments, the guide molecule is provided with one or more distinct RNA loop(s) or distinct sequence(s) that can recruit an adaptor protein. A guide molecule may be extended, without colliding with the CasPR protein by the insertion of distinct RNA loop(s) or distinct sequence(s) that may recruit adaptor proteins that can bind to the distinct RNA loop(s) or distinct sequence(s). Examples of modified guides and their use in recruiting effector domains to the CasPR complex are provided in Konermann (Nature 2015, 517(7536): 583-588). In particular embodiments, the aptamer is a minimal hairpin aptamer which selectively binds dimerized MS2 bacteriophage coat proteins in mammalian cells and is introduced into the guide molecule, such as in the stem-loop and/or in a tetraloop. In these embodiments, the adenosine deaminase protein is fused to MS2. The adenosine deaminase protein is then co-delivered together with the CasPR protein and corresponding guide RNA.

In some embodiments, the CasPR-ADAR base editing system described herein comprises (a) a CasPR protein, which is catalytically inactive; (b) a guide molecule which comprises a guide sequence; and (c) an adenosine deaminase protein or catalytic domain thereof; wherein the adenosine deaminase protein or catalytic domain thereof is covalently or non-covalently linked to the CasPR protein or the guide molecule or is adapted to link thereto after delivery; wherein the guide sequence is substantially complementary to the target sequence but comprises a non-pairing C corresponding to the A being targeted for deamination, resulting in a A-C mismatch in a DNA-RNA or RNA-RNA duplex formed by the guide sequence and the target sequence. For application in eukaryotic cells, the CasPR protein and/or the adenosine deaminase are preferably NLS-tagged.

In some embodiments, the components (a), (b) and (c) are delivered to the cell as a ribonucleoprotein complex. The ribonucleoprotein complex can be delivered via one or more lipid nanoparticles.

In some embodiments, the components (a), (b) and (c) are delivered to the cell as one or more RNA molecules, such as one or more guide RNAs and one or more mRNA molecules encoding the CasPR protein, the adenosine deaminase protein, and optionally the adaptor protein. The RNA molecules can be delivered via one or more lipid nanoparticles.

In some embodiments, the components (a), (b) and (c) are delivered to the cell as one or more DNA molecules. In some embodiments, the one or more DNA molecules are comprised within one or more vectors such as viral vectors (e.g., AAV). In some embodiments, the one or more DNA molecules comprise one or more regulatory elements operably configured to express the CasPR protein, the guide molecule, and the adenosine deaminase protein or catalytic domain thereof, optionally wherein the one or more regulatory elements comprise inducible promoters.

In some embodiments of the guide molecule is capable of hybridizing with a target sequence comprising the Adenine to be deaminated within a first DNA strand or a RNA strand at the target locus to form a DNA-RNA or RNA-RNA duplex which comprises a non-pairing Cytosine opposite to said Adenine. Upon duplex formation, the guide molecule forms a complex with the CasPR protein and directs the complex to bind said first DNA strand or said RNA strand at the target locus of interest. Details on the aspect of the guide of the CasPR-ADAR base editing system are provided herein below.

In some embodiments, a CasPR guide RNA having a canonical length (e.g., about 20-30 nt) is used to form a DNA-RNA or RNA-RNA duplex with the target DNA or RNA. In some embodiments, a CasPR guide molecule longer than the canonical length (e.g., >20 nt) is used to form a DNA-RNA or RNA-RNA duplex with the target DNA or RNA including outside of the Cas-guide RNA-target DNA complex. In certain example embodiments, the guide sequence has a length of about 15-120 nt, 20-100 nt, 25-80 nt, or 29-53 nt capable of forming a DNA-RNA or RNA-RNA duplex with said target sequence. In certain other example embodiments, the guide sequence has a length of about 40-50 nt capable of forming a DNA-RNA or RNA-RNA duplex with said target sequence. In certain example embodiments, the distance between said non-pairing C and the 5′ end of said guide sequence is 20-30 nucleotides. In certain example embodiments, the distance between said non-pairing C and the 3′ end of said guide sequence is 20-30 nucleotides.

In at least a first design, the CasPR-ADAR system comprises (a) an adenosine deaminase fused or linked to a CasPR protein, wherein the CasPR protein is catalytically inactive, and (b) a guide molecule comprising a guide sequence designed to introduce a A-C mismatch in a DNA-RNA or RNA-RNA duplex formed between the guide sequence and the target sequence. In some embodiments, the CasPR protein and/or the adenosine deaminase are NLS-tagged, on either the N- or C-terminus or both.

In at least a second design, the CasPR-ADAR system comprises (a) a CasPR protein that is catalytically inactive, (b) a guide molecule comprising a guide sequence designed to introduce a A-C mismatch in a DNA-RNA or RNA-RNA duplex formed between the guide sequence and the target sequence, and an aptamer sequence (e.g., MS2 RNA motif or PP7 RNA motif) capable of binding to an adaptor protein (e.g., MS2 coating protein or PP7 coat protein), and (c) an adenosine deaminase fused or linked to an adaptor protein, wherein the binding of the aptamer and the adaptor protein recruits the adenosine deaminase to the DNA-RNA or RNA-RNA duplex formed between the guide sequence and the target sequence for targeted deamination at the A of the A-C mismatch. In some embodiments, the adaptor protein and/or the adenosine deaminase are NLS-tagged, on either the N- or C-terminus or both. The CasPR protein can also be NLS-tagged.

The use of different aptamers and corresponding adaptor proteins also allows orthogonal gene editing to be implemented. In one example in which adenosine deaminase are used in combination with cytidine deaminase for orthogonal gene editing/deamination, sgRNA targeting different loci are modified with distinct RNA loops in order to recruit MS2-adenosine deaminase and PP7-cytidine deaminase (or PP7-adenosine deaminase and MS2-cytidine deaminase), respectively, resulting in orthogonal deamination of A or C at the target loci of interested, respectively. PP7 is the RNA-binding coat protein of the bacteriophage Pseudomonas. Like MS2, it binds a specific RNA sequence and secondary structure. The PP7 RNA-recognition motif is distinct from that of MS2. Consequently, PP7 and MS2 can be multiplexed to mediate distinct effects at different genomic loci simultaneously. For example, an sgRNA targeting locus A can be modified with MS2 loops, recruiting MS2-adenosine deaminase, while another sgRNA targeting locus B can be modified with PP7 loops, recruiting PP7-cytidine deaminase. In the same cell, orthogonal, locus-specific modifications are thus realized. This principle can be extended to incorporate other orthogonal RNA-binding proteins.

In at least a third design, the CasPR-ADAR CRISPR system comprises (a) an adenosine deaminase inserted into an internal loop or unstructured region of a CasPR protein, wherein the CasPR protein is catalytically inactive or a nickase, and (b) a guide molecule comprising a guide sequence designed to introduce a A-C mismatch in a DNA-RNA or RNA-RNA duplex formed between the guide sequence and the target sequence.

CasPR protein split sites that are suitable for insertion of adenosine deaminase can be identified with the help of a crystal structure. It should be readily apparent what the corresponding position for, for example, a sequence alignment. For other CasPR protein one can use the crystal structure of an ortholog if a relatively high degree of homology exists between the ortholog and the intended CasPR protein.

The split position may be located within a region or loop. Preferably, the split position occurs where an interruption of the amino acid sequence does not result in the partial or full destruction of a structural feature (e.g. alpha-helixes or b-sheets). Unstructured regions (regions that did not show up in the crystal structure because these regions are not structured enough to be “frozen” in a crystal) are often preferred options. Splits in all unstructured regions that are exposed on the surface of Cas are envisioned in the practice of the invention. The positions within the unstructured regions or outside loops may not need to be exactly the numbers provided above, but may vary by, for example 1, 2, 3, 4, 5, 6, 7, 8, 9, or even 10 amino acids either side of the position given above, depending on the size of the loop, so long as the split position still falls within an unstructured region of outside loop.

The CasPR-ADAR system described herein can be used to target a specific Adenine within a DNA sequence for deamination. For example, the guide molecule can form a complex with the CasPR protein and directs the complex to bind a target sequence at the target locus of interest. Because the guide sequence is designed to have a non-pairing C, the heteroduplex formed between the guide sequence and the target sequence comprises a A-C mismatch, which directs the adenosine deaminase to contact and deaminate the A opposite to the non-pairing C, converting it to a Inosine (I). Since Inosine (I) base pairs with C and functions like G in cellular process, the targeted deamination of A described herein are useful for correction of undesirable G-A and C-T mutations, as well as for obtaining desirable A-G and T-C mutations. In some embodiments, the guide may comprise one or more mismatches to increase specificity. For example, the guide may comprise one or more disfavorable guanine mismatches across from off-target adenosines.

### Base Excision Repair Inhibitor

In some embodiments, the AD-functionalized Class 1 CRISPR-Cas system further comprises a base excision repair (BER) inhibitor. Without wishing to be bound by any particular theory, cellular DNA-repair response to the presence of I:T pairing may be responsible for a decrease in nucleobase editing efficiency in cells. Alkyladenine DNA glycosylase (also known as DNA-3-methyladenine glycosylase, 3-alkyladenine DNA glycosylase, or N-methylpurine DNA glycosylase) catalyzes removal of hypoxanthine from DNA in cells, which may initiate base excision repair, with reversion of the I:T pair to a A:T pair as outcome.

In some embodiments, the BER inhibitor is an inhibitor of alkyladenine DNA glycosylase. In some embodiments, the BER inhibitor is an inhibitor of human alkyladenine DNA glycosylase. In some embodiments, the BER inhibitor is a polypeptide inhibitor. In some embodiments, the BER inhibitor is a protein that binds hypoxanthine. In some embodiments, the BER inhibitor is a protein that binds hypoxanthine in DNA. In some embodiments, the BER inhibitor is a catalytically inactive alkyladenine DNA glycosylase protein or binding domain thereof. In some embodiments, the BER inhibitor is a catalytically inactive alkyladenine DNA glycosylase protein or binding domain thereof that does not excise hypoxanthine from the DNA. Other proteins that are capable of inhibiting (e.g., sterically blocking) an alkyladenine DNA glycosylase base-excision repair enzyme are within the scope of this disclosure. Additionally, any proteins that block or inhibit base-excision repair as also within the scope of this disclosure.

Without wishing to be bound by any particular theory, base excision repair may be inhibited by molecules that bind the edited strand, block the edited base, inhibit alkyladenine DNA glycosylase, inhibit base excision repair, protect the edited base, and/or promote fixing of the non-edited strand. It is believed that the use of the BER inhibitor described herein can increase the editing efficiency of an adenosine deaminase that is capable of catalyzing a to I change.

Accordingly, in the first design of the AD-functionalized Class 1 CRISPR system discussed above, the CasPR protein or the adenosine deaminase can be fused to or linked to a BER inhibitor (e.g., an inhibitor of alkyladenine DNA glycosylase). In some embodiments, the BER inhibitor can be comprised in one of the following structures (dCasPR=dead CasPR):

[AD]-[optional linker]-[dCasPR]-[optional linker]-[BER inhibitor]; [AD]-[optional linker]-[BER inhibitor]-[optional linker]-[dCasPR]; [BER inhibitor]-[optional linker]-[AD]-[optional linker]-[dCasPR]; [BER inhibitor]-[optional linker]-[dCasPR]-[optional linker]-[AD]; [dCasPR]-[optional linker]-[AD]-[optional linker]-[BER inhibitor]; [dCasPR]-[optional linker]-[BER inhibitor]-[optional linker]-[AD].

Similarly, in the second design of the AD-functionalized Class 1 CRISPR system discussed above, the CasPR protein, the adenosine deaminase, or the adaptor protein can be fused to or linked to a BER inhibitor (e.g., an inhibitor of alkyladenine DNA glycosylase). In some embodiments, the BER inhibitor can be comprised in one of the following structures (dCasPR=dead CasPR):

[dCasPR]-[optional linker]-[BER inhibitor]; [BER inhibitor]-[optional linker]-[dCasPR]; [AD]-[optional linker]-[Adaptor]-[optional linker]-[BER inhibitor]; [AD]-[optional linker]-[BER inhibitor]-[optional linker]-[Adaptor]; [BER inhibitor]-[optional linker]-[AD]-[optional linker]-[Adaptor]; [BER inhibitor]-[optional linker]-[Adaptor]-[optional linker]-[AD]; [Adaptor]-[optional linker]-[AD]-[optional linker]-[BER inhibitor]; [Adaptor]-[optional linker]-[BER inhibitor]-[optional linker]-[AD].

In the third design of the AD-functionalized Class 1 CRISPR system discussed above, the BER inhibitor can be inserted into an internal loop or unstructured region of a CasPR protein.

### Cytidine Deaminase

In some embodiments, the deaminase is a cytidine deaminase. The term “cytidine deaminase” or “cytidine deaminase protein” or “cytidine deaminase activity” as used herein refers to a protein, a polypeptide, or one or more functional domain(s) of a protein or a polypeptide that is capable of catalyzing a hydrolytic deamination reaction that converts an cytosine (or an cytosine moiety of a molecule) to an uracil (or a uracil moiety of a molecule), as shown below. In some embodiments, the cytosine-containing molecule is an cytidine (C), and the uracil-containing molecule is an uridine (U). The cytosine-containing molecule can be deoxyribonucleic acid (DNA) or ribonucleic acid (RNA). In certain examples, a cytidine deaminase may be a cytidine deaminase acting on RNA (CDAR).

According to the present disclosure, cytidine deaminases that can be used in connection with the present disclosure include, but are not limited to, members of the enzyme family known as apolipoprotein B mRNA-editing complex (APOBEC) family deaminase, an activation-induced deaminase (AID), or a cytidine deaminase 1 (CDA1). In particular embodiments, the deaminase in an APOBEC 1 deaminase, an APOBEC2 deaminase, an APOBEC3A deaminase, an APOBEC3B deaminase, an APOBEC3C deaminase, and APOBEC3D deaminase, an APOBEC3E deaminase, an APOBEC3F deaminase an APOBEC3G deaminase, an APOBEC3H deaminase, or an APOBEC4 deaminase.

In the methods and systems of the present invention, the cytidine deaminase or engineered adenosine deaminase with cytidine deaminase activity is capable of targeting Cytosine in a DNA single strand. In certain example embodiments the cytidine deaminase activity may edit on a single strand present outside of the binding component, e.g., bound CasPR. In other example embodiments, the cytidine deaminase may edit at a localized bubble, such as a localized bubble formed by a mismatch at the target edit site but the guide sequence. In certain example embodiments the cytidine deaminase may contain mutations that help focus the area of activity such as those disclosed in Kim et al., Nature Biotechnology 35(4):371-377, 2017 (doi: 10. 1038/nbt.3803).

In some embodiments, the cytidine deaminase is derived from one or more metazoa species, including but not limited to, mammals, birds, frogs, squids, fish, flies and worms. In some embodiments, the cytidine deaminase is a human, primate, cow, dog rat or mouse cytidine deaminase.

In some embodiments, the cytidine deaminase is a human APOBEC, including hAPOBEC1 or hAPOBEC3. In some embodiments, the cytidine deaminase is a human AID.

In some embodiments, the cytidine deaminase protein recognizes and converts one or more target cytosine residue(s) in a single-stranded bubble of a RNA duplex into uracil residues (s). In some embodiments, the cytidine deaminase protein recognizes a binding window on the single-stranded bubble of a RNA duplex. In some embodiments, the binding window contains at least one target cytosine residue(s). In some embodiments, the binding window is in the range of about 3 bp to about 100 bp. In some embodiments, the binding window is in the range of about 5 bp to about 50 bp. In some embodiments, the binding window is in the range of about 10 bp to about 30 bp. In some embodiments, the binding window is about 1 bp, 2 bp, 3 bp, 5 bp, 7 bp, 10 bp, 15 bp, 20 bp, 25 bp, 30 bp, 40 bp, 45 bp, 50 bp, 55 bp, 60 bp, 65 bp, 70 bp, 75 bp, 80 bp, 85 bp, 90 bp, 95 bp, or 100 bp.

In some embodiments, the cytidine deaminase protein comprises one or more deaminase domains. Not intended to be bound by theory, it is contemplated that the deaminase domain functions to recognize and convert one or more target cytosine (C) residue(s) contained in a single-stranded bubble of a RNA duplex into (an) uracil (EG) residue (s). In some embodiments, the deaminase domain comprises an active center. In some embodiments, the active center comprises a zinc ion. In some embodiments, amino acid residues in or near the active center interact with one or more nucleotide(s) 5′ to a target cytosine residue. In some embodiments, amino acid residues in or near the active center interact with one or more nucleotide(s) 3′ to a target cytosine residue.

In some embodiments, the cytidine deaminase comprises human APOBEC 1 full protein (hAPOBEC1) or the deaminase domain thereof (hAPOBEC1-D) or a C-terminally truncated version thereof (hAPOBEC-T). In some embodiments, the cytidine deaminase is an APOBEC family member that is homologous to hAPOBEC1, hAPOBEC-D or hAPOBEC-T. In some embodiments, the cytidine deaminase comprises human AID1 full protein (hAID) or the deaminase domain thereof (hAID-D) or a C-terminally truncated version thereof (hAID-T). In some embodiments, the cytidine deaminase is an AID family member that is homologous to hAID, hAID-D or hAID-T. In some embodiments, the hAID-T is a hAID which is C-terminally truncated by about 20 amino acids.

In some embodiments, the cytidine deaminase comprises the wild-type amino acid sequence of a cytosine deaminase. In some embodiments, the cytidine deaminase comprises one or more mutations in the cytosine deaminase sequence, such that the editing efficiency, and/or substrate editing preference of the cytosine deaminase is changed according to specific needs.

Certain mutations of APOBEC1 and APOBEC3 proteins have been described in Kim et al., Nature Biotechnology 35(4):371-377, 2017 (doi: 10. 1038/nbt.3803); and Harris et al., Mol. Cell 10:1247-1253, 2002, each of which is incorporated herein by reference in its entirety.

In some embodiments, the cytidine deaminase is an APOBEC1 deaminase comprising one or more mutations at amino acid positions corresponding to W90, R118, H121, H122, R126, or R132 in rat APOBEC1, or an APOBEC3G deaminase comprising one or more mutations at amino acid positions corresponding to W285, R313, D316, D317, R320, or R326 in human APOBEC3G.

In some embodiments, the cytidine deaminase comprises a mutation at W90 of the rat APOBEC1 amino acid sequence, or a corresponding position in a homologous APOBEC protein, such as W285 of APOBEC3G. In some embodiments, W90 is replaced by an tyrosine or phenylalanine residue (W90Y or W90F).

In some embodiments, the cytidine deaminase comprises a mutation at R118 of the rat APOBEC 1 amino acid sequence, or a corresponding position in a homologous APOBEC protein. In some embodiments, R118 is replaced by an alanine residue (R118A).

In some embodiments, the cytidine deaminase comprises a mutation at H121 of the rat APOBEC 1 amino acid sequence, or a corresponding position in a homologous APOBEC protein. In some embodiments, H121 is replaced by an arginine residue (H121R).

In some embodiments, the cytidine deaminase comprises a mutation at H122 of the rat APOBEC 1 amino acid sequence, or a corresponding position in a homologous APOBEC protein. In some embodiments, H122 is replaced by an arginine residue (H122R).

In some embodiments, the cytidine deaminase comprises a mutation at R126 of the rat APOBEC 1 amino acid sequence, or a corresponding position in a homologous APOBEC protein, such as R320 of APOBEC3G. In some embodiments, R126 is replaced by an alanine residue (R126A) or by a glutamic acid (R126E).

In some embodiments, the cytidine deaminase comprises a mutation at R132 of the APOBEC1 amino acid sequence, or a corresponding position in a homologous APOBEC protein. In some embodiments, R132 is replaced by a glutamic acid residue (R132E).

In some embodiments, to narrow the width of the editing window, the cytidine deaminase may comprise one or more of the mutations: W90Y, W90F, R126E and R132E, based on amino acid sequence positions of rat APOBEC 1, and mutations in a homologous APOBEC protein corresponding to the above.

In some embodiments, to reduce editing efficiency, the cytidine deaminase may comprise one or more of the mutations: W90A, R118 A, R132E, based on amino acid sequence positions of rat APOBEC 1, and mutations in a homologous APOBEC protein corresponding to the above. In particular embodiments, it can be of interest to use a cytidine deaminase enzyme with reduced efficacy to reduce off-target effects.

In some embodiments, the cytidine deaminase is wild-type rat APOBEC 1 (rAPOBEC1, or a catalytic domain thereof. In some embodiments, the cytidine deaminase comprises one or more mutations in the rAPOBEC1 sequence, such that the editing efficiency, and/or substrate editing preference of rAPOBEC1 is changed according to specific needs.

rAPOBEC1 (SEQ ID NO: 111):
MSSETGPVAVDPTLRRRIEPHEFEVFFDPRELRKETCLLYEINWGGRHSIWRHTSQNTNKHVEVNFIEKF
TTERYFCPNTRCSITWFLSWSPCGECSRAITEFLSRYPHVTLFIYIARLYHHADPRNRQGLRDLISSGVT
IQIMTEQESGYCWRNFVNYSPSNEAHWPRYPHLWVRLYVLELYCIILGLPPCLNILRRKQPQLTFFTIAL
QSCHYQRLPPHILWATGLK

In some embodiments, the cytidine deaminase is wild-type human APOBEC 1 (hAPOBEC1) or a catalytic domain thereof. In some embodiments, the cytidine deaminase comprises one or more mutations in the hAPOBEC1 sequence, such that the editing efficiency, and/or substrate editing preference of hAPOBEC1 is changed according to specific needs.

APOBEC 1 (SEQ ID NO: 112):
MTSEKGPSTGDPTLRRRIEPWEFDVFYDPRELRKEACLLYEIKWGMSRKIWRSSGKNTINHVEVNFIKKF
TSERDFHPSMSCSITWFLSWSPCWECSQAIREFLSRHPGVTLVIYVARLFWHMDQQNRQGLRDLVNSGVT
IQIMRASEYYHCWRNFVNYPPGDEAHWPQYPPLWMMLYALELHCIILSLPPCLKISRRWQNHLTFFRLHL
QNCHYQTIPPHILLATGLIHPSVAWR

In some embodiments, the cytidine deaminase is wild-type human APOBEC3G (hAPOBEC3G) or a catalytic domain thereof. In some embodiments, the cytidine deaminase comprises one or more mutations in the hAPOBEC3G sequence, such that the editing efficiency, and/or substrate editing preference of hAPOBEC3G is changed according to specific needs.

hAPOBEC3G (SEQ ID NO: 113):
MELKYHPEMRFFHWFSKWRKLHRDQEYEVTWYISWSPCTKCTRDMATFLAEDPKVTLTIFVARLYYFWDP
DYQEALRSLCQKRDGPRATMKIMNYDEFQHCWSKFVYSQRELFEPWNNLPKYYILLHIMLGEILRHSMDP
PTFTFNFNNEPWVRGRHETYLCYEVERMHNDTWVLLNQRRGFLCNQAPHKHGFLEGRHAELCFLDVIPFW
KLDLDQDYRVTCFTSWSPCFSCAQEMAKFISKNKHVSLCIFTARIYDDQGRCQEGLRTLAEAGAKISIMT
YSEFKHCWDTFVDHQGCPFQPWDGLDEHSQDLSGRLRAILQNQEN

In some embodiments, the cytidine deaminase is wild-type Petromyzon marinus CDA1 (pmCDA1) or a catalytic domain thereof. In some embodiments, the cytidine deaminase comprises one or more mutations in the pmCDA1 sequence, such that the editing efficiency, and/or substrate editing preference of pmCDA1 is changed according to specific needs.

pmCDA1 (SEQ ID NO: 114):
MTDAEYVRIHEKLDIYTFKKQFFNNKKSVSHRCYVLFELKRRGERRACFWGYAVNKPQSGTERGIHAEIF
SIRKVEEYLRDNPGQFTINWYSSWSPCADCAEKILEWYNQELRGNGHTLKIWACKLYYEKNARNQIGLWN
LRDNGVGLNVMVSEHYQCCRKIFIQSSHNQLNENRWLEKTLKRAEKRRSELSIMIQVKILHTTKSPAV

In some embodiments, the cytidine deaminase is wild-type human AID (hAID) or a catalytic domain thereof. In some embodiments, the cytidine deaminase comprises one or more mutations in the pmCDA1 sequence, such that the editing efficiency, and/or substrate editing preference of pmCDA1 is changed according to specific needs.

hAID (SEQ ID NO: 115):
MDSLLMNRRKFLYQFKNVRWAKGRRETYLCYVVKRRDSATSFSLDFGYLRNKNGCHVELLFLRYISDWDL
DPGRCYRVTWFTSWSPCYDCARHVADFLRGNPYLSLRIFTARLYFCEDRKAEPEGLRRLHRAGVQIAIMT
FKDYFYCWNTFVENHERTFKAWEGLHENSVRLSRQLRRILLPLYEVDDLRDAFRILGLLD

In some embodiments, the cytidine deaminase is truncated version of hAID (hAID-DC) or a catalytic domain thereof. In some embodiments, the cytidine deaminase comprises one or more mutations in the hAID-DC sequence, such that the editing efficiency, and/or substrate editing preference of hAID-DC is changed according to specific needs.

hAID-DC (SEQ ID NO: 116):
MDSLLMNRRKFLYQFKNVRWAKGRRETYLCYVVKRRDSATSFSLDFGYLRNKNGCHVELLFLRYISDWDL
DPGRCYRVTWFTSWSPCYDCARHVADFLRGNPNLSLRIFTARLYFCEDRKAEPEGLRRLHRAGVQIAIMT
FKDYFYCWNTFVENHERTFKAWEGLHENSVRLSRQLRRILL

Additional embodiments of the cytidine deaminase are disclosed in WO2017-070632, titled “Nucleobase Editor and ETses Thereof,” which is incorporated herein by reference in its entirety.

In some embodiments, the cytidine deaminase has an efficient deamination window that encloses the nucleotides susceptible to deamination editing. Accordingly, in some embodiments, the “editing window width” refers to the number of nucleotide positions at a given target site for which editing efficiency of the cytidine deaminase exceeds the half-maximal value for that target site. In some embodiments, the cytidine deaminase has an editing window width in the range of about 1 to about 6 nucleotides. In some embodiments, the editing window width of the cytidine deaminase is 1, 2, 3, 4, 5, or 6 nucleotides.

Not intended to be bound by theory, it is contemplated that in some embodiments, the length of the linker sequence affects the editing window width. In some embodiments, the editing window width increases (e.g., from about 3 to about 6 nucleotides) as the linker length extends (e.g., from about 3 to about 21 amino acids). In a non-limiting example, a 16-residue linker offers an efficient deamination window of about 5 nucleotides. In some embodiments, the length of the guide RNA affects the editing window width. In some embodiments, shortening the guide RNA leads to a narrowed efficient deamination window of the cytidine deaminase.

In some embodiments, mutations to the cytidine deaminase affect the editing window width. In some embodiments, the cytidine deaminase component of the CD-functionalized CRISPR system comprises one or more mutations that reduce the catalytic efficiency of the cytidine deaminase, such that the deaminase is prevented from deamination of multiple cytidines per DNA binding event. In some embodiments, tryptophan at residue 90 (W90) of APOBEC1 or a corresponding tryptophan residue in a homologous sequence is mutated. In some embodiments, the catalytically inactive CasPR is fused to or linked to an APOBEC1 mutant that comprises a W90Y or W90F mutation. In some embodiments, tryptophan at residue 285 (W285) of APOBEC3G, or a corresponding tryptophan residue in a homologous sequence is mutated. In some embodiments, the catalytically inactive CasPR is fused to or linked to an APOBEC3G mutant that comprises a W285Y or W285F mutation.

In some embodiments, the cytidine deaminase component of CD-functionalized Class 1 CRISPR system comprises one or more mutations that reduce tolerance for non-optimal presentation of a cytidine to the deaminase active site. In some embodiments, the cytidine deaminase comprises one or more mutations that alter substrate binding activity of the deaminase active site. In some embodiments, the cytidine deaminase comprises one or more mutations that alter the conformation of DNA to be recognized and bound by the deaminase active site. In some embodiments, the cytidine deaminase comprises one or more mutations that alter the substrate accessibility to the deaminase active site. In some embodiments, arginine at residue 126 (R126) of APOBEC1 or a corresponding arginine residue in a homologous sequence is mutated. In some embodiments, the catalytically inactive CasPR is fused to or linked to an APOBEC1 that comprises a R126A or R126E mutation. In some embodiments, tryptophan at residue 320 (R320) of APOBEC3G, or a corresponding arginine residue in a homologous sequence is mutated. In some embodiments, the catalytically inactive CasPR is fused to or linked to an APOBEC3G mutant that comprises a R320A or R320E mutation. In some embodiments, arginine at residue 132 (R132) of APOBEC1 or a corresponding arginine residue in a homologous sequence is mutated. In some embodiments, the catalytically inactive CasPR is fused to or linked to an APOBEC1 mutant that comprises a R132E mutation.

In some embodiments, the APOBEC1 domain of the CD-functionalized Class 1 CRISPR system comprises one, two, or three mutations selected from W90Y, W90F, R126A, R126E, and R132E. In some embodiments, the APOBEC1 domain comprises double mutations of W90Y and R126E. In some embodiments, the APOBEC1 domain comprises double mutations of W90Y and R132E. In some embodiments, the APOBEC1 domain comprises double mutations of R126E and R132E. In some embodiments, the APOBEC1 domain comprises three mutations of W90Y, R126E and R132E.

In some embodiments, one or more mutations in the cytidine deaminase as disclosed herein reduce the editing window width to about 2 nucleotides. In some embodiments, one or more mutations in the cytidine deaminase as disclosed herein reduce the editing window width to about 1 nucleotide. In some embodiments, one or more mutations in the cytidine deaminase as disclosed herein reduce the editing window width while only minimally or modestly affecting the editing efficiency of the enzyme. In some embodiments, one or more mutations in the cytidine deaminase as disclosed herein reduce the editing window width without reducing the editing efficiency of the enzyme. In some embodiments, one or more mutations in the cytidine deaminase as disclosed herein enable discrimination of neighboring cytidine nucleotides, which would be otherwise edited with similar efficiency by the cytidine deaminase.

In some embodiments, the cytidine deaminase protein further comprises or is connected to one or more double-stranded RNA (dsRNA) binding motifs (dsRBMs) or domains (dsRBDs) for recognizing and binding to double-stranded nucleic acid substrates. In some embodiments, the interaction between the cytidine deaminase and the substrate is mediated by one or more additional protein factor(s), including a CASPR protein factor. In some embodiments, the interaction between the cytidine deaminase and the substrate is further mediated by one or more nucleic acid component s), including a guide RNA.

According to the present invention, the substrate of the cytidine deaminase is an RNA single strand bubble of a RNA duplex comprising a Cytosine of interest, made accessible to the cytidine deaminase upon binding of the guide molecule to its RNA target which then forms the CasPR complex with the CasPR enzyme, whereby the cytosine deaminase is fused to or is capable of binding to one or more components of the CasPR complex, i.e. the CasPR enzyme and/or the guide molecule. The particular features of the guide molecule and CasPR enzyme are detailed below.

The cytidine deaminase or catalytic domain thereof may be a human, a rat, or a lamprey cytidine deaminase protein or catalytic domain thereof.

The cytidine deaminase protein or catalytic domain thereof may be an apolipoprotein B mRNA-editing complex (APOBEC) family deaminase. The cytidine deaminase protein or catalytic domain thereof may be an activation-induced deaminase (AID). The cytidine deaminase protein or catalytic domain thereof may be a cytidine deaminase 1 (CDA1).

The cytidine deaminase protein or catalytic domain thereof may be an APOBEC 1 deaminase. The APOBEC 1 deaminase may comprise one or more mutations corresponding to W90A, W90Y, R118A, H121R, H122R, R126A, R126E, or R132E in rat APOBEC 1, or an APOBEC3G deaminase comprising one or more mutations corresponding to W285A, W285Y, R313A, D316R, D317R, R320A, R320E, or R326E in human APOBEC3G.

The system may further comprise a uracil glycosylase inhibitor (UGI). In some embodiments, the cytidine deaminase protein or catalytic domain thereof is delivered together with a uracil glycosylase inhibitor (UGI). The UGI may be linked (e.g., covalently linked) to the cytidine deaminase protein or catalytic domain thereof and/or a catalytically inactive CasPR.

### Base Editing Guide Molecule

In some embodiments, the guide sequence is an RNA sequence of between 10 to 50 nt in length, but more particularly of about 20-30 nt advantageously about 20 nt, 23-25 nt or 24 nt. In base editing embodiments, the guide sequence is selected so as to ensure that it hybridizes to the target sequence comprising the adenosine to be deaminated. This is described more in detail below. Selection can encompass further steps which increase efficacy and specificity of deamination.

In some embodiments, the guide sequence is about 20 nt to about 30 nt long and hybridizes to the target DNA strand to form an almost perfectly matched duplex, except for having a dA-C mismatch at the target adenosine site. Particularly, in some embodiments, the dA-C mismatch is located close to the center of the target sequence (and thus the center of the duplex upon hybridization of the guide sequence to the target sequence), thereby restricting the adenosine deaminase to a narrow editing window (e.g., about 4 bp wide). In some embodiments, the target sequence may comprise more than one target adenosine to be deaminated. In further embodiments the target sequence may further comprise one or more dA-C mismatch 3′ to the target adenosine site. In some embodiments, to avoid off-target editing at an unintended Adenine site in the target sequence, the guide sequence can be designed to comprise a non-pairing Guanine at a position corresponding to said unintended Adenine to introduce a dA-G mismatch, which is catalytically unfavorable for certain adenosine deaminases such as AD AR1 and ADAR2. See Wong et al., RNA 7:846-858 (2001), which is incorporated herein by reference in its entirety.

In some embodiments, a CasPR guide sequence having a canonical length (e.g., about 20-30 nt) is used to form a heteroduplex with the target RNA. In some embodiments, a CasPR guide molecule longer than the canonical length (e.g., >20 or 30 nt) is used to form a heteroduplex with the target RNA including outside of the CasPR-guide RNA-target RNA complex. This can be of interest where deamination of more than one adenine within a given stretch of nucleotides is of interest. In alternative embodiments, it is of interest to maintain the limitation of the canonical guide sequence length. In some embodiments, the guide sequence is designed to introduce a dA-C mismatch outside of the canonical length of CasPR guide, which may decrease steric hindrance by CasPR and increase the frequency of contact between the adenosine deaminase and the dA-C mismatch.

Mismatch distance is the number of bases between the 3′ end of the CasPR spacer and the mismatched nucleobase (e.g., cytidine), wherein the mismatched base is included as part of the mismatch distance calculation.

In some embodiment, the mismatch distance is 1-10 nt, or 1-9 nt, or 1-8 nt, or 2-8 nt, or 2-7 nt, or 2-6 nt, or 3-8 nt, or 3-7 nt, or 3-6 nt, or 3-5 nt, or about 2 nt, or about 3 nt, or about 4 nt, or about 5 nt, or about 6 nt, or about 7 nt, or about 8 nt. In one embodiment, the mismatch distance is 3-5 nt or 4 nt.

In some embodiment, the mismatch distance is 5-20 nt, or 7-18 nt, or 9-17 nt, or 11-16 nt, or 13-15 nt. In one embodiment, the mismatch distance is about 15 nt.

In certain embodiments, the mismatch distance depends on the length of the spacer, such as anywhere between 3 to (N-3) nt, wherein N is the length of the spacer.

### Linkers

The deaminase herein may be fused to a CasPR protein via a linker. It is further envisaged that RNA adenosine methylase (N(6)-methyladenosine) can be fused to the RNA targeting effector proteins of the invention and targeted to a transcript of interest. This methylase causes reversible methylation, has regulatory roles and may affect gene expression and cell fate decisions by modulating multiple RNA-related cellular pathways (Fu et al., Nat Rev Genet. 15(5):293-306, 2014).

ADAR or other RNA modification enzymes may be linked (e.g., fused) to CasPR or a dead CasPR protein via a linker, e.g., to the C- or the N-terminus of CasPR or dead CasPR.

The term “linker” as used in reference to a fusion protein refers to a molecule which joins the proteins to form a fusion protein. Generally, such molecules have no specific biological activity other than to join or to preserve some minimum distance or other spatial relationship between the proteins. However, in certain embodiments, the linker may be selected to influence some property of the linker and/or the fusion protein such as the folding, net charge, or hydrophobicity of the linker.

Suitable linkers for use in the methods of the present invention are well known to those of skill in the art and include, but are not limited to, straight or branched-chain carbon linkers, heterocyclic carbon linkers, or peptide linkers. However, as used herein the linker may also be a covalent bond (carbon-carbon bond or carbon-heteroatom bond). In particular embodiments, the linker is used to separate the CasPR protein and the nucleotide deaminase by a distance sufficient to ensure that each protein retains its required functional property. Preferred peptide linker sequences adopt a flexible extended conformation and do not exhibit a propensity for developing an ordered secondary structure. In certain embodiments, the linker can be a chemical moiety which can be monomeric, dimeric, multimeric or polymeric. Preferably, the linker comprises amino acids. Typical amino acids in flexible linkers include Gly, Asn and Ser. Accordingly, in particular embodiments, the linker comprises a combination of one or more of Gly, Asn and Ser amino acids. Other near neutral amino acids, such as Thr and Ala, also may be used in the linker sequence. Exemplary linkers are disclosed in Maratea et al. (1985), Gene 40: 39-46; Murphy et al. (1986) Proc. Nat'l. Acad. Sci. USA 83: 8258-62; U.S. Pat. Nos. 4,935,233; and 4,751,180. For example, GlySer linkers GGS, GGGS (SEQ ID NO: 117) or GSG can be used. GGS, GSG, GGGS (SEQ ID NO: 117) or GGGGS (SEQ ID NO: 118) linkers can be used in repeats of 3 (such as (GGS)₃ (SEQ ID NO: 119), (GGGGS)₃ (SEQ ID NO: 120)) or 5, 6, 7, 9 or even 12 or more, to provide suitable lengths. In some cases, the linker may be (GGGGS)₃₋₁₅ (SEQ ID NO: 121), For example, in some cases, the linker may be (GGGGS)₃₋₁₅ (SEQ ID NO: 121), e.g., GGGGS (SEQ ID NO: 118), (GGGGS)₂ (SEQ ID NO: 122), (GGGGS)₃ (SEQ ID NO: 120), (GGGGS)₄ (SEQ ID NO: 123), (GGGGS)₅ (SEQ ID NO: 124), (GGGGS)₆ (SEQ ID NO: 125), (GGGGS)₇ (SEQ ID NO: 126), (GGGGS)₈ (SEQ ID NO: 127), (GGGGS)₉ (SEQ ID NO: 128), (GGGGS)₁₀ (SEQ ID NO: 129), or (GGGGS)₁₁ (SEQ ID NO: 130).

In particular embodiments, linkers such as (GGGGS)₃ (SEQ ID NO: 120) are used. (GGGGS)₆ (SEQ ID NO: 125), (GGGGS)₉ (SEQ ID NO: 128) or (GGGGS)₁₂ (SEQ ID NO: 131) may be used as alternatives. Other alternatives are (GGGGS) (SEQ ID NO: 118), (GGGGS)₂ (SEQ ID NO: 122), (GGGGS)₄ (SEQ ID NO: 123), (GGGGS)₅ (SEQ ID NO: 124), (GGGGS)₇ (SEQ ID NO: 126), (GGGGS)₈ (SEQ ID NO: 127), (GGGGS)₁₀ (SEQ ID NO: 129), or (GGGGS)₁₁ (SEQ ID NO: 130). In yet a further embodiment, LEPGEKPYKCPECGKSFSQSGALTRHQRTHTR (SEQ ID NO: 132) is used as a linker. In yet an additional embodiment, the linker is an XTEN linker. In particular embodiments, the CasPR protein is linked to the deaminase protein or its catalytic domain by means of an LEPGEKPYKCPECGKSFSQSGALTRHQRTHTR (SEQ ID NO: 132) linker. In further particular embodiments, the CasPR protein is linked C-terminally to the N-terminus of a deaminase protein or its catalytic domain by means of an LEPGEKPYKCPECGKSFSQSGALTRHQRTHTR (SEQ ID NO: 132) linker. In addition, N- and C-terminal NLSs can also function as linker (e.g., PKKKRKVEASSPKKRKVEAS (SEQ ID NO: 133)).

A nucleotide deaminase or other RNA modification enzyme may be linked to CasPR or a dead CasPR via one or more amino acids. In some cases, the nucleotide deaminase may be linked to the CasPR or a dead CasPR via one or more amino acids 411-429, 114-124, 197-241, and 607-624. The amino acid position may correspond to a CasPR ortholog disclosed herein.

### 7. CasPR- or CasPR Fusion-Guide RNA Complex

In one aspect, the invention provides a subject CasPR in complex with a guide RNA (crRNA) comprising (a) a guide sequence that is substantially complementary to a target sequence (e.g., a target mRNA), and (2) a direct repeat sequence or elements thereof for binding to the CasPR. Optionally, the CasPR is further fused to a heterologous functional domain such as ADAR for RNA base editing. The CasPR may substantially lack endonuclease activity but substantially preserves the ability of wild-type CasPR for binding to the DR sequence.

Thus the invention provides a non-naturally occurring or engineered composition comprising: (i) a CasPR protein, and (ii) a crRNA, wherein the crRNA comprises a) a guide sequence that is capable of hybridizing to a target RNA sequence, and b) a direct repeat sequence, whereby there is formed a complex comprising the CasPR protein complexed with the DR sequence of the crRNA which is hybridized to the target RNA sequence through the guide sequence. The complex can be formed in vitro or ex vivo and introduced into a cell or contacted with RNA; or can be formed in vivo. In certain embodiments, the CasPR is a dCasPR that substantially lack endonuclease activity.

In some embodiments, a non-naturally occurring or engineered composition of the invention comprises two or more crRNAs.

In some embodiments, a non-naturally occurring or engineered composition of the invention comprises a guide sequence that hybridizes to a target RNA sequence in a prokaryotic cell. In some embodiments, a non-naturally occurring or engineered composition of the invention comprises a guide sequence that hybridizes to a target RNA sequence in a eukaryotic cell.

In some embodiment, the CasPR protein comprises one or more nuclear localization signals (NLSs).

In certain embodiments, the CasPR protein of the invention is, or in, or comprises, or consists essentially of, or consists of, or involves or relates to such a protein derived from or as set forth in the section above concerning CasPR, and comprising one or more derivatives, variants, nuclease dead mutations, functional fragments thereof as described herein elsewhere.

In some embodiment of the non-naturally occurring or engineered composition of the invention, the CasPR protein is associated with one or more heterologous functional domains. The association can be by direct linkage (e.g., fusion or conjugation) of the effector protein to the heterologous functional domain, or by association with the crRNA. In a non-limiting example, the crRNA comprises an added or inserted sequence that can be associated with a functional domain of interest, including, for example, an aptamer or a nucleotide that binds to a nucleic acid binding adapter protein.

In certain non-limiting embodiments, a non-naturally occurring or engineered composition of the invention comprises a functional domain cleaves the target RNA sequence.

In certain non-limiting embodiments, the non-naturally occurring or engineered composition of the invention comprises a functional domain that modifies transcription or translation of the target RNA sequence.

In some embodiment of the composition of the invention, the CasPR protein is associated with one or more functional domains; and the effector protein contains one or more mutations within an endonuclease domain, whereby the complex can deliver an epigenetic modifier or a transcriptional or translational activation or repression signal. The complex can be formed in vitro or ex vivo and introduced into a cell or contacted with RNA; or can be formed in vivo.

The invention also provides a vector system, which comprises one or more vectors comprising: a first regulatory element operably linked to a nucleotide sequence encoding the CasPR protein, and a second regulatory element operably linked to a nucleotide sequence encoding the crRNA. When appropriate, the nucleotide sequence encoding the CasPR protein is codon optimized for expression in a eukaryotic cell.

In some embodiment, the vector system of the invention comprises in a single vector. In some embodiments, the one or more vectors comprise viral vectors, such as one or more retroviral, lentiviral, adenoviral, adeno-associated viral (AAV) or herpes simplex viral vectors.

The invention also provides a delivery system configured to deliver a CasPR protein and one or more nucleic acid components of a non-naturally occurring or engineered composition comprising: (i) a CasPR protein according to the invention as described herein, and (ii) a crRNA, wherein the crRNA comprises (a) a guide sequence that hybridizes to a target RNA sequence in a cell, and b) a direct repeat sequence, wherein the CasPR protein forms a complex with the crRNA, wherein the guide sequence directs sequence-specific binding to the target RNA sequence, whereby there is formed a complex comprising the CasPR protein complexed with the DR of the crRNA that is hybridized to the target RNA sequence through the guide sequence. The complex can be formed in vitro or ex vivo and introduced into a cell or contacted with RNA; or can be formed in vivo.

In some embodiment of the delivery system of the invention, the system comprises one or more vectors or one or more polynucleotide molecules, the one or more vectors or polynucleotide molecules comprising one or more polynucleotide molecules encoding the CasPR protein and one or more nucleic acid components of the non-naturally occurring or engineered composition.

In some embodiment, the delivery system of the invention comprises a delivery vehicle comprising liposome(s), particle(s), exosome(s), microvesicle(s), a gene-gun or one or more viral vector(s).

In some embodiment, the non-naturally occurring or engineered composition vector system, or delivery system of the invention is for use in a therapeutic method of treatment or in a research program.

The invention further provides a method of modifying expression of a target gene of interest, the method comprising contacting a target RNA with one or more non-naturally occurring or engineered compositions comprising: (i) a CasPR protein according to the invention as described herein, and (ii) a crRNA, wherein the crRNA comprises (a) a guide sequence that hybridizes to a target RNA sequence in a cell, and (b) a direct repeat sequence, wherein the CasPR protein forms a complex with the DR sequence of the crRNA, wherein the guide sequence directs sequence-specific binding to the target RNA sequence in a cell, whereby there is formed a complex comprising the CasPR protein complexed with the guide sequence that is hybridized to the target RNA sequence, whereby expression of the target locus of interest is modified. The complex can be formed in vitro or ex vivo and introduced into a cell or contacted with RNA; or can be formed in vivo.

In some embodiment, the method of modifying expression of a target gene of interest comprises increasing or decreasing expression of the target RNA.

In some embodiment of the method of modifying expression of a target gene of interest, the target gene is in a prokaryotic cell. In some embodiment of the method of modifying expression of a target gene of interest, the target gene is in a eukaryotic cell.

The invention provides a cell comprising a modified target of interest, wherein the target of interest has been modified according to any of the method disclosed herein. In some embodiment of the invention, the cell is a prokaryotic cell. In some embodiment of the invention, the cell is a eukaryotic cell. In some embodiment, modification of the target of interest in a cell results in: a cell comprising altered expression of at least one gene product; a cell comprising altered expression of at least one gene product, wherein the expression of the at least one gene product is increased; or a cell comprising altered expression of at least one gene product, wherein the expression of the at least one gene product is decreased. In some embodiment, the cell is a mammalian cell or a human cell.

The invention also provides a cell line of or comprising a cell disclosed herein or a cell modified by any of the methods disclosed herein, or progeny thereof.

The invention further provides a multicellular organism comprising one or more cells disclosed herein or one or more cells modified according to any of the methods disclosed herein.

The invention provides a plant or animal model comprising one or more cells disclosed herein or one or more cells modified according to any of the methods disclosed herein.

The invention provides a gene product from a cell or the cell line or the organism or the plant or animal model disclosed herein.

In some embodiment, the amount of gene product expressed is greater than or less than the amount of gene product from a cell that does not have altered expression.

### 8. Polynucleotides

The invention also provides nucleic acids encoding the CasPR proteins and guide RNAs (e.g., a crRNA) described herein.

In some embodiments, the nucleic acid is a synthetic nucleic acid. In some embodiments, the nucleic acid is a DNA molecule. In some embodiments, the nucleic acid is an RNA molecule (e.g., an mRNA molecule encoding the CasPR, variant or derivative including dCasPR, or functional fragment thereof). In some embodiments, the mRNA is capped, polyadenylated, substituted with 5-methyl cytidine, substituted with pseudouridine, or a combination thereof.

In some embodiments, the nucleic acid (e.g., DNA) is operably linked to a regulatory element (e.g., a promoter) in order to control the expression of the nucleic acid. In some embodiments, the promoter is a constitutive promoter. In some embodiments, the promoter is an inducible promoter. In some embodiments, the promoter is a cell-specific promoter. In some embodiments, the promoter is an organism-specific promoter.

Suitable promoters are known in the art and include, for example, a pol I promoter, a pol II promoter, a pol III promoter, a T7 promoter, a U6 promoter, a H1 promoter, retroviral Rous sarcoma virus LTR promoter, a cytomegalovirus (CMV) promoter, a SV40 promoter, a dihydrofolate reductase promoter, and a 3-actin promoter. For example, a U6 promoter can be used to regulate the expression of a guide RNA molecule described herein.

In one aspect, the present disclosure provides nucleic acid sequences that are at least 50%, 55%, 60%, 65%, 70%, 75%, 80%, 85%, 90%, 91%, 92%, 93%, 94%, 95%, 96%, 97%, 98%, 99%, or 100% identical to the nucleic acid sequences described herein, i.e., nucleic acid sequences encoding the CasPR proteins (e.g., SEQ ID NOs: 1-11), derivatives, functional fragments, or guide/crRNA, including the DR sequences and transcripts of SEQ ID NOs: 12-33.

In another aspect, the present disclosure also provides nucleic acid sequences encoding amino acid sequences that are at least 50%, 55%, 60%, 65%, 70%, 75%, 80%, 85%, 90%, 91%, 92%, 93%, 94%, 95%, 96%, 97%, 98%, 99%, or 100% identical to the amino acid sequences described herein, such as SEQ ID NOs: 1-11.

In some embodiments, the nucleic acid sequences have at least a portion (e.g., at least 1, 2, 3, 4, 5, 6, 7, 8, 9, 10, 11, 12, 13, 14, 15, 20, 30, 40, 50, 60, 70, 80, 90, or 100 nucleotides, e.g., contiguous or non-contiguous nucleotides) that is the same as the sequences described herein. In some embodiments, the nucleic acid sequences have at least a portion (e.g., at least 1, 2, 3, 4, 5, 6, 7, 8, 9, 10, 11, 12, 13, 14, 15, 20, 30, 40, 50, 60, 70, 80, 90, or 100 nucleotides, e.g., contiguous or non-contiguous nucleotides) that is different from the sequences described herein.

In related embodiments, the invention provides amino acid sequences having at least a portion (e.g., at least 1, 2, 3, 4, 5, 6, 7, 8, 9, 10, 11, 12, 13, 14, 15, 20, 30, 40, 50, 60, 70, 80, 90, or 100 amino acid residues, e.g., contiguous or non-contiguous amino acid residues) that is the same as the sequences described herein. In some embodiments, the amino acid sequences have at least a portion (e.g., at least 1, 2, 3, 4, 5, 6, 7, 8, 9, 10, 11, 12, 13, 14, 15, 20, 30, 40, 50, 60, 70, 80, 90, or 100 amino acid residues, e.g., contiguous or non-contiguous amino acid residues) that is different from the sequences described herein.

To determine the percent identity of two amino acid sequences, or of two nucleic acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). In general, the length of a reference sequence aligned for comparison purposes should be at least 80% of the length of the reference sequence, and in some embodiments is at least 90%, 95%, or 100% of the length of the reference sequence. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences, taking into account the number of gaps, and the length of each gap, which need to be introduced for optimal alignment of the two sequences. For purposes of the present disclosure, the comparison of sequences and determination of percent identity between two sequences can be accomplished using a Blossum 62 scoring matrix with a gap penalty of 12, a gap extend penalty of 4, and a frameshift gap penalty of 5.

The proteins described herein (e.g., CasPR proteins) can be delivered or used as either nucleic acid molecules or polypeptides.

In certain embodiments, the nucleic acid molecule encoding the CasPR proteins, derivatives or functional fragments thereof are codon-optimized for expression in a host cell or organism. The host cell may include established cell lines (such as 293T cells) or isolated primary cells. The nucleic acid can be codon optimized for use in any organism of interest, in particular human cells or bacteria. For example, the nucleic acid can be codon-optimized for any prokaryotes (such as E. coli), or any eukaryotes such as human and other non-human eukaryotes including yeast, worm, insect, plants and algae (including food crop, rice, corn, vegetables, fruits, trees, grasses), vertebrate, fish, non-human mammal (e.g., mice, rats, rabbits, dogs, birds (such as chicken), livestock (cow or cattle, pig, horse, sheep, goat etc.), or non-human primates). Codon usage tables are readily available, for example, at the “Codon Usage Database” available at www.kazusa.orjp/codon/, and these tables can be adapted in a number of ways. See Nakamura et al., Nucl. Acids Res. 28:292, 2000 (incorporated herein by reference in its entirety). Computer algorithms for codon optimizing a particular sequence for expression in a particular host cell are also available, such as Gene Forge (Aptagen; Jacobus, Pa.).

An example of a codon optimized sequence, is in this instance a sequence optimized for expression in a eukaryote, e.g., humans (i.e. being optimized for expression in humans), or for another eukaryote, animal or mammal as herein discussed; see, e.g., SaCas9 human codon optimized sequence in WO 2014/093622 (PCT/US2013/074667). Whilst this is preferred, it will be appreciated that other examples are possible and codon optimization for a host species other than human, or for codon optimization for specific organs is known. In general, codon optimization refers to a process of modifying a nucleic acid sequence for enhanced expression in the host cells of interest by replacing at least one codon (e.g. about or more than about 1, 2, 3, 4, 5, 10, 15, 20, 25, 50, or more codons) of the native sequence with codons that are more frequently or most frequently used in the genes of that host cell while maintaining the native amino acid sequence. Various species exhibit particular bias for certain codons of a particular amino acid. Codon bias (differences in codon usage between organisms) often correlates with the efficiency of translation of messenger RNA (mRNA), which is in turn believed to be dependent on, among other things, the properties of the codons being translated and the availability of particular transfer RNA (tRNA) molecules. The predominance of selected tRNAs in a cell is generally a reflection of the codons used most frequently in peptide synthesis. Accordingly, genes can be tailored for optimal gene expression in a given organism based on codon optimization. Codon usage tables are readily available, for example, at the “Codon Usage Database” available at www.kazusa.orjp/codon/ and these tables can be adapted in a number of ways. See Nakamura, Y., et al. “Codon usage tabulated from the international DNA sequence databases: status for the year 2000” Nucl. Acids Res. 28:292 (2000). Computer algorithms for codon optimizing a particular sequence for expression in a particular host cell are also available, such as Gene Forge (Aptagen; Jacobus, PA), are also available. In some embodiments, one or more codons (e.g., 1, 2, 3, 4, 5, 10, 15, 20, 25, 50, or more, or all codons) in a sequence encoding a CasPR correspond to the most frequently used codon for a particular amino acid.

### 9. Vectors and Cells

In some embodiments, the polynucleotide(s) or nucleic acid(s) of the invention are present in a vector (e.g., a viral vector or a phage).

The term “vector” as used herein generally refers to a nucleic acid molecule capable of transporting another nucleic acid to which it has been linked. Vectors include, but are not limited to, nucleic acid molecules that are single-stranded, double-stranded, or partially double-stranded; nucleic acid molecules that comprise one or more free ends, no free ends (e.g., circular); nucleic acid molecules that comprise DNA, RNA, or both; and other varieties of polynucleotides known in the art.

In certain embodiments, the vector can be a cloning vector, or an expression vector. The vectors can be plasmids, phagemids, Cosmids, etc. The vectors may include one or more regulatory elements that allow for the propagation of the vector in a cell of interest (e.g., a bacterial cell or a mammalian cell). In some embodiments, the vector includes a nucleic acid encoding a single component of a CasPR system described herein. In some embodiments, the vector includes multiple nucleic acids, each encoding a component of a CasPR system described herein.

In certain embodiments, the vector is a “plasmid,” which refers to a circular double stranded DNA loop into which additional DNA segments can be inserted, such as by standard molecular cloning techniques.

In certain embodiments, the vector is a viral vector, wherein virally-derived DNA or RNA sequences are present in the vector for packaging into a virus (e.g., retroviruses, lentiviruses, replication defective retroviruses, adenoviruses, replication defective adenoviruses, HSV, and adeno-associated viruses (AAV)). Viral vectors also include polynucleotides carried by a virus for transfection into a host cell.

In certain embodiments, the vector is capable of autonomous replication in a host cell into which they are introduced (e.g., bacterial vectors having a bacterial origin of replication and episomal mammalian vectors). In certain embodiments, the vector (e.g., non-episomal mammalian vectors) is integrated into the genome of a host cell upon introduction into the host cell, and thereby are replicated along with the host genome. In certain embodiments, the vector, referred to herein as “expression vector,” is capable of directing the expression of genes to which they are operatively-linked. Vectors for and that result in expression in a eukaryotic cell are “eukaryotic expression vectors.”

In certain embodiments, the vector is a recombinant expression vector that comprises a nucleic acid of the invention in a form suitable for expression of the nucleic acid in a host cell. The recombinant expression vector may include one or more regulatory elements, which may be selected on the basis of the host cells to be used for expression, that is operatively-linked to the nucleic acid sequence to be expressed. Here, “operably linked” means that the nucleotide sequence of interest is linked to the regulatory element(s) in a manner that allows for expression of the nucleotide sequence (e.g., in an in vitro transcription/translation system or in a host cell when the vector is introduced into the host cell).

The term “regulatory element” include promoters, enhancers, internal ribosomal entry sites (IRES), and other expression control elements (e.g., transcription termination signals, such as polyadenylation signals and poly-U sequences). Such regulatory elements are described, for example, in Goeddel, GENE EXPRESSION TECHNOLOGY: METHODS IN ENZYMOLOGY 185, Academic Press, San Diego, Calif. (1990). Regulatory elements include those that direct constitutive expression of a nucleotide sequence in many types of host cell and those that direct expression of the nucleotide sequence only in certain host cells (e.g., tissue-specific regulatory sequences). A tissue-specific promoter may direct expression primarily in a desired tissue of interest, such as muscle, neuron, bone, skin, blood, specific organs (e.g., liver, pancreas), or particular cell types (e.g., lymphocytes). Regulatory elements may also direct expression in a temporal-dependent manner, such as in a cell-cycle dependent or developmental stage-dependent manner, which may or may not also be tissue or cell-type specific.

In some embodiments, a vector comprises one or more pol III promoter (e.g., 1, 2, 3, 4, 5, or more pol III promoters), one or more pol II promoters (e.g., 1, 2, 3, 4, 5, or more pol II promoters), one or more pol I promoters (e.g., 1, 2, 3, 4, 5, or more pol I promoters), or combinations thereof. Examples of pol III promoters include, but are not limited to, U6 and H1 promoters. Examples of pol II promoters include, but are not limited to, the retroviral Rous sarcoma virus (RSV) LTR promoter (optionally with the RSV enhancer), the cytomegalovirus (CMV) promoter (optionally with the CMV enhancer) [see, e.g., Boshart et al, Cell, 41:521-530 (1985)], the SV40 promoter, the dihydrofolate reductase promoter, the β-actin promoter, the phosphoglycerol kinase (PGK) promoter, and the EF1a promoter.

Also encompassed by the term “regulatory element” are enhancer elements, such as WPRE; CMV enhancers; the R-U5′ segment in LTR of HTLV-I (Mol. Cell. Biol., Vol. 8(1), p. 466-472, 1988); SV40 enhancer; and the intron sequence between exons 2 and 3 of rabbit b-globin (Proc. Natl. Acad. Sci. USA., Vol. 78(3), p. 1527-31, 1981).

It will be appreciated by those skilled in the art that the design of the expression vector can depend on such factors as the choice of the host cell to be transformed, the level of expression desired, etc. A vector can be introduced into host cells to thereby produce transcripts, proteins, or peptides, including fusion proteins or peptides, encoded by nucleic acids as described herein (e.g., CasPR transcripts, proteins, fragments, mutants thereof, fusion proteins thereof, guide RNA linked to DR sequence, etc.).

In certain embodiments, the vector is a lentiviral or AAV vector, which can be selected for targeting particular types of cells (e.g., with tissue and/or cell type-specific tropism).

In a related aspect, the invention also provides a cell comprising any of the CasPR system of the invention, including one or more of: the CasPR protein (including wild-type, variant/derivative (including dCasPR), functional fragment) or fusions with a heterologous functional domain such as RNA deaminase, crRNA or guide RNA comprising a guide sequence and DR sequence, complex of the invention, polynucleotide encoding the protein or crRNA, or vector of the invention comprising the polynucleotide of the invention.

In certain embodiments, the cell is a prokaryote.

In certain embodiments, the cell is a eukaryote. When the cell is a eukaryote, the complex in the eukaryotic cell can be a naturally existing CasPR complex in a prokaryote from which the CasPR is isolated.

### 10. General Methods of Use

In another aspect, the present disclosure discloses methods of using the compositions and systems herein.

In general, the methods include modifying a target nucleic acid by introducing in a cell or organism that comprises the target nucleic acid the CasPR protein or fusions thereof, guide/crRNA, polynucleotide(s) encoding the CasPR protein and/or crRNA, or the vector or vector system comprising the polynucleotide(s), such that the CasPR protein/crRNA modifies the target nucleic acid in the cell or organism.

In some embodiments, the target nucleic acid comprises an RNA encoded by a genomic locus, and the CasPR protein modifies gene product encoded by the genomic locus or expression of the gene product. The target nucleic acid is DNA or RNA and wherein one or more nucleotides in the target nucleic acid may be base edited. The target nucleic acid may be DNA or RNA and wherein the target nucleic acid is cleaved.

In some embodiments, the methods may further comprise visualizing activity and, optionally, using a detectable label. The method may also comprise detecting binding of one or more components of the CasPR system to the target nucleic acid.

In some embodiments, at least one guide polynucleotide comprises a mismatch for base editing. The mismatch may be up- or downstream of a single nucleotide variation on the one or more guide sequences.

In certain embodiments, the guide RNA is designed such that the mismatch is located on position 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 of the spacer sequence (starting at the 5′ end). In certain embodiments, the guide RNA is designed such that the mismatch is located on position 1, 2, 3, 4, 5, 6, 7, 8, or 9 of the spacer sequence (starting at the 5′ end). In certain embodiments, the guide RNA is designed such that the mismatch is located on position 10, 11, 12, 13, 14, 15, 16, 17, 18, or 19 of the spacer sequence (starting at the 5′ end). In certain embodiments, the guide RNA is designed such that the mismatch is located on position 20, 21, 22, 23, 24, 25, 26, 27, 28, or 29 of the spacer sequence (starting at the 5′ end). In certain embodiments, the guide RNA is designed such that the mismatch is located on position 14, 15, 16, or 17 of the spacer sequence (starting at the 5′ end). In certain embodiments, the guide RNA is designed such that the mismatch is located on position 15 of the spacer sequence (starting at the 5′ end). In certain embodiments, the mismatch distance depends on the length of the spacer, such as anywhere between 3 to (N-3) nt, wherein N is the length of the spacer.

In certain embodiments, the method and system of the invention provides compositions and methods for transcript tracking, which, for example, allows one to visualize transcripts in cells, tissues, organs or animals, providing important spatio-temporal information regarding RNA dynamics and function.

In certain embodiments, the method and system of the invention provides compositions and methods for nucleic acid targeting, using either wild-type CasPR of the invention, or orthologs, paralogs, variants, derivatives (including dCasPR), or functional fragments thereof.

A number of diseases have been demonstrated to be treatable by mRNA targeting. Examples of mRNA targets (and corresponding disease treatments) are VEGF, VEGF-R1 and RTP801 (in the treatment of AMD and/or DME), Caspase 2 (in the treatment of NAION), ADRB2 (in the treatment of intraocular pressure), TRPVI (in the treatment of Dry eye syndrome, Syk kinase (in the treatment of asthma), Apo B (in the treatment of hypercholesterolemia), PLK1, KSP and VEGF (in the treatment of solid tumors), Ber-Abl (in the treatment of CML) (Burnett and Rossi, Chem Biol. 19(1):60-71, 2012). Similarly, RNA targeting has been demonstrated to be effective in the treatment of RNA-virus mediated diseases such as HIV (targeting of HIV Tet and Rev), RSV (targeting of RSV nucleocapsid) and HCV (targeting of miR-122) (Burnett and Rossi, Chem Biol. 19(1):60-71, 2012).

In certain embodiments, the RNA targeting CasPR effector protein of the invention can be used for mutation specific or allele specific knockdown. Guide RNA's can be designed that specifically target a sequence in the transcribed mRNA comprising a mutation or an allele-specific sequence. Such specific knockdown is particularly suitable for therapeutic applications relating to disorders associated with mutated or allele-specific gene products. For example, most cases of familial hypobet alipoproteinemia (FHBL) are caused by mutations in the ApoB gene. This gene encodes two versions of the apolipoprotein B protein: a short version (ApoB-48) and a longer version (AroB-100). Several ApoB gene mutations that lead to FHBL cause both versions of ApoB to be abnormally short. Specifically targeting and knockdown of mutated ApoB mRNA transcripts with an RNA targeting effector protein of the invention may be beneficial in treatment of FHBL. As another example, Huntington's disease (HD) is caused by an expansion of CAG triplet repeats in the gene coding for the Huntingtin protein, which results in an abnormal protein. Specifically targeting and knockdown of mutated or allele-specific mRNA transcripts encoding the Huntingtin protein with an RNA targeting effector protein of the invention may be beneficial in treatment of HD.

Apart from a direct effect on gene expression through cleavage of the mRNA, RNA targeting can also be used to impact specific aspects of the RNA processing within the cell, which may allow a more subtle modulation of gene expression. Generally, modulation can for instance be mediated by interfering with binding of proteins to the RNA, such as for instance blocking binding of proteins, or recruiting RNA binding proteins. Indeed, modulations can be ensured at different levels such as splicing, transport, localization, translation and turnover of the mRNA. Similarly in the context of therapy, it can be envisaged to address (pathogenic) malfunctioning at each of these levels by using RNA-specific targeting molecules. In these embodiments, it is in many cases preferred that the RNA targeting protein is a “dead” CasPR that has lost the ability to cut the RNA target but maintains its ability to bind thereto, such as the mutated forms of CasPR described herein.

In certain embodiments, the CasPR RNA targeting effector proteins described herein can for instance be used to block or promote slicing, include or exclude exons and influence the expression of specific isoforms and/or stimulate the expression of alternative protein products.

In certain embodiments, the CasPR RNA targeting effector proteins described herein can for instance be used in RNA editing, such as converting adenosine (A) to inosine (I) and/or cytidine (C) to uracil (U), resulting in an RNA sequence which is different from that encoded by the (mutant) genome. A classic example of A-I editing is the glutamate receptor GluR-B mRNA, whereby the change results in modified conductance properties of the channel (Higuchi et al., Cell 75:1361-70, 1993). Also, in humans, a heterozygous functional-null mutation in the ADAR1 gene leads to a skin disease, human pigmentary genodermatosis (Miyamura et al., Am J Hum Genet. 73:693-9, 2003). Thus, the CasPR RNA targeting effector proteins of the present invention can be used to correct malfunctioning RNA modification.

In certain embodiments, the CasPR RNA targeting effector proteins described herein can for instance be used to interfere with or promote the interaction between the RNA-binding proteins and RNA. Examples of diseases which have been linked to defective proteins involved in polyadenylation are oculopharyngeal muscular dystrophy (OPMD) (Brais et al., Nat Genet. 18:164-7, 1998).

In certain embodiments, the CasPR RNA targeting effector proteins described herein can for instance be used to target localization elements to the RNA of interest. The CasPR effector proteins can be designed to bind the target transcript and shuttle them to a location in the cell determined by its peptide signal tag. More particularly for instance, a CasPR RNA targeting effector protein fused to a nuclear localization signal (NLS) can be used to alter RNA localization. Further examples of localization signals include the zipcode binding protein (ZBP1) which ensures localization of b-actin to the cytoplasm in several asymmetric cell types, KDEL retention sequence (localization to endoplasmic reticulum), nuclear export signal (localization to cytoplasm), mitochondrial targeting signal (localization to mitochondria), peroxisomal targeting signal (localization to peroxisome) and m6A marking/YTHDF2 (localization to p-bodies). Other approaches that are envisaged are fusion of the RNA targeting effector protein with proteins of known localization (for instance membrane, synapse). Alternatively, the CasPR effector protein according to the invention may for instance be used in localization-dependent knockdown. By fusing the CasPR effector protein to an appropriate localization signal, the effector is targeted to a particular cellular compartment. Only target RNAs residing in this compartment will effectively be targeted, whereas otherwise identical targets, but residing in a different cellular compartment will not be targeted, such that a localization dependent knockdown can be established.

In certain embodiments, the CasPR RNA targeting effector proteins described herein can for instance be used to bring translation initiation factors, such as EIF4G in the vicinity of the 5′ untranslated repeat (5′ LTR) of a messenger RNA of interest to drive translation (as described in De Gregorio et al., EMBO J. 18(17):4865-74, 1999, for a non-reprogrammable RNA binding protein). As another example GLD2, a cytoplasmic poly(A) polymerase, can be recruited to the target mRNA by a CasPR RNA targeting effector protein. This would allow for directed polyadenylation of the target mRNA thereby stimulating translation. Similarly, the CasPR RNA targeting effector proteins can be used to block translational repressors of mRNA, such as ZBP1 (Huttelmaier et al., Nature 438:512-5, 2005). In addition, fusing the CasPR RNA targeting effector proteins to a protein that stabilizes mRNAs, e.g., by preventing degradation thereof such as RNase inhibitors, it is possible to increase protein production from the transcripts of interest. Further, the CasPR RNA targeting effector proteins can be used to repress translation by binding in the 5′ UTR regions of a RNA transcript and preventing the ribosome from forming and beginning translation. The CasPR RNA targeting effector protein can also be used to recruit Caf1, a component of the CCR4-NOT deadenylase complex, to the target mRNA, resulting in deadenylation or the target transcript and inhibition of protein translation. In certain embodiments, the CasPR RNA targeting effector protein of the invention can be used to increase or decrease translation of therapeutically relevant proteins. Examples of therapeutic applications wherein the RNA targeting effector protein can be used to downregulate or upregulate translation are in amyotrophic lateral sclerosis (ALS) and cardiovascular disorders. Reduced levels of the glial glutamate transporter EAAT2 have been reported in ALS motor cortex and spinal cord, as well as multiple abnormal EAAT2 mRNA transcripts in ALS brain tissue. Loss of the EAAT2 protein and function thought to be the main cause of excitotoxicity in ALS. Restoration of EAAT2 protein levels and function may provide therapeutic benefit. Hence, the CasPR RNA targeting effector protein can be beneficially used to upregulate the expression of EAAT2 protein, e.g., by blocking translational repressors or stabilizing mRNA as described above. Apolipoprotein A1 is the major protein component of high density lipoprotein (HDL) and ApoA1 and HDL are generally considered as atheroprotective. The CasPR RNA targeting effector protein can be beneficially used to upregulate the expression of ApoA1, e.g., by blocking translational repressors or stabilizing mRNA as described above.

In certain embodiments, the CasPR RNA targeting effector protein can interfere with or promote the activity of proteins acting to stabilize mRNA transcripts, such that mRNA turnover is affected. For instance, recruitment of human TTP to the target RNA using the CasPR RNA targeting effector protein would allow for adenylate-uridylate-rich element (AU-rich element) mediated translational repression and target degradation. AU-rich elements are found in the 3′ UTR of many mRNAs that code for proto-oncogenes, nuclear transcription factors, and cytokines and promote RNA stability. As another example, the RNA targeting effector protein can be fused to HuR, another mRNA stabilization protein (Hinman et al., Cell Mol Life Sci 65:3168-81, 2008), and recruit it to a target transcript to prolong its lifetime or stabilize short-lived mRNA. In certain embodiments, the CasPR RNA targeting effector protein can be used to promote degradation of target transcripts.

In certain embodiments, the CasPR RNA targeting effector protein can be used to interfere with the binding of RNA-binding proteins at one or more locations.

In certain embodiments, the CasPR RNA targeting effector protein can be used to direct folding of (m)RNA and/or capture the correct tertiary structure thereof.

In certain embodiments, the CasPR RNA targeting effector protein can be used to modulating cellular status, in RNA detection, in vitro APEX labeling, interrogate function of lncRNA and other nuclear RNAs, identification of RNA binding proteins, assembly of complexes on RNA and substrate shuttling, synthetic biology, or protein splicing/inteins,

### 11. Method of Using Base Editing

### Regulation of Post-translational Modification of Gene Products

In some cases, base editing may be used for regulating post-translational modification of a gene products. In some cases, an amino acid residue that is a post-translational modification site may be mutated by base editing to an amino residue that cannot be modified. Examples of such post-translational modifications include disulfide bond formation, glycosylation, lipidation, acetylation, phosphorylation, methylation, ubiquitination, sumoylation, or any combinations thereof.

In some embodiments, the base editors herein may regulate Stat3/IRF-5 pathway, e.g., for reduction of inflammation. For example, phosphorylation on Tyr705 of Stat3, Thr10, Ser158, Ser309, Ser317, Ser451, and/or Ser462 of IRF-5 may be involved with interleukin signaling. Base editors herein may be used to mutate one or more of these procreation sites for regulating immunity, autoimmunity, and/or inflammation.

In some embodiments, the base editors herein may regulate insulin receptor substrate (IRS) pathway. For example, phosphorylation on Ser265, Ser302, Ser325, Ser336, Ser358, Ser407, and/or Ser408 may be involved in regulating (e.g., inhibit) ISR pathway. Alternatively or additionally, Serine 307 in mouse (or Serine 312 in human) may be mutated so the phosphorylation may be regulated. For example, Serine 307 phosphorylation may lead to degradation of IRS-1 and reduce MAPK signaling. Serine 307 phosphorylation may be induced under insulin insensitivity conditions, such as insulin overstimulation and/or TNFa treatment. In some examples, S307F mutation may be generated for stabilizing the interaction between IRS-1 and other components in the pathway. Base editors herein may be used to mutate one or more of these procreation sites for regulating IRS pathway.

### Regulation of Stability of Gene Products

In some embodiments, base editing may be used for regulating the stability of gene products. For example, one or more amino acid residues that regulate protein degradation rates may be mutated by the base editors herein. In some cases, such amino acid residues may be in a degron. A degron may refer to a portion of a protein involved in regulating the degradation rate of the protein. Degrons may include short amino acid sequences, structural motifs, and exposed amino acids (e.g., lysine or arginine). Some protein may comprise multiple degrons. The degrons be ubiquitin-dependent (e.g., regulating protein degradation based on ubiquitination of the protein) or ubiquitin-independent.

In some cases, the based editing may be used to mutate one or more amino acid residues in a signal peptide for protein degradation. In some examples, the signal peptide may be a PEST sequence, which is a peptide sequence that is rich in proline (P), glutamic acid (E), serine (S), and threonine (T). For example, the stability of NANOG, which comprises a PEST sequence, may be increased, e.g., to promote embryonic stem cell pluripotency.

In some examples, the base editors may be used for mutating SMN2 (e.g., to generate S270A mutilation) to increase stability of the SMN2 protein, which is involved in spinal muscular atrophy. Other mutations in SMN2 that may be generated by based editors include those described in Cho S. et al., Genes Dev. 2010 Mar. 1; 24(5): 438-442. In certain examples, the base editors may be used for generating mutations on IκBa, as described in Fortmann K T et al., J Mol Biol. 28; 427(17): 2748-2756, 2015. Target sites in degrons may be identified by computational tools, e.g., the online tools provided on slim.ucd.ie/apc/index.php. Other targets include Cdc25A phosphatase.

### Examples of Genes Targetable by Base Editors

In some examples, the base editors may be used for modifying PCSK9. The base editors may introduce stop codons and/or disease-associated mutations that reduce PCSK9 activity. The base editing may introduce one or more of the following mutations in PCSK9: R46L, R46A, A53V, A53A, E57K, Y142X, L253F, R237W, H391N, N425S, A443T, I474V, I474A, Q554E, Q619P, E670G, E670A, C679X, H417Q, R469W, E482G, F515L, and/or H553R.

In some examples, the base editors may be used for modifying ApoE. The base editors may target ApoE in synthetic model and/or patient-derived neurons (e.g., those derived from iPSC). The targeting may be tested by sequencing.

In some examples, the base editors may be used for modifying Stat1/3. The base editor may target Y705 and/or S727 for reducing Stat1/3 activation. The base editing may be tested by luciferase-based promoter. Targeting Stat1/3 by base editing may block monocyte to macrophage differentiation, and inflammation in response to ox-LDL stimulation of macrophages.

In some examples, the base editors may be used for modifying TFEB (transcription factor for EB). The base editor may target one or more amino acid residues that regulate translocation of the TFEB. In some cases, the base editor may target one or more amino acid residues that regulate autophagy.

In some examples, the base editors may be used for modifying ornithine carbamoyl transferase (OTC). Such modification may be used for correct ornithine carbamoyl transferase deficiency. For example, base editing may correct Leu45Pro mutation by converting nucleotide 134C to U.

In some examples, the base editors may be used for modifying Lipin1. The base editor may target one or more serine's that can be phosphorylated by mTOR. Base editing of Lipin1 may regulate lipid accumulation. The base editors may target Lipin1 in 3T3L1 preadipocyte model. Effects of the base editing may be tested by measuring reduction of lipid accumulation (e.g., via oil red).

### 12. Delivery

The compositions of the invention, including the various CasPR proteins (including wild-type CasPR such as Cas5d, Cas6, Csf5, variants and derivatives thereof (including endonuclease dead CasPR) and functional fragments thereof) or fusions thereof with a heterologous functional domain, as well as guide RNA/crRNA comprising the guide sequence and DR repeat sequences, or polynucleotides encoding the same, or vectors comprising the polynucleotide (collectively referred to herein as the subject “CasPR system”), can be delivered/introduced to a target cell, tissue, organ, organism, or individual/subject/patient, using art-recognized means. The cell or organisms may be a eukaryotic cell or organism. The cell or organisms is an animal cell or organism. The cell or organisms is a plant cell or organism.

In some embodiments, the subject CasPR system is introduced via delivery by liposomes, nanoparticles, exosomes, microvesicles, nucleic acid nanoassemblies, a gene gun, an implantable device, or the vector system herein. In some embodiments, the CasPR protein is associated with one or more functional domains. In some embodiments, the target nucleic acid comprises an RNA (such as mRNA) or a genomic locus, and the engineered CasPR protein modifies gene product encoded by the genomic locus or expression of the gene product (e.g., mRNA). In some embodiments, the target nucleic acid is DNA or RNA, and wherein one or more nucleotides in the target nucleic acid are base edited. In some embodiments, the target nucleic acid is DNA or RNA and wherein the target nucleic acid is cleaved. In some embodiments, the method further comprises visualizing activity and, optionally, using a detectable label. In some embodiments, the method further comprises detecting binding of one or more components of the CasPR system to the target nucleic acid. In some embodiments, said cell or organisms is a eukaryotic cell or organism. In some embodiments, said cell or organisms is an animal cell or organism. In some embodiments, said cell or organisms is a plant cell or organism.

Examples of nucleic acid nanoassemblies include DNA origami and RNA origami, e.g., those described in U.S. Pat. No. 8,554,489, US20160103951, WO2017189914, and WO2017189870, which are incorporated by reference in their entireties. A gene gun may include a biolistic particle delivery system, which is a device for delivering exogenous DNA (transgenes) to cells. The payload may be an elemental particle of a heavy metal coated with DNA (typically plasmid DNA). An example of delivery components in CasPR systems is described in Svitashev et al., Nat Commun. 7:13274, 2016.

The nanoparticle may be a colloidal metal. The colloidal metal material may include water-insoluble metal particles or metallic compounds dispersed in a liquid, a hydrosol, or a metalsol. The colloidal metal may be selected from the metals in groups IA, IB, IIB and IIIB of the periodic table, as well as the transition metals, especially those of group VIII. Preferred metals include gold, silver, aluminum, ruthenium, zinc, iron, nickel and calcium. Other suitable metals also include the following in all of their various oxidation states: lithium, sodium, magnesium, potassium, scandium, titanium, vanadium, chromium, manganese, cobalt, copper, gallium, strontium, niobium, molybdenum, palladium, indium, tin, tungsten, rhenium, platinum, and gadolinium. The metals are preferably provided in ionic form, derived from an appropriate metal compound, for example the Al³⁺, Ru³⁺, Zn²⁺, Fe³⁺, Ni²⁺ and Ca²⁺ ions.

In another embodiment, the delivery is via liposomes or lipofection formulations and the like, and can be prepared by methods known to those skilled in the art. Such methods are described, for example, in WO 2016205764 and U.S. Pat. Nos. 5,593,972; 5,589,466; and 5,580,859; each of which is incorporated herein by reference in its entirety.

In some embodiments, the delivery is via nanoparticles or exosomes. For example, exosomes have been shown to be particularly useful in delivery RNA.

The subject CasPR system can also be delivered by various delivery systems such as vectors, e.g., plasmids and viral delivery vectors, using any suitable means in the art. Such methods include (and are not limited to) electroporation, lipofection, microinjection, transfection, sonication, gene gun, etc.

In certain embodiments, the CasPR proteins and/or any of the RNAs (e.g., guide RNAs or crRNAs) can be delivered using suitable vectors, e.g., plasmids or viral vectors, such as adeno-associated viruses (AAV), lentiviruses, adenoviruses, retroviral vectors, and other viral vectors, or combinations thereof. The proteins and one or more crRNAs can be packaged into one or more vectors, e.g., plasmids or viral vectors. For bacterial applications, the nucleic acids encoding any of the components of the CasPR systems described herein can be delivered to the bacteria using a phage. Exemplary phages, include, but are not limited to, T4 phage, Mu, λ phage, T5 phage, T7 phage, T3 phage, Φ29, M13, MS2, Qβ, and ΦX174.

In some embodiments, the vectors, e.g., plasmids or viral vectors, are delivered to the tissue of interest by, e.g., intramuscular injection, intravenous administration, transdermal administration, intranasal administration, oral administration, or mucosal administration. Such delivery may be either via a single dose, or multiple doses. One skilled in the art understands that the actual dosage to be delivered herein may vary greatly depending upon a variety of factors, such as the vector choices, the target cells, organisms, tissues, the general conditions of the subject to be treated, the degrees of transformation/modification sought, the administration routes, the administration modes, the types of transformation/modification sought, etc.

In certain embodiments, the delivery is via adenoviruses, which can be at a single dose containing at least 1×10⁵ particles (also referred to as particle units, pu) of adenoviruses. In some embodiments, the dose preferably is at least about 1×10⁶ particles, at least about 1×10⁷ particles, at least about 1×10⁸ particles, and at least about 1×10⁹ particles of the adenoviruses. The delivery methods and the doses are described, e.g., in WO 2016205764 A1 and U.S. Pat. No. 8,454,972 B2, both of which are incorporated herein by reference in the entirety.

In some embodiments, the delivery is via plasmids. The dosage can be a sufficient number of plasmids to elicit a response. In some cases, suitable quantities of plasmid DNA in plasmid compositions can be from about 0.1 to about 2 mg. Plasmids will generally include (i) a promoter; (ii) a sequence encoding a nucleic acid-targeting CRISPR-associated proteins and/or an accessory protein, each operably linked to a promoter (e.g., the same promoter or a different promoter); (iii) a selectable marker; (iv) an origin of replication; and (v) a transcription terminator downstream of and operably linked to (ii). The plasmids can also encode the RNA components of a CRISPR complex, but one or more of these may instead be encoded on different vectors. The frequency of administration is within the ambit of the medical or veterinary practitioner (e.g., physician, veterinarian), or a person skilled in the art.

Further means of introducing one or more components of the CasPR systems to the cell is by using cell penetrating peptides (CPP). In some embodiments, a cell penetrating peptide is linked to the CasPR proteins. In some embodiments, the CasPR proteins and/or guide RNAs are coupled to one or more CPPs to effectively transport them inside cells (e.g., plant protoplasts). In some embodiments, the CasPR proteins and/or guide RNA(s) are encoded by one or more circular or non-circular DNA molecules that are coupled to one or more CPPs for cell delivery.

CPPs are short peptides of fewer than 35 amino acids derived either from proteins or from chimeric sequences capable of transporting biomolecules across cell membrane in a receptor independent manner. CPPs can be cationic peptides, peptides having hydrophobic sequences, amphipathic peptides, peptides having proline-rich and anti-microbial sequences, and chimeric or bipartite peptides. Examples of CPPs include, e.g., Tat (which is a nuclear transcriptional activator protein required for viral replication by HIV type 1), penetratin, Kaposi fibroblast growth factor (FGF) signal peptide sequence, integrin β3 signal peptide sequence, polyarginine peptide Args sequence, Guanine rich-molecular transporters, and sweet arrow peptide. CPPs and methods of using them are described, e.g., in Hallbrink et al., “Prediction of cell-penetrating peptides,” Methods Mol. Biol., 2015; 1324:39-58; Ramakrishna et al., “Gene disruption by cell-penetrating peptide-mediated delivery of Cas9 protein and guide RNA,” Genome Res., 2014 June; 24(6):1020-7; and WO 2016205764 A1; each of which is incorporated herein by reference in its entirety.

Various delivery methods for the CasPR systems described herein are also described, e.g., in U.S. Pat. No. 8,795,965, EP 3009511, WO 2016205764, and WO 2017070605; each of which is incorporated herein by reference in its entirety.

### 13. Pharmaceutical Composition

In another aspect, the present disclosure provides a pharmaceutical composition comprising the CasPR system, including the various CasPR proteins (including wild-type CasPR such as Cas5d, Cas6, Csf5, variants and derivatives thereof (including endonuclease dead CasPR) and functional fragments thereof) or fusions thereof with a heterologous functional domain, as well as guide RNA/crRNA comprising the guide sequence and DR repeat sequences, or polynucleotides encoding the same, or vectors comprising the polynucleotide, formulated for delivery by liposomes, nanoparticles, exosomes, microvesicles, nucleic acid nanoassemblies, a gene gun, or an implantable device.

### 14. Kits

Another aspect of the invention provides a kit, comprising any two or more components of the subject CasPR system described herein, such as the CasPR proteins, derivatives, functional fragments or the various fusions or adducts thereof, guide RNA/crRNA, complexes thereof, vectors encompassing the same, or host encompassing the same.

In certain embodiments, the kit further comprise an instruction to use the components encompassed therein, and/or instructions for combining with additional components that may be available elsewhere.

In certain embodiments, the kit further comprise one or more nucleotides, such as nucleotide(s) corresponding to those useful to insert the guide RNA coding sequence into a vector and operably linking the coding sequence to one or more control elements of the vector.

In certain embodiments, the kit further comprise one or more buffers that may be used to dissolve any of the components, and/or to provide suitable reaction conditions for one or more of the components. Such buffers may include one or more of PBS, HEPES, Tris, MOPS, Na₂CO₃, NaHCO₃, NaB, or combinations thereof. In certain embodiments, the reaction condition includes a proper pH, such as a basic pH. In certain embodiments, the pH is between 7-10.

In certain embodiments, any one or more of the kit components may be stored in a suitable container.

All publications, published patent documents, and patent applications cited herein are hereby incorporated by reference to the same extent as though each individual publication, published patent document, or patent application was specifically and individually indicated as being incorporated by reference.

### EXAMPLES

### Example 1 Class I CRISPR/Cas Systems and Pre-crRNA Processing Enzymes (CasPR) Therefor

CRISPR-Cas systems can be broadly divided into two classes—Class 1 systems and Class 2 systems. One of the major differences between here two systems is that the Class 1 systems use a complex of multiple Cas proteins to degrade foreign nucleic acids (DNA or RNA), while Class 2 systems use a single large Cas protein for the same purpose.

Presently, the Class 1 system is further divided into types I, III, and IV; and the Class 2 system is further divided into types II, V, and VI. These 6 system types are additionally divided into 19 subtypes. Classification is also based on the complement of cas genes that are present. Most CRISPR-Cas systems have a Cas1 protein. Many prokaryotes contain multiple CRISPR-Cas systems, suggesting that they are compatible and may share components.

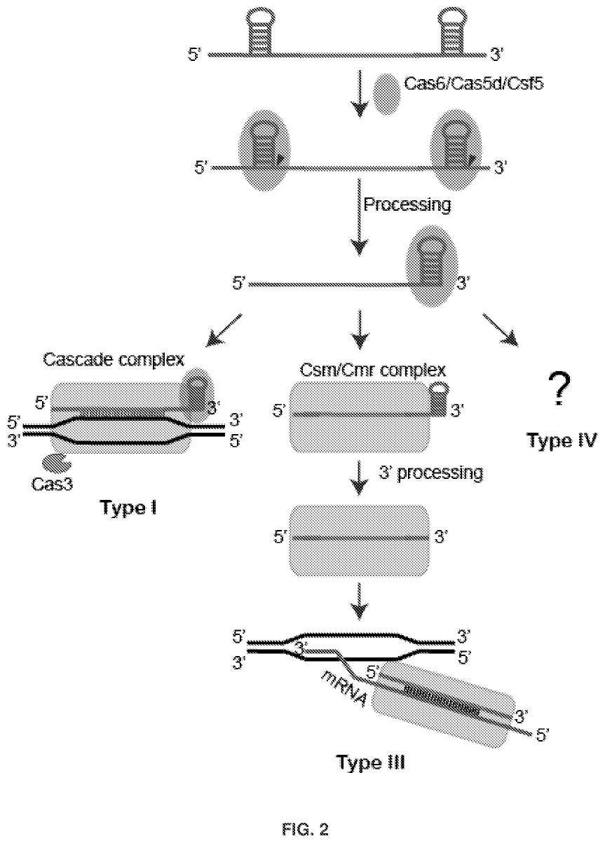

Class 1 type I effector enzymes mainly forms CASCADE complexes, Class 1 type III enzymes mainly forms CSM/CMR complexes, while the structure and function of the Class 1 type IV proteins are not fully understood. See FIG. 2.

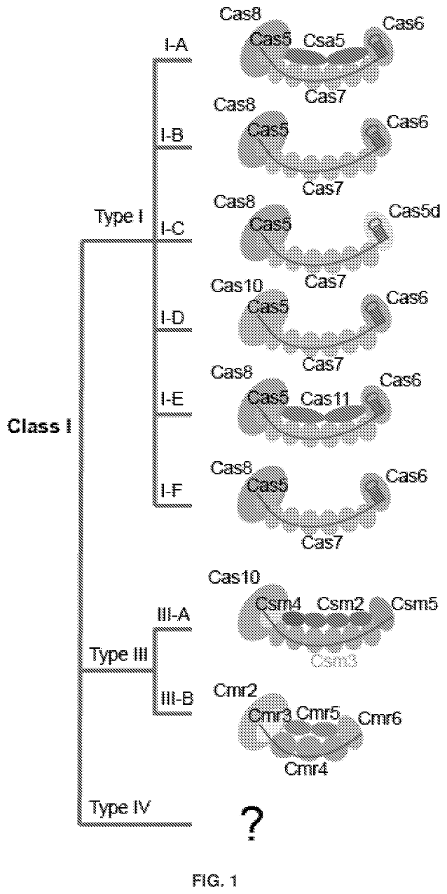

CRISPR-RNA (crRNA), which later guides the Cas nuclease to the target polynucleotides, must initially be generated from the CRISPR sequence. The crRNA is initially transcribed as part of a single long transcript (pre-crRNA) encompassing much of the CRISPR array. This pre-crRNA transcript is then cleaved by the various Cas proteins to form crRNAs. The mechanism to produce crRNAs differs among CRISPR/Cas systems. The Cas protein responsible for pre-crRNA processing/maturation is Cas5d for the Class 1 type I subtype I-C family, is Csf5 for the Class 1 type IV family, and is Cas6 for the other Class 1 types or subtypes. For simplicity, these Cas proteins responsible for Class 1 pre-crRNA processing are collectively called CasPR. See FIG. 1.

To study the functional characteristics of the various CasPR proteins for the Class 1 systems, 1 or 2 representative Cas proteins were selected from each type or subtype, including SsoCas6 (I-A), MmCas6 (I-B), SpCas5d (I-C1), BhCas5d (I-C2), SaCas6 (I-D), EcCas6e (I-E), PaCas6f (I-F), MtCas6 (III-A), PfCas6 (III-B), PaCsf5 (IV-A1), and MtCsf5 (IV-A2).

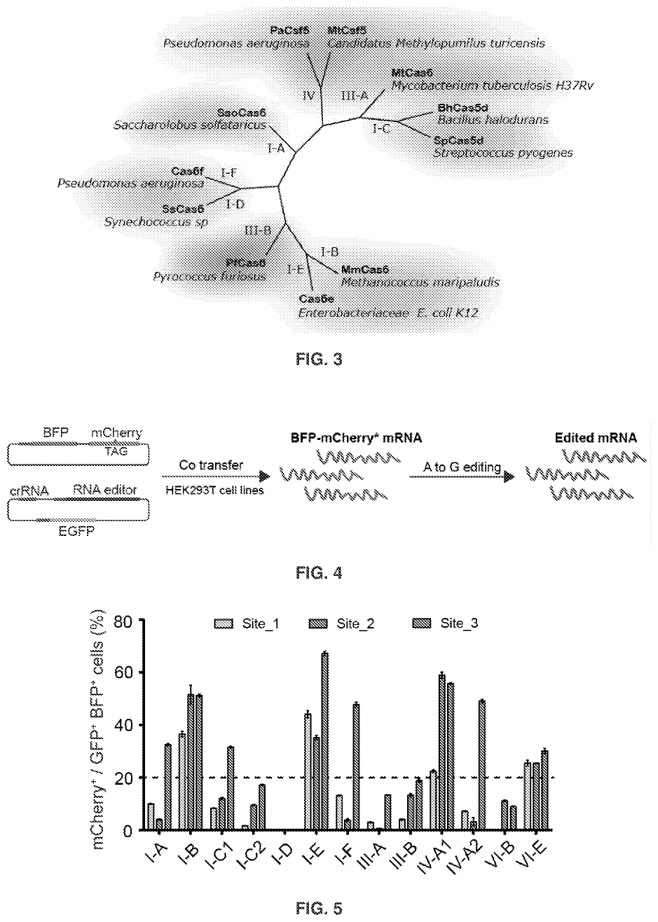

MAFFT and MEGA8 were then used to conduct multi-sequence alignments for 11 CasPR proteins to generate a phylogenetic tree (FIG. 3).

To study whether the CasPR proteins have mRNA targeting activity, the CasPR proteins were codon optimized for mammalian expression, to generate the codon-optimized sequences in SEQ ID NOs: 34-44.

SsoCas6 (I-A):
(SEQ ID NO: 34)
ATGCCTCTGATCTTCAAGATCGGCTATAACGTGATCCCCCTGCAGGACGTGATCCTGCCCACCC
CTTCCAGCAAGGTGCTGAAGTACCTGATCCAGAGCGGCAAGCTGATCCCCAGCCTGAAGGACCT
GATCACCAGCCGGGACAAGTACAAGCCAATCTTCATCTCCCACCTGGGCTTCAACCAGCGGAGG
ATTTTCCAGACCAACGGCAATCTGAAAACCATCACCAAGGGCAGTAGACTGAGCTCCATCATCG
CCTTCAGCACCCAGGCCAACGTGCTGTCCGAGGTGGCCGATGAAGGGATCTTCGAAACCGTGTA
CGGAAAGTTCCACATCATGATCGAAAGCATCGAGATCGTGGAGGTGGAAAAGCTGAAGGAGGAG
GTGGAGAAGCACATGAACGACAACATCAGAGTGAGATTCGTGTCTCCCACACTGCTGAGCTCCA
AGGTGCTGCTGCCCCCCAGCCTGTCCGAAAGATACAAGAAGATCCACGCCGGGTACAGCACCCT
GCCCAGCGTGGGCCTGATCGTGGCCTACGCCTACAACGTGTACTGCAATCTGATCGGCAAGAAG
GAAGTGGAAGTGCGGGCCTTCAAGTTTGGAATCCTGAGCAACGCCCTGTCCAGAATCATCGGCT
ACGACCTGCACCCTGTGACCGTGGCCATCGGCGAGGACAGCAAGGGGAATCTGAGAAAGGCTCG
GGGCGTGATGGGCTGGATCGAGTTCGACATCCCCGACGAAAGACTGAAGCGGCGGGCCCTGAAC
TATCTGCTGACCAGCAGCTACCTGGGCATCGGGAGATCTCGGGGCATCGGCTTCGGCGAGATCC
GGCTGGAGTTCCGGAAGATTGAAGAGAAGGAGGGA
MmCas6 (I-B):
(SEQ ID NO: 35)
ATGGACCTGGAGTACATGCACATCTCCTACCCTAACATCCTGCTGAACATGCGGGACGGCAGCA
AGCTGCGGGGCTACTTCGCCAAGAAGTACATCGACGAAGAGATTGTGCACAACCACAGAGACAA
CGCCTTTGTGTACAAGTACCCCCAGATCCAGTTTAAGATCATCGATAGAAGCCCCCTGATCATC
GGCATTGGCTCTCTGGGCATCAATTTCCTGGAGAGCAAGCGGATCTTCTTCGAGAAGGAACTGA
TTATCAGCAACGACACCAACGACATCACCGAGGTGAACGTGCACAAGGACATGGATCACTTCGG
CACGACCGACAAGATCCTGAAGTACCAGTTCAAGACCCCTTGGATGGCACTGAACGCCAAGAAT
AGCGAGATCTACAAGAACTCTGACGAGATCGACCGGGAGGAGTTCCTGAAGAGAGTGCTGATTG
GGAATATCCTGAGCATGTCTAAGAGCCTGGGCTATACCATCGAAGAAAAGCTGAAGGTGAAGAT
TAACCTGAAGGAAGTGCCCGTGAAGTTCAAGAACCAGAACATGGTGGGCTTTCGGGGCGAGTTC
TACATCAACTTCGACATCCCTCAGTATCTGGGCATCGGCCGGAATGTGTCCCGGGGATTCGGCA
CAGTGGTGAAGGTG
SpCas5d (I-C1):
(SEQ ID NO: 36)
ATGAGAAATGAAGTGCAGTTCGAGCTGTTCGGCGACTACGCCCTGTTCACCGACCCCCTGACCA
AGATCGGCGGCGAAAAGCTGAGCTACAGCGTGCCTACCTACCAGGCCCTGAAGGGCATCGCCGA
GAGCATCTACTGGAAGCCCACCATCGTGTTCGTGATCGACGAACTGCGGGTCATGAAGCCCATT
CAGATGGAGTCTAAGGGCGTGAGGCCCATCGAGTACGGCGGCGGCAACACCCTGGCCCACTACA
CCTACCTGAAGGATGTGCACTACCAGGTGAAGGCCCACTTCGAGTTCAACCTGCACCGGCCCGA
CCTGGCCTTCGATAGAAACGAGGGCAAGCACTACTCCATCCTGCAGAGAAGCCTGAAGGCCGGC
GGCAGAAGAGATATTTTCCTGGGCGCCCGGGAGTGCCAGGGCTACGTGGCCCCCTGCGAGTTCG
GCAGCGGCGACGGCTTCTACGACGGCCAGGGCAAGTACCACCTGGGAACCATGGTGCACGGTTT
CAACTACCCCGACGAAACCGGACAGCACCAGCTGGATGTGAGACTGTGGTCTGCCGTCATGGAA
AACGGCTACATCCAGTTCCCCCGCCCTGAGGACTGCCCCATCGTGCGGCCTGTGAAGGAGATGG
AACCCAAGATCTTCAACCCCGACAACGTGCAGTCCGCCGAACAGCTGCTGCACGACCTGGGCGG
CGAA
BhCas5d (I-C2):
(SEQ ID NO: 37)
ATGTACAGAAGCCGGGACTTCTACGTGAGAGTGTCCGGCCAGCGGGCCCTGTTCACCAACCCCG
CCACCAAGGGCGGCTCCGAACGGAGCTCCTACTCCGTGCCTACCCGGCAGGCCCTGAACGGGAT
TGTGGACGCCATCTACTACAAGCCCACGTTCACCAACATCGTGACCGAGGTGAAGGTGATTAAC
CAGATCCAGACCGAACTGCAGGGCGTGCGGGCCCTGCTGCATGACTACAGCGCCGACCTGAGCT
ACGTGTCCTACCTGAGCGACGTGGTGTACCTGATTAAGTTTCATTTCGTGTGGAACGAGGATAG
AAAGGACCTGAATAGCGACCGGCTGCCAGCCAAGCATGAGGCCATCATGGAGCGGTCTATCCGG
AAGGGCGGCAGACGGGACGTGTTCCTGGGCACCAGAGAATGCCTGGGCCTGCTGGACGACATCA
GCCAGGAAGAATACGAAACCACAGTGAGCTATTACAATGGGGTGAACATCGACCTGGGCATCAT
GTTCCACAGCTTCGCTTACCCCAAGGACAAGAAAACCCCCCTGAAGTCCTACTTCACAAAGACC
GTGATGAAGAACGGCGTGATCACCTTCAAGGCCCAGTCCGAATGCGATATTGTGAACACCCTGA
GCTCCTACGCCTTCAAGGCCCCCGAGGAGATCAAGAGCGTGAACGACGAGTGCATGGAGTACGA
CGCCATGGAGAAGGGCGAAAAC
SaCas6 (I-D):
ATGCCCAACGATCCCTACAGCCTGTACTCCATCGTGATCGAACTGGGCGCCGCCGAAAAGGGAT
TCCCCACAGGCATCCTGGGCAGAAGCCTGCATAGCCAGGTGCTGCAGTGGTTCAAGCAGGATAA
CCCCTTCCTGGCCACCGAGCTGCACCAGAGCCAGATCTCCCCCTTCTCCATCTCTCCACTGATG
GGCAAGCGGCACGCCAAGCTGACCAAGGCCGGCGACCGGCTGTTCTTTCGGATCTGCCTGCTGA
GAGGAGATCTGCTGCAGCCCCTGCTGAACGGCATTGAGCAGACCGTGAACCAGAGCGTGTGCCT
GGACAAGTTCCGGTTCCGGCTGTGCCAGACCCACATCCTGCCCGGCAGCCACCCTCTGGCTGGC
GCCTCCCACTATAGCCTGATCAGCCAGACCCCAGTGAGCTCCAAGATTACCCTGGACTTCAAGA
GTTCTACCTCCTTCAAGGTGGACCGGAAGATCATCCAAGTGTTCCCTCTGGGCGAACACGTGTT
CAACAGCCTGCTCAGACGCTGGAATAACTTCGCCCCCGAGGACCTGCACTTCTCTCAGGTGGAC
TGGAGCATCCCCATCGCCGCATTCGACGTGAAAACCATCCCCATCCACCTGAAGAAGGTCGAGA
TCGGCGCACAGGGCTGGGTGACCTACATCTTCCCCAACACAGAACAGGCCAAGATCGCCTCCGT
GCTGAGCGAATTCGCCTTCTTCAGCGGAGTGGGACGGAAAACCACCATGGGCATGGGCCAGGTG
CAGGTGCGGTCC
(SEQ ID NO: 38)
EcCasbe (I-E):
(SEQ ID NO: 39)
ATGTACCTGAGCAAGGTGATCATCGCCAGAGCCTGGAGCAGAGACCTGTACCAGCTGCACCAGG
GCCTGTGGCACCTGTTCCCCAACCGGCCCGACGCCGCCCGGGATTTCCTGTTCCACGTGGAGAA
GAGAAACACCCCGGAAGGCTGCCACGTGCTGCTGCAGAGCGCACAGATGCCTGTGAGCACCGCC
GTGGCCACCGTGATCAAGACCAAGCAGGTGGAGTTCCAGCTGCAGGTGGGCGTGCCCCTGTATT
TCAGGCTGCGGGCGAATCCCATCAAGACCATCCTGGACAACCAGAAGCGGCTGGACAGCAAGGG
CAACATCAAGAGGTGCAGAGTGCCTCTGATCAAGGAGGCCGAACAGATCGCCTGGCTGCAGCGG
AAGCTGGGCAATGCCGCCAGAGTGGAGGACGTGCACCCCATCAGCGAGCGGCCCCAGTACTTCT
CCGGCGACGGAAAGAGCGGAAAGATCCAGACCGTGTGCTTCGAGGGCGTGCTGACCATCAACGA
CGCACCCGCCCTGATCGACCTCGTGCAGCAGGGGATCGGCCCTGCCAAGTCCATGGGCTGCGGA
CTGCTGTCCCTGGCCCCCCTG
PaCas6f (I-F):
(SEQ ID NO: 40)
ATGGACCACTACCTGGACATTAGACTGCGCCCTGACCCAGAGTTCCCTCCTGCCCAGCTGATGT
CTGTGCTGTTTGGCAAGCTGCACCAGGCCCTGGTGGCCCAGGGCGGTGACAGAATCGGAGTGTC
TTTCCCTGATCTGGACGAATCTAGATCTAGACTGGGAGAGAGACTGAGAATCCACGCGTCTGCC
GACGACCTGAGAGCTCTGCTGGCCAGACCATGGCTGGAAGGACTGCGCGACCACCTGCAGTTCG
GTGAACCTGCCGTGGTGCCTCACCCAACTCCATACAGACAGGTGAGTAGAGTGCAGGCAAAGTC
TAATCCAGAGAGACTGAGACGCAGACTGATGAGAAGGCATGACCTGTCCGAAGAAGAAGCCAGA
AAGAGAATCCCAGACACAGTGGCCAGAGCCCTGGATCTGCCTTTTGTGACCCTGAGAAGCCAGT
CTACCGGCCAGCACTTCAGACTGTTTATTCGCCACGGACCACTGCAGGTGACCGCCGAAGAGGG
AGGTTTTACCTGCTACGGACTGAGCAAGGGAGGTTTCGTGCCTTGGTTC
MtCas6 (III-A):
(SEQ ID NO: 41)
ATGGCCGCCAGAAGAGGCGGAATCCGGAGAACCGACCTGCTGCGGAGGTCTGGCCAGCCTCGGG
GCAGACACCGGGCCTCCGCCGCCGAGAGCGGCCTGACATGGATCTCCCCTACCCTGATCCTGGT
GGGCTTCAGCCACAGGGGCGATAGGAGAATGACCGAGCACCTGTCCAGACTGACCCTGACCCTG
GAAGTGGATGCCCCCCTGGAGAGAGCCCGGGTGGCCACCCTGGGCCCCCACCTGCATGGCGTGC
TGATGGAGTCTATCCCCGCCGACTACGTGCAGACACTGCACACAGTGCCGGTGAACCCTTACAG
CCAGTACGCTCTGGCCCGGAGCACCACCAGCCTGGAGTGGAAGATCTCCACCCTGACAAATGAG
GCCCGGCAGCAGATCGTCGGCCCCATCAACGACGCCGCCTTCGCCGGCTTCCGGCTGCGGGCCA
GCGGCATCGCCACCCAGGTGACAAGCAGAAGCCTGGAGCAGAACCCCCTGTCCCAGTTTGCCAG
AATCTTCTACGCCAGGCCCGAAACCCGCAAGTTCAGAGTGGAGTTCCTGACCCCCACCGCCTTC
AAGCAGAGCGGCGAGTACGTGTTTTGGCCCGATCCCAGACTGGTGTTCCAGTCCCTGGCCCAGA
AGTACGGCGCCATCGTGGACGGAGAAGAGCCCGACCCCGGCCTGATCGCCGAGTTTGGCCAGTC
CGTGAGACTGAGCGCCTTCAGAGTGGCCAGCGCCCCTTTTGCCGTGGGCGCCGCCAGGGTGCCC
GGATTCACCGGCAGCGCCACCTTCACCGTGCGGGGAGTGGACACCTTCGCCAGCTACATCGCCG
CTCTGCTGTGGTTCGGCGAGTTCAGCGGATGCGGCATCAAGGCCTCCATGGGAATGGGCGCCAT
CCGGGTGCAGCCTCTGGCCCCCCGGGAGAAGTGCGTGCCCAAGCCC
PfCas6 (III-B):
(SEQ ID NO: 42)
ATGAGATTCCTGATCAGACTGGTGCCCGAGGACAAGGACAGAGCCTTCAAGGTGCCTTACAACC
ACCAGTACTATCTGCAGGGCCTGATCTACAACGCCATCAAGTCCTCCAACCCCAAGCTGGCCAC
CTACCTGCACGAGGTGAAGGGCCCCAAGCTGTTCACCTACAGCCTGTTCATGGCCGAAAAGCGG
GAGCACCCTAAGGGCCTGCCCTACTTTCTGGGCTACAAGAAGGGCTTCTTCTACTTCAGCACCT
GCGTGCCCGAGATCGCCGAGGCCCTGGTGAACGGCCTGCTGATGAATCCCGAGGTGCGGCTGTG
GGACGAGAGATTCTACCTGCACGAAATCAAGGTCCTGCGGGAGCCCAAGAAGTTCAACGGCAGC
ACCTTCGTGACCCTGAGCCCCATCGCCGTGACCGTGGTGAGAAAGGGCAAGTCCTACGACGTGC
CCCCCATGGAAAAGGAGTTCTACAGCATTATCAAGGATGACCTGCAGGACAAGTACGTGATGGC
CTACGGCGACAAGCCCCCCAGTGAGTTCGAGATGGAAGTGCTGATCGCCAAGCCCAAGCGGTTC
CGGATCAAGCCCGGCATCTATCAGACCGCCTGGCACCTGGTGTTTCGGGCCTACGGCAATGACG
ACCTGCTGAAGGTGGGCTACGAAGTGGGATTCGGGGAGAAGAACTCCCTGGGATTCGGAATGGT
CAAGGTGGAGGGCAACAAGACCACCAAGGAAGCCGAAGAACAGGAGAAGATCACCTTCAACTCC
CGGGAAGAGCTGAAAACAGGCGTG
PaCsf5 (IV-A1):
(SEQ ID NO: 43)
ATGTTCGTGACCCAGGTGATCTTCAACATCGGCGAACGGACGTACCCCGACAGGGCTCGGGCTA
TGGTGGCCGAGCTGATGGATGGCGTCCAGCCTGGCCTGGTGGCCACCCTGATGAACTACATCCC
CGGCACCAGCACGAGCCGGACAGAGTTCCCCACCGTGCAGTTCGGCGGCGCCAGCGACGGCTTT
TGCCTGCTGGGCTTCGGCGACGGCGGCGGCGCCATCGTGAGAGATGCCGTGCCCCTGATCCACG
CCGCCCTGGCAAGGCGGATGCCTGATCGGATCATCCAGGTGGAACACAAGGAGCACAGCCTGTC
CGCCGAGGCCCGGCCCTACGTGCTGAGCTACACCGTGCCTCGGATGGTGGTGCAGAAGAAGCAG
CGGCACGCCGAGAGACTGCTGCACGAAGCCGAGGGAAAGGCTCACCTGGAGGGCCTGTTCCTGC
GGAGCCTGCAGAGGCAGGCCGCCGCCGTGGGCCTGCCCCTGCCCGAGAACCTGGAGGTGGAGTT
CAAGGGAGCCGTGGGCGACTTCGCCGCAAAGCACAATCCAAATAGCAAGGTGGCCTACCGGGGA
CTGAGAGGCGCCGTGTTCGATGTGAACGCCAGACTGGGCGGCATCTGGACCGCCGGATTCATGC
TGAGCAAGGGCTACGGCCAGTTTAACGCCACCCACCAGCTGAGCGGCGCCGTGAACGCTCTGTC
CGAA
MtCsf5 (IV-A2):
(SEQ ID NO: 44)
ATGCACCAGACCCTGATCCGGATCAACTGGCCCAAGGGATTCAAGTGCCCCCCTGCCGAGTTCC
GGGAAAAGCTGGCCAAGAGCGAGATGTTCCCCCCCGAGTTCTTCCACTACGGCACGGAACTGGC
CGTGTGGGACAAGCAGACCGCCGAGGTGGAGGGCAAGATCAAGACCGTGTCCAAGGAGAAGATC
ATCAAGACCTTTGACAAGCCCATCCCCCTGAATGGCCGGGCCCCGGTCAGAGTGATCGGCGGCC
AGGCCTGGGCCGGCGTGATCGCCGACCCCGAGATGGAGGGCATGCTGATCCCACACCTGGGGAG
CATCCTGAAGGTGGCCAGCAGCGCGGCCGGATGCGCAGTGAAGATCGAACTGGAACAGAGAAAG
TTCGGCATCAGCTACACCGAGTACCCCGTGAAGTACAACCTGCGGGAGCTGGTGCTGAAGAGAA
GATGCGAGGACGCCCGGTCTACCGATATCGAGAGCCTGATTGCCGATAGAATCTGGGGCGGCGT
GTCCGGCGAGAGCTACTATGGCATCGACGGCACATGCGCCAAGTTTGGCTTCGAACCCCCCAGC
AGAGAGCAGCTGGAGCTGCGGATCTTCCCCATGAAGAACATCGGACTGCACATGAAGTCCAGCG
ACGGACTGTCCAAGGAGTACATGAGCCTGATTGACGCCGAGGTGTGGATGAACGCTAAGCTGGA
AGGAGTGTGGCAGGTGGGCAACCTGATCAGCAGGGGCTACGGCCGGTTCATCAAGTCTATCGGC
GCCCAGTCC

### Example 2 Single Base RNA Base-Editing Using a CasPR-ADAR2DD* Fusion Protein

The example demonstrates that CasPR fusion proteins can be used for single base RNA base-editing. Specifically, a series of fusion proteins were generated, by fusing a mutant ADAR2DD with high base-editing fidelity (the ADAR2DD double mutant E488Q/T375A)—ADAR2DD*—to the C-terminus of each of the 11 CasPRs, to generate the high fidelity A→G single base RNA editors CasPR-ADAR2DD*. See SEQ ID NOs: 45-55. These fusions were expressed in mammalian cells under the strong CMV promoter with polyA tail.

To directly observe and measure the RNA base editing activity of these CasPR-ADAR2DD* fusions, a TAG stop codon was introduced into the wild-type mCherry coding sequence to disrupted mCherry translation. Translation of this mutant mCherry coding sequence does not lead to the production of a fluorescent mCherry protein. Only when the A in the TAG stop codon is corrected by the RNA base editor to G will the premature translation termination of mCherry be overcome, leading to the production of the red fluorescent mCherry reporter protein.

To control for transfection efficiency, a coding sequence for the blue fluorescent protein BFP was linked to the mCherry coding sequence via the P2A coding sequence (see FIG. 4). As shown in FIG. 4, HEK293T cells were co-transfected by two plasmids, one encoding the BFP-mCherry reporter, the other encoding the RNA base-editor together with a compatible crRNA under the transcriptional control of a U6 promoter as well as an EGFP reporter (see FIG. 4 and below) to direct the base-editor to the mCherry mutant mRNA. The BFP signal would always be present regardless of whether the linked mCherry is a mutant (no red fluorescent) or a corrected wild-type (red fluorescent).

To determine whether the CasPR-ADAR2DD* fusions had stable editing activity, three different stop codons were introduced into the mCherry reporter (see SEQ ID NOs: 56-58).

To design crRNA that targets the TAG mutant stop codon, the spacer sequence of the crRNA was designed to be located at the 5′-end of the direct repeat (DR) sequence, and the C-A mismatch between the spacer sequence and the target sequence (on the mutant mCherry mRNA) was designed to be the 5^{th} base from the 5′-end, which is far away from the DR sequence. The length of the spacer was designed to be 30 bases (see SEQ ID NOs: 59-61).

After co-transfecting into HEK293T cells the plasmid encoding the EGFP reporter, the CasPR-ADAR2DD* fusion, and the crRNA targeting the TAG stop codon(s), and the plasmid encoding the corresponding BFP-P2A-mCherry mutant reporter sequence, the transfected 293T cells were cultured at 37° C. under 5% CO₂ for 24 hours. Cells co-expressing EGFP and BFP were successfully co-transfected by both plasmids, and were isolated by FACS sorting. mCherry expression in these isolated cells were then determined. The results were summarized in FIG. 5.

The results showed that, with the exception of I-D, all CasPR-ADAR2DD* fusion proteins exhibited single base RNA base-editing activity. Among them, I-B, I-E, and IV-A1 had the highest activities.

SsoCas6 (I-A):
(SEQ ID NO: 45)
ATGCCCAAGAAGAAGCGGAAGGTGATGCCTCTGATCTTCAAGATCGGCTATAACGTGATCCCCC
TGCAGGACGTGATCCTGCCCACCCCTTCCAGCAAGGTGCTGAAGTACCTGATCCAGAGCGGCAA
GCTGATCCCCAGCCTGAAGGACCTGATCACCAGCCGGGACAAGTACAAGCCAATCTTCATCTCC
CACCTGGGCTTCAACCAGCGGAGGATTTTCCAGACCAACGGCAATCTGAAAACCATCACCAAGG
GCAGTAGACTGAGCTCCATCATCGCCTTCAGCACCCAGGCCAACGTGCTGTCCGAGGTGGCCGA
TGAAGGGATCTTCGAAACCGTGTACGGAAAGTTCCACATCATGATCGAAAGCATCGAGATCGTG
GAGGTGGAAAAGCTGAAGGAGGAGGTGGAGAAGCACATGAACGACAACATCAGAGTGAGATTCG
TGTCTCCCACACTGCTGAGCTCCAAGGTGCTGCTGCCCCCCAGCCTGTCCGAAAGATACAAGAA
GATCCACGCCGGGTACAGCACCCTGCCCAGCGTGGGCCTGATCGTGGCCTACGCCTACAACGTG
TACTGCAATCTGATCGGCAAGAAGGAAGTGGAAGTGCGGGCCTTCAAGTTTGGAATCCTGAGCA
ACGCCCTGTCCAGAATCATCGGCTACGACCTGCACCCTGTGACCGTGGCCATCGGCGAGGACAG
CAAGGGGAATCTGAGAAAGGCTCGGGGCGTGATGGGCTGGATCGAGTTCGACATCCCCGACGAA
AGACTGAAGCGGCGGGCCCTGAACTATCTGCTGACCAGCAGCTACCTGGGCATCGGGAGATCTC
GGGGCATCGGCTTCGGCGAGATCCGGCTGGAGTTCCGGAAGATTGAAGAGAAGGAGGGACCCAA
GAAGAAGCGGAAGGTGGGTGGAGGCGGAGGTTCTGGGGGAGGAGGTAGTGGCGGTGGTGGTTCA
GGAGGCGGCGGAAGCCAGCTGCATTTACCGCAGGTTTTAGCTGACGCTGTCTCACGCCTGGTCA
TAGGTAAGTTTGGTGACCTGACCGACAACTTCTCCTCCCCTCACGCTCGCAGAATAGGTCTGGC
TGGAGTCGTCATGACAACAGGCACAGATGTTAAAGATGCCAAGGTGATATGTGTTTCTACAGGA
GCAAAATGTATTAATGGTGAATACCTAAGTGATCGTGGCCTTGCATTAAATGACTGCCATGCAG
AAATAGTATCTCGGAGATCCTTGCTCAGATTTCTTTATACACAACTTGAGCTTTACTTAAATAA
CGAGGATGATCAAAAAAGATCCATCTTTCAGAAATCAGAGCGAGGGGGGTTTAGGCTGAAGGAG
AATATACAGTTTCATCTGTACATCAGCACCTCTCCCTGTGGAGATGCCAGAATCTTCTCACCAC
ATGAGGCAATCCTGGAAGAACCAGCAGATAGACACCCAAATCGTAAAGCAAGAGGACAGCTACG
GACCAAAATAGAGGCTGGTCAGGGGACGATTCCAGTGCGCAACAATGCGAGCATCCAAACGTGG
GACGGGGTGCTGCAAGGGGAGCGGCTGCTCACCATGTCCTGCAGTGACAAGATTGCACGCTGGA
ACGTGGTGGGCATCCAGGGATCACTGCTCAGCATTTTCGTGGAGCCCATTTACTTCTCGAGCAT
CATCCTGGGCAGCCTTTACCACGGGGACCACCTTTCCAGGGCCATGTACCAGCGGATCTCCAAC
ATAGAGGACCTGCCACCTCTCTACACCCTCAACAAGCCTTTGCTCACAGGCATCAGCAATGCAG
AAGCACGGCAGCCAGGGAAGGCCCCCATATTCAGTGTCAACTGGACGGTAGGCGACTCCGCTAT
TGAGGTCATCAACGCCACGACTGGGAAGGGAGAGCTGGGCCGCGCGTCCCGCCTGTGTAAGCAC
GCGTTGTACTGTCGCTGGATGCGTGTGCACGGCAAGGTTCCCTCCCACTTACTACGCTCCAAGA
TTACCAAGCCCAACGTGTACCATGAGACAAAGCTGGCGGCAAAGGAGTACCAGGCCGCCAAGGC
GCGTCTGTTCACAGCCTTCATCAAGGCGGGGCTGGGGGCCTGGGTGGAGAAGCCCACCGAGCAG
GACCAGTTCTCACTCACGTAA
MmCas6 (I-B):
(SEQ ID NO: 46)
ATGCCCAAGAAGAAGCGGAAGGTGATGGACCTGGAGTACATGCACATCTCCTACCCTAACATCC
TGCTGAACATGCGGGACGGCAGCAAGCTGCGGGGCTACTTCGCCAAGAAGTACATCGACGAAGA
GATTGTGCACAACCACAGAGACAACGCCTTTGTGTACAAGTACCCCCAGATCCAGTTTAAGATC
ATCGATAGAAGCCCCCTGATCATCGGCATTGGCTCTCTGGGCATCAATTTCCTGGAGAGCAAGC
GGATCTTCTTCGAGAAGGAACTGATTATCAGCAACGACACCAACGACATCACCGAGGTGAACGT
GCACAAGGACATGGATCACTTCGGCACGACCGACAAGATCCTGAAGTACCAGTTCAAGACCCCT
TGGATGGCACTGAACGCCAAGAATAGCGAGATCTACAAGAACTCTGACGAGATCGACCGGGAGG
AGTTCCTGAAGAGAGTGCTGATTGGGAATATCCTGAGCATGTCTAAGAGCCTGGGCTATACCAT
CGAAGAAAAGCTGAAGGTGAAGATTAACCTGAAGGAAGTGCCCGTGAAGTTCAAGAACCAGAAC
ATGGTGGGCTTTCGGGGCGAGTTCTACATCAACTTCGACATCCCTCAGTATCTGGGCATCGGCC
GGAATGTGTCCCGGGGATTCGGCACAGTGGTGAAGGTGCCCAAGAAGAAGCGGAAGGTGGGTGG
AGGCGGAGGTTCTGGGGGAGGAGGTAGTGGCGGTGGTGGTTCAGGAGGCGGCGGAAGCCAGCTG
CATTTACCGCAGGTTTTAGCTGACGCTGTCTCACGCCTGGTCATAGGTAAGTTTGGTGACCTGA
CCGACAACTTCTCCTCCCCTCACGCTCGCAGAATAGGTCTGGCTGGAGTCGTCATGACAACAGG
CACAGATGTTAAAGATGCCAAGGTGATATGTGTTTCTACAGGAGCAAAATGTATTAATGGTGAA
TACCTAAGTGATCGTGGCCTTGCATTAAATGACTGCCATGCAGAAATAGTATCTCGGAGATCCT
TGCTCAGATTTCTTTATACACAACTTGAGCTTTACTTAAATAACGAGGATGATCAAAAAAGATC
CATCTTTCAGAAATCAGAGCGAGGGGGGTTTAGGCTGAAGGAGAATATACAGTTTCATCTGTAC
ATCAGCACCTCTCCCTGTGGAGATGCCAGAATCTTCTCACCACATGAGGCAATCCTGGAAGAAC
CAGCAGATAGACACCCAAATCGTAAAGCAAGAGGACAGCTACGGACCAAAATAGAGGCTGGTCA
GGGGACGATTCCAGTGCGCAACAATGCGAGCATCCAAACGTGGGACGGGGTGCTGCAAGGGGAG
CGGCTGCTCACCATGTCCTGCAGTGACAAGATTGCACGCTGGAACGTGGTGGGCATCCAGGGAT
CACTGCTCAGCATTTTCGTGGAGCCCATTTACTTCTCGAGCATCATCCTGGGCAGCCTTTACCA
CGGGGACCACCTTTCCAGGGCCATGTACCAGCGGATCTCCAACATAGAGGACCTGCCACCTCTC
TACACCCTCAACAAGCCTTTGCTCACAGGCATCAGCAATGCAGAAGCACGGCAGCCAGGGAAGG
CCCCCATATTCAGTGTCAACTGGACGGTAGGCGACTCCGCTATTGAGGTCATCAACGCCACGAC
TGGGAAGGGAGAGCTGGGCCGCGCGTCCCGCCTGTGTAAGCACGCGTTGTACTGTCGCTGGATG
CGTGTGCACGGCAAGGTTCCCTCCCACTTACTACGCTCCAAGATTACCAAGCCCAACGTGTACC
ATGAGACAAAGCTGGCGGCAAAGGAGTACCAGGCCGCCAAGGCGCGTCTGTTCACAGCCTTCAT
CAAGGCGGGGCTGGGGGCCTGGGTGGAGAAGCCCACCGAGCAGGACCAGTTCTCACTCACGTAA
SpCas5d (I-C1):
(SEQ ID NO: 47)
ATGCCCAAGAAGAAGCGGAAGGTGATGAGAAATGAAGTGCAGTTCGAGCTGTTCGGCGACTACG
CCCTGTTCACCGACCCCCTGACCAAGATCGGCGGCGAAAAGCTGAGCTACAGCGTGCCTACCTA
CCAGGCCCTGAAGGGCATCGCCGAGAGCATCTACTGGAAGCCCACCATCGTGTTCGTGATCGAC
GAACTGCGGGTCATGAAGCCCATTCAGATGGAGTCTAAGGGCGTGAGGCCCATCGAGTACGGCG
GCGGCAACACCCTGGCCCACTACACCTACCTGAAGGATGTGCACTACCAGGTGAAGGCCCACTT
CGAGTTCAACCTGCACCGGCCCGACCTGGCCTTCGATAGAAACGAGGGCAAGCACTACTCCATC
CTGCAGAGAAGCCTGAAGGCCGGCGGCAGAAGAGATATTTTCCTGGGCGCCCGGGAGTGCCAGG
GCTACGTGGCCCCCTGCGAGTTCGGCAGCGGCGACGGCTTCTACGACGGCCAGGGCAAGTACCA
CCTGGGAACCATGGTGCACGGTTTCAACTACCCCGACGAAACCGGACAGCACCAGCTGGATGTG
AGACTGTGGTCTGCCGTCATGGAAAACGGCTACATCCAGTTCCCCCGCCCTGAGGACTGCCCCA
TCGTGCGGCCTGTGAAGGAGATGGAACCCAAGATCTTCAACCCCGACAACGTGCAGTCCGCCGA
ACAGCTGCTGCACGACCTGGGCGGCGAACCCAAGAAGAAGCGGAAGGTGGGTGGAGGCGGAGGT
TCTGGGGGAGGAGGTAGTGGCGGTGGTGGTTCAGGAGGCGGCGGAAGCCAGCTGCATTTACCGC
AGGTTTTAGCTGACGCTGTCTCACGCCTGGTCATAGGTAAGTTTGGTGACCTGACCGACAACTT
CTCCTCCCCTCACGCTCGCAGAATAGGTCTGGCTGGAGTCGTCATGACAACAGGCACAGATGTT
AAAGATGCCAAGGTGATATGTGTTTCTACAGGAGCAAAATGTATTAATGGTGAATACCTAAGTG
ATCGTGGCCTTGCATTAAATGACTGCCATGCAGAAATAGTATCTCGGAGATCCTTGCTCAGATT
TCTTTATACACAACTTGAGCTTTACTTAAATAACGAGGATGATCAAAAAAGATCCATCTTTCAG
AAATCAGAGCGAGGGGGGTTTAGGCTGAAGGAGAATATACAGTTTCATCTGTACATCAGCACCT
CTCCCTGTGGAGATGCCAGAATCTTCTCACCACATGAGGCAATCCTGGAAGAACCAGCAGATAG
ACACCCAAATCGTAAAGCAAGAGGACAGCTACGGACCAAAATAGAGGCTGGTCAGGGGACGATT
CCAGTGCGCAACAATGCGAGCATCCAAACGTGGGACGGGGTGCTGCAAGGGGAGCGGCTGCTCA
CCATGTCCTGCAGTGACAAGATTGCACGCTGGAACGTGGTGGGCATCCAGGGATCACTGCTCAG
CATTTTCGTGGAGCCCATTTACTTCTCGAGCATCATCCTGGGCAGCCTTTACCACGGGGACCAC
CTTTCCAGGGCCATGTACCAGCGGATCTCCAACATAGAGGACCTGCCACCTCTCTACACCCTCA
ACAAGCCTTTGCTCACAGGCATCAGCAATGCAGAAGCACGGCAGCCAGGGAAGGCCCCCATATT
CAGTGTCAACTGGACGGTAGGCGACTCCGCTATTGAGGTCATCAACGCCACGACTGGGAAGGGA
GAGCTGGGCCGCGCGTCCCGCCTGTGTAAGCACGCGTTGTACTGTCGCTGGATGCGTGTGCACG
GCAAGGTTCCCTCCCACTTACTACGCTCCAAGATTACCAAGCCCAACGTGTACCATGAGACAAA
GCTGGCGGCAAAGGAGTACCAGGCCGCCAAGGCGCGTCTGTTCACAGCCTTCATCAAGGCGGGG
CTGGGGGCCTGGGTGGAGAAGCCCACCGAGCAGGACCAGTTCTCACTCACGTAA
BhCas5d (I-C2):
(SEQ ID NO: 48)
ATGCCCAAGAAGAAGCGGAAGGTGATGTACAGAAGCCGGGACTTCTACGTGAGAGTGTCCGGCC
AGCGGGCCCTGTTCACCAACCCCGCCACCAAGGGCGGCTCCGAACGGAGCTCCTACTCCGTGCC
TACCCGGCAGGCCCTGAACGGGATTGTGGACGCCATCTACTACAAGCCCACGTTCACCAACATC
GTGACCGAGGTGAAGGTGATTAACCAGATCCAGACCGAACTGCAGGGCGTGCGGGCCCTGCTGC
ATGACTACAGCGCCGACCTGAGCTACGTGTCCTACCTGAGCGACGTGGTGTACCTGATTAAGTT
TCATTTCGTGTGGAACGAGGATAGAAAGGACCTGAATAGCGACCGGCTGCCAGCCAAGCATGAG
GCCATCATGGAGCGGTCTATCCGGAAGGGCGGCAGACGGGACGTGTTCCTGGGCACCAGAGAAT
GCCTGGGCCTGCTGGACGACATCAGCCAGGAAGAATACGAAACCACAGTGAGCTATTACAATGG
GGTGAACATCGACCTGGGCATCATGTTCCACAGCTTCGCTTACCCCAAGGACAAGAAAACCCCC
CTGAAGTCCTACTTCACAAAGACCGTGATGAAGAACGGCGTGATCACCTTCAAGGCCCAGTCCG
AATGCGATATTGTGAACACCCTGAGCTCCTACGCCTTCAAGGCCCCCGAGGAGATCAAGAGCGT
GAACGACGAGTGCATGGAGTACGACGCCATGGAGAAGGGCGAAAACCCCAAGAAGAAGCGGAAG
GTGGGTGGAGGCGGAGGTTCTGGGGGAGGAGGTAGTGGCGGTGGTGGTTCAGGAGGCGGCGGAA
GCCAGCTGCATTTACCGCAGGTTTTAGCTGACGCTGTCTCACGCCTGGTCATAGGTAAGTTTGG
TGACCTGACCGACAACTTCTCCTCCCCTCACGCTCGCAGAATAGGTCTGGCTGGAGTCGTCATG
ACAACAGGCACAGATGTTAAAGATGCCAAGGTGATATGTGTTTCTACAGGAGCAAAATGTATTA
ATGGTGAATACCTAAGTGATCGTGGCCTTGCATTAAATGACTGCCATGCAGAAATAGTATCTCG
GAGATCCTTGCTCAGATTTCTTTATACACAACTTGAGCTTTACTTAAATAACGAGGATGATCAA
AAAAGATCCATCTTTCAGAAATCAGAGCGAGGGGGGTTTAGGCTGAAGGAGAATATACAGTTTC
ATCTGTACATCAGCACCTCTCCCTGTGGAGATGCCAGAATCTTCTCACCACATGAGGCAATCCT
GGAAGAACCAGCAGATAGACACCCAAATCGTAAAGCAAGAGGACAGCTACGGACCAAAATAGAG
GCTGGTCAGGGGACGATTCCAGTGCGCAACAATGCGAGCATCCAAACGTGGGACGGGGTGCTGC
AAGGGGAGCGGCTGCTCACCATGTCCTGCAGTGACAAGATTGCACGCTGGAACGTGGTGGGCAT
CCAGGGATCACTGCTCAGCATTTTCGTGGAGCCCATTTACTTCTCGAGCATCATCCTGGGCAGC
CTTTACCACGGGGACCACCTTTCCAGGGCCATGTACCAGCGGATCTCCAACATAGAGGACCTGC
CACCTCTCTACACCCTCAACAAGCCTTTGCTCACAGGCATCAGCAATGCAGAAGCACGGCAGCC
AGGGAAGGCCCCCATATTCAGTGTCAACTGGACGGTAGGCGACTCCGCTATTGAGGTCATCAAC
GCCACGACTGGGAAGGGAGAGCTGGGCCGCGCGTCCCGCCTGTGTAAGCACGCGTTGTACTGTC
GCTGGATGCGTGTGCACGGCAAGGTTCCCTCCCACTTACTACGCTCCAAGATTACCAAGCCCAA
CGTGTACCATGAGACAAAGCTGGCGGCAAAGGAGTACCAGGCCGCCAAGGCGCGTCTGTTCACA
GCCTTCATCAAGGCGGGGCTGGGGGCCTGGGTGGAGAAGCCCACCGAGCAGGACCAGTTCTCAC
TCACGTAA
SaCas6 (I-D):
(SEQ ID NO: 49)
ATGCCCAAGAAGAAGCGGAAGGTGATGCCCAACGATCCCTACAGCCTGTACTCCATCGTGATCG
AACTGGGCGCCGCCGAAAAGGGATTCCCCACAGGCATCCTGGGCAGAAGCCTGCATAGCCAGGT
GCTGCAGTGGTTCAAGCAGGATAACCCCTTCCTGGCCACCGAGCTGCACCAGAGCCAGATCTCC
CCCTTCTCCATCTCTCCACTGATGGGCAAGCGGCACGCCAAGCTGACCAAGGCCGGCGACCGGC
TGTTCTTTCGGATCTGCCTGCTGAGAGGAGATCTGCTGCAGCCCCTGCTGAACGGCATTGAGCA
GACCGTGAACCAGAGCGTGTGCCTGGACAAGTTCCGGTTCCGGCTGTGCCAGACCCACATCCTG
CCCGGCAGCCACCCTCTGGCTGGCGCCTCCCACTATAGCCTGATCAGCCAGACCCCAGTGAGCT
CCAAGATTACCCTGGACTTCAAGAGTTCTACCTCCTTCAAGGTGGACCGGAAGATCATCCAAGT
GTTCCCTCTGGGCGAACACGTGTTCAACAGCCTGCTCAGACGCTGGAATAACTTCGCCCCCGAG
GACCTGCACTTCTCTCAGGTGGACTGGAGCATCCCCATCGCCGCATTCGACGTGAAAACCATCC
CCATCCACCTGAAGAAGGTCGAGATCGGCGCACAGGGCTGGGTGACCTACATCTTCCCCAACAC
AGAACAGGCCAAGATCGCCTCCGTGCTGAGCGAATTCGCCTTCTTCAGCGGAGTGGGACGGAAA
ACCACCATGGGCATGGGCCAGGTGCAGGTGCGGTCCCCCAAGAAGAAGCGGAAGGTGGGTGGAG
GCGGAGGTTCTGGGGGAGGAGGTAGTGGCGGTGGTGGTTCAGGAGGCGGCGGAAGCCAGCTGCA
TTTACCGCAGGTTTTAGCTGACGCTGTCTCACGCCTGGTCATAGGTAAGTTTGGTGACCTGACC
GACAACTTCTCCTCCCCTCACGCTCGCAGAATAGGTCTGGCTGGAGTCGTCATGACAACAGGCA
CAGATGTTAAAGATGCCAAGGTGATATGTGTTTCTACAGGAGCAAAATGTATTAATGGTGAATA
CCTAAGTGATCGTGGCCTTGCATTAAATGACTGCCATGCAGAAATAGTATCTCGGAGATCCTTG
CTCAGATTTCTTTATACACAACTTGAGCTTTACTTAAATAACGAGGATGATCAAAAAAGATCCA
TCTTTCAGAAATCAGAGCGAGGGGGGTTTAGGCTGAAGGAGAATATACAGTTTCATCTGTACAT
CAGCACCTCTCCCTGTGGAGATGCCAGAATCTTCTCACCACATGAGGCAATCCTGGAAGAACCA
GCAGATAGACACCCAAATCGTAAAGCAAGAGGACAGCTACGGACCAAAATAGAGGCTGGTCAGG
GGACGATTCCAGTGCGCAACAATGCGAGCATCCAAACGTGGGACGGGGTGCTGCAAGGGGAGCG
GCTGCTCACCATGTCCTGCAGTGACAAGATTGCACGCTGGAACGTGGTGGGCATCCAGGGATCA
CTGCTCAGCATTTTCGTGGAGCCCATTTACTTCTCGAGCATCATCCTGGGCAGCCTTTACCACG
GGGACCACCTTTCCAGGGCCATGTACCAGCGGATCTCCAACATAGAGGACCTGCCACCTCTCTA
CACCCTCAACAAGCCTTTGCTCACAGGCATCAGCAATGCAGAAGCACGGCAGCCAGGGAAGGCC
CCCATATTCAGTGTCAACTGGACGGTAGGCGACTCCGCTATTGAGGTCATCAACGCCACGACTG
GGAAGGGAGAGCTGGGCCGCGCGTCCCGCCTGTGTAAGCACGCGTTGTACTGTCGCTGGATGCG
TGTGCACGGCAAGGTTCCCTCCCACTTACTACGCTCCAAGATTACCAAGCCCAACGTGTACCAT
GAGACAAAGCTGGCGGCAAAGGAGTACCAGGCCGCCAAGGCGCGTCTGTTCACAGCCTTCATCA
AGGCGGGGCTGGGGGCCTGGGTGGAGAAGCCCACCGAGCAGGACCAGTTCTCACTCACGTAA
EcCas6e (I-E):
(SEQ ID NO: 50)
ATGCCTAAGAAGAAGCGGAAGGTGTACCTGAGCAAGGTGATCATCGCCAGAGCCTGGAGCAGAG
ACCTGTACCAGCTGCACCAGGGCCTGTGGCACCTGTTCCCCAACCGGCCCGACGCCGCCCGGGA
TTTCCTGTTCCACGTGGAGAAGAGAAACACCCCGGAAGGCTGCCACGTGCTGCTGCAGAGCGCA
CAGATGCCTGTGAGCACCGCCGTGGCCACCGTGATCAAGACCAAGCAGGTGGAGTTCCAGCTGC
AGGTGGGCGTGCCCCTGTATTTCAGGCTGCGGGCGAATCCCATCAAGACCATCCTGGACAACCA
GAAGCGGCTGGACAGCAAGGGCAACATCAAGAGGTGCAGAGTGCCTCTGATCAAGGAGGCCGAA
CAGATCGCCTGGCTGCAGCGGAAGCTGGGCAATGCCGCCAGAGTGGAGGACGTGCACCCCATCA
GCGAGCGGCCCCAGTACTTCTCCGGCGACGGAAAGAGCGGAAAGATCCAGACCGTGTGCTTCGA
GGGCGTGCTGACCATCAACGACGCACCCGCCCTGATCGACCTCGTGCAGCAGGGGATCGGCCCT
GCCAAGTCCATGGGCTGCGGACTGCTGTCCCTGGCCCCCCTGCCCAAGAAGAAGCGGAAGGTGG
GTGGAGGCGGAGGTTCTGGGGGAGGAGGTAGTGGCGGTGGTGGTTCAGGAGGCGGCGGAAGCCA
GCTGCATTTACCGCAGGTTTTAGCTGACGCTGTCTCACGCCTGGTCATAGGTAAGTTTGGTGAC
CTGACCGACAACTTCTCCTCCCCTCACGCTCGCAGAATAGGTCTGGCTGGAGTCGTCATGACAA
CAGGCACAGATGTTAAAGATGCCAAGGTGATATGTGTTTCTACAGGAGCAAAATGTATTAATGG
TGAATACCTAAGTGATCGTGGCCTTGCATTAAATGACTGCCATGCAGAAATAGTATCTCGGAGA
TCCTTGCTCAGATTTCTTTATACACAACTTGAGCTTTACTTAAATAACGAGGATGATCAAAAAA
GATCCATCTTTCAGAAATCAGAGCGAGGGGGGTTTAGGCTGAAGGAGAATATACAGTTTCATCT
GTACATCAGCACCTCTCCCTGTGGAGATGCCAGAATCTTCTCACCACATGAGGCAATCCTGGAA
GAACCAGCAGATAGACACCCAAATCGTAAAGCAAGAGGACAGCTACGGACCAAAATAGAGGCTG
GTCAGGGGACGATTCCAGTGCGCAACAATGCGAGCATCCAAACGTGGGACGGGGTGCTGCAAGG
GGAGCGGCTGCTCACCATGTCCTGCAGTGACAAGATTGCACGCTGGAACGTGGTGGGCATCCAG
GGATCACTGCTCAGCATTTTCGTGGAGCCCATTTACTTCTCGAGCATCATCCTGGGCAGCCTTT
ACCACGGGGACCACCTTTCCAGGGCCATGTACCAGCGGATCTCCAACATAGAGGACCTGCCACC
TCTCTACACCCTCAACAAGCCTTTGCTCACAGGCATCAGCAATGCAGAAGCACGGCAGCCAGGG
AAGGCCCCCATATTCAGTGTCAACTGGACGGTAGGCGACTCCGCTATTGAGGTCATCAACGCCA
CGACTGGGAAGGGAGAGCTGGGCCGCGCGTCCCGCCTGTGTAAGCACGCGTTGTACTGTCGCTG
GATGCGTGTGCACGGCAAGGTTCCCTCCCACTTACTACGCTCCAAGATTACCAAGCCCAACGTG
TACCATGAGACAAAGCTGGCGGCAAAGGAGTACCAGGCCGCCAAGGCGCGTCTGTTCACAGCCT
TCATCAAGGCGGGGCTGGGGGCCTGGGTGGAGAAGCCCACCGAGCAGGACCAGTTCTCACTCAC
GTAA
PaCas6f (I-F):
(SEQ ID NO: 51)
ATGCCTAAGAAGAAGAGAAAGGTGGACCACTACCTGGACATTAGACTGCGCCCTGACCCAGAGT
TCCCTCCTGCCCAGCTGATGTCTGTGCTGTTTGGCAAGCTGCACCAGGCCCTGGTGGCCCAGGG
CGGTGACAGAATCGGAGTGTCTTTCCCTGATCTGGACGAATCTAGATCTAGACTGGGAGAGAGA
CTGAGAATCCACGCGTCTGCCGACGACCTGAGAGCTCTGCTGGCCAGACCATGGCTGGAAGGAC
TGCGCGACCACCTGCAGTTCGGTGAACCTGCCGTGGTGCCTCACCCAACTCCATACAGACAGGT
GAGTAGAGTGCAGGCAAAGTCTAATCCAGAGAGACTGAGACGCAGACTGATGAGAAGGCATGAC
CTGTCCGAAGAAGAAGCCAGAAAGAGAATCCCAGACACAGTGGCCAGAGCCCTGGATCTGCCTT
TTGTGACCCTGAGAAGCCAGTCTACCGGCCAGCACTTCAGACTGTTTATTCGCCACGGACCACT
GCAGGTGACCGCCGAAGAGGGAGGTTTTACCTGCTACGGACTGAGCAAGGGAGGTTTCGTGCCT
TGGTTCCCCAAGAAGAAGCGGAAGGTGGGTGGAGGCGGAGGTTCTGGGGGAGGAGGTAGTGGCG
GTGGTGGTTCAGGAGGCGGCGGAAGCCAGCTGCATTTACCGCAGGTTTTAGCTGACGCTGTCTC
ACGCCTGGTCATAGGTAAGTTTGGTGACCTGACCGACAACTTCTCCTCCCCTCACGCTCGCAGA
ATAGGTCTGGCTGGAGTCGTCATGACAACAGGCACAGATGTTAAAGATGCCAAGGTGATATGTG
TTTCTACAGGAGCAAAATGTATTAATGGTGAATACCTAAGTGATCGTGGCCTTGCATTAAATGA
CTGCCATGCAGAAATAGTATCTCGGAGATCCTTGCTCAGATTTCTTTATACACAACTTGAGCTT
TACTTAAATAACGAGGATGATCAAAAAAGATCCATCTTTCAGAAATCAGAGCGAGGGGGGTTTA
GGCTGAAGGAGAATATACAGTTTCATCTGTACATCAGCACCTCTCCCTGTGGAGATGCCAGAAT
CTTCTCACCACATGAGGCAATCCTGGAAGAACCAGCAGATAGACACCCAAATCGTAAAGCAAGA
GGACAGCTACGGACCAAAATAGAGGCTGGTCAGGGGACGATTCCAGTGCGCAACAATGCGAGCA
TCCAAACGTGGGACGGGGTGCTGCAAGGGGAGCGGCTGCTCACCATGTCCTGCAGTGACAAGAT
TGCACGCTGGAACGTGGTGGGCATCCAGGGATCACTGCTCAGCATTTTCGTGGAGCCCATTTAC
TTCTCGAGCATCATCCTGGGCAGCCTTTACCACGGGGACCACCTTTCCAGGGCCATGTACCAGC
GGATCTCCAACATAGAGGACCTGCCACCTCTCTACACCCTCAACAAGCCTTTGCTCACAGGCAT
CAGCAATGCAGAAGCACGGCAGCCAGGGAAGGCCCCCATATTCAGTGTCAACTGGACGGTAGGC
GACTCCGCTATTGAGGTCATCAACGCCACGACTGGGAAGGGAGAGCTGGGCCGCGCGTCCCGCC
TGTGTAAGCACGCGTTGTACTGTCGCTGGATGCGTGTGCACGGCAAGGTTCCCTCCCACTTACT
ACGCTCCAAGATTACCAAGCCCAACGTGTACCATGAGACAAAGCTGGCGGCAAAGGAGTACCAG
GCCGCCAAGGCGCGTCTGTTCACAGCCTTCATCAAGGCGGGGCTGGGGGCCTGGGTGGAGAAGC
CCACCGAGCAGGACCAGTTCTCACTCACGTAA
MtCas6 (III-A):
(SEQ ID NO: 52)
ATGCCCAAGAAGAAGCGGAAGGTGATGGCCGCCAGAAGAGGCGGAATCCGGAGAACCGACCTGC
TGCGGAGGTCTGGCCAGCCTCGGGGCAGACACCGGGCCTCCGCCGCCGAGAGCGGCCTGACATG
GATCTCCCCTACCCTGATCCTGGTGGGCTTCAGCCACAGGGGCGATAGGAGAATGACCGAGCAC
CTGTCCAGACTGACCCTGACCCTGGAAGTGGATGCCCCCCTGGAGAGAGCCCGGGTGGCCACCC
TGGGCCCCCACCTGCATGGCGTGCTGATGGAGTCTATCCCCGCCGACTACGTGCAGACACTGCA
CACAGTGCCGGTGAACCCTTACAGCCAGTACGCTCTGGCCCGGAGCACCACCAGCCTGGAGTGG
AAGATCTCCACCCTGACAAATGAGGCCCGGCAGCAGATCGTCGGCCCCATCAACGACGCCGCCT
TCGCCGGCTTCCGGCTGCGGGCCAGCGGCATCGCCACCCAGGTGACAAGCAGAAGCCTGGAGCA
GAACCCCCTGTCCCAGTTTGCCAGAATCTTCTACGCCAGGCCCGAAACCCGCAAGTTCAGAGTG
GAGTTCCTGACCCCCACCGCCTTCAAGCAGAGCGGCGAGTACGTGTTTTGGCCCGATCCCAGAC
TGGTGTTCCAGTCCCTGGCCCAGAAGTACGGCGCCATCGTGGACGGAGAAGAGCCCGACCCCGG
CCTGATCGCCGAGTTTGGCCAGTCCGTGAGACTGAGCGCCTTCAGAGTGGCCAGCGCCCCTTTT
GCCGTGGGCGCCGCCAGGGTGCCCGGATTCACCGGCAGCGCCACCTTCACCGTGCGGGGAGTGG
ACACCTTCGCCAGCTACATCGCCGCTCTGCTGTGGTTCGGCGAGTTCAGCGGATGCGGCATCAA
GGCCTCCATGGGAATGGGCGCCATCCGGGTGCAGCCTCTGGCCCCCCGGGAGAAGTGCGTGCCC
AAGCCCCCCAAGAAGAAGCGGAAGGTGGGTGGAGGCGGAGGTTCTGGGGGAGGAGGTAGTGGCG
GTGGTGGTTCAGGAGGCGGCGGAAGCCAGCTGCATTTACCGCAGGTTTTAGCTGACGCTGTCTC
ACGCCTGGTCATAGGTAAGTTTGGTGACCTGACCGACAACTTCTCCTCCCCTCACGCTCGCAGA
ATAGGTCTGGCTGGAGTCGTCATGACAACAGGCACAGATGTTAAAGATGCCAAGGTGATATGTG
TTTCTACAGGAGCAAAATGTATTAATGGTGAATACCTAAGTGATCGTGGCCTTGCATTAAATGA
CTGCCATGCAGAAATAGTATCTCGGAGATCCTTGCTCAGATTTCTTTATACACAACTTGAGCTT
TACTTAAATAACGAGGATGATCAAAAAAGATCCATCTTTCAGAAATCAGAGCGAGGGGGGTTTA
GGCTGAAGGAGAATATACAGTTTCATCTGTACATCAGCACCTCTCCCTGTGGAGATGCCAGAAT
CTTCTCACCACATGAGGCAATCCTGGAAGAACCAGCAGATAGACACCCAAATCGTAAAGCAAGA
GGACAGCTACGGACCAAAATAGAGGCTGGTCAGGGGACGATTCCAGTGCGCAACAATGCGAGCA
TCCAAACGTGGGACGGGGTGCTGCAAGGGGAGCGGCTGCTCACCATGTCCTGCAGTGACAAGAT
TGCACGCTGGAACGTGGTGGGCATCCAGGGATCACTGCTCAGCATTTTCGTGGAGCCCATTTAC
TTCTCGAGCATCATCCTGGGCAGCCTTTACCACGGGGACCACCTTTCCAGGGCCATGTACCAGC
GGATCTCCAACATAGAGGACCTGCCACCTCTCTACACCCTCAACAAGCCTTTGCTCACAGGCAT
CAGCAATGCAGAAGCACGGCAGCCAGGGAAGGCCCCCATATTCAGTGTCAACTGGACGGTAGGC
GACTCCGCTATTGAGGTCATCAACGCCACGACTGGGAAGGGAGAGCTGGGCCGCGCGTCCCGCC
TGTGTAAGCACGCGTTGTACTGTCGCTGGATGCGTGTGCACGGCAAGGTTCCCTCCCACTTACT
ACGCTCCAAGATTACCAAGCCCAACGTGTACCATGAGACAAAGCTGGCGGCAAAGGAGTACCAG
GCCGCCAAGGCGCGTCTGTTCACAGCCTTCATCAAGGCGGGGCTGGGGGCCTGGGTGGAGAAGC
CCACCGAGCAGGACCAGTTCTCACTCACGTAA
PfCas6 (III-B):
(SEQ ID NO: 53)
ATGCCCAAGAAGAAGCGGAAGGTGATGAGATTCCTGATCAGACTGGTGCCCGAGGACAAGGACA
GAGCCTTCAAGGTGCCTTACAACCACCAGTACTATCTGCAGGGCCTGATCTACAACGCCATCAA
GTCCTCCAACCCCAAGCTGGCCACCTACCTGCACGAGGTGAAGGGCCCCAAGCTGTTCACCTAC
AGCCTGTTCATGGCCGAAAAGCGGGAGCACCCTAAGGGCCTGCCCTACTTTCTGGGCTACAAGA
AGGGCTTCTTCTACTTCAGCACCTGCGTGCCCGAGATCGCCGAGGCCCTGGTGAACGGCCTGCT
GATGAATCCCGAGGTGCGGCTGTGGGACGAGAGATTCTACCTGCACGAAATCAAGGTCCTGCGG
GAGCCCAAGAAGTTCAACGGCAGCACCTTCGTGACCCTGAGCCCCATCGCCGTGACCGTGGTGA
GAAAGGGCAAGTCCTACGACGTGCCCCCCATGGAAAAGGAGTTCTACAGCATTATCAAGGATGA
CCTGCAGGACAAGTACGTGATGGCCTACGGCGACAAGCCCCCCAGTGAGTTCGAGATGGAAGTG
CTGATCGCCAAGCCCAAGCGGTTCCGGATCAAGCCCGGCATCTATCAGACCGCCTGGCACCTGG
TGTTTCGGGCCTACGGCAATGACGACCTGCTGAAGGTGGGCTACGAAGTGGGATTCGGGGAGAA
GAACTCCCTGGGATTCGGAATGGTCAAGGTGGAGGGCAACAAGACCACCAAGGAAGCCGAAGAA
CAGGAGAAGATCACCTTCAACTCCCGGGAAGAGCTGAAAACAGGCGTGCCCAAGAAGAAGCGGA
AGGTGGGTGGAGGCGGAGGTTCTGGGGGAGGAGGTAGTGGCGGTGGTGGTTCAGGAGGCGGCGG
AAGCCAGCTGCATTTACCGCAGGTTTTAGCTGACGCTGTCTCACGCCTGGTCATAGGTAAGTTT
GGTGACCTGACCGACAACTTCTCCTCCCCTCACGCTCGCAGAATAGGTCTGGCTGGAGTCGTCA
TGACAACAGGCACAGATGTTAAAGATGCCAAGGTGATATGTGTTTCTACAGGAGCAAAATGTAT
TAATGGTGAATACCTAAGTGATCGTGGCCTTGCATTAAATGACTGCCATGCAGAAATAGTATCT
CGGAGATCCTTGCTCAGATTTCTTTATACACAACTTGAGCTTTACTTAAATAACGAGGATGATC
AAAAAAGATCCATCTTTCAGAAATCAGAGCGAGGGGGGTTTAGGCTGAAGGAGAATATACAGTT
TCATCTGTACATCAGCACCTCTCCCTGTGGAGATGCCAGAATCTTCTCACCACATGAGGCAATC
CTGGAAGAACCAGCAGATAGACACCCAAATCGTAAAGCAAGAGGACAGCTACGGACCAAAATAG
AGGCTGGTCAGGGGACGATTCCAGTGCGCAACAATGCGAGCATCCAAACGTGGGACGGGGTGCT
GCAAGGGGAGCGGCTGCTCACCATGTCCTGCAGTGACAAGATTGCACGCTGGAACGTGGTGGGC
ATCCAGGGATCACTGCTCAGCATTTTCGTGGAGCCCATTTACTTCTCGAGCATCATCCTGGGCA
GCCTTTACCACGGGGACCACCTTTCCAGGGCCATGTACCAGCGGATCTCCAACATAGAGGACCT
GCCACCTCTCTACACCCTCAACAAGCCTTTGCTCACAGGCATCAGCAATGCAGAAGCACGGCAG
CCAGGGAAGGCCCCCATATTCAGTGTCAACTGGACGGTAGGCGACTCCGCTATTGAGGTCATCA
ACGCCACGACTGGGAAGGGAGAGCTGGGCCGCGCGTCCCGCCTGTGTAAGCACGCGTTGTACTG
TCGCTGGATGCGTGTGCACGGCAAGGTTCCCTCCCACTTACTACGCTCCAAGATTACCAAGCCC
AACGTGTACCATGAGACAAAGCTGGCGGCAAAGGAGTACCAGGCCGCCAAGGCGCGTCTGTTCA
CAGCCTTCATCAAGGCGGGGCTGGGGGCCTGGGTGGAGAAGCCCACCGAGCAGGACCAGTTCTC
ACTCACGTAA
PaCsf5 (IV-A1):
(SEQ ID NO: 54)
ATGCCTAAGAAGAAGCGGAAGGTGTTCGTGACCCAGGTGATCTTCAACATCGGCGAACGGACGT
ACCCCGACAGGGCTCGGGCTATGGTGGCCGAGCTGATGGATGGCGTCCAGCCTGGCCTGGTGGC
CACCCTGATGAACTACATCCCCGGCACCAGCACGAGCCGGACAGAGTTCCCCACCGTGCAGTTC
GGCGGCGCCAGCGACGGCTTTTGCCTGCTGGGCTTCGGCGACGGCGGCGGCGCCATCGTGAGAG
ATGCCGTGCCCCTGATCCACGCCGCCCTGGCAAGGCGGATGCCTGATCGGATCATCCAGGTGGA
ACACAAGGAGCACAGCCTGTCCGCCGAGGCCCGGCCCTACGTGCTGAGCTACACCGTGCCTCGG
ATGGTGGTGCAGAAGAAGCAGCGGCACGCCGAGAGACTGCTGCACGAAGCCGAGGGAAAGGCTC
ACCTGGAGGGCCTGTTCCTGCGGAGCCTGCAGAGGCAGGCCGCCGCCGTGGGCCTGCCCCTGCC
CGAGAACCTGGAGGTGGAGTTCAAGGGAGCCGTGGGCGACTTCGCCGCAAAGCACAATCCAAAT
AGCAAGGTGGCCTACCGGGGACTGAGAGGCGCCGTGTTCGATGTGAACGCCAGACTGGGCGGCA
TCTGGACCGCCGGATTCATGCTGAGCAAGGGCTACGGCCAGTTTAACGCCACCCACCAGCTGAG
CGGCGCCGTGAACGCTCTGTCCGAACCCAAGAAGAAGCGGAAGGTGGGTGGAGGCGGAGGTTCT
GGGGGAGGAGGTAGTGGCGGTGGTGGTTCAGGAGGCGGCGGAAGCCAGCTGCATTTACCGCAGG
TTTTAGCTGACGCTGTCTCACGCCTGGTCATAGGTAAGTTTGGTGACCTGACCGACAACTTCTC
CTCCCCTCACGCTCGCAGAATAGGTCTGGCTGGAGTCGTCATGACAACAGGCACAGATGTTAAA
GATGCCAAGGTGATATGTGTTTCTACAGGAGCAAAATGTATTAATGGTGAATACCTAAGTGATC
GTGGCCTTGCATTAAATGACTGCCATGCAGAAATAGTATCTCGGAGATCCTTGCTCAGATTTCT
TTATACACAACTTGAGCTTTACTTAAATAACGAGGATGATCAAAAAAGATCCATCTTTCAGAAA
TCAGAGCGAGGGGGGTTTAGGCTGAAGGAGAATATACAGTTTCATCTGTACATCAGCACCTCTC
CCTGTGGAGATGCCAGAATCTTCTCACCACATGAGGCAATCCTGGAAGAACCAGCAGATAGACA
CCCAAATCGTAAAGCAAGAGGACAGCTACGGACCAAAATAGAGGCTGGTCAGGGGACGATTCCA
GTGCGCAACAATGCGAGCATCCAAACGTGGGACGGGGTGCTGCAAGGGGAGCGGCTGCTCACCA
TGTCCTGCAGTGACAAGATTGCACGCTGGAACGTGGTGGGCATCCAGGGATCACTGCTCAGCAT
TTTCGTGGAGCCCATTTACTTCTCGAGCATCATCCTGGGCAGCCTTTACCACGGGGACCACCTT
TCCAGGGCCATGTACCAGCGGATCTCCAACATAGAGGACCTGCCACCTCTCTACACCCTCAACA
AGCCTTTGCTCACAGGCATCAGCAATGCAGAAGCACGGCAGCCAGGGAAGGCCCCCATATTCAG
TGTCAACTGGACGGTAGGCGACTCCGCTATTGAGGTCATCAACGCCACGACTGGGAAGGGAGAG
CTGGGCCGCGCGTCCCGCCTGTGTAAGCACGCGTTGTACTGTCGCTGGATGCGTGTGCACGGCA
AGGTTCCCTCCCACTTACTACGCTCCAAGATTACCAAGCCCAACGTGTACCATGAGACAAAGCT
GGCGGCAAAGGAGTACCAGGCCGCCAAGGCGCGTCTGTTCACAGCCTTCATCAAGGCGGGGCTG
GGGGCCTGGGTGGAGAAGCCCACCGAGCAGGACCAGTTCTCACTCACGTAA
MtCsf5 (IV-A2):
(SEQ ID NO: 55)
ATGCCCAAGAAGAAGAGAAAGGTGCACCAGACCCTGATCCGGATCAACTGGCCCAAGGGATTCA
AGTGCCCCCCTGCCGAGTTCCGGGAAAAGCTGGCCAAGAGCGAGATGTTCCCCCCCGAGTTCTT
CCACTACGGCACGGAACTGGCCGTGTGGGACAAGCAGACCGCCGAGGTGGAGGGCAAGATCAAG
ACCGTGTCCAAGGAGAAGATCATCAAGACCTTTGACAAGCCCATCCCCCTGAATGGCCGGGCCC
CGGTCAGAGTGATCGGCGGCCAGGCCTGGGCCGGCGTGATCGCCGACCCCGAGATGGAGGGCAT
GCTGATCCCACACCTGGGGAGCATCCTGAAGGTGGCCAGCAGCGCGGCCGGATGCGCAGTGAAG
ATCGAACTGGAACAGAGAAAGTTCGGCATCAGCTACACCGAGTACCCCGTGAAGTACAACCTGC
GGGAGCTGGTGCTGAAGAGAAGATGCGAGGACGCCCGGTCTACCGATATCGAGAGCCTGATTGC
CGATAGAATCTGGGGCGGCGTGTCCGGCGAGAGCTACTATGGCATCGACGGCACATGCGCCAAG
TTTGGCTTCGAACCCCCCAGCAGAGAGCAGCTGGAGCTGCGGATCTTCCCCATGAAGAACATCG
GACTGCACATGAAGTCCAGCGACGGACTGTCCAAGGAGTACATGAGCCTGATTGACGCCGAGGT
GTGGATGAACGCTAAGCTGGAAGGAGTGTGGCAGGTGGGCAACCTGATCAGCAGGGGCTACGGC
CGGTTCATCAAGTCTATCGGCGCCCAGTCCCCCAAGAAGAAGCGGAAGGTGGGTGGAGGCGGAG
GTTCTGGGGGAGGAGGTAGTGGCGGTGGTGGTTCAGGAGGCGGCGGAAGCCAGCTGCATTTACC
GCAGGTTTTAGCTGACGCTGTCTCACGCCTGGTCATAGGTAAGTTTGGTGACCTGACCGACAAC
TTCTCCTCCCCTCACGCTCGCAGAATAGGTCTGGCTGGAGTCGTCATGACAACAGGCACAGATG
TTAAAGATGCCAAGGTGATATGTGTTTCTACAGGAGCAAAATGTATTAATGGTGAATACCTAAG
TGATCGTGGCCTTGCATTAAATGACTGCCATGCAGAAATAGTATCTCGGAGATCCTTGCTCAGA
TTTCTTTATACACAACTTGAGCTTTACTTAAATAACGAGGATGATCAAAAAAGATCCATCTTTC
AGAAATCAGAGCGAGGGGGGTTTAGGCTGAAGGAGAATATACAGTTTCATCTGTACATCAGCAC
CTCTCCCTGTGGAGATGCCAGAATCTTCTCACCACATGAGGCAATCCTGGAAGAACCAGCAGAT
AGACACCCAAATCGTAAAGCAAGAGGACAGCTACGGACCAAAATAGAGGCTGGTCAGGGGACGA
TTCCAGTGCGCAACAATGCGAGCATCCAAACGTGGGACGGGGTGCTGCAAGGGGAGCGGCTGCT
CACCATGTCCTGCAGTGACAAGATTGCACGCTGGAACGTGGTGGGCATCCAGGGATCACTGCTC
AGCATTTTCGTGGAGCCCATTTACTTCTCGAGCATCATCCTGGGCAGCCTTTACCACGGGGACC
ACCTTTCCAGGGCCATGTACCAGCGGATCTCCAACATAGAGGACCTGCCACCTCTCTACACCCT
CAACAAGCCTTTGCTCACAGGCATCAGCAATGCAGAAGCACGGCAGCCAGGGAAGGCCCCCATA
TTCAGTGTCAACTGGACGGTAGGCGACTCCGCTATTGAGGTCATCAACGCCACGACTGGGAAGG
GAGAGCTGGGCCGCGCGTCCCGCCTGTGTAAGCACGCGTTGTACTGTCGCTGGATGCGTGTGCA
CGGCAAGGTTCCCTCCCACTTACTACGCTCCAAGATTACCAAGCCCAACGTGTACCATGAGACA
AAGCTGGCGGCAAAGGAGTACCAGGCCGCCAAGGCGCGTCTGTTCACAGCCTTCATCAAGGCGG
GGCTGGGGGCCTGGGTGGAGAAGCCCACCGAGCAGGACCAGTTCTCACTCACGTAA
BFP-P2A-MCHERRY_TAG_1:
(SEQ ID NO: 56)
ATGAGCGAGCTGATTAAGGAGAACATGCACATGAAGCTGTATATGGAGGGCACCGTGGACAACC
ATCACTTCAAGTGCACATCCGAGGGCGAAGGCAAGCCCTACGAGGGCACCCAGACCATGAGAAT
CAAGGTGGTCGAGGGCGGCCCTCTCCCCTTCGCCTTCGACATCCTGGCTACTAGCTTCCTCTAC
GGCAGCAAGACCTTCATCAACCACACCCAGGGCATCCCCGACTTCTTCAAGCAGTCCTTCCCTG
AGGGCTTCACATGGGAGAGAGTCACCACATACGAGGACGGGGGCGTGCTGACCGCTACCCAGGA
CACCAGCCTCCAGGACGGCTGCCTCATCTACAACGTCAAGATCAGAGGGGTGAACTTCACATCC
AACGGCCCTGTGATGCAGAAGAAAACACTCGGCTGGGAGGCCTTCACCGAGACACTGTACCCCG
CTGACGGCGGCCTGGAAGGCAGAAACGACATGGCCCTGAAGCTCGTGGGCGGGAGCCATCTGAT
CGCAAACATCAAGACCACATATAGATCCAAGAAACCCGCTAAGAACCTCAAGATGCCTGGCGTC
TACTATGTGGACTACAGACTGGAAAGAATCAAGGAGGCCAACAACGAGACATACGTCGAGCAGC
ACGAGGTGGCAGTGGCCAGATACTGCGACCTCCCTAGCAAACTGGGGCACAAGCTGAATGGCGC
CACTAACTTCTCCCTGTTGAAACAAGCAGGGGATGTCGAAGAGAATCCCGGGCCAATGGTGAGC
AAGGGCGAGGAGGATAACATGGCCATCATCAAGGAGTTCATGCGCTTCAAGGTGCACATGGAGG
GCTCCGTGAACGGCCACGAGTTCGAGATCGAGGGCGAGGGCGAGGGCCGCCCCTACGAGGGCAC
CCAGACCGCCAAGCTGAAGGTGACCAAGGGTGGCCCCCTGCCCTTCGCCTAGGACATCCTGTCC
CCTCAGTTCATGTACGGCTCCAAGGCCTACGTGAAGCACCCCGCCGACATCCCCGACTACTTGA
AGCTGTCCTTCCCCGAGGGCTTCAAGTGGGAGCGCGTGATGAACTTCGAGGACGGCGGCGTGGT
GACCGTGACCCAGGACTCCTCCCTGCAGGACGGCGAGTTCATCTACAAGGTGAAGCTGCGCGGC
ACCAACTTCCCCTCCGACGGCCCCGTAATGCAGAAGAAAACCATGGGCTGGGAGGCCTCCTCCG
AGCGGATGTACCCCGAGGACGGCGCCCTGAAGGGCGAGATCAAGCAGAGGCTGAAGCTGAAGGA
CGGCGGCCACTACGACGCTGAGGTCAAGACCACCTACAAGGCCAAGAAGCCCGTGCAGCTGCCC
GGCGCCTACAACGTCAACATCAAGTTGGACATCACCTCCCACAACGAGGACTACACCATCGTGG
AACAGTACGAACGCGCCGAGGGCCGCCACTCCACCGGCGGCATGGACGAGCTGTACAAGTAA
BFP-P2A-MCHERRY_TAG_2:
(SEQ ID NO: 57)
ATGAGCGAGCTGATTAAGGAGAACATGCACATGAAGCTGTATATGGAGGGCACCGTGGACAACC
ATCACTTCAAGTGCACATCCGAGGGCGAAGGCAAGCCCTACGAGGGCACCCAGACCATGAGAAT
CAAGGTGGTCGAGGGCGGCCCTCTCCCCTTCGCCTTCGACATCCTGGCTACTAGCTTCCTCTAC
GGCAGCAAGACCTTCATCAACCACACCCAGGGCATCCCCGACTTCTTCAAGCAGTCCTTCCCTG
AGGGCTTCACATGGGAGAGAGTCACCACATACGAGGACGGGGGCGTGCTGACCGCTACCCAGGA
CACCAGCCTCCAGGACGGCTGCCTCATCTACAACGTCAAGATCAGAGGGGTGAACTTCACATCC
AACGGCCCTGTGATGCAGAAGAAAACACTCGGCTGGGAGGCCTTCACCGAGACACTGTACCCCG
CTGACGGCGGCCTGGAAGGCAGAAACGACATGGCCCTGAAGCTCGTGGGCGGGAGCCATCTGAT
CGCAAACATCAAGACCACATATAGATCCAAGAAACCCGCTAAGAACCTCAAGATGCCTGGCGTC
TACTATGTGGACTACAGACTGGAAAGAATCAAGGAGGCCAACAACGAGACATACGTCGAGCAGC
ACGAGGTGGCAGTGGCCAGATACTGCGACCTCCCTAGCAAACTGGGGCACAAGCTGAATGGCGC
CACTAACTTCTCCCTGTTGAAACAAGCAGGGGATGTCGAAGAGAATCCCGGGCCAATGGTGAGC
AAGGGCGAGGAGGATAACATGGCCATCATCAAGGAGTTCATGCGCTTCAAGGTGCACATGGAGG
GCTCCGTGAACGGCCACGAGTTCGAGATCGAGGGCGAGGGCGAGGGCCGCCCCTACGAGGGCAC
CCAGACCGCCAAGCTGAAGGTGACCAAGGGTGGCCCCCTGCCCTTCGCCTGGGACATCCTGTCC
CCTCAGTTCATGTACGGCTCCAAGGCCTACGTGAAGCACCCCGCCGACATCCCCGACTACTTGA
AGCTGTCCTTCCCCGAGGGCTTCAAGTAGGAGCGCGTGATGAACTTCGAGGACGGCGGCGTGGT
GACCGTGACCCAGGACTCCTCCCTGCAGGACGGCGAGTTCATCTACAAGGTGAAGCTGCGCGGC
ACCAACTTCCCCTCCGACGGCCCCGTAATGCAGAAGAAAACCATGGGCTGGGAGGCCTCCTCCG
AGCGGATGTACCCCGAGGACGGCGCCCTGAAGGGCGAGATCAAGCAGAGGCTGAAGCTGAAGGA
CGGCGGCCACTACGACGCTGAGGTCAAGACCACCTACAAGGCCAAGAAGCCCGTGCAGCTGCCC
GGCGCCTACAACGTCAACATCAAGTTGGACATCACCTCCCACAACGAGGACTACACCATCGTGG
AACAGTACGAACGCGCCGAGGGCCGCCACTCCACCGGCGGCATGGACGAGCTGTACAAGTAA
BFP-P2A-MCHERRY_TAG_3:
(SEQ ID NO: 58)
ATGAGCGAGCTGATTAAGGAGAACATGCACATGAAGCTGTATATGGAGGGCACCGTGGACAACC
ATCACTTCAAGTGCACATCCGAGGGCGAAGGCAAGCCCTACGAGGGCACCCAGACCATGAGAAT
CAAGGTGGTCGAGGGCGGCCCTCTCCCCTTCGCCTTCGACATCCTGGCTACTAGCTTCCTCTAC
GGCAGCAAGACCTTCATCAACCACACCCAGGGCATCCCCGACTTCTTCAAGCAGTCCTTCCCTG
AGGGCTTCACATGGGAGAGAGTCACCACATACGAGGACGGGGGCGTGCTGACCGCTACCCAGGA
CACCAGCCTCCAGGACGGCTGCCTCATCTACAACGTCAAGATCAGAGGGGTGAACTTCACATCC
AACGGCCCTGTGATGCAGAAGAAAACACTCGGCTGGGAGGCCTTCACCGAGACACTGTACCCCG
CTGACGGCGGCCTGGAAGGCAGAAACGACATGGCCCTGAAGCTCGTGGGCGGGAGCCATCTGAT
CGCAAACATCAAGACCACATATAGATCCAAGAAACCCGCTAAGAACCTCAAGATGCCTGGCGTC
TACTATGTGGACTACAGACTGGAAAGAATCAAGGAGGCCAACAACGAGACATACGTCGAGCAGC
ACGAGGTGGCAGTGGCCAGATACTGCGACCTCCCTAGCAAACTGGGGCACAAGCTGAATGGCGC
CACTAACTTCTCCCTGTTGAAACAAGCAGGGGATGTCGAAGAGAATCCCGGGCCAATGGTGAGC
AAGGGCGAGGAGGATAACATGGCCATCATCAAGGAGTTCATGCGCTTCAAGGTGCACATGGAGG
GCTCCGTGAACGGCCACGAGTTCGAGATCGAGGGCGAGGGCGAGGGCCGCCCCTACGAGGGCAC
CCAGACCGCCAAGCTGAAGGTGACCAAGGGTGGCCCCCTGCCCTTCGCCTGGGACATCCTGTCC
CCTCAGTTCATGTACGGCTCCAAGGCCTACGTGAAGCACCCCGCCGACATCCCCGACTACTTGA
AGCTGTCCTTCCCCGAGGGCTTCAAGTGGGAGCGCGTGATGAACTTCGAGGACGGCGGCGTGGT
GACCGTGACCCAGGACTCCTCCCTGCAGGACGGCGAGTTCATCTACAAGGTGAAGCTGCGCGGC
ACCAACTTCCCCTCCGACGGCCCCGTAATGCAGAAGAAAACCATGGGCTAGGAGGCCTCCTCCG
AGCGGATGTACCCCGAGGACGGCGCCCTGAAGGGCGAGATCAAGCAGAGGCTGAAGCTGAAGGA
CGGCGGCCACTACGACGCTGAGGTCAAGACCACCTACAAGGCCAAGAAGCCCGTGCAGCTGCCC
GGCGCCTACAACGTCAACATCAAGTTGGACATCACCTCCCACAACGAGGACTACACCATCGTGG
AACAGTACGAACGCGCCGAGGGCCGCCACTCCACCGGCGGCATGGACGAGCTGTACAAGTAA
SPACER-TAG1-C5:
(SEQ ID NO: 59)
GTCCCAGGCGAAGGGCAGGGGGCCACCCTT
SPACER-TAG2-C5:
(SEQ ID NO: 60)
CTCCCACTTGAAGCCCTCGGGGAAGGACAG
SPACER-TAG3-C5:
(SEQ ID NO: 61)
CTCCCAGCCCATGGTTTTCTTCTGCATTAC

### Example 3 Determination of the Optimal Mismatch Position of CasPR-ADAR2DD* Fusion Protein

This example illustrates the optimization of the mismatch base location for the I-B, I-E, and IV-A1-based CasPR-ADAR2DD* RNA base editor. Specifically, the mismatch between the crRNA and target RNA was designed to be at the 2^{nd}, 5^{th}, 8^{th}, 11^{th}, 14^{th}, 17^{th}, 20^{th}, 23^{rd}, 26^{th} and 29^{th} bases, in a spacer sequence of about 30 bases (see SEQ ID NOs: 62-91). Then the crRNA coding sequence under the transcription control of a U6 promoter, as well as a corresponding CasPR-ADAR2DD* coding sequence were constructed on the same plasmid vector.

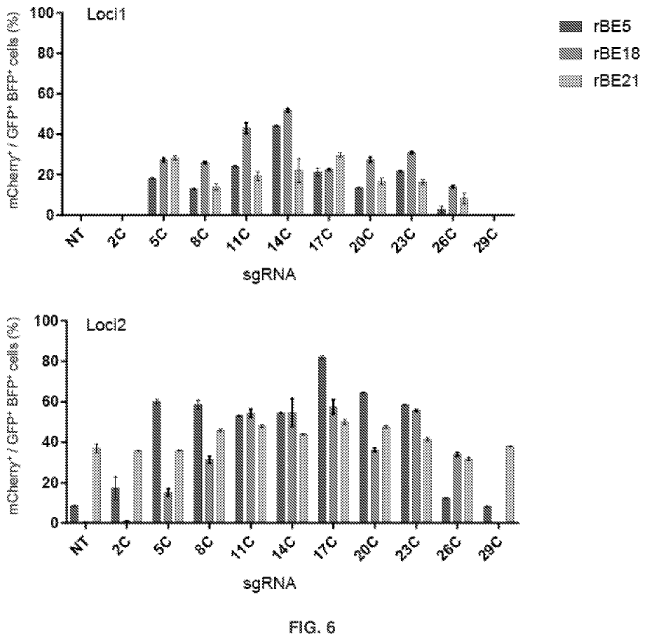

Using the assay method described in Example 2, it was found that all three RNA base editors exhibited more stable editor activity when the mismatch position is located between the 5^{th} and the 26^{th} base, particularly between the 11^{th} and 20^{th} base on the crRNA (see FIG. 6). Based editors based on I-E and IV-A1 appear to function more efficiently with higher stability than that of I-B based editors.

SPACER-TAG1-C2:
(SEQ ID NO: 62)
CCAGGCGAAGGGCAGGGGGCCACCCTTGGT
SPACER-TAG1-C5:
(SEQ ID NO: 63)
GTCCCAGGCGAAGGGCAGGGGGCCACCCTT
SPACER-TAG1-C8:
(SEQ ID NO: 64)
GATGTCCCAGGCGAAGGGCAGGGGGCCACC
SPACER-TAG1-C11:
(SEQ ID NO: 65)
CAGGATGTCCCAGGCGAAGGGCAGGGGGCC
SPACER-TAG1-C14:
(SEQ ID NO: 66)
GGACAGGATGTCCCAGGCGAAGGGCAGGGG
SPACER-TAG1-C17:
(SEQ ID NO: 67)
AGGGGACAGGATGTCCCAGGCGAAGGGCAG
SPACER-TAG1-C20:
(SEQ ID NO: 68)
CTGAGGGGACAGGATGTCCCAGGCGAAGGG
SPACER-TAG1-C23:
(SEQ ID NO: 69)
GAACTGAGGGGACAGGATGTCCCAGGCGAA
SPACER-TAG1-C26:
(SEQ ID NO: 70)
CATGAACTGAGGGGACAGGATGTCCCAGGC
SPACER-TAG1-C29:
(SEQ ID NO: 71)
GTACATGAACTGAGGGGACAGGATGTCCCA
SPACER-TAG2-C2:
(SEQ ID NO: 72)
CCACTTGAAGCCCTCGGGGAAGGACAGCTT
SPACER-TAG2-C5:
(SEQ ID NO: 73)
CTCCCACTTGAAGCCCTCGGGGAAGGACAG
SPACER-TAG2-C8:
(SEQ ID NO: 74)
GCGCTCCCACTTGAAGCCCTCGGGGAAGGA
SPACER-TAG2-C11:
(SEQ ID NO: 75)
CACGCGCTCCCACTTGAAGCCCTCGGGGAA
SPACER-TAG2-C14:
(SEQ ID NO: 76)
CATCACGCGCTCCCACTTGAAGCCCTCGGG
SPACER-TAG2-C17:
(SEQ ID NO: 77)
GTTCATCACGCGCTCCCACTTGAAGCCCTC
SPACER-TAG2-C20:
(SEQ ID NO: 78)
GAAGTTCATCACGCGCTCCCACTTGAAGCC
SPACER-TAG2-C23:
(SEQ ID NO: 79)
CTCGAAGTTCATCACGCGCTCCCACTTGAA
SPACER-TAG2-C26:
(SEQ ID NO: 80)
GTCCTCGAAGTTCATCACGCGCTCCCACTT
SPACER-TAG2-C29:
(SEQ ID NO: 81)
GCCGTCCTCGAAGTTCATCACGCGCTCCCA
SPACER-TAG3-C2:
(SEQ ID NO: 82)
CCAGCCCATGGTTTTCTTCTGCATTACGGG
SPACER-TAG3-C5:
(SEQ ID NO: 83)
CTCCCAGCCCATGGTTTTCTTCTGCATTAC
SPACER-TAG3-C8:
(SEQ ID NO: 84)
GGCCTCCCAGCCCATGGTTTTCTTCTGCAT
SPACER-TAG3-C11:
(SEQ ID NO: 85)
GGAGGCCTCCCAGCCCATGGTTTTCTTCTG
SPACER-TAG3-C14:
(SEQ ID NO: 86)
GGAGGAGGCCTCCCAGCCCATGGTTTTCTT
SPACER-TAG3-C17:
(SEQ ID NO: 87)
CTCGGAGGAGGCCTCCCAGCCCATGGTTTT
SPACER-TAG3-C20:
(SEQ ID NO: 88)
CCGCTCGGAGGAGGCCTCCCAGCCCATGGT
SPACER-TAG3-C23:
(SEQ ID NO: 89)
CATCCGCTCGGAGGAGGCCTCCCAGCCCAT
SPACER-TAG3-C26:
(SEQ ID NO: 90)
GTACATCCGCTCGGAGGAGGCCTCCCAGCC
SPACER-TAG3-C29:
(SEQ ID NO: 91)
GGGGTACATCCGCTCGGAGGAGGCCTCCCA

### Example 4 Exon Skipping of Exon 51 of DMD Using PaCsf5-Based RNA Based Editor

The example demonstrates that PaCsf5 (a CasPR) can target and inhibit pre-mRNA processing/splicing.

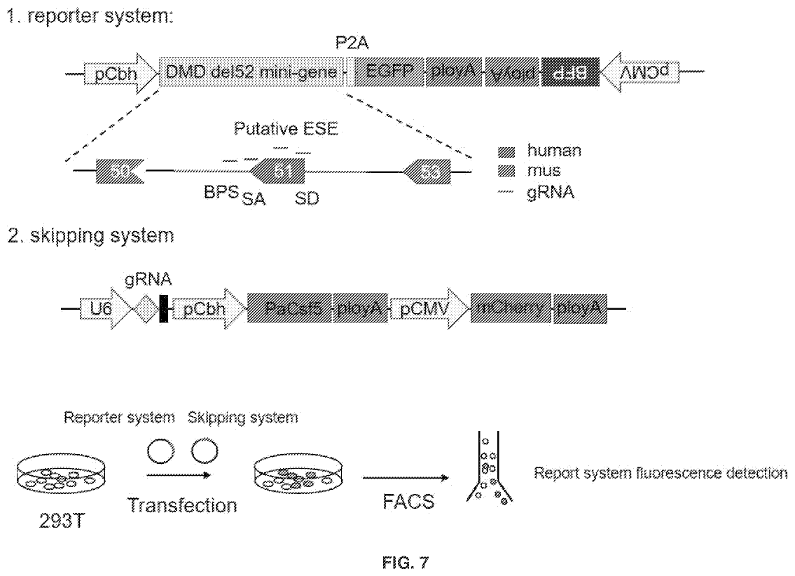

In this example, a reporter plasmid based on DMD disease model was constructed. As shown in FIG. 7, a DMD minigene was fused to an EGFP coding sequence in a transcription unit with PolyA signal under the transcriptional control of the pCbh promoter. Meanwhile, on the same reporter plasmid vector, a BFP coding sequence with PolyA signal was put under the control of a CMV promoter. When exon 51 of the DMD minigene is normally spliced, the EGFP coding sequence is out of frame and no functional EGFP is produced. However, when exon 51 splicing is suppressed, EGFP coding sequence is in frame, and the EGFP reporter fluorescent protein is produced.

Meanwhile, on a separate exon-skipping plasmid, codon optimized PaCsf5 coding sequence, as well as the coding sequence for a crRNA targeting the DMD minigene were put under the transcriptional control of the pCbh promoter and the U6 promoter, respectively. Downstream from the PaCsf5 transcription unit is a mCherry/RFP coding sequence under the transcriptional control of a CMV promoter. In all, 19 targeting crRNA sequences were designed (see SEQ ID NOs: 92-110) around exon 51 of the DMD minigene.

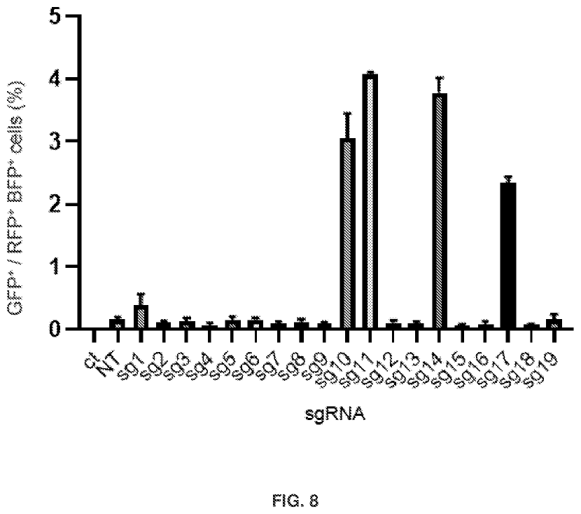

The reporter plasmid and the exon skipping plasmid were co-transfected into HEK293T cells, which were then cultured at 37° C. under 5% CO₂ for 24 hours. Cells co-transfected with both plasmids, thus expressing both RFP and BFP, were isolated via FACS sorting. The percentage of EGFP expressing cells in the isolated cells was summarized in FIG. 8. The results showed that several crRNA (e.g., sg10, 11, 14, and 17) can be used by PaCsf5 to suppress pre-mRNA splicing of exon 51.

SG1:
(SEQ ID NO: 92)
ACAATAAGTCAAATTTAATTGA
SG2:
(SEQ ID NO: 93)
ATAACAATAAGTCAAATTTAAT
SG3:
(SEQ ID NO: 94)
TAGGAGCTAAAATATTTTGGGT
SG4:
(SEQ ID NO: 95)
CTGAGTAGGAGCTAAAATATTT
SG5:
(SEQ ID NO: 96)
CAGTCTGAGTAGGAGCTAAAAT
SG6:
(SEQ ID NO: 97)
AGTAACAGTCTGAGTAGGAGCT
SG7:
(SEQ ID NO: 98)
GTGTCACCAGAGTAACAGTCTG
SG8:
(SEQ ID NO: 99)
CACCAGAGTAACAGTCTGAGTA
SG9:
(SEQ ID NO: 100)
TCCTTAGTAACCACAGGTTGTG
SG10:
(SEQ ID NO: 101)
ATCAAGGAAGATGGCATTTCTA
SG11:
(SEQ ID NO: 102)
ATGGCATTTCTAGTTTGGAGAT
SG12:
(SEQ ID NO: 103)
CAGAGCAGGTACCTCCAACATC
SG13:
(SEQ ID NO: 104)
TCTGTCCAAGCCCGGTTGAAAT
SG14:
(SEQ ID NO: 105)
GCAGAGAAAGCCAGTCGGTAAG
SG15:
(SEQ ID NO: 106)
CCATCACCCTCTGTGATTTTAT
SG16:
(SEQ ID NO: 107)
TCATCTCGTTGATATCCTCAAG
SG17:
(SEQ ID NO: 108)
CTCCAACATCAAGGAAGATGGC
SG18:
(SEQ ID NO: 109)
CATTTTTTCTCATACCTTCTGC
SG19:
(SEQ ID NO: 110)
TCTCATACCTTCTGCTTGATGA

1. A CasPR (CRISPR-associated Protein for Class 1 pre-crRNA processing) fusion protein, comprising a CasPR (or a homolog, an ortholog, a paralog, a variant, a derivative, or a functional fragment thereof) fused to a heterologous functional domain.

2. The fusion protein of claim 1, wherein the CasPR is Cas5d, Cas6, or Csf5.

3. The fusion protein of claim 1, wherein the CasPR is MtCas6 (I-A) (SEQ ID NO: 1), MmCas6 (I-B) (SEQ ID NO: 2), SpCas5d (I-C1) (SEQ ID NO: 3), BhCas5d (I-C2) (SEQ ID NO: 4), SaCas6 (I-D) (SEQ ID NO: 5), EcCas6e (I-E) (SEQ ID NO: 6), PaCas6f (I-F) (SEQ ID NO: 7), MtCas6 (III-A) (SEQ ID NO: 8), PfCas6 (III-B) (SEQ ID NO: 9), PaCsf5 (IV-A1) (SEQ ID NO: 10), or MtCsf5 (IV-A2) (SEQ ID NO: 11).

4. The fusion protein of claim 1, wherein the heterologous functional domain comprises: a nuclear localization signal (NLS), a reporter protein or a detection label (e.g., GST, HRP, CAT, GFP, HcRed, DsRed, CFP, YFP, BFP), a localization signal, a protein targeting moiety, a DNA binding domain (e.g., MBP, Lex A DBD, Gal4 DBD), an epitope tag (e.g., His, myc, V5, FLAG, HA, VSV-G, Trx, etc), a transcription activation domain (e.g., VP64 or VPR), a transcription inhibition domain (e.g., KRAB moiety or SID moiety), a nuclease (e.g., FokI), a deamination domain (e.g., ADAR1, ADAR2, APOBEC, AID, or TAD), a methylase, a demethylase, a transcription release factor, an HDAC, a polypeptide having ssRNA cleavage activity, a polypeptide having dsRNA cleavage activity, a polypeptide having ssDNA cleavage activity, a polypeptide having dsDNA cleavage activity, a DNA or RNA ligase, or any combination thereof.

5. The fusion protein of claim 1, wherein the heterologous functional domain comprises an RNA base editor.

6. The fusion protein of claim 5, wherein the RNA base editor edits A→G single base change.

7. The fusion protein of claim 5, wherein the RNA base editor edits C→U single base change.

8. The fusion protein of claim 5, wherein the RNA base editor comprises ADAR2DD or a derivative thereof.

9. (canceled)

10. The fusion protein of claim 5, having the amino acid sequence of any one of SEQ ID NOs: 45-55.

11.-14. (canceled)

15. A protein having the amino acid sequence of:
(1) any one of SEQ ID NOs: 1-11;
(2) any one of SEQ ID NOs: 1-11, except for 1, 2, 3, 4, 5, 6, 7, 8, 9, or 10 amino acid substitutions, additions, or deletions, wherein the protein maintains the ability of one of SEQ ID NOs: 1-11 for binding to a direct repeat sequence of a Class 1, type I, III, or IV CRISPR system (e.g., any one of SEQ ID NOs: 12-33); or,
(3) at least 80%, 85%, 90%, 91%, 92%, 93%, 94%, 95%, 96%, 97%, 98%, 99% or more sequence identity with any one of SEQ ID NOs: 1-11, wherein the protein maintains the ability of one of the CasPRs of SEQ ID NOs: 1-11 for binding to a direct repeat sequence of a Class 1, type I, III, or IV CRISPR system (e.g., any one of SEQ ID NOs: 12-33);
optionally, with the proviso that the protein is not any one of SEQ ID NOs: 1-11.

16. A CasPR complex, comprising:
(1) a guide RNA comprising a guide sequence capable of hybridizing to a target RNA, and a direct repeat (DR) sequence 3′ (or 5′) to the guide sequence; and,
(2) a CasPR having an amino acid sequence of any one of SEQ ID NOs: 1-11, or a homolog, an ortholog, a paralog, a variant, a derivative, or a functional fragment thereof, wherein the CasPR (or homolog, ortholog, paralog, variant, derivative, or functional fragment thereof) is fused or conjugated to a heterologous functional domain;
wherein the CasPR (or homolog, ortholog, paralog, variant, derivative, or functional fragment thereof) is capable of (i) binding to the guide sequence and (ii) targeting the target RNA.

17. The CasPR complex of claim 16, wherein the DR sequence has substantially the same secondary structure as the secondary structure of any one of SEQ ID NOs: 12-33, or wherein the DR sequence is encoded by any one of SEQ ID NOs: 12-33, or a functional portion thereof that binds to a cognate wild-type CasPR.

18. (canceled)

19. The CasPR complex of claim 16, wherein the target RNA is encoded by a eukaryotic DNA.

20.-25. (canceled)

26. The CasPR complex of claim 16, wherein the derivative is capable of binding to the guide sequence hybridized to the target RNA, but has no RNase catalytic activity due to a mutation in the RNase catalytic site of the CasPR.

27. (canceled)

28. The CasPR complex of claim 16, wherein the CasPR is a Cas5d, a Cas6, or a Csf5, such as MtCas6 (I-A), MmCas6 (I-B), SpCas5d (I-C1), BhCas5d (I-C2), SaCas6 (I-D), EcCas6e (I-E), PaCas6f (I-F), MtCas6 (III-A), PfCas6 (III-B), PaCsf5 (IV-A1), or MtCsf5 (IV-A2).

29. The CasPR complex of claim 16, wherein the heterologous functional domain comprises an RNA base-editing domain.

30. The CasPR complex of claim 29, wherein the RNA base-editing domain comprises an adenosine deaminase and/or a cytidine deaminase, such as a cytidine deaminase acting on RNA (CDAR), such as a double-stranded RNA-specific adenosine deaminase (ADAR) (e.g., ADAR1 or ADAR2), apolipoprotein B mRNA editing enzyme, catalytic polypeptide-like (APOBEC, such as APOBEC1, APOBEC2, APOBEC3A, APOBEC3B, APOBEC3C, APOBEC3D, APOBEC3E, APOBEC3F, APOBEC3G, APOBEC3H, and APOBEC4), activation-induced cytidine deaminase (AID), a cytidine deaminase 1 (CDA1), or a mutant thereof.

31. The CasPR complex of claim 30, wherein the ADAR comprises E488Q/T375G double mutation or comprises ADAR2DD.

32.-34. (canceled)

35. The CasPR complex of claim 16, wherein targeting of the target RNA results in a modification of the target RNA, and wherein the modification of the target RNA is deamination of an adenosine (A) to an inosine (I), and/or deamination of a cytidine (C) to a uracil (U).

36.-59. (canceled)

60. A method of modifying a target RNA, the method comprising contacting the target RNA with the CasPR complex of claim 16, wherein the guide sequence is complementary to at least 15 nucleotides of the target RNA; wherein the CasPR, the ortholog, the paralog, the variant, the derivative, or the functional fragment associates with the guide sequence to form the complex; wherein the complex binds to the target RNA; and wherein upon binding of the complex to the target RNA, the CasPR, the ortholog, the paralog, the variant, the derivative, or the functional fragment modifies the target RNA.

61. (canceled)

62. The method of claim 60, wherein the target RNA is modified by deamination by a derivative comprising a double-stranded RNA-specific adenosine and/or cytidine deaminase.

63.-77. (canceled)


SEQUENCE LISTING (149 sequences)

SEQ ID NO: 1 - protein, 289 aa, Saccharolobus solfataricus
MPLIFKIGYNVIPLQDVILPTPSSKVLKYLIQSGKLIPSLKDLITSRDKYKPIFISHLGFNQRRIFQTNGNLKTITKGSRLSSIIAFSTQANVLSEVADEGIFETVYGKFHIMIESIEIVEVEKLKEEVEKHMNDNIRVRFVSPTLLSSKVLLPPSLSERYKKIHAGYSTLPSVGLIVAYAYNVYCNLIGKKEVEVRAFKFGILSNALSRIIGYDLHPVTVAIGEDSKGNLRKARGVMGWIEFDIPDERLKRRALNYLLTSSYLGIGRSRGIGFGEIRLEFRKIEEKEG

SEQ ID NO: 2 - protein, 218 aa, Methanococcus maripaludis
MDLEYMHISYPNILLNMRDGSKLRGYFAKKYIDEEIVHNHRDNAFVYKYPQIQFKIIDRSPLIIGIGSLGINFLESKRIFFEKELIISNDTNDITEVNVHKDMDHFGTTDKILKYQFKTPWMALNAKNSEIYKNSDEIDREEFLKRVLIGNILSMSKSLGYTIEEKLKVKINLKEVPVKFKNQNMVGFRGEFYINFDIPQYLGIGRNVSRGFGTVVKV

SEQ ID NO: 3 - protein, 242 aa, Streptococcus pyogenes
MYRSRDFYVRVSGQRALFTNPATKGGSERSSYSVPTRQALNGIVDAIYYKPTFTNIVTEVKVINQIQTELQGVRALLHDYSADLSYVSYLSDVVYLIKFHFVWNEDRKDLNSDRLPAKHEAIMERSIRKGGRRDVFLGTRECLGLLDDISQEEYETTVSYYNGVNIDLGIMFHSFAYPKDKKTPLKSYFTKTVMKNGVITFKAQSECDIVNTLSSYAFKAPEEIKSVNDECMEYDAMEKGEN

SEQ ID NO: 4 - protein, 236 aa, Bacillus halodurans
MRNEVQFELFGDYALFTDPLTKIGGEKLSYSVPTYQALKGIAESIYWKPTIVFVIDELRVMKPIQMESKGVRPIEYGGGNTLAHYTYLKDVHYQVKAHFEFNLHRPDLAFDRNEGKHYSILQRSLKAGGRRDIFLGARECQGYVAPCEFGSGDGFYDGQGKYHLGTMVHGFNYPDETGQHQLDVRLWSAVMENGYIQFPRPEDCPIVRPVKEMEPKIFNPDNVQSAEQLLHDLGGE

SEQ ID NO: 5 - protein, 260 aa, Saccharolobus solfataricus
MPNDPYSLYSIVIELGAAEKGFPTGILGRSLHSQVLQWFKQDNPFLATELHQSQISPFSISPLMGKRHAKLTKAGDRLFFRICLLRGDLLQPLLNGIEQTVNQSVCLDKFRFRLCQTHILPGSHPLAGASHYSLISQTPVSSKITLDFKSSTSFKVDRKIIQVFPLGEHVFNSLLRRWNNFAPEDLHFSQVDWSIPIAAFDVKTIPIHLKKVEIGAQGWVTYIFPNTEQAKIASVLSEFAFFSGVGRKTTMGMGQVQVRS

SEQ ID NO: 6 - protein, 199 aa, Escherichia coli
  misc_feature: Enterobacteriaceae  E. coli K12
MYLSKVIIARAWSRDLYQLHQGLWHLFPNRPDAARDFLFHVEKRNTPEGCHVLLQSAQMPVSTAVATVIKTKQVEFQLQVGVPLYFRLRANPIKTILDNQKRLDSKGNIKRCRVPLIKEAEQIAWLQRKLGNAARVEDVHPISERPQYFSGDGKSGKIQTVCFEGVLTINDAPALIDLVQQGIGPAKSMGCGLLSLAPL

SEQ ID NO: 7 - protein, 187 aa, Pseudomonas aeruginosa
MDHYLDIRLRPDPEFPPAQLMSVLFGKLHQALVAQGGDRIGVSFPDLDESRSRLGERLRIHASADDLRALLARPWLEGLRDHLQFGEPAVVPHPTPYRQVSRVQAKSNPERLRRRLMRRHDLSEEEARKRIPDTVARALDLPFVTLRSQSTGQHFRLFIRHGPLQVTAEEGGFTCYGLSKGGFVPWF

SEQ ID NO: 8 - protein, 314 aa, Mycobacterium tuberculosis
  misc_feature: Mycobacterium tuberculosis H37Rv
MAARRGGIRRTDLLRRSGQPRGRHRASAAESGLTWISPTLILVGFSHRGDRRMTEHLSRLTLTLEVDAPLERARVATLGPHLHGVLMESIPADYVQTLHTVPVNPYSQYALARSTTSLEWKISTLTNEARQQIVGPINDAAFAGFRLRASGIATQVTSRSLEQNPLSQFARIFYARPETRKFRVEFLTPTAFKQSGEYVFWPDPRLVFQSLAQKYGAIVDGEEPDPGLIAEFGQSVRLSAFRVASAPFAVGAARVPGFTGSATFTVRGVDTFASYIAALLWFGEFSGCGIKASMGMGAIRVQPLAPREKCVPKP

SEQ ID NO: 9 - protein, 264 aa, Pyrococcus furiosus
MRFLIRLVPEDKDRAFKVPYNHQYYLQGLIYNAIKSSNPKLATYLHEVKGPKLFTYSLFMAEKREHPKGLPYFLGYKKGFFYFSTCVPEIAEALVNGLLMNPEVRLWDERFYLHEIKVLREPKKFNGSTFVTLSPIAVTVVRKGKSYDVPPMEKEFYSIIKDDLQDKYVMAYGDKPPSEFEMEVLIAKPKRFRIKPGIYQTAWHLVFRAYGNDDLLKVGYEVGFGEKNSLGFGMVKVEGNKTTKEAEEQEKITFNSREELKTGV

SEQ ID NO: 10 - protein, 236 aa, Pseudomonas aeruginosa
MFVTQVIFNIGERTYPDRARAMVAELMDGVQPGLVATLMNYIPGTSTSRTEFPTVQFGGASDGFCLLGFGDGGGAIVRDAVPLIHAALARRMPDRIIQVEHKEHSLSAEARPYVLSYTVPRMVVQKKQRHAERLLHEAEGKAHLEGLFLRSLQRQAAAVGLPLPENLEVEFKGAVGDFAAKHNPNSKVAYRGLRGAVFDVNARLGGIWTAGFMLSKGYGQFNATHQLSGAVNALSE

SEQ ID NO: 11 - protein, 259 aa, Methylopumilus turicensis
MHQTLIRINWPKGFKCPPAEFREKLAKSEMFPPEFFHYGTELAVWDKQTAEVEGKIKTVSKEKIIKTFDKPIPLNGRAPVRVIGGQAWAGVIADPEMEGMLIPHLGSILKVASSAAGCAVKIELEQRKFGISYTEYPVKYNLRELVLKRRCEDARSTDIESLIADRIWGGVSGESYYGIDGTCAKFGFEPPSREQLELRIFPMKNIGLHMKSSDGLSKEYMSLIDAEVWMNAKLEGVWQVGNLISRGYGRFIKSIGAQS

SEQ ID NO: 12 - DNA, 24 bp, Saccharolobus solfataricus
gataatctcttatagaattgaaag

SEQ ID NO: 13 - DNA, 37 bp, Methanococcus maripaludis
ctaaaagaataacttgcaaaataacaagcattgaaac

SEQ ID NO: 14 - DNA, 32 bp, Streptococcus pyogenes
gtctcacccttcatgggtgagtggattgaaat

SEQ ID NO: 15 - DNA, 32 bp, Bacillus halodurans
gtcgcactcttcatgggtgcgtggattgaaat

SEQ ID NO: 16 - DNA, 37 bp, Saccharolobus solfataricus
gtttcagtcccgtagtcgggatttagtggttggaaag

SEQ ID NO: 17 - DNA, 29 bp, Escherichia coli
  misc_feature: Enterobacteriaceae  E. coli K12
gagttccccgcgccagcggggataaaccg

SEQ ID NO: 18 - DNA, 28 bp, Pseudomonas aeruginosa
gttcactgccgtataggcagctaagaaa

SEQ ID NO: 19 - DNA, 36 bp, Mycobacterium tuberculosis
  misc_feature: Mycobacterium tuberculosis H37Rv
gtcgtcagacccaaaaccccgagaggggacggaaac

SEQ ID NO: 20 - DNA, 30 bp, Pyrococcus furiosus
gttacaataagactaaaatagaattgaaag

SEQ ID NO: 21 - DNA, 29 bp, Pseudomonas aeruginosa
gtatttcccgcgtgcgcgggggtgagcgg

SEQ ID NO: 22 - DNA, 37 bp, Methylopumilus turicensis
tattggatacacccactcattggtgggtggttagaac

SEQ ID NO: 23 - RNA, 24 nt, Saccharolobus solfataricus
gauaaucucuuauagaauugaaag

SEQ ID NO: 24 - RNA, 37 nt, Methanococcus maripaludis
cuaaaagaauaacuugcaaaauaacaagcauugaaac

SEQ ID NO: 25 - RNA, 32 nt, Streptococcus pyogenes
gucucacccuucaugggugaguggauugaaau

SEQ ID NO: 26 - RNA, 32 nt, Bacillus halodurans
gucgcacucuucaugggugcguggauugaaau

SEQ ID NO: 27 - RNA, 37 nt, Saccharolobus solfataricus
guuucagucccguagucgggauuuagugguuggaaag

SEQ ID NO: 28 - RNA, 29 nt, Escherichia coli
  misc_feature: Enterobacteriaceae  E. coli K12
gaguuccccgcgccagcggggauaaaccg

SEQ ID NO: 29 - RNA, 28 nt, Pseudomonas aeruginosa
guucacugccguauaggcagcuaagaaa

SEQ ID NO: 30 - RNA, 36 nt, Mycobacterium tuberculosis
  misc_feature: Mycobacterium tuberculosis H37Rv
gucgucagacccaaaaccccgagaggggacggaaac

SEQ ID NO: 31 - RNA, 30 nt, Pyrococcus furiosus
guuacaauaagacuaaaauagaauugaaag

SEQ ID NO: 32 - RNA, 29 nt, Pseudomonas aeruginosa
guauuucccgcgugcgcgggggugagcgg

SEQ ID NO: 33 - RNA, 37 nt, Methylopumilus turicensis
uauuggauacacccacucauuggugggugguuagaac

SEQ ID NO: 34 - DNA, 867 bp, Artificial Sequence
  CasPR protein, codon optimized for mammalian expression
atgcctctgatcttcaagatcggctataacgtgatccccctgcaggacgtgatcctgcccaccccttccagcaaggtgctgaagtacctgatccagagcggcaagctgatccccagcctgaaggacctgatcaccagccgggacaagtacaagccaatcttcatctcccacctgggcttcaaccagcggaggattttccagaccaacggcaatctgaaaaccatcaccaagggcagtagactgagctccatcatcgccttcagcacccaggccaacgtgctgtccgaggtggccgatgaagggatcttcgaaaccgtgtacggaaagttccacatcatgatcgaaagcatcgagatcgtggaggtggaaaagctgaaggaggaggtggagaagcacatgaacgacaacatcagagtgagattcgtgtctcccacactgctgagctccaaggtgctgctgccccccagcctgtccgaaagatacaagaagatccacgccgggtacagcaccctgcccagcgtgggcctgatcgtggcctacgcctacaacgtgtactgcaatctgatcggcaagaaggaagtggaagtgcgggccttcaagtttggaatcctgagcaacgccctgtccagaatcatcggctacgacctgcaccctgtgaccgtggccatcggcgaggacagcaaggggaatctgagaaaggctcggggcgtgatgggctggatcgagttcgacatccccgacgaaagactgaagcggcgggccctgaactatctgctgaccagcagctacctgggcatcgggagatctcggggcatcggcttcggcgagatccggctggagttccggaagattgaagagaaggaggga

SEQ ID NO: 35 - DNA, 654 bp, Artificial Sequence
  CasPR protein, codon optimized for mammalian expression
atggacctggagtacatgcacatctcctaccctaacatcctgctgaacatgcgggacggcagcaagctgcggggctacttcgccaagaagtacatcgacgaagagattgtgcacaaccacagagacaacgcctttgtgtacaagtacccccagatccagtttaagatcatcgatagaagccccctgatcatcggcattggctctctgggcatcaatttcctggagagcaagcggatcttcttcgagaaggaactgattatcagcaacgacaccaacgacatcaccgaggtgaacgtgcacaaggacatggatcacttcggcacgaccgacaagatcctgaagtaccagttcaagaccccttggatggcactgaacgccaagaatagcgagatctacaagaactctgacgagatcgaccgggaggagttcctgaagagagtgctgattgggaatatcctgagcatgtctaagagcctgggctataccatcgaagaaaagctgaaggtgaagattaacctgaaggaagtgcccgtgaagttcaagaaccagaacatggtgggctttcggggcgagttctacatcaacttcgacatccctcagtatctgggcatcggccggaatgtgtcccggggattcggcacagtggtgaaggtg

SEQ ID NO: 36 - DNA, 708 bp, Artificial Sequence
  CasPR protein, codon optimized for mammalian expression
atgagaaatgaagtgcagttcgagctgttcggcgactacgccctgttcaccgaccccctgaccaagatcggcggcgaaaagctgagctacagcgtgcctacctaccaggccctgaagggcatcgccgagagcatctactggaagcccaccatcgtgttcgtgatcgacgaactgcgggtcatgaagcccattcagatggagtctaagggcgtgaggcccatcgagtacggcggcggcaacaccctggcccactacacctacctgaaggatgtgcactaccaggtgaaggcccacttcgagttcaacctgcaccggcccgacctggccttcgatagaaacgagggcaagcactactccatcctgcagagaagcctgaaggccggcggcagaagagatattttcctgggcgcccgggagtgccagggctacgtggccccctgcgagttcggcagcggcgacggcttctacgacggccagggcaagtaccacctgggaaccatggtgcacggtttcaactaccccgacgaaaccggacagcaccagctggatgtgagactgtggtctgccgtcatggaaaacggctacatccagttcccccgccctgaggactgccccatcgtgcggcctgtgaaggagatggaacccaagatcttcaaccccgacaacgtgcagtccgccgaacagctgctgcacgacctgggcggcgaa

SEQ ID NO: 37 - DNA, 726 bp, Artificial Sequence
  CasPR protein, codon optimized for mammalian expression
atgtacagaagccgggacttctacgtgagagtgtccggccagcgggccctgttcaccaaccccgccaccaagggcggctccgaacggagctcctactccgtgcctacccggcaggccctgaacgggattgtggacgccatctactacaagcccacgttcaccaacatcgtgaccgaggtgaaggtgattaaccagatccagaccgaactgcagggcgtgcgggccctgctgcatgactacagcgccgacctgagctacgtgtcctacctgagcgacgtggtgtacctgattaagtttcatttcgtgtggaacgaggatagaaaggacctgaatagcgaccggctgccagccaagcatgaggccatcatggagcggtctatccggaagggcggcagacgggacgtgttcctgggcaccagagaatgcctgggcctgctggacgacatcagccaggaagaatacgaaaccacagtgagctattacaatggggtgaacatcgacctgggcatcatgttccacagcttcgcttaccccaaggacaagaaaacccccctgaagtcctacttcacaaagaccgtgatgaagaacggcgtgatcaccttcaaggcccagtccgaatgcgatattgtgaacaccctgagctcctacgccttcaaggcccccgaggagatcaagagcgtgaacgacgagtgcatggagtacgacgccatggagaagggcgaaaac

SEQ ID NO: 38 - DNA, 780 bp, Artificial Sequence
  CasPR protein, codon optimized for mammalian expression
atgcccaacgatccctacagcctgtactccatcgtgatcgaactgggcgccgccgaaaagggattccccacaggcatcctgggcagaagcctgcatagccaggtgctgcagtggttcaagcaggataaccccttcctggccaccgagctgcaccagagccagatctcccccttctccatctctccactgatgggcaagcggcacgccaagctgaccaaggccggcgaccggctgttctttcggatctgcctgctgagaggagatctgctgcagcccctgctgaacggcattgagcagaccgtgaaccagagcgtgtgcctggacaagttccggttccggctgtgccagacccacatcctgcccggcagccaccctctggctggcgcctcccactatagcctgatcagccagaccccagtgagctccaagattaccctggacttcaagagttctacctccttcaaggtggaccggaagatcatccaagtgttccctctgggcgaacacgtgttcaacagcctgctcagacgctggaataacttcgcccccgaggacctgcacttctctcaggtggactggagcatccccatcgccgcattcgacgtgaaaaccatccccatccacctgaagaaggtcgagatcggcgcacagggctgggtgacctacatcttccccaacacagaacaggccaagatcgcctccgtgctgagcgaattcgccttcttcagcggagtgggacggaaaaccaccatgggcatgggccaggtgcaggtgcggtcc

SEQ ID NO: 39 - DNA, 597 bp, Artificial Sequence
  CasPR protein, codon optimized for mammalian expression
atgtacctgagcaaggtgatcatcgccagagcctggagcagagacctgtaccagctgcaccagggcctgtggcacctgttccccaaccggcccgacgccgcccgggatttcctgttccacgtggagaagagaaacaccccggaaggctgccacgtgctgctgcagagcgcacagatgcctgtgagcaccgccgtggccaccgtgatcaagaccaagcaggtggagttccagctgcaggtgggcgtgcccctgtatttcaggctgcgggcgaatcccatcaagaccatcctggacaaccagaagcggctggacagcaagggcaacatcaagaggtgcagagtgcctctgatcaaggaggccgaacagatcgcctggctgcagcggaagctgggcaatgccgccagagtggaggacgtgcaccccatcagcgagcggccccagtacttctccggcgacggaaagagcggaaagatccagaccgtgtgcttcgagggcgtgctgaccatcaacgacgcacccgccctgatcgacctcgtgcagcaggggatcggccctgccaagtccatgggctgcggactgctgtccctggcccccctg

SEQ ID NO: 40 - DNA, 561 bp, Artificial Sequence
  CasPR protein, codon optimized for mammalian expression
atggaccactacctggacattagactgcgccctgacccagagttccctcctgcccagctgatgtctgtgctgtttggcaagctgcaccaggccctggtggcccagggcggtgacagaatcggagtgtctttccctgatctggacgaatctagatctagactgggagagagactgagaatccacgcgtctgccgacgacctgagagctctgctggccagaccatggctggaaggactgcgcgaccacctgcagttcggtgaacctgccgtggtgcctcacccaactccatacagacaggtgagtagagtgcaggcaaagtctaatccagagagactgagacgcagactgatgagaaggcatgacctgtccgaagaagaagccagaaagagaatcccagacacagtggccagagccctggatctgccttttgtgaccctgagaagccagtctaccggccagcacttcagactgtttattcgccacggaccactgcaggtgaccgccgaagagggaggttttacctgctacggactgagcaagggaggtttcgtgccttggttc

SEQ ID NO: 41 - DNA, 942 bp, Artificial Sequence
  CasPR protein, codon optimized for mammalian expression
atggccgccagaagaggcggaatccggagaaccgacctgctgcggaggtctggccagcctcggggcagacaccgggcctccgccgccgagagcggcctgacatggatctcccctaccctgatcctggtgggcttcagccacaggggcgataggagaatgaccgagcacctgtccagactgaccctgaccctggaagtggatgcccccctggagagagcccgggtggccaccctgggcccccacctgcatggcgtgctgatggagtctatccccgccgactacgtgcagacactgcacacagtgccggtgaacccttacagccagtacgctctggcccggagcaccaccagcctggagtggaagatctccaccctgacaaatgaggcccggcagcagatcgtcggccccatcaacgacgccgccttcgccggcttccggctgcgggccagcggcatcgccacccaggtgacaagcagaagcctggagcagaaccccctgtcccagtttgccagaatcttctacgccaggcccgaaacccgcaagttcagagtggagttcctgacccccaccgccttcaagcagagcggcgagtacgtgttttggcccgatcccagactggtgttccagtccctggcccagaagtacggcgccatcgtggacggagaagagcccgaccccggcctgatcgccgagtttggccagtccgtgagactgagcgccttcagagtggccagcgccccttttgccgtgggcgccgccagggtgcccggattcaccggcagcgccaccttcaccgtgcggggagtggacaccttcgccagctacatcgccgctctgctgtggttcggcgagttcagcggatgcggcatcaaggcctccatgggaatgggcgccatccgggtgcagcctctggccccccgggagaagtgcgtgcccaagccc

SEQ ID NO: 42 - DNA, 792 bp, Artificial Sequence
  CasPR protein, codon optimized for mammalian expression
atgagattcctgatcagactggtgcccgaggacaaggacagagccttcaaggtgccttacaaccaccagtactatctgcagggcctgatctacaacgccatcaagtcctccaaccccaagctggccacctacctgcacgaggtgaagggccccaagctgttcacctacagcctgttcatggccgaaaagcgggagcaccctaagggcctgccctactttctgggctacaagaagggcttcttctacttcagcacctgcgtgcccgagatcgccgaggccctggtgaacggcctgctgatgaatcccgaggtgcggctgtgggacgagagattctacctgcacgaaatcaaggtcctgcgggagcccaagaagttcaacggcagcaccttcgtgaccctgagccccatcgccgtgaccgtggtgagaaagggcaagtcctacgacgtgccccccatggaaaaggagttctacagcattatcaaggatgacctgcaggacaagtacgtgatggcctacggcgacaagccccccagtgagttcgagatggaagtgctgatcgccaagcccaagcggttccggatcaagcccggcatctatcagaccgcctggcacctggtgtttcgggcctacggcaatgacgacctgctgaaggtgggctacgaagtgggattcggggagaagaactccctgggattcggaatggtcaaggtggagggcaacaagaccaccaaggaagccgaagaacaggagaagatcaccttcaactcccgggaagagctgaaaacaggcgtg

SEQ ID NO: 43 - DNA, 708 bp, Artificial Sequence
  CasPR protein, codon optimized for mammalian expression
atgttcgtgacccaggtgatcttcaacatcggcgaacggacgtaccccgacagggctcgggctatggtggccgagctgatggatggcgtccagcctggcctggtggccaccctgatgaactacatccccggcaccagcacgagccggacagagttccccaccgtgcagttcggcggcgccagcgacggcttttgcctgctgggcttcggcgacggcggcggcgccatcgtgagagatgccgtgcccctgatccacgccgccctggcaaggcggatgcctgatcggatcatccaggtggaacacaaggagcacagcctgtccgccgaggcccggccctacgtgctgagctacaccgtgcctcggatggtggtgcagaagaagcagcggcacgccgagagactgctgcacgaagccgagggaaaggctcacctggagggcctgttcctgcggagcctgcagaggcaggccgccgccgtgggcctgcccctgcccgagaacctggaggtggagttcaagggagccgtgggcgacttcgccgcaaagcacaatccaaatagcaaggtggcctaccggggactgagaggcgccgtgttcgatgtgaacgccagactgggcggcatctggaccgccggattcatgctgagcaagggctacggccagtttaacgccacccaccagctgagcggcgccgtgaacgctctgtccgaa

SEQ ID NO: 44 - DNA, 777 bp, Artificial Sequence
  CasPR protein, codon optimized for mammalian expression
atgcaccagaccctgatccggatcaactggcccaagggattcaagtgcccccctgccgagttccgggaaaagctggccaagagcgagatgttcccccccgagttcttccactacggcacggaactggccgtgtgggacaagcagaccgccgaggtggagggcaagatcaagaccgtgtccaaggagaagatcatcaagacctttgacaagcccatccccctgaatggccgggccccggtcagagtgatcggcggccaggcctgggccggcgtgatcgccgaccccgagatggagggcatgctgatcccacacctggggagcatcctgaaggtggccagcagcgcggccggatgcgcagtgaagatcgaactggaacagagaaagttcggcatcagctacaccgagtaccccgtgaagtacaacctgcgggagctggtgctgaagagaagatgcgaggacgcccggtctaccgatatcgagagcctgattgccgatagaatctggggcggcgtgtccggcgagagctactatggcatcgacggcacatgcgccaagtttggcttcgaaccccccagcagagagcagctggagctgcggatcttccccatgaagaacatcggactgcacatgaagtccagcgacggactgtccaaggagtacatgagcctgattgacgccgaggtgtggatgaacgctaagctggaaggagtgtggcaggtgggcaacctgatcagcaggggctacggccggttcatcaagtctatcggcgcccagtcc

SEQ ID NO: 45 - DNA, 2133 bp, Artificial Sequence
  fusion  nucleotide
atgcccaagaagaagcggaaggtgatgcctctgatcttcaagatcggctataacgtgatccccctgcaggacgtgatcctgcccaccccttccagcaaggtgctgaagtacctgatccagagcggcaagctgatccccagcctgaaggacctgatcaccagccgggacaagtacaagccaatcttcatctcccacctgggcttcaaccagcggaggattttccagaccaacggcaatctgaaaaccatcaccaagggcagtagactgagctccatcatcgccttcagcacccaggccaacgtgctgtccgaggtggccgatgaagggatcttcgaaaccgtgtacggaaagttccacatcatgatcgaaagcatcgagatcgtggaggtggaaaagctgaaggaggaggtggagaagcacatgaacgacaacatcagagtgagattcgtgtctcccacactgctgagctccaaggtgctgctgccccccagcctgtccgaaagatacaagaagatccacgccgggtacagcaccctgcccagcgtgggcctgatcgtggcctacgcctacaacgtgtactgcaatctgatcggcaagaaggaagtggaagtgcgggccttcaagtttggaatcctgagcaacgccctgtccagaatcatcggctacgacctgcaccctgtgaccgtggccatcggcgaggacagcaaggggaatctgagaaaggctcggggcgtgatgggctggatcgagttcgacatccccgacgaaagactgaagcggcgggccctgaactatctgctgaccagcagctacctgggcatcgggagatctcggggcatcggcttcggcgagatccggctggagttccggaagattgaagagaaggagggacccaagaagaagcggaaggtgggtggaggcggaggttctgggggaggaggtagtggcggtggtggttcaggaggcggcggaagccagctgcatttaccgcaggttttagctgacgctgtctcacgcctggtcataggtaagtttggtgacctgaccgacaacttctcctcccctcacgctcgcagaataggtctggctggagtcgtcatgacaacaggcacagatgttaaagatgccaaggtgatatgtgtttctacaggagcaaaatgtattaatggtgaatacctaagtgatcgtggccttgcattaaatgactgccatgcagaaatagtatctcggagatccttgctcagatttctttatacacaacttgagctttacttaaataacgaggatgatcaaaaaagatccatctttcagaaatcagagcgaggggggtttaggctgaaggagaatatacagtttcatctgtacatcagcacctctccctgtggagatgccagaatcttctcaccacatgaggcaatcctggaagaaccagcagatagacacccaaatcgtaaagcaagaggacagctacggaccaaaatagaggctggtcaggggacgattccagtgcgcaacaatgcgagcatccaaacgtgggacggggtgctgcaaggggagcggctgctcaccatgtcctgcagtgacaagattgcacgctggaacgtggtgggcatccagggatcactgctcagcattttcgtggagcccatttacttctcgagcatcatcctgggcagcctttaccacggggaccacctttccagggccatgtaccagcggatctccaacatagaggacctgccacctctctacaccctcaacaagcctttgctcacaggcatcagcaatgcagaagcacggcagccagggaaggcccccatattcagtgtcaactggacggtaggcgactccgctattgaggtcatcaacgccacgactgggaagggagagctgggccgcgcgtcccgcctgtgtaagcacgcgttgtactgtcgctggatgcgtgtgcacggcaaggttccctcccacttactacgctccaagattaccaagcccaacgtgtaccatgagacaaagctggcggcaaaggagtaccaggccgccaaggcgcgtctgttcacagccttcatcaaggcggggctgggggcctgggtggagaagcccaccgagcaggaccagttctcactcacgtaa

SEQ ID NO: 46 - DNA, 1920 bp, Artificial Sequence
  fusion nucleotide
atgcccaagaagaagcggaaggtgatggacctggagtacatgcacatctcctaccctaacatcctgctgaacatgcgggacggcagcaagctgcggggctacttcgccaagaagtacatcgacgaagagattgtgcacaaccacagagacaacgcctttgtgtacaagtacccccagatccagtttaagatcatcgatagaagccccctgatcatcggcattggctctctgggcatcaatttcctggagagcaagcggatcttcttcgagaaggaactgattatcagcaacgacaccaacgacatcaccgaggtgaacgtgcacaaggacatggatcacttcggcacgaccgacaagatcctgaagtaccagttcaagaccccttggatggcactgaacgccaagaatagcgagatctacaagaactctgacgagatcgaccgggaggagttcctgaagagagtgctgattgggaatatcctgagcatgtctaagagcctgggctataccatcgaagaaaagctgaaggtgaagattaacctgaaggaagtgcccgtgaagttcaagaaccagaacatggtgggctttcggggcgagttctacatcaacttcgacatccctcagtatctgggcatcggccggaatgtgtcccggggattcggcacagtggtgaaggtgcccaagaagaagcggaaggtgggtggaggcggaggttctgggggaggaggtagtggcggtggtggttcaggaggcggcggaagccagctgcatttaccgcaggttttagctgacgctgtctcacgcctggtcataggtaagtttggtgacctgaccgacaacttctcctcccctcacgctcgcagaataggtctggctggagtcgtcatgacaacaggcacagatgttaaagatgccaaggtgatatgtgtttctacaggagcaaaatgtattaatggtgaatacctaagtgatcgtggccttgcattaaatgactgccatgcagaaatagtatctcggagatccttgctcagatttctttatacacaacttgagctttacttaaataacgaggatgatcaaaaaagatccatctttcagaaatcagagcgaggggggtttaggctgaaggagaatatacagtttcatctgtacatcagcacctctccctgtggagatgccagaatcttctcaccacatgaggcaatcctggaagaaccagcagatagacacccaaatcgtaaagcaagaggacagctacggaccaaaatagaggctggtcaggggacgattccagtgcgcaacaatgcgagcatccaaacgtgggacggggtgctgcaaggggagcggctgctcaccatgtcctgcagtgacaagattgcacgctggaacgtggtgggcatccagggatcactgctcagcattttcgtggagcccatttacttctcgagcatcatcctgggcagcctttaccacggggaccacctttccagggccatgtaccagcggatctccaacatagaggacctgccacctctctacaccctcaacaagcctttgctcacaggcatcagcaatgcagaagcacggcagccagggaaggcccccatattcagtgtcaactggacggtaggcgactccgctattgaggtcatcaacgccacgactgggaagggagagctgggccgcgcgtcccgcctgtgtaagcacgcgttgtactgtcgctggatgcgtgtgcacggcaaggttccctcccacttactacgctccaagattaccaagcccaacgtgtaccatgagacaaagctggcggcaaaggagtaccaggccgccaaggcgcgtctgttcacagccttcatcaaggcggggctgggggcctgggtggagaagcccaccgagcaggaccagttctcactcacgtaa

SEQ ID NO: 47 - DNA, 1974 bp, Artificial Sequence
  fusion nucleotide
atgcccaagaagaagcggaaggtgatgagaaatgaagtgcagttcgagctgttcggcgactacgccctgttcaccgaccccctgaccaagatcggcggcgaaaagctgagctacagcgtgcctacctaccaggccctgaagggcatcgccgagagcatctactggaagcccaccatcgtgttcgtgatcgacgaactgcgggtcatgaagcccattcagatggagtctaagggcgtgaggcccatcgagtacggcggcggcaacaccctggcccactacacctacctgaaggatgtgcactaccaggtgaaggcccacttcgagttcaacctgcaccggcccgacctggccttcgatagaaacgagggcaagcactactccatcctgcagagaagcctgaaggccggcggcagaagagatattttcctgggcgcccgggagtgccagggctacgtggccccctgcgagttcggcagcggcgacggcttctacgacggccagggcaagtaccacctgggaaccatggtgcacggtttcaactaccccgacgaaaccggacagcaccagctggatgtgagactgtggtctgccgtcatggaaaacggctacatccagttcccccgccctgaggactgccccatcgtgcggcctgtgaaggagatggaacccaagatcttcaaccccgacaacgtgcagtccgccgaacagctgctgcacgacctgggcggcgaacccaagaagaagcggaaggtgggtggaggcggaggttctgggggaggaggtagtggcggtggtggttcaggaggcggcggaagccagctgcatttaccgcaggttttagctgacgctgtctcacgcctggtcataggtaagtttggtgacctgaccgacaacttctcctcccctcacgctcgcagaataggtctggctggagtcgtcatgacaacaggcacagatgttaaagatgccaaggtgatatgtgtttctacaggagcaaaatgtattaatggtgaatacctaagtgatcgtggccttgcattaaatgactgccatgcagaaatagtatctcggagatccttgctcagatttctttatacacaacttgagctttacttaaataacgaggatgatcaaaaaagatccatctttcagaaatcagagcgaggggggtttaggctgaaggagaatatacagtttcatctgtacatcagcacctctccctgtggagatgccagaatcttctcaccacatgaggcaatcctggaagaaccagcagatagacacccaaatcgtaaagcaagaggacagctacggaccaaaatagaggctggtcaggggacgattccagtgcgcaacaatgcgagcatccaaacgtgggacggggtgctgcaaggggagcggctgctcaccatgtcctgcagtgacaagattgcacgctggaacgtggtgggcatccagggatcactgctcagcattttcgtggagcccatttacttctcgagcatcatcctgggcagcctttaccacggggaccacctttccagggccatgtaccagcggatctccaacatagaggacctgccacctctctacaccctcaacaagcctttgctcacaggcatcagcaatgcagaagcacggcagccagggaaggcccccatattcagtgtcaactggacggtaggcgactccgctattgaggtcatcaacgccacgactgggaagggagagctgggccgcgcgtcccgcctgtgtaagcacgcgttgtactgtcgctggatgcgtgtgcacggcaaggttccctcccacttactacgctccaagattaccaagcccaacgtgtaccatgagacaaagctggcggcaaaggagtaccaggccgccaaggcgcgtctgttcacagccttcatcaaggcggggctgggggcctgggtggagaagcccaccgagcaggaccagttctcactcacgtaa

SEQ ID NO: 48 - DNA, 1992 bp, Artificial Sequence
  fusion nucleotide
atgcccaagaagaagcggaaggtgatgtacagaagccgggacttctacgtgagagtgtccggccagcgggccctgttcaccaaccccgccaccaagggcggctccgaacggagctcctactccgtgcctacccggcaggccctgaacgggattgtggacgccatctactacaagcccacgttcaccaacatcgtgaccgaggtgaaggtgattaaccagatccagaccgaactgcagggcgtgcgggccctgctgcatgactacagcgccgacctgagctacgtgtcctacctgagcgacgtggtgtacctgattaagtttcatttcgtgtggaacgaggatagaaaggacctgaatagcgaccggctgccagccaagcatgaggccatcatggagcggtctatccggaagggcggcagacgggacgtgttcctgggcaccagagaatgcctgggcctgctggacgacatcagccaggaagaatacgaaaccacagtgagctattacaatggggtgaacatcgacctgggcatcatgttccacagcttcgcttaccccaaggacaagaaaacccccctgaagtcctacttcacaaagaccgtgatgaagaacggcgtgatcaccttcaaggcccagtccgaatgcgatattgtgaacaccctgagctcctacgccttcaaggcccccgaggagatcaagagcgtgaacgacgagtgcatggagtacgacgccatggagaagggcgaaaaccccaagaagaagcggaaggtgggtggaggcggaggttctgggggaggaggtagtggcggtggtggttcaggaggcggcggaagccagctgcatttaccgcaggttttagctgacgctgtctcacgcctggtcataggtaagtttggtgacctgaccgacaacttctcctcccctcacgctcgcagaataggtctggctggagtcgtcatgacaacaggcacagatgttaaagatgccaaggtgatatgtgtttctacaggagcaaaatgtattaatggtgaatacctaagtgatcgtggccttgcattaaatgactgccatgcagaaatagtatctcggagatccttgctcagatttctttatacacaacttgagctttacttaaataacgaggatgatcaaaaaagatccatctttcagaaatcagagcgaggggggtttaggctgaaggagaatatacagtttcatctgtacatcagcacctctccctgtggagatgccagaatcttctcaccacatgaggcaatcctggaagaaccagcagatagacacccaaatcgtaaagcaagaggacagctacggaccaaaatagaggctggtcaggggacgattccagtgcgcaacaatgcgagcatccaaacgtgggacggggtgctgcaaggggagcggctgctcaccatgtcctgcagtgacaagattgcacgctggaacgtggtgggcatccagggatcactgctcagcattttcgtggagcccatttacttctcgagcatcatcctgggcagcctttaccacggggaccacctttccagggccatgtaccagcggatctccaacatagaggacctgccacctctctacaccctcaacaagcctttgctcacaggcatcagcaatgcagaagcacggcagccagggaaggcccccatattcagtgtcaactggacggtaggcgactccgctattgaggtcatcaacgccacgactgggaagggagagctgggccgcgcgtcccgcctgtgtaagcacgcgttgtactgtcgctggatgcgtgtgcacggcaaggttccctcccacttactacgctccaagattaccaagcccaacgtgtaccatgagacaaagctggcggcaaaggagtaccaggccgccaaggcgcgtctgttcacagccttcatcaaggcggggctgggggcctgggtggagaagcccaccgagcaggaccagttctcactcacgtaa

SEQ ID NO: 49 - DNA, 2046 bp, Artificial Sequence
  fusion nucleotide
atgcccaagaagaagcggaaggtgatgcccaacgatccctacagcctgtactccatcgtgatcgaactgggcgccgccgaaaagggattccccacaggcatcctgggcagaagcctgcatagccaggtgctgcagtggttcaagcaggataaccccttcctggccaccgagctgcaccagagccagatctcccccttctccatctctccactgatgggcaagcggcacgccaagctgaccaaggccggcgaccggctgttctttcggatctgcctgctgagaggagatctgctgcagcccctgctgaacggcattgagcagaccgtgaaccagagcgtgtgcctggacaagttccggttccggctgtgccagacccacatcctgcccggcagccaccctctggctggcgcctcccactatagcctgatcagccagaccccagtgagctccaagattaccctggacttcaagagttctacctccttcaaggtggaccggaagatcatccaagtgttccctctgggcgaacacgtgttcaacagcctgctcagacgctggaataacttcgcccccgaggacctgcacttctctcaggtggactggagcatccccatcgccgcattcgacgtgaaaaccatccccatccacctgaagaaggtcgagatcggcgcacagggctgggtgacctacatcttccccaacacagaacaggccaagatcgcctccgtgctgagcgaattcgccttcttcagcggagtgggacggaaaaccaccatgggcatgggccaggtgcaggtgcggtcccccaagaagaagcggaaggtgggtggaggcggaggttctgggggaggaggtagtggcggtggtggttcaggaggcggcggaagccagctgcatttaccgcaggttttagctgacgctgtctcacgcctggtcataggtaagtttggtgacctgaccgacaacttctcctcccctcacgctcgcagaataggtctggctggagtcgtcatgacaacaggcacagatgttaaagatgccaaggtgatatgtgtttctacaggagcaaaatgtattaatggtgaatacctaagtgatcgtggccttgcattaaatgactgccatgcagaaatagtatctcggagatccttgctcagatttctttatacacaacttgagctttacttaaataacgaggatgatcaaaaaagatccatctttcagaaatcagagcgaggggggtttaggctgaaggagaatatacagtttcatctgtacatcagcacctctccctgtggagatgccagaatcttctcaccacatgaggcaatcctggaagaaccagcagatagacacccaaatcgtaaagcaagaggacagctacggaccaaaatagaggctggtcaggggacgattccagtgcgcaacaatgcgagcatccaaacgtgggacggggtgctgcaaggggagcggctgctcaccatgtcctgcagtgacaagattgcacgctggaacgtggtgggcatccagggatcactgctcagcattttcgtggagcccatttacttctcgagcatcatcctgggcagcctttaccacggggaccacctttccagggccatgtaccagcggatctccaacatagaggacctgccacctctctacaccctcaacaagcctttgctcacaggcatcagcaatgcagaagcacggcagccagggaaggcccccatattcagtgtcaactggacggtaggcgactccgctattgaggtcatcaacgccacgactgggaagggagagctgggccgcgcgtcccgcctgtgtaagcacgcgttgtactgtcgctggatgcgtgtgcacggcaaggttccctcccacttactacgctccaagattaccaagcccaacgtgtaccatgagacaaagctggcggcaaaggagtaccaggccgccaaggcgcgtctgttcacagccttcatcaaggcggggctgggggcctgggtggagaagcccaccgagcaggaccagttctcactcacgtaa

SEQ ID NO: 50 - DNA, 1860 bp, Artificial Sequence
  fusion nucleotide
atgcctaagaagaagcggaaggtgtacctgagcaaggtgatcatcgccagagcctggagcagagacctgtaccagctgcaccagggcctgtggcacctgttccccaaccggcccgacgccgcccgggatttcctgttccacgtggagaagagaaacaccccggaaggctgccacgtgctgctgcagagcgcacagatgcctgtgagcaccgccgtggccaccgtgatcaagaccaagcaggtggagttccagctgcaggtgggcgtgcccctgtatttcaggctgcgggcgaatcccatcaagaccatcctggacaaccagaagcggctggacagcaagggcaacatcaagaggtgcagagtgcctctgatcaaggaggccgaacagatcgcctggctgcagcggaagctgggcaatgccgccagagtggaggacgtgcaccccatcagcgagcggccccagtacttctccggcgacggaaagagcggaaagatccagaccgtgtgcttcgagggcgtgctgaccatcaacgacgcacccgccctgatcgacctcgtgcagcaggggatcggccctgccaagtccatgggctgcggactgctgtccctggcccccctgcccaagaagaagcggaaggtgggtggaggcggaggttctgggggaggaggtagtggcggtggtggttcaggaggcggcggaagccagctgcatttaccgcaggttttagctgacgctgtctcacgcctggtcataggtaagtttggtgacctgaccgacaacttctcctcccctcacgctcgcagaataggtctggctggagtcgtcatgacaacaggcacagatgttaaagatgccaaggtgatatgtgtttctacaggagcaaaatgtattaatggtgaatacctaagtgatcgtggccttgcattaaatgactgccatgcagaaatagtatctcggagatccttgctcagatttctttatacacaacttgagctttacttaaataacgaggatgatcaaaaaagatccatctttcagaaatcagagcgaggggggtttaggctgaaggagaatatacagtttcatctgtacatcagcacctctccctgtggagatgccagaatcttctcaccacatgaggcaatcctggaagaaccagcagatagacacccaaatcgtaaagcaagaggacagctacggaccaaaatagaggctggtcaggggacgattccagtgcgcaacaatgcgagcatccaaacgtgggacggggtgctgcaaggggagcggctgctcaccatgtcctgcagtgacaagattgcacgctggaacgtggtgggcatccagggatcactgctcagcattttcgtggagcccatttacttctcgagcatcatcctgggcagcctttaccacggggaccacctttccagggccatgtaccagcggatctccaacatagaggacctgccacctctctacaccctcaacaagcctttgctcacaggcatcagcaatgcagaagcacggcagccagggaaggcccccatattcagtgtcaactggacggtaggcgactccgctattgaggtcatcaacgccacgactgggaagggagagctgggccgcgcgtcccgcctgtgtaagcacgcgttgtactgtcgctggatgcgtgtgcacggcaaggttccctcccacttactacgctccaagattaccaagcccaacgtgtaccatgagacaaagctggcggcaaaggagtaccaggccgccaaggcgcgtctgttcacagccttcatcaaggcggggctgggggcctgggtggagaagcccaccgagcaggaccagttctcactcacgtaa

SEQ ID NO: 51 - DNA, 1824 bp, Artificial Sequence
  fusion nucleotide
atgcctaagaagaagagaaaggtggaccactacctggacattagactgcgccctgacccagagttccctcctgcccagctgatgtctgtgctgtttggcaagctgcaccaggccctggtggcccagggcggtgacagaatcggagtgtctttccctgatctggacgaatctagatctagactgggagagagactgagaatccacgcgtctgccgacgacctgagagctctgctggccagaccatggctggaaggactgcgcgaccacctgcagttcggtgaacctgccgtggtgcctcacccaactccatacagacaggtgagtagagtgcaggcaaagtctaatccagagagactgagacgcagactgatgagaaggcatgacctgtccgaagaagaagccagaaagagaatcccagacacagtggccagagccctggatctgccttttgtgaccctgagaagccagtctaccggccagcacttcagactgtttattcgccacggaccactgcaggtgaccgccgaagagggaggttttacctgctacggactgagcaagggaggtttcgtgccttggttccccaagaagaagcggaaggtgggtggaggcggaggttctgggggaggaggtagtggcggtggtggttcaggaggcggcggaagccagctgcatttaccgcaggttttagctgacgctgtctcacgcctggtcataggtaagtttggtgacctgaccgacaacttctcctcccctcacgctcgcagaataggtctggctggagtcgtcatgacaacaggcacagatgttaaagatgccaaggtgatatgtgtttctacaggagcaaaatgtattaatggtgaatacctaagtgatcgtggccttgcattaaatgactgccatgcagaaatagtatctcggagatccttgctcagatttctttatacacaacttgagctttacttaaataacgaggatgatcaaaaaagatccatctttcagaaatcagagcgaggggggtttaggctgaaggagaatatacagtttcatctgtacatcagcacctctccctgtggagatgccagaatcttctcaccacatgaggcaatcctggaagaaccagcagatagacacccaaatcgtaaagcaagaggacagctacggaccaaaatagaggctggtcaggggacgattccagtgcgcaacaatgcgagcatccaaacgtgggacggggtgctgcaaggggagcggctgctcaccatgtcctgcagtgacaagattgcacgctggaacgtggtgggcatccagggatcactgctcagcattttcgtggagcccatttacttctcgagcatcatcctgggcagcctttaccacggggaccacctttccagggccatgtaccagcggatctccaacatagaggacctgccacctctctacaccctcaacaagcctttgctcacaggcatcagcaatgcagaagcacggcagccagggaaggcccccatattcagtgtcaactggacggtaggcgactccgctattgaggtcatcaacgccacgactgggaagggagagctgggccgcgcgtcccgcctgtgtaagcacgcgttgtactgtcgctggatgcgtgtgcacggcaaggttccctcccacttactacgctccaagattaccaagcccaacgtgtaccatgagacaaagctggcggcaaaggagtaccaggccgccaaggcgcgtctgttcacagccttcatcaaggcggggctgggggcctgggtggagaagcccaccgagcaggaccagttctcactcacgtaa

SEQ ID NO: 52 - DNA, 2208 bp, Artificial Sequence
  fusion nucleotide
atgcccaagaagaagcggaaggtgatggccgccagaagaggcggaatccggagaaccgacctgctgcggaggtctggccagcctcggggcagacaccgggcctccgccgccgagagcggcctgacatggatctcccctaccctgatcctggtgggcttcagccacaggggcgataggagaatgaccgagcacctgtccagactgaccctgaccctggaagtggatgcccccctggagagagcccgggtggccaccctgggcccccacctgcatggcgtgctgatggagtctatccccgccgactacgtgcagacactgcacacagtgccggtgaacccttacagccagtacgctctggcccggagcaccaccagcctggagtggaagatctccaccctgacaaatgaggcccggcagcagatcgtcggccccatcaacgacgccgccttcgccggcttccggctgcgggccagcggcatcgccacccaggtgacaagcagaagcctggagcagaaccccctgtcccagtttgccagaatcttctacgccaggcccgaaacccgcaagttcagagtggagttcctgacccccaccgccttcaagcagagcggcgagtacgtgttttggcccgatcccagactggtgttccagtccctggcccagaagtacggcgccatcgtggacggagaagagcccgaccccggcctgatcgccgagtttggccagtccgtgagactgagcgccttcagagtggccagcgccccttttgccgtgggcgccgccagggtgcccggattcaccggcagcgccaccttcaccgtgcggggagtggacaccttcgccagctacatcgccgctctgctgtggttcggcgagttcagcggatgcggcatcaaggcctccatgggaatgggcgccatccgggtgcagcctctggccccccgggagaagtgcgtgcccaagccccccaagaagaagcggaaggtgggtggaggcggaggttctgggggaggaggtagtggcggtggtggttcaggaggcggcggaagccagctgcatttaccgcaggttttagctgacgctgtctcacgcctggtcataggtaagtttggtgacctgaccgacaacttctcctcccctcacgctcgcagaataggtctggctggagtcgtcatgacaacaggcacagatgttaaagatgccaaggtgatatgtgtttctacaggagcaaaatgtattaatggtgaatacctaagtgatcgtggccttgcattaaatgactgccatgcagaaatagtatctcggagatccttgctcagatttctttatacacaacttgagctttacttaaataacgaggatgatcaaaaaagatccatctttcagaaatcagagcgaggggggtttaggctgaaggagaatatacagtttcatctgtacatcagcacctctccctgtggagatgccagaatcttctcaccacatgaggcaatcctggaagaaccagcagatagacacccaaatcgtaaagcaagaggacagctacggaccaaaatagaggctggtcaggggacgattccagtgcgcaacaatgcgagcatccaaacgtgggacggggtgctgcaaggggagcggctgctcaccatgtcctgcagtgacaagattgcacgctggaacgtggtgggcatccagggatcactgctcagcattttcgtggagcccatttacttctcgagcatcatcctgggcagcctttaccacggggaccacctttccagggccatgtaccagcggatctccaacatagaggacctgccacctctctacaccctcaacaagcctttgctcacaggcatcagcaatgcagaagcacggcagccagggaaggcccccatattcagtgtcaactggacggtaggcgactccgctattgaggtcatcaacgccacgactgggaagggagagctgggccgcgcgtcccgcctgtgtaagcacgcgttgtactgtcgctggatgcgtgtgcacggcaaggttccctcccacttactacgctccaagattaccaagcccaacgtgtaccatgagacaaagctggcggcaaaggagtaccaggccgccaaggcgcgtctgttcacagccttcatcaaggcggggctgggggcctgggtggagaagcccaccgagcaggaccagttctcactcacgtaa

SEQ ID NO: 53 - DNA, 2058 bp, Artificial Sequence
  fusion nucleotide
atgcccaagaagaagcggaaggtgatgagattcctgatcagactggtgcccgaggacaaggacagagccttcaaggtgccttacaaccaccagtactatctgcagggcctgatctacaacgccatcaagtcctccaaccccaagctggccacctacctgcacgaggtgaagggccccaagctgttcacctacagcctgttcatggccgaaaagcgggagcaccctaagggcctgccctactttctgggctacaagaagggcttcttctacttcagcacctgcgtgcccgagatcgccgaggccctggtgaacggcctgctgatgaatcccgaggtgcggctgtgggacgagagattctacctgcacgaaatcaaggtcctgcgggagcccaagaagttcaacggcagcaccttcgtgaccctgagccccatcgccgtgaccgtggtgagaaagggcaagtcctacgacgtgccccccatggaaaaggagttctacagcattatcaaggatgacctgcaggacaagtacgtgatggcctacggcgacaagccccccagtgagttcgagatggaagtgctgatcgccaagcccaagcggttccggatcaagcccggcatctatcagaccgcctggcacctggtgtttcgggcctacggcaatgacgacctgctgaaggtgggctacgaagtgggattcggggagaagaactccctgggattcggaatggtcaaggtggagggcaacaagaccaccaaggaagccgaagaacaggagaagatcaccttcaactcccgggaagagctgaaaacaggcgtgcccaagaagaagcggaaggtgggtggaggcggaggttctgggggaggaggtagtggcggtggtggttcaggaggcggcggaagccagctgcatttaccgcaggttttagctgacgctgtctcacgcctggtcataggtaagtttggtgacctgaccgacaacttctcctcccctcacgctcgcagaataggtctggctggagtcgtcatgacaacaggcacagatgttaaagatgccaaggtgatatgtgtttctacaggagcaaaatgtattaatggtgaatacctaagtgatcgtggccttgcattaaatgactgccatgcagaaatagtatctcggagatccttgctcagatttctttatacacaacttgagctttacttaaataacgaggatgatcaaaaaagatccatctttcagaaatcagagcgaggggggtttaggctgaaggagaatatacagtttcatctgtacatcagcacctctccctgtggagatgccagaatcttctcaccacatgaggcaatcctggaagaaccagcagatagacacccaaatcgtaaagcaagaggacagctacggaccaaaatagaggctggtcaggggacgattccagtgcgcaacaatgcgagcatccaaacgtgggacggggtgctgcaaggggagcggctgctcaccatgtcctgcagtgacaagattgcacgctggaacgtggtgggcatccagggatcactgctcagcattttcgtggagcccatttacttctcgagcatcatcctgggcagcctttaccacggggaccacctttccagggccatgtaccagcggatctccaacatagaggacctgccacctctctacaccctcaacaagcctttgctcacaggcatcagcaatgcagaagcacggcagccagggaaggcccccatattcagtgtcaactggacggtaggcgactccgctattgaggtcatcaacgccacgactgggaagggagagctgggccgcgcgtcccgcctgtgtaagcacgcgttgtactgtcgctggatgcgtgtgcacggcaaggttccctcccacttactacgctccaagattaccaagcccaacgtgtaccatgagacaaagctggcggcaaaggagtaccaggccgccaaggcgcgtctgttcacagccttcatcaaggcggggctgggggcctgggtggagaagcccaccgagcaggaccagttctcactcacgtaa

SEQ ID NO: 54 - DNA, 1971 bp, Artificial Sequence
  fusion nucleotide
atgcctaagaagaagcggaaggtgttcgtgacccaggtgatcttcaacatcggcgaacggacgtaccccgacagggctcgggctatggtggccgagctgatggatggcgtccagcctggcctggtggccaccctgatgaactacatccccggcaccagcacgagccggacagagttccccaccgtgcagttcggcggcgccagcgacggcttttgcctgctgggcttcggcgacggcggcggcgccatcgtgagagatgccgtgcccctgatccacgccgccctggcaaggcggatgcctgatcggatcatccaggtggaacacaaggagcacagcctgtccgccgaggcccggccctacgtgctgagctacaccgtgcctcggatggtggtgcagaagaagcagcggcacgccgagagactgctgcacgaagccgagggaaaggctcacctggagggcctgttcctgcggagcctgcagaggcaggccgccgccgtgggcctgcccctgcccgagaacctggaggtggagttcaagggagccgtgggcgacttcgccgcaaagcacaatccaaatagcaaggtggcctaccggggactgagaggcgccgtgttcgatgtgaacgccagactgggcggcatctggaccgccggattcatgctgagcaagggctacggccagtttaacgccacccaccagctgagcggcgccgtgaacgctctgtccgaacccaagaagaagcggaaggtgggtggaggcggaggttctgggggaggaggtagtggcggtggtggttcaggaggcggcggaagccagctgcatttaccgcaggttttagctgacgctgtctcacgcctggtcataggtaagtttggtgacctgaccgacaacttctcctcccctcacgctcgcagaataggtctggctggagtcgtcatgacaacaggcacagatgttaaagatgccaaggtgatatgtgtttctacaggagcaaaatgtattaatggtgaatacctaagtgatcgtggccttgcattaaatgactgccatgcagaaatagtatctcggagatccttgctcagatttctttatacacaacttgagctttacttaaataacgaggatgatcaaaaaagatccatctttcagaaatcagagcgaggggggtttaggctgaaggagaatatacagtttcatctgtacatcagcacctctccctgtggagatgccagaatcttctcaccacatgaggcaatcctggaagaaccagcagatagacacccaaatcgtaaagcaagaggacagctacggaccaaaatagaggctggtcaggggacgattccagtgcgcaacaatgcgagcatccaaacgtgggacggggtgctgcaaggggagcggctgctcaccatgtcctgcagtgacaagattgcacgctggaacgtggtgggcatccagggatcactgctcagcattttcgtggagcccatttacttctcgagcatcatcctgggcagcctttaccacggggaccacctttccagggccatgtaccagcggatctccaacatagaggacctgccacctctctacaccctcaacaagcctttgctcacaggcatcagcaatgcagaagcacggcagccagggaaggcccccatattcagtgtcaactggacggtaggcgactccgctattgaggtcatcaacgccacgactgggaagggagagctgggccgcgcgtcccgcctgtgtaagcacgcgttgtactgtcgctggatgcgtgtgcacggcaaggttccctcccacttactacgctccaagattaccaagcccaacgtgtaccatgagacaaagctggcggcaaaggagtaccaggccgccaaggcgcgtctgttcacagccttcatcaaggcggggctgggggcctgggtggagaagcccaccgagcaggaccagttctcactcacgtaa

SEQ ID NO: 55 - DNA, 2040 bp, Artificial Sequence
  fusion nucleotide
atgcccaagaagaagagaaaggtgcaccagaccctgatccggatcaactggcccaagggattcaagtgcccccctgccgagttccgggaaaagctggccaagagcgagatgttcccccccgagttcttccactacggcacggaactggccgtgtgggacaagcagaccgccgaggtggagggcaagatcaagaccgtgtccaaggagaagatcatcaagacctttgacaagcccatccccctgaatggccgggccccggtcagagtgatcggcggccaggcctgggccggcgtgatcgccgaccccgagatggagggcatgctgatcccacacctggggagcatcctgaaggtggccagcagcgcggccggatgcgcagtgaagatcgaactggaacagagaaagttcggcatcagctacaccgagtaccccgtgaagtacaacctgcgggagctggtgctgaagagaagatgcgaggacgcccggtctaccgatatcgagagcctgattgccgatagaatctggggcggcgtgtccggcgagagctactatggcatcgacggcacatgcgccaagtttggcttcgaaccccccagcagagagcagctggagctgcggatcttccccatgaagaacatcggactgcacatgaagtccagcgacggactgtccaaggagtacatgagcctgattgacgccgaggtgtggatgaacgctaagctggaaggagtgtggcaggtgggcaacctgatcagcaggggctacggccggttcatcaagtctatcggcgcccagtcccccaagaagaagcggaaggtgggtggaggcggaggttctgggggaggaggtagtggcggtggtggttcaggaggcggcggaagccagctgcatttaccgcaggttttagctgacgctgtctcacgcctggtcataggtaagtttggtgacctgaccgacaacttctcctcccctcacgctcgcagaataggtctggctggagtcgtcatgacaacaggcacagatgttaaagatgccaaggtgatatgtgtttctacaggagcaaaatgtattaatggtgaatacctaagtgatcgtggccttgcattaaatgactgccatgcagaaatagtatctcggagatccttgctcagatttctttatacacaacttgagctttacttaaataacgaggatgatcaaaaaagatccatctttcagaaatcagagcgaggggggtttaggctgaaggagaatatacagtttcatctgtacatcagcacctctccctgtggagatgccagaatcttctcaccacatgaggcaatcctggaagaaccagcagatagacacccaaatcgtaaagcaagaggacagctacggaccaaaatagaggctggtcaggggacgattccagtgcgcaacaatgcgagcatccaaacgtgggacggggtgctgcaaggggagcggctgctcaccatgtcctgcagtgacaagattgcacgctggaacgtggtgggcatccagggatcactgctcagcattttcgtggagcccatttacttctcgagcatcatcctgggcagcctttaccacggggaccacctttccagggccatgtaccagcggatctccaacatagaggacctgccacctctctacaccctcaacaagcctttgctcacaggcatcagcaatgcagaagcacggcagccagggaaggcccccatattcagtgtcaactggacggtaggcgactccgctattgaggtcatcaacgccacgactgggaagggagagctgggccgcgcgtcccgcctgtgtaagcacgcgttgtactgtcgctggatgcgtgtgcacggcaaggttccctcccacttactacgctccaagattaccaagcccaacgtgtaccatgagacaaagctggcggcaaaggagtaccaggccgccaaggcgcgtctgttcacagccttcatcaaggcggggctgggggcctgggtggagaagcccaccgagcaggaccagttctcactcacgtaa

SEQ ID NO: 56 - DNA, 1470 bp, Artificial Sequence
  fusion nucleotide
atgagcgagctgattaaggagaacatgcacatgaagctgtatatggagggcaccgtggacaaccatcacttcaagtgcacatccgagggcgaaggcaagccctacgagggcacccagaccatgagaatcaaggtggtcgagggcggccctctccccttcgccttcgacatcctggctactagcttcctctacggcagcaagaccttcatcaaccacacccagggcatccccgacttcttcaagcagtccttccctgagggcttcacatgggagagagtcaccacatacgaggacgggggcgtgctgaccgctacccaggacaccagcctccaggacggctgcctcatctacaacgtcaagatcagaggggtgaacttcacatccaacggccctgtgatgcagaagaaaacactcggctgggaggccttcaccgagacactgtaccccgctgacggcggcctggaaggcagaaacgacatggccctgaagctcgtgggcgggagccatctgatcgcaaacatcaagaccacatatagatccaagaaacccgctaagaacctcaagatgcctggcgtctactatgtggactacagactggaaagaatcaaggaggccaacaacgagacatacgtcgagcagcacgaggtggcagtggccagatactgcgacctccctagcaaactggggcacaagctgaatggcgccactaacttctccctgttgaaacaagcaggggatgtcgaagagaatcccgggccaatggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctaggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaaaaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtaa

SEQ ID NO: 57 - DNA, 1470 bp, Artificial Sequence
  fusion nucleotide
atgagcgagctgattaaggagaacatgcacatgaagctgtatatggagggcaccgtggacaaccatcacttcaagtgcacatccgagggcgaaggcaagccctacgagggcacccagaccatgagaatcaaggtggtcgagggcggccctctccccttcgccttcgacatcctggctactagcttcctctacggcagcaagaccttcatcaaccacacccagggcatccccgacttcttcaagcagtccttccctgagggcttcacatgggagagagtcaccacatacgaggacgggggcgtgctgaccgctacccaggacaccagcctccaggacggctgcctcatctacaacgtcaagatcagaggggtgaacttcacatccaacggccctgtgatgcagaagaaaacactcggctgggaggccttcaccgagacactgtaccccgctgacggcggcctggaaggcagaaacgacatggccctgaagctcgtgggcgggagccatctgatcgcaaacatcaagaccacatatagatccaagaaacccgctaagaacctcaagatgcctggcgtctactatgtggactacagactggaaagaatcaaggaggccaacaacgagacatacgtcgagcagcacgaggtggcagtggccagatactgcgacctccctagcaaactggggcacaagctgaatggcgccactaacttctccctgttgaaacaagcaggggatgtcgaagagaatcccgggccaatggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtaggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaaaaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtaa

SEQ ID NO: 58 - DNA, 1470 bp, Artificial Sequence
  fusion nucleotide
atgagcgagctgattaaggagaacatgcacatgaagctgtatatggagggcaccgtggacaaccatcacttcaagtgcacatccgagggcgaaggcaagccctacgagggcacccagaccatgagaatcaaggtggtcgagggcggccctctccccttcgccttcgacatcctggctactagcttcctctacggcagcaagaccttcatcaaccacacccagggcatccccgacttcttcaagcagtccttccctgagggcttcacatgggagagagtcaccacatacgaggacgggggcgtgctgaccgctacccaggacaccagcctccaggacggctgcctcatctacaacgtcaagatcagaggggtgaacttcacatccaacggccctgtgatgcagaagaaaacactcggctgggaggccttcaccgagacactgtaccccgctgacggcggcctggaaggcagaaacgacatggccctgaagctcgtgggcgggagccatctgatcgcaaacatcaagaccacatatagatccaagaaacccgctaagaacctcaagatgcctggcgtctactatgtggactacagactggaaagaatcaaggaggccaacaacgagacatacgtcgagcagcacgaggtggcagtggccagatactgcgacctccctagcaaactggggcacaagctgaatggcgccactaacttctccctgttgaaacaagcaggggatgtcgaagagaatcccgggccaatggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaaaaccatgggctaggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtaa

SEQ ID NO: 59 - DNA, 30 bp, Artificial Sequence
  SPACER-TAG
gtcccaggcgaagggcagggggccaccctt

SEQ ID NO: 60 - DNA, 30 bp, Artificial Sequence
  SPACER-TAG
ctcccacttgaagccctcggggaaggacag

SEQ ID NO: 61 - DNA, 30 bp, Artificial Sequence
  SPACER-TAG
ctcccagcccatggttttcttctgcattac

SEQ ID NO: 62 - DNA, 30 bp, Artificial Sequence
  SPACER-TAG
ccaggcgaagggcagggggccacccttggt

SEQ ID NO: 63 - DNA, 30 bp, Artificial Sequence; identical to SEQ ID NO: 59
  SPACER-TAG

SEQ ID NO: 64 - DNA, 30 bp, Artificial Sequence
  SPACER-TAG
gatgtcccaggcgaagggcagggggccacc

SEQ ID NO: 65 - DNA, 30 bp, Artificial Sequence
  SPACER-TAG
caggatgtcccaggcgaagggcagggggcc

SEQ ID NO: 66 - DNA, 30 bp, Artificial Sequence
  SPACER-TAG
ggacaggatgtcccaggcgaagggcagggg

SEQ ID NO: 67 - DNA, 30 bp, Artificial Sequence
  SPACER-TAG
aggggacaggatgtcccaggcgaagggcag

SEQ ID NO: 68 - DNA, 30 bp, Artificial Sequence
  SPACER-TAG
ctgaggggacaggatgtcccaggcgaaggg

SEQ ID NO: 69 - DNA, 30 bp, Artificial Sequence
  SPACER-TAG
gaactgaggggacaggatgtcccaggcgaa

SEQ ID NO: 70 - DNA, 30 bp, Artificial Sequence
  SPACER-TAG
catgaactgaggggacaggatgtcccaggc

SEQ ID NO: 71 - DNA, 30 bp, Artificial Sequence
  SPACER-TAG
gtacatgaactgaggggacaggatgtccca

SEQ ID NO: 72 - DNA, 30 bp, Artificial Sequence
  SPACER-TAG
ccacttgaagccctcggggaaggacagctt

SEQ ID NO: 73 - DNA, 30 bp, Artificial Sequence; identical to SEQ ID NO: 60
  SPACER-TAG

SEQ ID NO: 74 - DNA, 30 bp, Artificial Sequence
  SPACER-TAG
gcgctcccacttgaagccctcggggaagga

SEQ ID NO: 75 - DNA, 30 bp, Artificial Sequence
  SPACER-TAG
cacgcgctcccacttgaagccctcggggaa

SEQ ID NO: 76 - DNA, 30 bp, Artificial Sequence
  SPACER-TAG
catcacgcgctcccacttgaagccctcggg

SEQ ID NO: 77 - DNA, 30 bp, Artificial Sequence
  SPACER-TAG
gttcatcacgcgctcccacttgaagccctc

SEQ ID NO: 78 - DNA, 30 bp, Artificial Sequence
  SPACER-TAG
gaagttcatcacgcgctcccacttgaagcc

SEQ ID NO: 79 - DNA, 30 bp, Artificial Sequence
  SPACER-TAG
ctcgaagttcatcacgcgctcccacttgaa

SEQ ID NO: 80 - DNA, 30 bp, Artificial Sequence
  SPACER-TAG
gtcctcgaagttcatcacgcgctcccactt

SEQ ID NO: 81 - DNA, 30 bp, Artificial Sequence
  SPACER-TAG
gccgtcctcgaagttcatcacgcgctccca

SEQ ID NO: 82 - DNA, 30 bp, Artificial Sequence
  SPACER-TAG
ccagcccatggttttcttctgcattacggg

SEQ ID NO: 83 - DNA, 30 bp, Artificial Sequence; identical to SEQ ID NO: 61
  SPACER-TAG

SEQ ID NO: 84 - DNA, 30 bp, Artificial Sequence
  SPACER-TAG
ggcctcccagcccatggttttcttctgcat

SEQ ID NO: 85 - DNA, 30 bp, Artificial Sequence
  SPACER-TAG
ggaggcctcccagcccatggttttcttctg

SEQ ID NO: 86 - DNA, 30 bp, Artificial Sequence
  SPACER-TAG
ggaggaggcctcccagcccatggttttctt

SEQ ID NO: 87 - DNA, 30 bp, Artificial Sequence
  SPACER-TAG
ctcggaggaggcctcccagcccatggtttt

SEQ ID NO: 88 - DNA, 30 bp, Artificial Sequence
  SPACER-TAG
ccgctcggaggaggcctcccagcccatggt

SEQ ID NO: 89 - DNA, 30 bp, Artificial Sequence
  SPACER-TAG
catccgctcggaggaggcctcccagcccat

SEQ ID NO: 90 - DNA, 30 bp, Artificial Sequence
  SPACER-TAG
gtacatccgctcggaggaggcctcccagcc

SEQ ID NO: 91 - DNA, 30 bp, Artificial Sequence
  SPACER-TAG
ggggtacatccgctcggaggaggcctccca

SEQ ID NO: 92 - DNA, 22 bp, Homo sapiens
acaataagtcaaatttaattga

SEQ ID NO: 93 - DNA, 22 bp, Homo sapiens
ataacaataagtcaaatttaat

SEQ ID NO: 94 - DNA, 22 bp, Homo sapiens
taggagctaaaatattttgggt

SEQ ID NO: 95 - DNA, 22 bp, Homo sapiens
ctgagtaggagctaaaatattt

SEQ ID NO: 96 - DNA, 22 bp, Homo sapiens
cagtctgagtaggagctaaaat

SEQ ID NO: 97 - DNA, 22 bp, Homo sapiens
agtaacagtctgagtaggagct

SEQ ID NO: 98 - DNA, 22 bp, Homo sapiens
gtgtcaccagagtaacagtctg

SEQ ID NO: 99 - DNA, 22 bp, Homo sapiens
caccagagtaacagtctgagta

SEQ ID NO: 100 - DNA, 22 bp, Homo sapiens
tccttagtaaccacaggttgtg

SEQ ID NO: 101 - DNA, 22 bp, Homo sapiens
atcaaggaagatggcatttcta

SEQ ID NO: 102 - DNA, 22 bp, Homo sapiens
atggcatttctagtttggagat

SEQ ID NO: 103 - DNA, 22 bp, Homo sapiens
cagagcaggtacctccaacatc

SEQ ID NO: 104 - DNA, 22 bp, Homo sapiens
tctgtccaagcccggttgaaat

SEQ ID NO: 105 - DNA, 22 bp, Homo sapiens
gcagagaaagccagtcggtaag

SEQ ID NO: 106 - DNA, 22 bp, Homo sapiens
ccatcaccctctgtgattttat

SEQ ID NO: 107 - DNA, 22 bp, Homo sapiens
tcatctcgttgatatcctcaag

SEQ ID NO: 108 - DNA, 22 bp, Homo sapiens
ctccaacatcaaggaagatggc

SEQ ID NO: 109 - DNA, 22 bp, Homo sapiens
cattttttctcataccttctgc

SEQ ID NO: 110 - DNA, 22 bp, Homo sapiens
tctcataccttctgcttgatga

SEQ ID NO: 111 - protein, 229 aa, Artificial Sequence [sequence omitted]

SEQ ID NO: 112 - protein, 236 aa, Artificial Sequence
  Synthetic APOBEC 1
MTSEKGPSTGDPTLRRRIEPWEFDVFYDPRELRKEACLLYEIKWGMSRKIWRSSGKNTTNHVEVNFIKKFTSERDFHPSMSCSITWFLSWSPCWECSQAIREFLSRHPGVTLVIYVARLFWHMDQQNRQGLRDLVNSGVTIQIMRASEYYHCWRNFVNYPPGDEAHWPQYPPLWMMLYALELHCIILSLPPCLKISRRWQNHLTFFRLHLQNCHYQTIPPHILLATGLIHPSVAWR

SEQ ID NO: 113 - protein, 325 aa, Artificial Sequence
  Synthetic hAPOBEC3G
MELKYHPEMRFFHWFSKWRKLHRDQEYEVTWYISWSPCTKCTRDMATFLAEDPKVTLTIFVARLYYFWDPDYQEALRSLCQKRDGPRATMKIMNYDEFQHCWSKFVYSQRELFEPWNNLPKYYILLHIMLGEILRHSMDPPTFTFNFNNEPWVRGRHETYLCYEVERMHNDTWVLLNQRRGFLCNQAPHKHGFLEGRHAELCFLDVIPFWKLDLDQDYRVTCFTSWSPCFSCAQEMAKFISKNKHVSLCIFTARIYDDQGRCQEGLRTLAEAGAKISIMTYSEFKHCWDTFVDHQGCPFQPWDGLDEHSQDLSGRLRAILQNQEN

SEQ ID NO: 114 - protein, 208 aa, Artificial Sequence
  Synthetic pmCDAl
MTDAEYVRIHEKLDIYTFKKQFFNNKKSVSHRCYVLFELKRRGERRACFWGYAVNKPQSGTERGIHAEIFSIRKVEEYLRDNPGQFTINWYSSWSPCADCAEKILEWYNQELRGNGHTLKIWACKLYYEKNARNQIGLWNLRDNGVGLNVMVSEHYQCCRKIFIQSSHNQLNENRWLEKTLKRAEKRRSELSIMIQVKILHTTKSPAV

SEQ ID NO: 115 - protein, 200 aa, Artificial Sequence
  Synthetic hAID
MDSLLMNRRKFLYQFKNVRWAKGRRETYLCYVVKRRDSATSFSLDFGYLRNKNGCHVELLFLRYISDWDLDPGRCYRVTWFTSWSPCYDCARHVADFLRGNPYLSLRIFTARLYFCEDRKAEPEGLRRLHRAGVQIAIMTFKDYFYCWNTFVENHERTFKAWEGLHENSVRLSRQLRRILLPLYEVDDLRDAFRTLGLLD

SEQ ID NO: 116 - protein, 181 aa, Artificial Sequence
  Synthetic hAID-DC
MDSLLMNRRKFLYQFKNVRWAKGRRETYLCYVVKRRDSATSFSLDFGYLRNKNGCHVELLFLRYISDWDLDPGRCYRVTWFTSWSPCYDCARHVADFLRGNPNLSLRIFTARLYFCEDRKAEPEGLRRLHRAGVQIAIMTFKDYFYCWNTFVENHERTFKAWEGLHENSVRLSRQLRRILL

SEQ ID NO: 117 - protein, 4 aa, Artificial Sequence
  Synthetic
GGGS

SEQ ID NO: 118 - protein, 5 aa, Artificial Sequence
  Synthetic
GGGGS

SEQ ID NO: 119 - protein, 9 aa, Artificial Sequence
  Synthetic
GGSGGSGGS

SEQ ID NO: 120 - protein, 15 aa, Artificial Sequence
  Synthetic
GGGGSGGGGSGGGGS

SEQ ID NO: 121 - protein, 75 aa, Artificial Sequence
  Synthetic
  VARIANT (16)..(75): “GGGGS” may or may not be present
GGGGSGGGGSGGGGSGGGGSGGGGSGGGGSGGGGSGGGGSGGGGSGGGGSGGGGSGGGGSGGGGSGGGGSGGGGS

SEQ ID NO: 122 - protein, 10 aa, Artificial Sequence
  Synthetic
GGGGSGGGGS

SEQ ID NO: 123 - protein, 20 aa, Artificial Sequence
  Synthetic
GGGGSGGGGSGGGGSGGGGS

SEQ ID NO: 124 - protein, 25 aa, Artificial Sequence
  Synthetic
GGGGSGGGGSGGGGSGGGGSGGGGS

SEQ ID NO: 125 - protein, 30 aa, Artificial Sequence
  Synthetic
GGGGSGGGGSGGGGSGGGGSGGGGSGGGGS

SEQ ID NO: 126 - protein, 35 aa, Artificial Sequence
  Synthetic
GGGGSGGGGSGGGGSGGGGSGGGGSGGGGSGGGGS

SEQ ID NO: 127 - protein, 40 aa, Artificial Sequence
  Synthetic
GGGGSGGGGSGGGGSGGGGSGGGGSGGGGSGGGGSGGGGS

SEQ ID NO: 128 - protein, 45 aa, Artificial Sequence
  Synthetic
GGGGSGGGGSGGGGSGGGGSGGGGSGGGGSGGGGSGGGGSGGGGS

SEQ ID NO: 129 - protein, 50 aa, Artificial Sequence
  Synthetic
GGGGSGGGGSGGGGSGGGGSGGGGSGGGGSGGGGSGGGGSGGGGSGGGGS

SEQ ID NO: 130 - protein, 55 aa, Artificial Sequence
  Synthetic
GGGGSGGGGSGGGGSGGGGSGGGGSGGGGSGGGGSGGGGSGGGGSGGGGSGGGGS

SEQ ID NO: 131 - protein, 60 aa, Artificial Sequence
  Synthetic
GGGGSGGGGSGGGGSGGGGSGGGGSGGGGSGGGGSGGGGSGGGGSGGGGSGGGGSGGGGS

SEQ ID NO: 132 - protein, 32 aa, Artificial Sequence
  Synthetic
LEPGEKPYKCPECGKSFSQSGALTRHQRTHTR

SEQ ID NO: 133 - protein, 20 aa, Artificial Sequence
  Synthetic
PKKKRKVEASSPKKRKVEAS

SEQ ID NO: 134 - protein, 7 aa, Unknown
  SV40 virus large T-antigen
PKKKRKV

SEQ ID NO: 135 - protein, 16 aa, Unknown
  NLS from nucleoplasmin
KRPAATKKAGQAKKKK

SEQ ID NO: 136 - protein, 9 aa, Unknown
  c-myc NLS
PAAKRVKLD

SEQ ID NO: 137 - protein, 11 aa, Unknown
  c-myc NLS
RQRRNELKRSP

SEQ ID NO: 138 - protein, 38 aa, Unknown
  hRNPAl M9 NLS
NQSSNFGPMKGGNFGGRSSGPYGGGGQYFAKPRNQGGY

SEQ ID NO: 139 - protein, 40 aa, Unknown
  NV of the IBB domain from importin-alpha
RMRIZFKNKGKDTAELRRRRVEVSVELRKAKKDEQILKRR

SEQ ID NO: 140 - protein, 8 aa, Unknown
  Myoma T protein
VSRKRPRP

SEQ ID NO: 141 - protein, 8 aa, Unknown
  Myoma T protein
PPKKARED

SEQ ID NO: 142 - protein, 8 aa, Homo sapiens
  misc_feature: human p53
PQPKKKPL

SEQ ID NO: 143 - protein, 12 aa, Mus musculus
  misc_feature: mouse c-abl IV
SALIKKKKKMAP

SEQ ID NO: 144 - protein, 5 aa, Unknown
  influenza virus NS1
DRLRR

SEQ ID NO: 145 - protein, 7 aa, Unknown
  influenza virus NS1
PKQKKRK

SEQ ID NO: 146 - protein, 10 aa, Unknown
  Hepatitis virus delta antigen
RKLKKKIKKL

SEQ ID NO: 147 - protein, 10 aa, Mus musculus
  misc_feature: mouse Mxl protein
REKKKFLKRR

SEQ ID NO: 148 - protein, 20 aa, Homo sapiens
  misc_feature: human poly(ADP-ribose) polymerase
KRKGDEVDGVDEVAKKKSKK

SEQ ID NO: 149 - protein, 17 aa, Homo sapiens
  misc_feature: steroid hormone receptors (human) glucocorticoid
RKCLQAGMNLEARKTKK